(19) United States
(12) Patent Application Publication
(10) Pub. No.: US 2015/0031693 A1
(43) Pub. Date: Jan. 29, 2015

(54) PAK INHIBITORS FOR THE TREATMENT OF FRAGILE X SYNDROME

(71) Applicants: AFRAXIS HOLDINGS, INC., La Jolla, CA (US); The United States of America, as Represented by the Secretary, Dept. of Health and Human Services, Bethesda, MD (US)

(72) Inventors: John C. Mckew, Boyds, MD (US); Wenwei Huang, Rockville, MD (US); David Campbell, San Diego, CA (US); Sergio G. Durön, San Diego, CA (US); Mark Behnke, Poolesville, MD (US); Min Shen, Boyds, MD (US)

(73) Assignee: AFRAXIS HOLDINGS, INC., La Jolla, CA (US)

(21) Appl. No.: 14/356,118

(22) PCT Filed: Nov. 2, 2012
(86) PCT No.: PCT/US12/63426

§ 371 Date: May 2, 2014

Related U.S. Application Data
(60) Provisional application No. 61/555,902, filed on Nov. 4, 2011.

(51) Int. Cl. C07D 471/04

(52) U.S. Cl. CPC C07D 471/04
USPC 514/234.2; 544/279; 514/255.05; 514/264.11; 544/117; 544/230

(57) ABSTRACT

Provided herein are PAK inhibitors and methods of utilizing PAK inhibitors for the treatment of Fragile X syndrome.

### CROSS-REFERENCE TO RELATED APPLICATION

This application claims the benefit of priority of U.S. Provisional Application No. 61/555,902, filed on Nov. 4, 2011, the content of which is incorporated herein to the extent as provided herein.

### STATEMENT OF GOVERNMENT INTEREST

This invention was created in the performance of a Cooperative Research and Development Agreement with the National Institute of Health, an Agency of the Department of Health and Human Services. The Government of the United States has certain rights in this invention.

### BACKGROUND OF THE INVENTION

Fragile X syndrome (FXS) is a genetic syndrome that is the most common known single-gene cause of autism and the most common inherited cause of mental retardation among boys. It results in a spectrum of intellectual disability ranging from mild to severe as well as physical characteristics such as an elongated face, large or protruding ears, and large testes (macroorchidism), and behavioral characteristics such as stereotypic movements (e.g. hand-flapping), and social anxiety.

Fragile X syndrome is associated with the expansion of the CGG trinucleotide repeat affecting the Fragile X mental retardation 1 (FMR1) gene on the X chromosome, resulting in a failure to express the Fragile X mental retardation protein (FMRP), which is required for normal neural development. Depending on the length of the CGG repeat, an allele may be classified as normal (unaffected by the syndrome), a premutation (at risk of Fragile X associated disorders), or full mutation (usually affected by the syndrome). A definitive diagnosis of Fragile X syndrome is made through genetic testing to determine the number of CGG repeats. Testing for premutation carriers can also be carried out to allow for genetic counseling.

### SUMMARY OF THE INVENTION

Described herein are compounds, compositions and methods for treating an individual suffering from Fragile X syndrome (FXS), by administering to an individual a therapeutically effective amount of an inhibitor of a p21-activated kinase (PAK), e.g., an inhibitor of PAK1, PAK2, PAK3 or PAK4, as described herein. PAK activation is shown to play a key role in spine morphogenesis. In some instances, attenuation of PAK activity reduces, prevents or reverses defects in spine morphogenesis. In some embodiments, inhibitors of one or more of Group I PAKs (PAK1, PAK2 and/or PAK3) and/or Group II PAKs (PAK4, PAK5 and/or PAK6) are administered to rescue defects in spine morphogenesis in individuals suffering from a condition in which dendritic spine morphology, density, and/or function are aberrant, including but not limited to abnormal spine density, spine size, spine shape, spine plasticity, spine motility or spine plasticity leading to improvements in synaptic function, cognition and/or behavior.

In one aspect is a compound having the structure of Formula I, Formula II, or Formula III, or a pharmaceutically acceptable salt or N-oxide thereof:

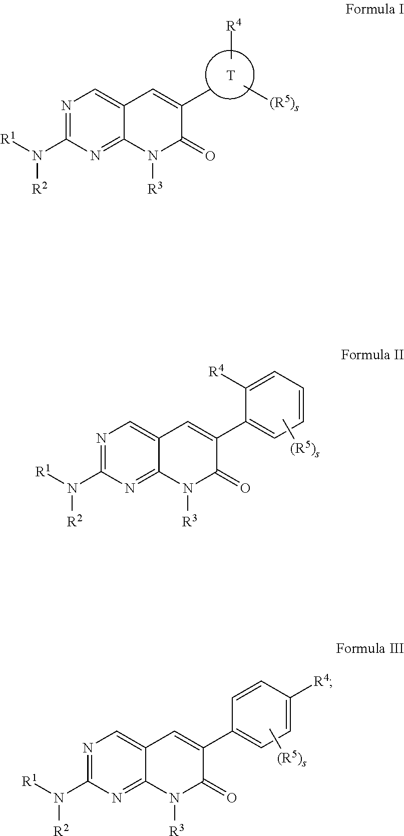

wherein:
ring T is an aryl or heteroaryl ring;
R¹ is H, or substituted or unsubstituted alkyl;
R² is alkyl substituted with —OH, —OMe, —SH, —SMe, or halogen;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted heteroaryl attached to ring T or the phenyl ring via a carbon atom of R⁴, or substituted or unsubstituted heterocycloalkyl attached to ring T or the phenyl ring via a carbon atom of R⁴;
each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

In some embodiments is a compound having the structure of Formula I.

In one refinement, the compound having the structure of Formula I has the structure of Formula Ia:

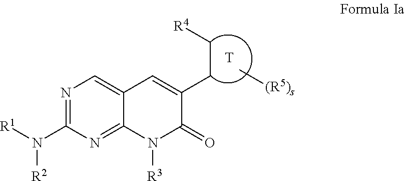

In another refinement, the compound of Formula I has the structure of Formula Ib:

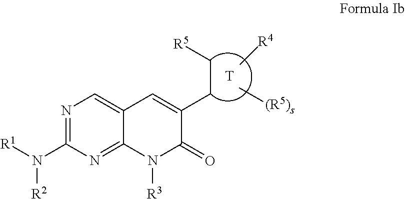

wherein s is 0-3.

In one embodiment is a compound of Formula I wherein ring T is aryl. In a refinement, aryl is phenyl. In another refinement, aryl is naphthalene.

In some embodiment, ring T in the compound of Formula I is selected from pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl.

In some embodiments is a compound having the structure of Formula II. In some embodiments is a compound having the structure of Formula III.

In one refinement, the compound having the structure of Formula III has the structure of Formula IIIa:

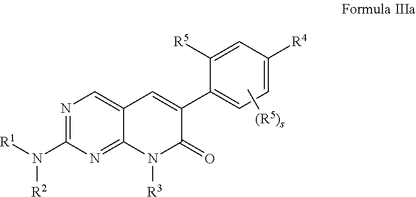

wherein s is 0-3.

In another refinement, the compound having the structure of Formula III has the structure of Formula IIIb:

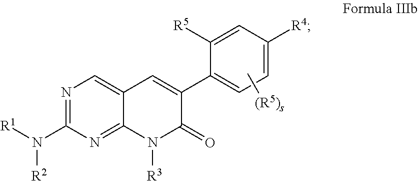

wherein s is 0-2.

In another aspect is a compound having the structure of Formula IV, or a pharmaceutically acceptable salt or N-oxide thereof

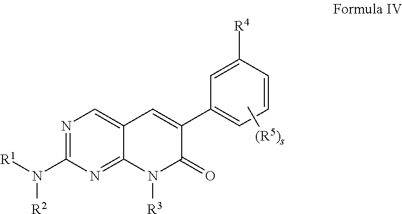

wherein:
R¹ is H, or substituted or unsubstituted alkyl;
R² is substituted with —OH, —OMe, —SH, —SMe, or halogen;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted 6-membered monocyclic heteroaryl ring attached to the phenyl ring via a carbon atom of R⁴, substituted or unsubstituted bicyclic heteroaryl ring attached to the phenyl via a carbon atom of R⁴, or substituted or unsubstituted heterocycloalkyl attached to the phenyl ring via a carbon atom of R⁴;
each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

In some embodiments, R⁴ in Formula IV is a substituted or unsubstituted C-linked 6-membered monocyclic heteroaryl ring or a substituted or unsubstituted C-linked bicyclic heteroaryl ring. In a refinement, R⁴ is selected from pyridine, pyridazinyl, pyrimidinyl, pyrazinyl, indolyl, benzofuranyl, benzimidazolyl, indazolyl, pyrrolopyridinyl, or imidazopyridinyl.

In some embodiments, R⁴ in Formula I-IV is a substituted or unsubstituted C-linked heteroaryl. In a refinement, R⁴ is selected from pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl.

In some embodiments, R⁴ in Formula I-IV is a C-linked heterocycloalkyl. In a refinement, the heterocycloalkyl is selected from pyrrolidinyl, tetrahydrofuranyl, piperidinyl, tetrahydropyranyl, tetrahydrothiopyranyl, morpholinyl, or piperazinyl.

In some embodiment, each R⁵ in Formula I-IV is independently selected from halogen, —CN, —OH, —OCF₃, —OCF₃, —OCF₂H, —CF₃, —SR⁸, —N(R¹⁰)₂, a substituted or unsubstituted alkyl, or a substituted or unsubstituted alkoxy.

In some embodiment, each R⁵ in Formula I-IV is independently selected from halogen, —N(R¹⁰)₂, or a substituted or unsubstituted alkyl.

In some embodiments, s in Formula I-IV is 0. In some embodiments, s in Formula I-IV is 1. In some embodiments, s in Formula I-IV is 2.

In some embodiments, R³ in Formula I-IV is H. In some embodiment, R³ in Formula I-IV is a substituted or unsubstituted alkoxy, or a substituted or unsubstituted amino. In some embodiment, R³ in Formula I-IV is a substituted or unsubstituted alkyl, or a substituted or unsubstituted heteroalkyl.

In some embodiments, R³ in Formula I-IV is a substituted or unsubstituted cycloalkyl, or a substituted or unsubstituted heterocycloalkyl. In a refinement, the cycloalkyl is cyclopropyl, cyclobutyl, cyclopentyl, cyclohexyl or cycloheptyl. In another refinement, the heterocycloalkyl is pyrrolidinyl, tetrahydrofuranyl, piperidinyl, tetrahydropyranyl, tetrahydrothiopyranyl, morpholinyl, or piperazinyl.

In some embodiments, R³ in Formula I-IV is a substituted or unsubstituted cycloalkylalkyl, or a substituted or unsubstituted heterocycloalkylalkyl.

In some embodiments, R³ in Formula I-IV is a substituted or unsubstituted aryl, or a substituted or unsubstituted heteroaryl. In a refinement, the aryl is phenyl. In another refinement, the heteroaryl is pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, or imidazopyridinyl.

In some embodiments, R³ in Formula I-IV is a substituted or unsubstituted arylalkyl, or a substituted or unsubstituted heteroarylalkyl.

In some embodiments, R² in Formula I-IV is C₁-C₄alkyl substituted with hydroxy or C₁-C₄alkyl substituted with methoxy. In some embodiments, R² in Formula I-IV is —CH(CH₂CH₂OH)₂.

In some embodiment, R¹ in Formula I-IV is H. In some embodiment, R¹ in Formula I-IV is substituted or unsubstituted alkyl.

In another aspect is a compound selected from:

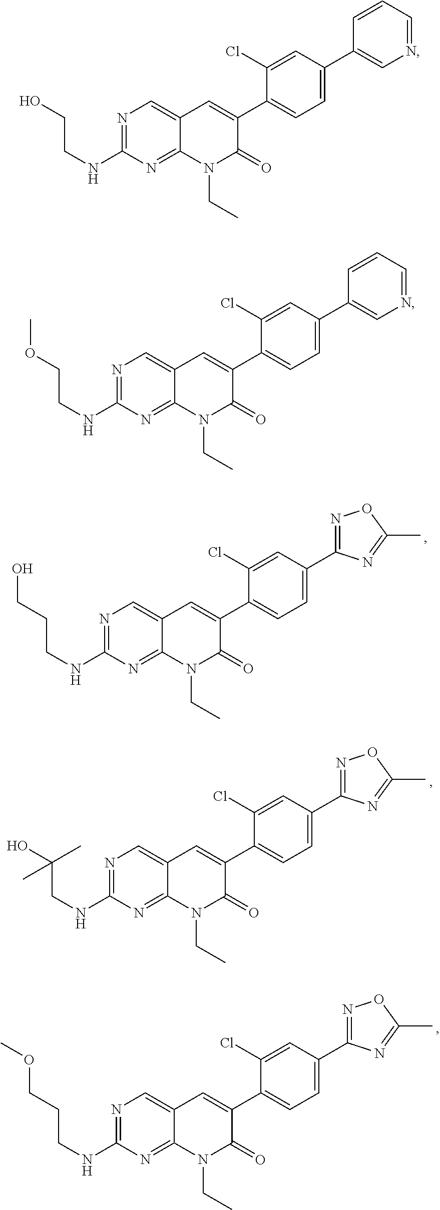

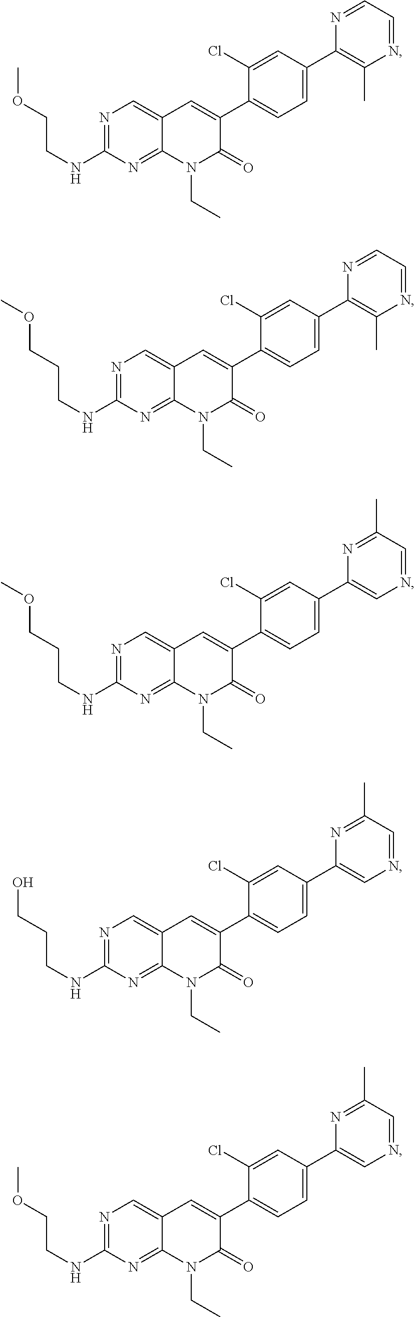 [SMILES: CCn1c(=O)c(-c2ccc(-c3nccnc3C)cc2Cl)cc2cnc(NCCOC)nc21 ; CCn1c(=O)c(-c2ccc(-c3nccnc3C)cc2Cl)cc2cnc(NCCCOC)nc21 ; CCn1c(=O)c(-c2ccc(-c3cncc(C)n3)cc2Cl)cc2cnc(NCCCOC)nc21 ; CCn1c(=O)c(-c2ccc(-c3cncc(C)n3)cc2Cl)cc2cnc(NCCCO)nc21 ; CCn1c(=O)c(-c2ccc(-c3cncc(C)n3)cc2Cl)cc2cnc(NCCOC)nc21]

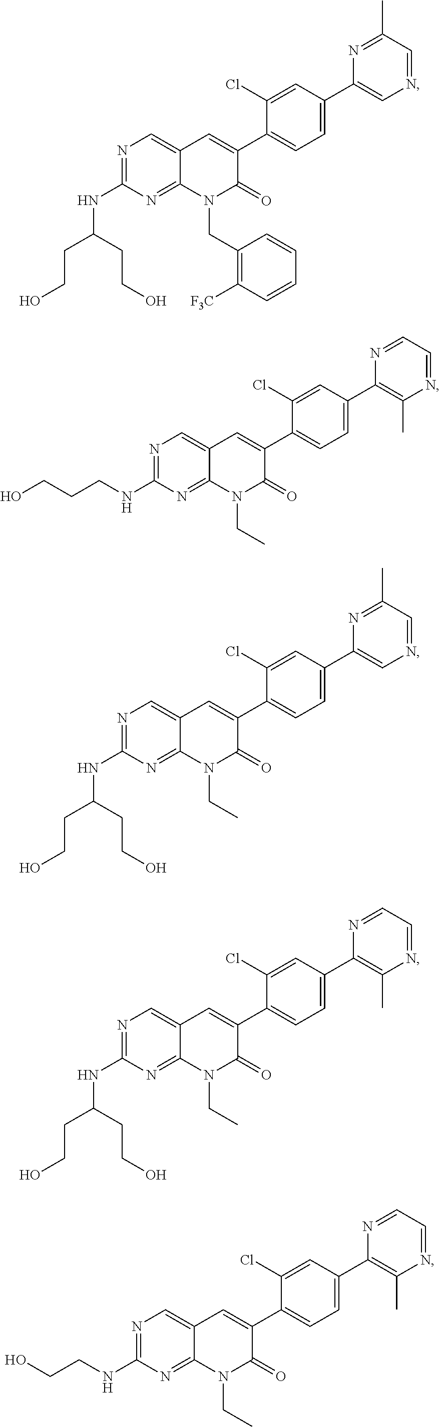 [SMILES: Cc1cncc(-c2ccc(-c3cc4cnc(NC(CCO)CCO)nc4n(Cc4ccccc4C(F)(F)F)c3=O)c(Cl)c2)n1 ; CCn1c(=O)c(-c2ccc(-c3nccnc3C)cc2Cl)cc2cnc(NCCCO)nc21 ; CCn1c(=O)c(-c2ccc(-c3cncc(C)n3)cc2Cl)cc2cnc(NC(CCO)CCO)nc21 ; CCn1c(=O)c(-c2ccc(-c3nccnc3C)cc2Cl)cc2cnc(NC(CCO)CCO)nc21 ; CCn1c(=O)c(-c2ccc(-c3nccnc3C)cc2Cl)cc2cnc(NCCO)nc21]

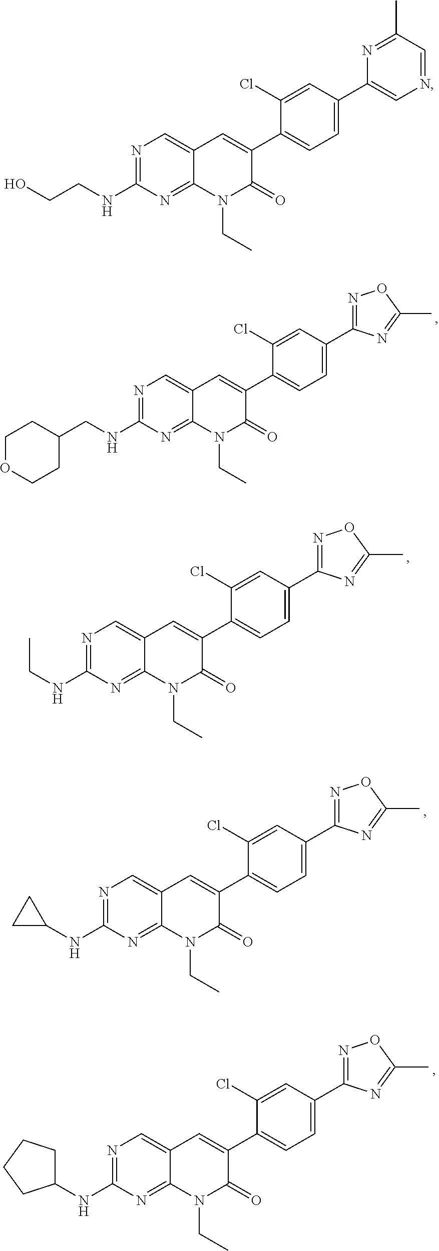 [SMILES: CCn1c(=O)c(-c2ccc(-c3cncc(C)n3)cc2Cl)cc2cnc(NCCO)nc21 ; CCn1c(=O)c(-c2ccc(-c3noc(C)n3)cc2Cl)cc2cnc(NCC3CCOCC3)nc21 ; CCNc1ncc2cc(-c3ccc(-c4noc(C)n4)cc3Cl)c(=O)n(CC)c2n1 ; CCn1c(=O)c(-c2ccc(-c3noc(C)n3)cc2Cl)cc2cnc(NC3CC3)nc21 ; CCn1c(=O)c(-c2ccc(-c3noc(C)n3)cc2Cl)cc2cnc(NC3CCCC3)nc21]

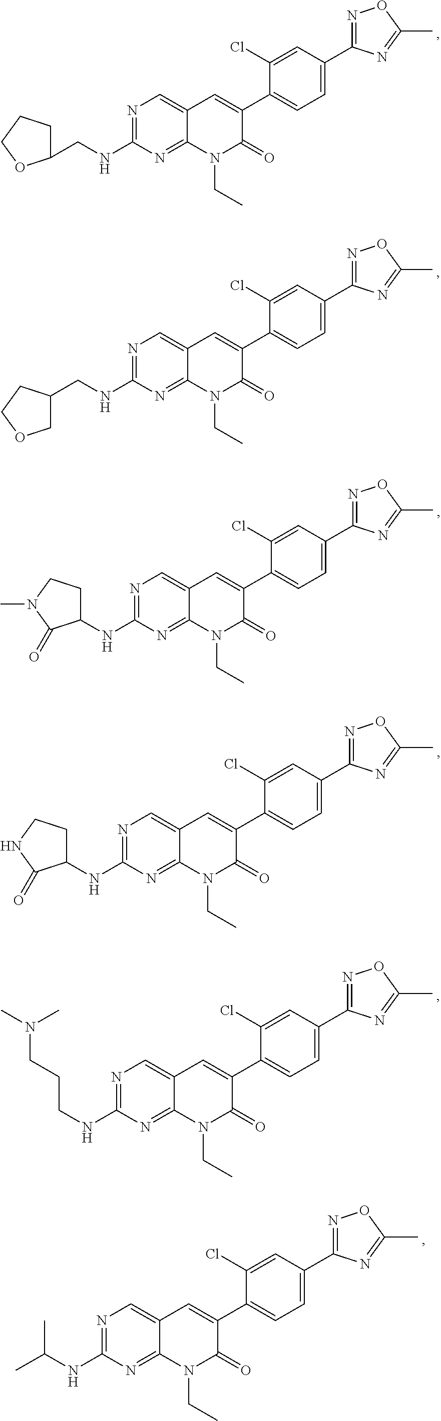

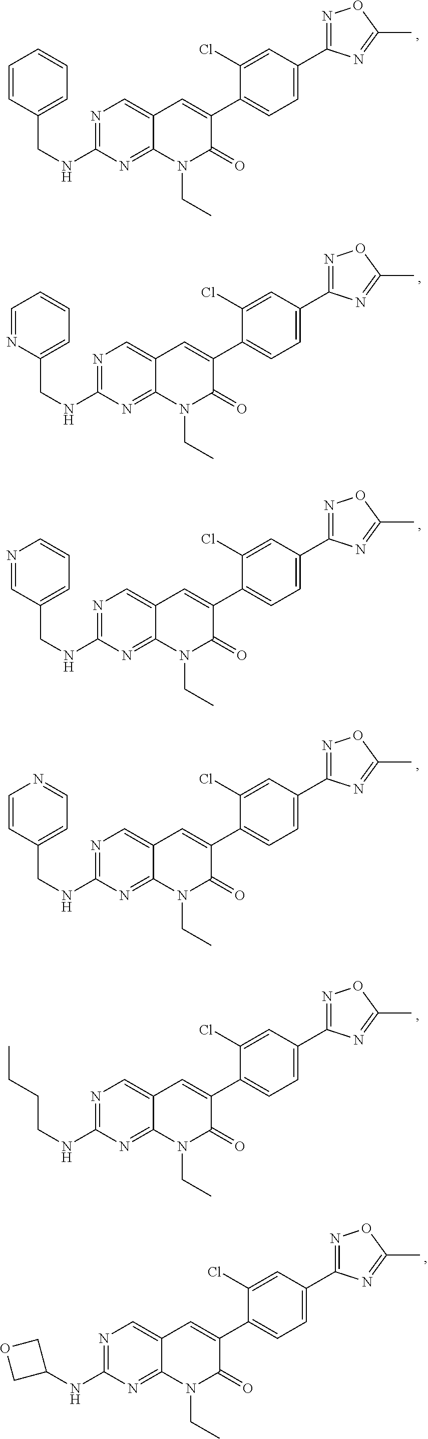

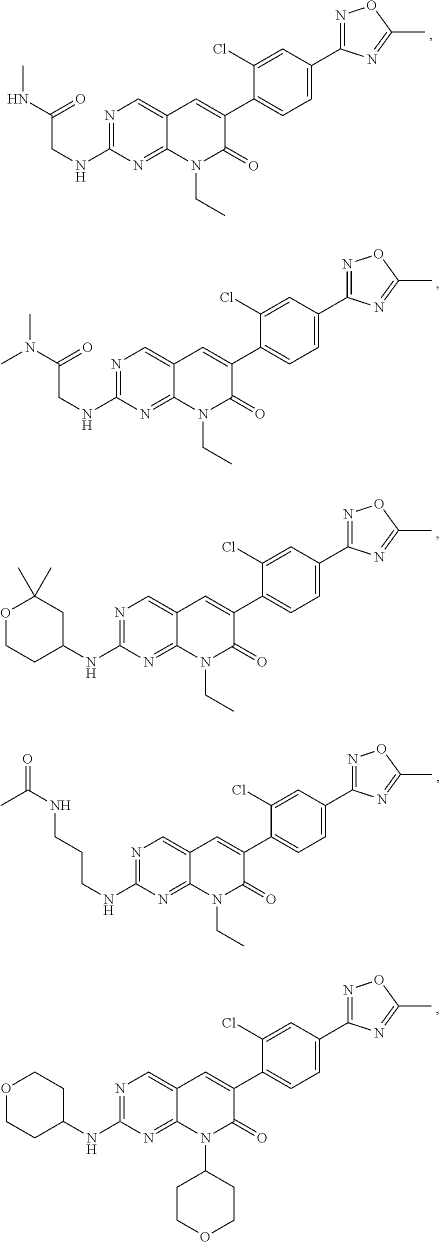

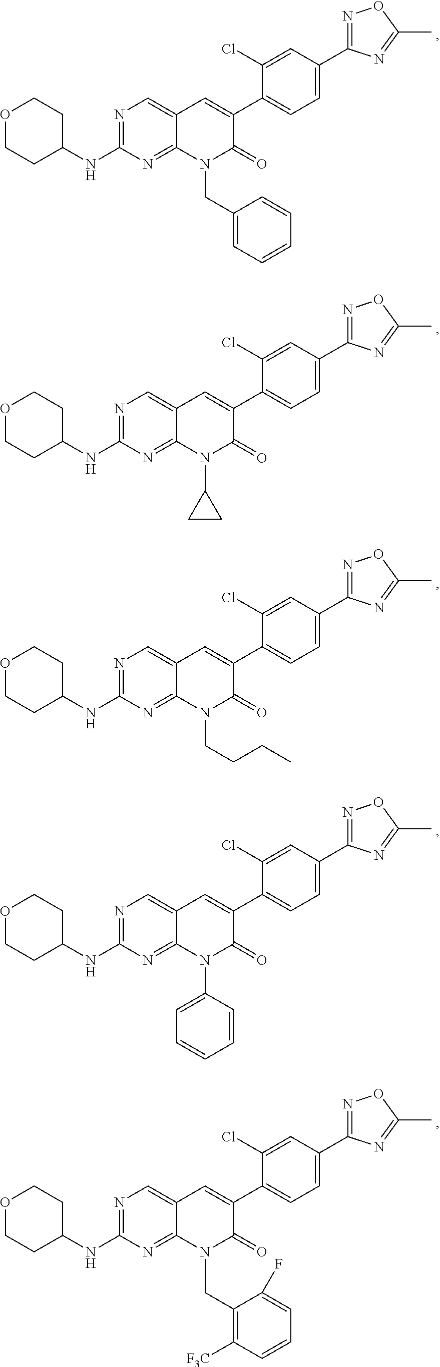 [SMILES: Cc1nc(-c2ccc(-c3cc4cnc(NC5CCOCC5)nc4n(Cc4ccccc4)c3=O)c(Cl)c2)no1 ; Cc1nc(-c2ccc(-c3cc4cnc(NC5CCOCC5)nc4n(C4CC4)c3=O)c(Cl)c2)no1 ; CCCCn1c(=O)c(-c2ccc(-c3noc(C)n3)cc2Cl)cc2cnc(NC3CCOCC3)nc21 ; Cc1nc(-c2ccc(-c3cc4cnc(NC5CCOCC5)nc4n(-c4ccccc4)c3=O)c(Cl)c2)no1 ; Cc1nc(-c2ccc(-c3cc4cnc(NC5CCOCC5)nc4n(Cc4c(F)cccc4C(F)(F)F)c3=O)c(Cl)c2)no1]

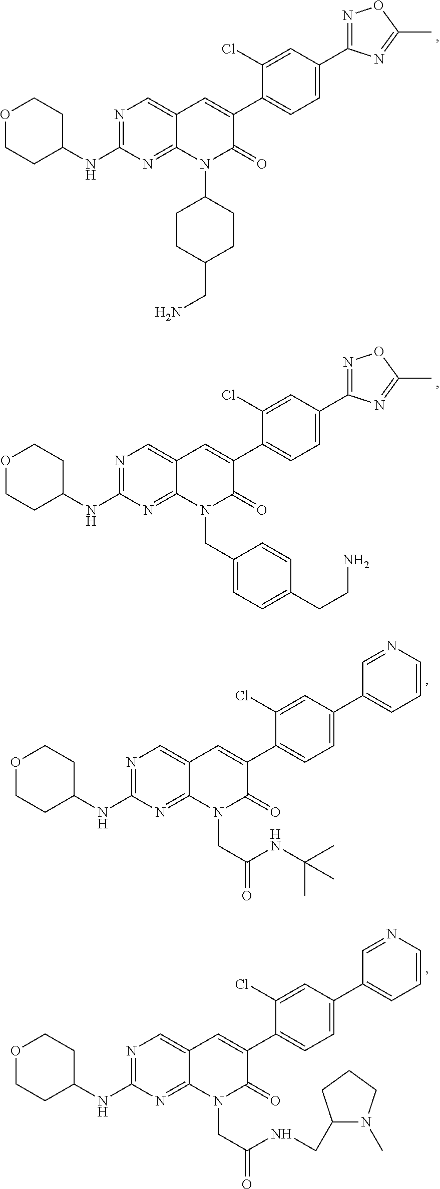 [SMILES: Cc1nc(-c2ccc(-c3cc4cnc(NC5CCOCC5)nc4n(C4CCC(CN)CC4)c3=O)c(Cl)c2)no1 ; Cc1nc(-c2ccc(-c3cc4cnc(NC5CCOCC5)nc4n(Cc4ccc(CCN)cc4)c3=O)c(Cl)c2)no1 ; CC(C)(C)NC(=O)Cn1c(=O)c(-c2ccc(-c3cccnc3)cc2Cl)cc2cnc(NC3CCOCC3)nc21 ; CN1CCCC1CNC(=O)Cn1c(=O)c(-c2ccc(-c3cccnc3)cc2Cl)cc2cnc(NC3CCOCC3)nc21]

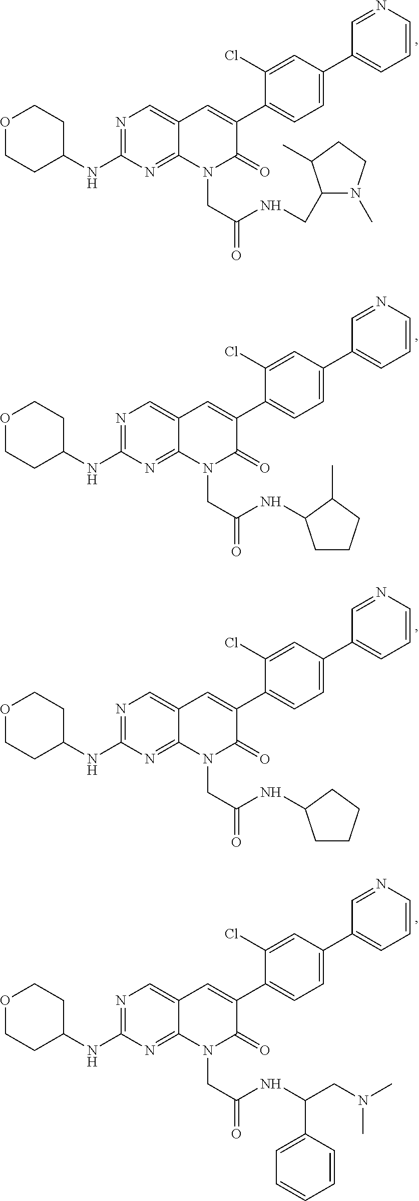 [SMILES: CC1CCN(C)C1CNC(=O)Cn1c(=O)c(-c2ccc(-c3cccnc3)cc2Cl)cc2cnc(NC3CCOCC3)nc21 ; CC1CCCC1NC(=O)Cn1c(=O)c(-c2ccc(-c3cccnc3)cc2Cl)cc2cnc(NC3CCOCC3)nc21 ; O=C(Cn1c(=O)c(-c2ccc(-c3cccnc3)cc2Cl)cc2cnc(NC3CCOCC3)nc21)NC1CCCC1 ; CN(C)CC(NC(=O)Cn1c(=O)c(-c2ccc(-c3cccnc3)cc2Cl)cc2cnc(NC3CCOCC3)nc21)c1ccccc1]

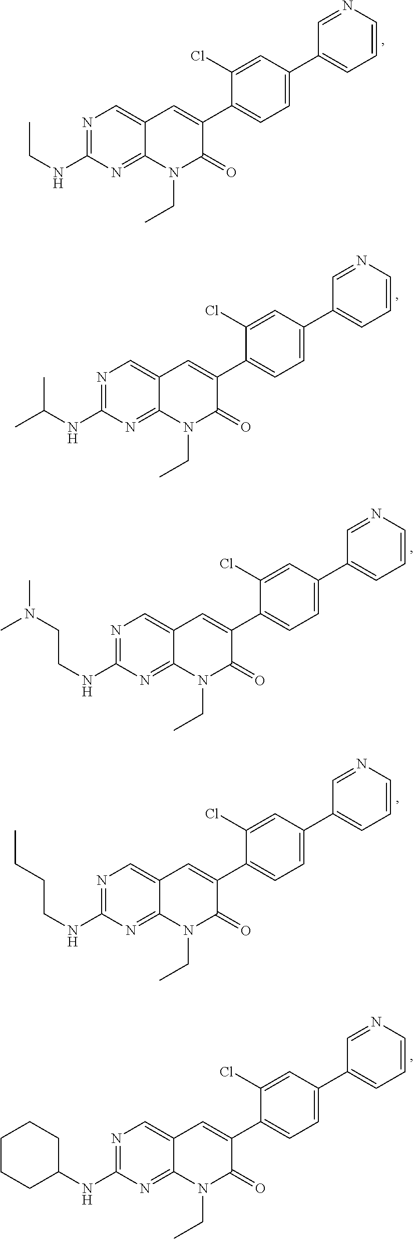 [SMILES: CCNc1ncc2cc(-c3ccc(-c4cccnc4)cc3Cl)c(=O)n(CC)c2n1 ; CCn1c(=O)c(-c2ccc(-c3cccnc3)cc2Cl)cc2cnc(NC(C)C)nc21 ; CCn1c(=O)c(-c2ccc(-c3cccnc3)cc2Cl)cc2cnc(NCCN(C)C)nc21 ; CCCCNc1ncc2cc(-c3ccc(-c4cccnc4)cc3Cl)c(=O)n(CC)c2n1 ; CCn1c(=O)c(-c2ccc(-c3cccnc3)cc2Cl)cc2cnc(NC3CCCCC3)nc21]

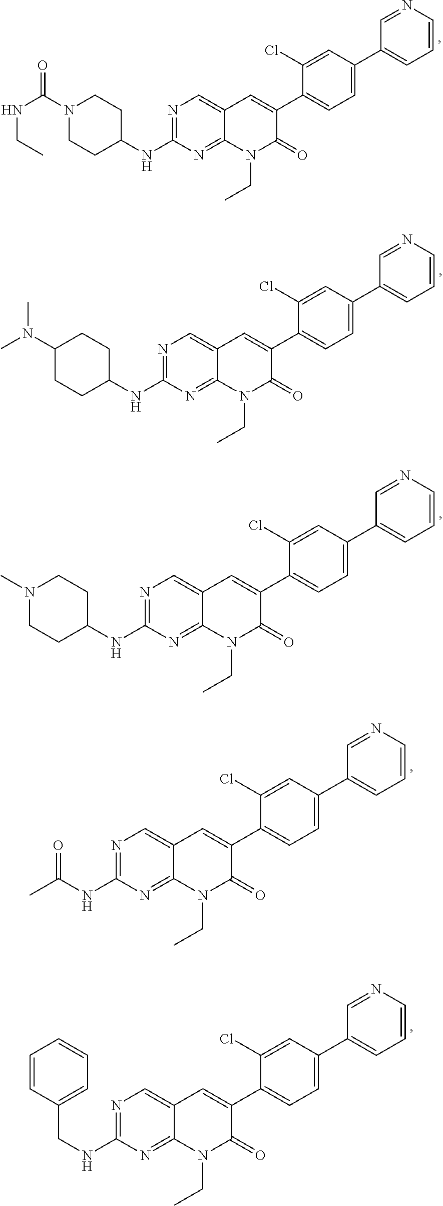 [SMILES: CCNC(=O)N1CCC(Nc2ncc3cc(-c4ccc(-c5cccnc5)cc4Cl)c(=O)n(CC)c3n2)CC1 ; CCn1c(=O)c(-c2ccc(-c3cccnc3)cc2Cl)cc2cnc(NC3CCC(N(C)C)CC3)nc21 ; CCn1c(=O)c(-c2ccc(-c3cccnc3)cc2Cl)cc2cnc(NC3CCN(C)CC3)nc21 ; CCn1c(=O)c(-c2ccc(-c3cccnc3)cc2Cl)cc2cnc(NC(C)=O)nc21 ; CCn1c(=O)c(-c2ccc(-c3cccnc3)cc2Cl)cc2cnc(NCc3ccccc3)nc21]

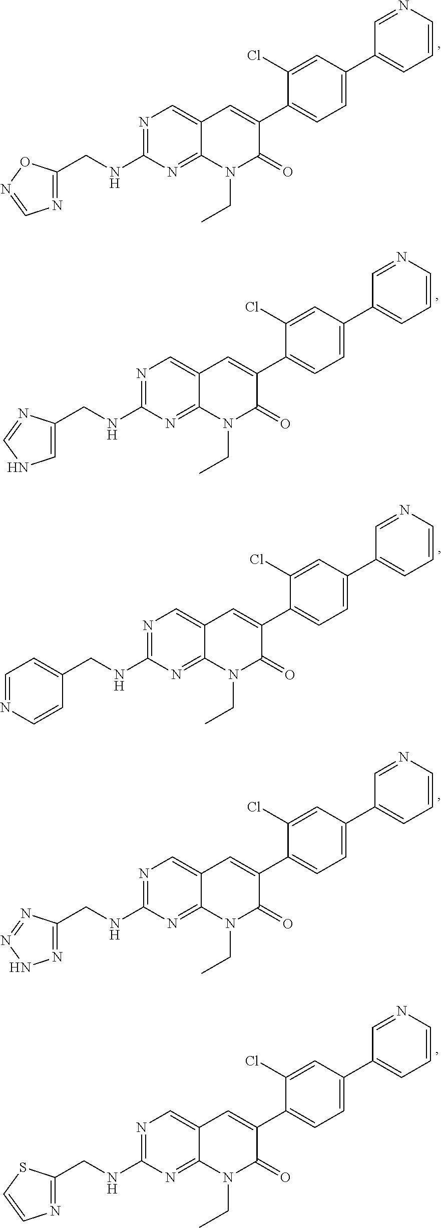 [SMILES: CCn1c(=O)c(-c2ccc(-c3cccnc3)cc2Cl)cc2cnc(NCc3ncno3)nc21 ; CCn1c(=O)c(-c2ccc(-c3cccnc3)cc2Cl)cc2cnc(NCc3c[nH]cn3)nc21 ; CCn1c(=O)c(-c2ccc(-c3cccnc3)cc2Cl)cc2cnc(NCc3ccncc3)nc21 ; CCn1c(=O)c(-c2ccc(-c3cccnc3)cc2Cl)cc2cnc(NCc3nn[nH]n3)nc21 ; CCn1c(=O)c(-c2ccc(-c3cccnc3)cc2Cl)cc2cnc(NCc3nccs3)nc21]

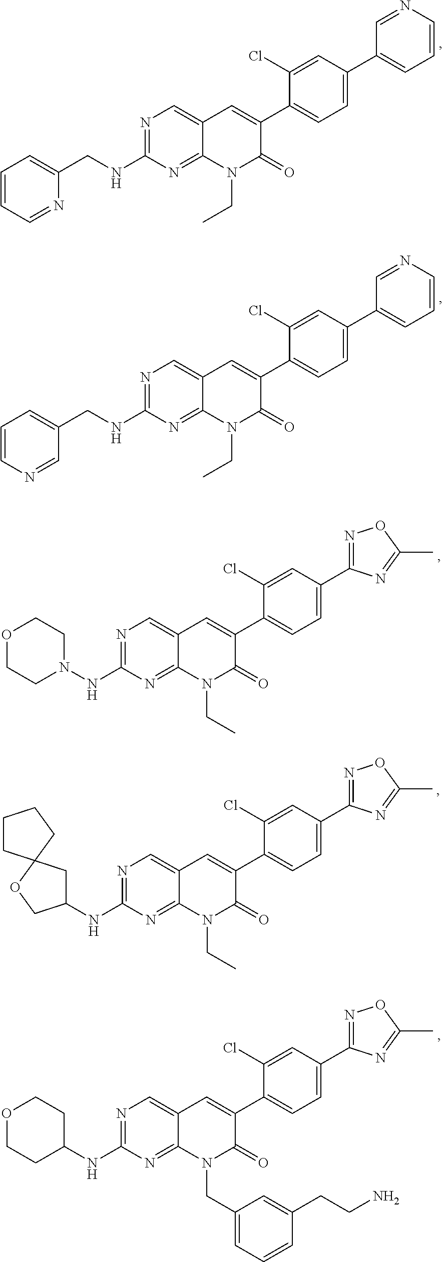 [SMILES: CCn1c(=O)c(-c2ccc(-c3cccnc3)cc2Cl)cc2cnc(NCc3ccccn3)nc21 ; CCn1c(=O)c(-c2ccc(-c3cccnc3)cc2Cl)cc2cnc(NCc3cccnc3)nc21 ; CCn1c(=O)c(-c2ccc(-c3noc(C)n3)cc2Cl)cc2cnc(NN3CCOCC3)nc21 ; CCn1c(=O)c(-c2ccc(-c3noc(C)n3)cc2Cl)cc2cnc(NC3COC4(CCCC4)C3)nc21 ; Cc1nc(-c2ccc(-c3cc4cnc(NC5CCOCC5)nc4n(Cc4cccc(CCN)c4)c3=O)c(Cl)c2)no1]

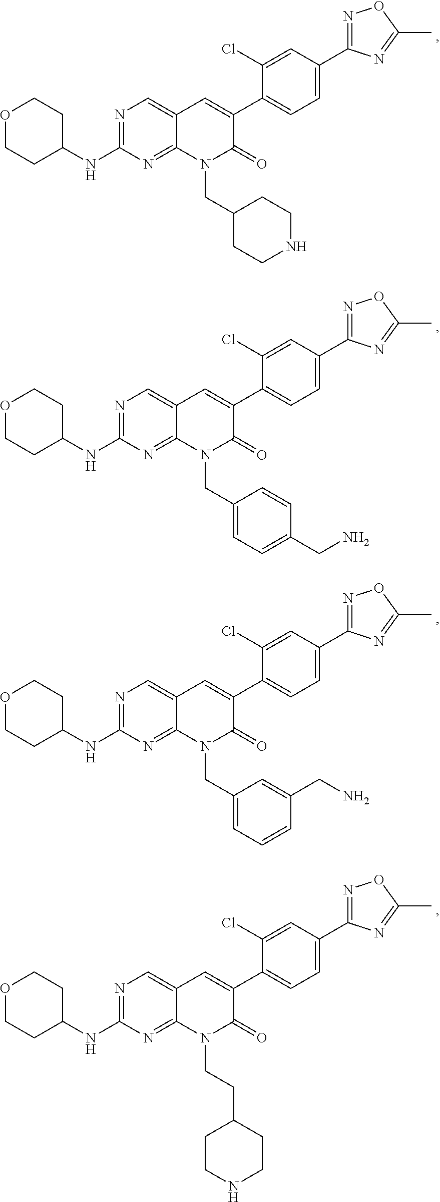 [SMILES: Cc1nc(-c2ccc(-c3cc4cnc(NC5CCOCC5)nc4n(CC4CCNCC4)c3=O)c(Cl)c2)no1 ; Cc1nc(-c2ccc(-c3cc4cnc(NC5CCOCC5)nc4n(Cc4ccc(CN)cc4)c3=O)c(Cl)c2)no1 ; Cc1nc(-c2ccc(-c3cc4cnc(NC5CCOCC5)nc4n(Cc4cccc(CN)c4)c3=O)c(Cl)c2)no1 ; Cc1nc(-c2ccc(-c3cc4cnc(NC5CCOCC5)nc4n(CCC4CCNCC4)c3=O)c(Cl)c2)no1]

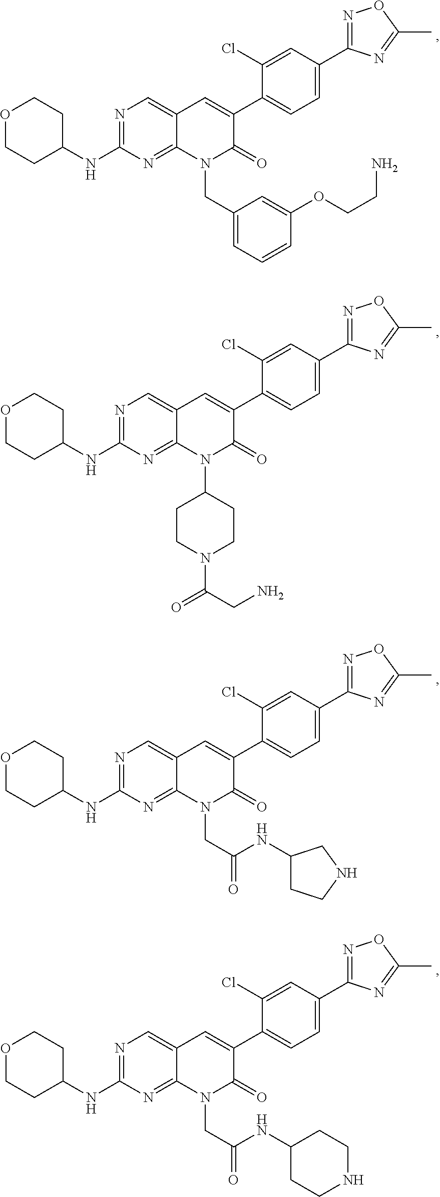 [SMILES: Cc1nc(-c2ccc(-c3cc4cnc(NC5CCOCC5)nc4n(Cc4cccc(OCCN)c4)c3=O)c(Cl)c2)no1 ; Cc1nc(-c2ccc(-c3cc4cnc(NC5CCOCC5)nc4n(C4CCN(C(=O)CN)CC4)c3=O)c(Cl)c2)no1 ; Cc1nc(-c2ccc(-c3cc4cnc(NC5CCOCC5)nc4n(CC(=O)NC4CCNC4)c3=O)c(Cl)c2)no1 ; Cc1nc(-c2ccc(-c3cc4cnc(NC5CCOCC5)nc4n(CC(=O)NC4CCNCC4)c3=O)c(Cl)c2)no1]

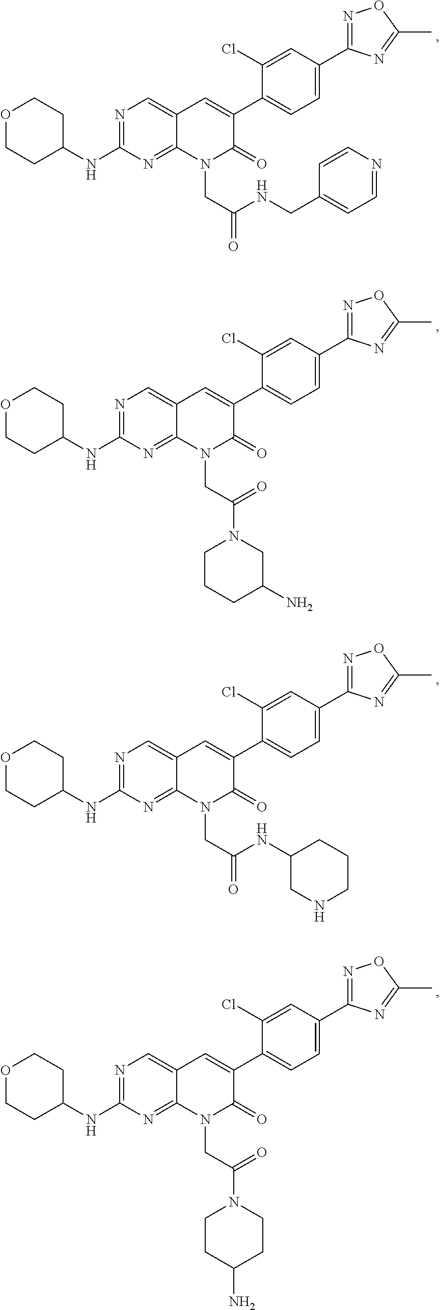 [SMILES: Cc1nc(-c2ccc(-c3cc4cnc(NC5CCOCC5)nc4n(CC(=O)NCc4ccncc4)c3=O)c(Cl)c2)no1 ; Cc1nc(-c2ccc(-c3cc4cnc(NC5CCOCC5)nc4n(CC(=O)N4CCCC(N)C4)c3=O)c(Cl)c2)no1 ; Cc1nc(-c2ccc(-c3cc4cnc(NC5CCOCC5)nc4n(CC(=O)NC4CCCNC4)c3=O)c(Cl)c2)no1 ; Cc1nc(-c2ccc(-c3cc4cnc(NC5CCOCC5)nc4n(CC(=O)N4CCC(N)CC4)c3=O)c(Cl)c2)no1]

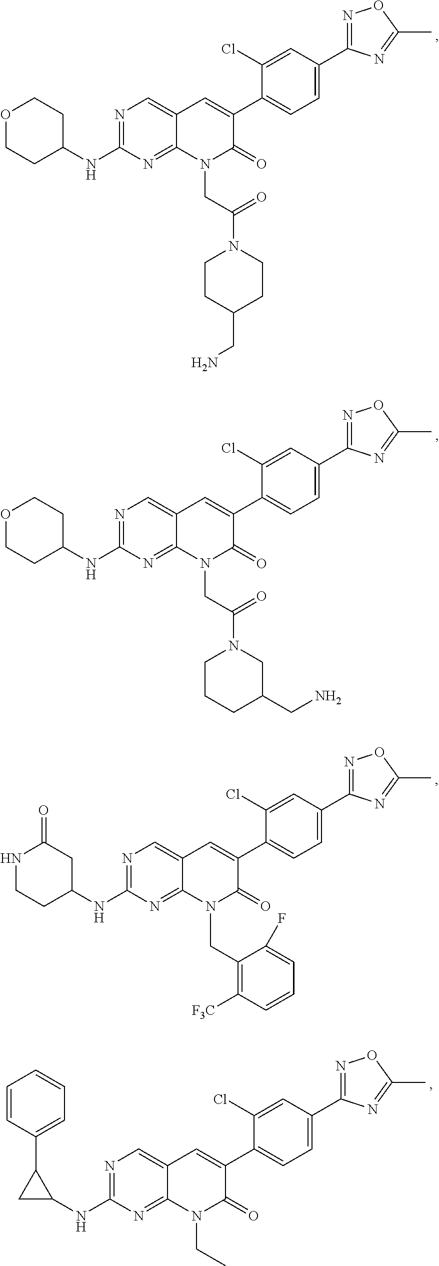 [SMILES: Cc1nc(-c2ccc(-c3cc4cnc(NC5CCOCC5)nc4n(CC(=O)N4CCC(CN)CC4)c3=O)c(Cl)c2)no1 ; Cc1nc(-c2ccc(-c3cc4cnc(NC5CCOCC5)nc4n(CC(=O)N4CCCC(CN)C4)c3=O)c(Cl)c2)no1 ; Cc1nc(-c2ccc(-c3cc4cnc(NC5CCNC(=O)C5)nc4n(Cc4c(F)cccc4C(F)(F)F)c3=O)c(Cl)c2)no1 ; CCn1c(=O)c(-c2ccc(-c3noc(C)n3)cc2Cl)cc2cnc(NC3CC3c3ccccc3)nc21]

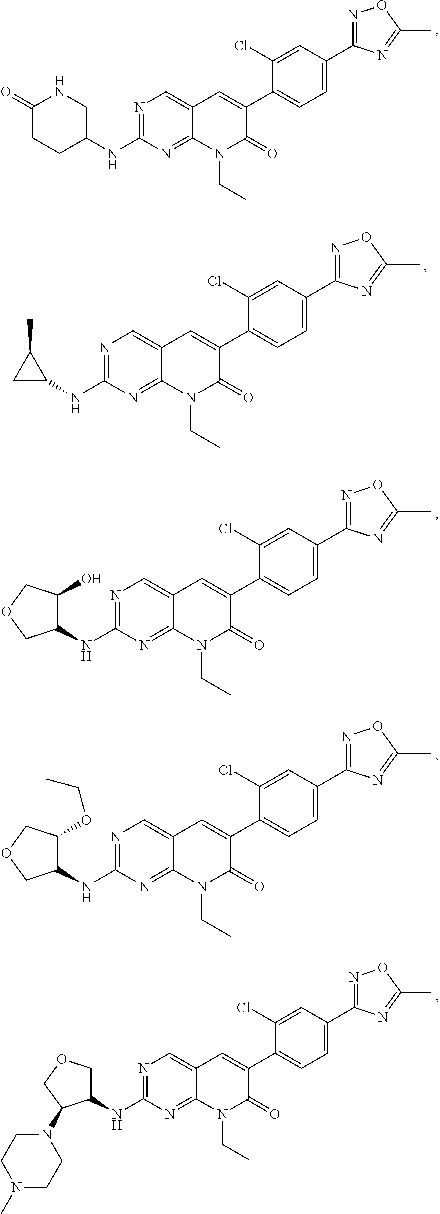

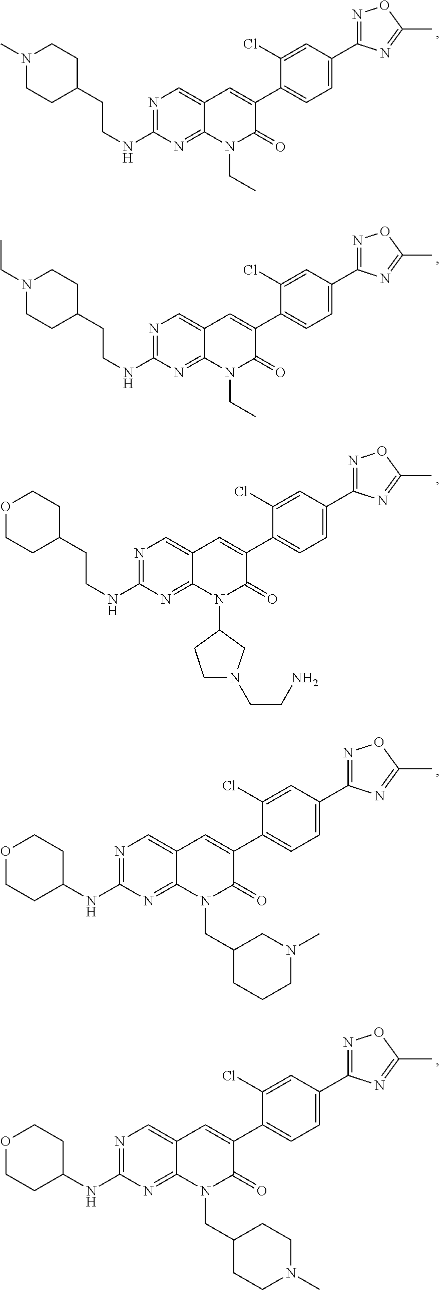

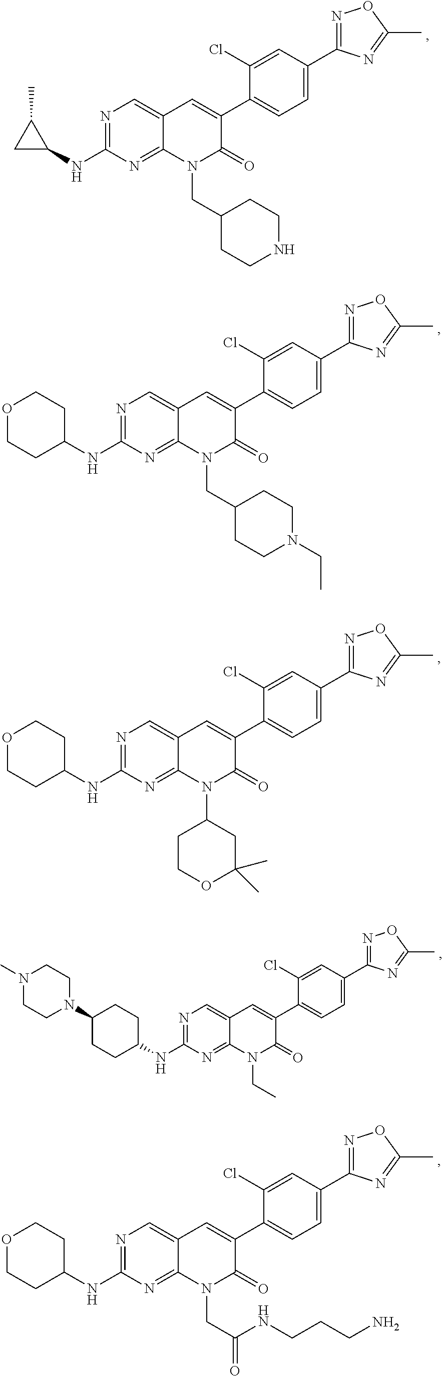

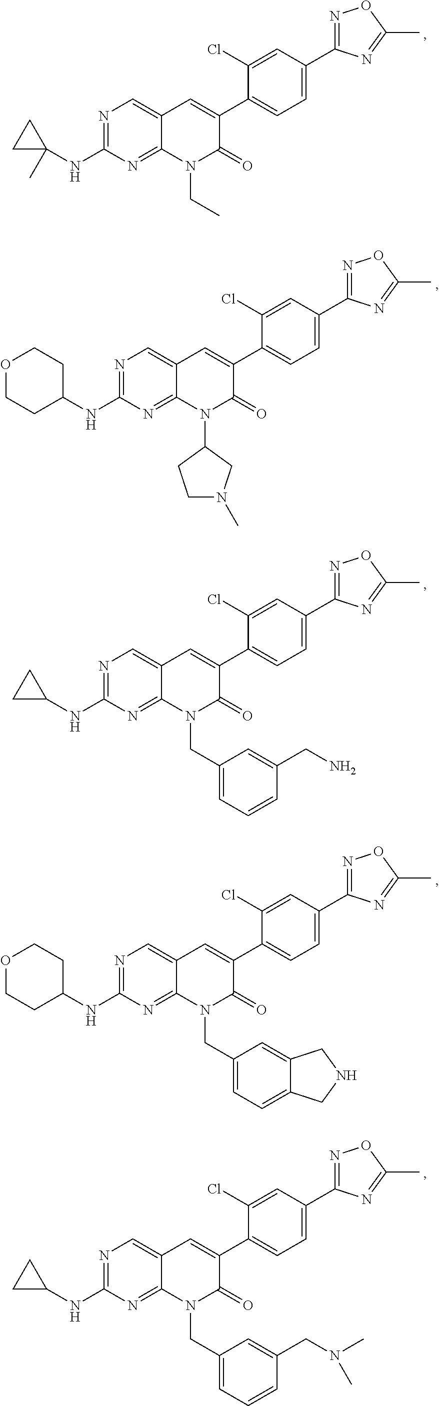 [SMILES: CCn1c(=O)c(-c2ccc(-c3noc(C)n3)cc2Cl)cc2cnc(NC3(C)CC3)nc21 ; Cc1nc(-c2ccc(-c3cc4cnc(NC5CCOCC5)nc4n(C4CCN(C)C4)c3=O)c(Cl)c2)no1 ; Cc1nc(-c2ccc(-c3cc4cnc(NC5CC5)nc4n(Cc4cccc(CN)c4)c3=O)c(Cl)c2)no1 ; Cc1nc(-c2ccc(-c3cc4cnc(NC5CCOCC5)nc4n(Cc4ccc5c(c4)CNC5)c3=O)c(Cl)c2)no1 ; Cc1nc(-c2ccc(-c3cc4cnc(NC5CC5)nc4n(Cc4cccc(CN(C)C)c4)c3=O)c(Cl)c2)no1]

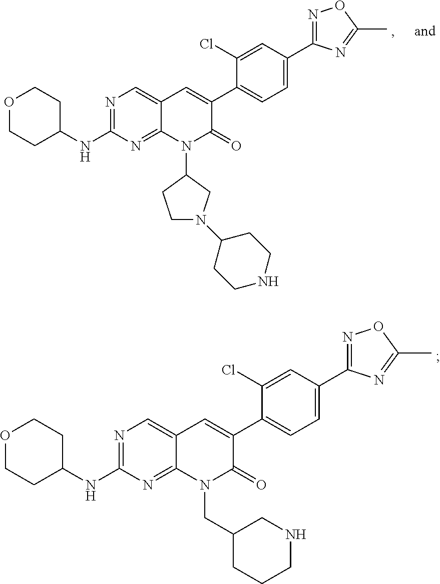 [SMILES: Cc1nc(-c2ccc(-c3cc4cnc(NC5CCOCC5)nc4n(C4CCN(C5CCNCC5)C4)c3=O)c(Cl)c2)no1 ; Cc1nc(-c2ccc(-c3cc4cnc(NC5CCOCC5)nc4n(CC4CCCNC4)c3=O)c(Cl)c2)no1]

or a pharmaceutically acceptable salt or N-oxide thereof.

Provided herein are pharmaceutical compositions comprising a therapeutically effective amount of a compound of Formula I-IV, or a pharmaceutically acceptable salt or N-oxide thereof, and a pharmaceutically acceptable carrier, wherein the compound of Formula I-IV is as described herein.

Provided herein, in some embodiments, are methods for treating Fragile X Syndrome, wherein the method comprises administering to an individual in need thereof a therapeutically effective amount of a compound having the structure of Formula A, Formula B, or Formula C, or a pharmaceutically acceptable salt or N-oxide thereof:

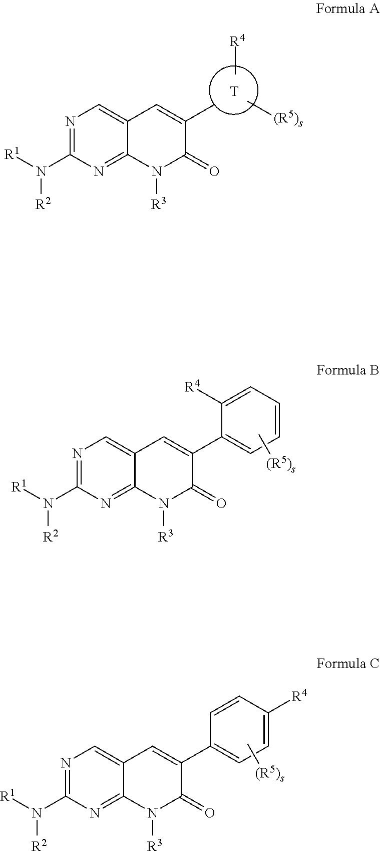

wherein:
ring T is an aryl or heteroaryl ring;
R¹ is H, or substituted or unsubstituted alkyl;
R² is substituted or unsubstituted alkyl, substituted or unsubstituted alkoxy, substituted or unsubstituted aralkoxy, substituted or unsubstituted heteroalkyl, substituted or unsubstituted cycloalkyl, substituted or unsubstituted cycloalkylalkyl, substituted or unsubstituted heterocycloalkyl, substituted or unsubstituted heterocycloalkylalkyl, spiro-cycloakyl-heterocycloalkyl, -alkylene-S(═O)R⁹, -alkylene-S(═O)₂R⁹, —S(═O)₂R⁹;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted heteroaryl attached to ring T or the phenyl ring via a carbon atom of R⁴, or substituted or unsubstituted heterocycloalkyl attached to ring T or the phenyl ring via a carbon atom of R⁴;
each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and s is 0-4.

In some embodiments, administration of a therapeutically effective amount of the compound of Formula A-C normalizes or partially normalizes aberrant synaptic plasticity associated with Fragile X syndrome. In some embodiment, administration of a therapeutically effective amount of the compound of Formula A-C normalizes or partially normalizes aberrant long term depression (LTD) associated with Fragile X syndrome. In some embodiments, administration of a therapeutically effective amount of the compound of Formula A-C normalizes or partially normalizes aberrant long term potentiation (LTP) associated with Fragile X syndrome.

### BRIEF DESCRIPTION OF THE DRAWINGS

The features of the present disclosure are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 describes illustrative shapes of dendritic spines.

FIG. 2 describes modulation of dendritic spine head diameter by a small molecule PAK inhibitor.

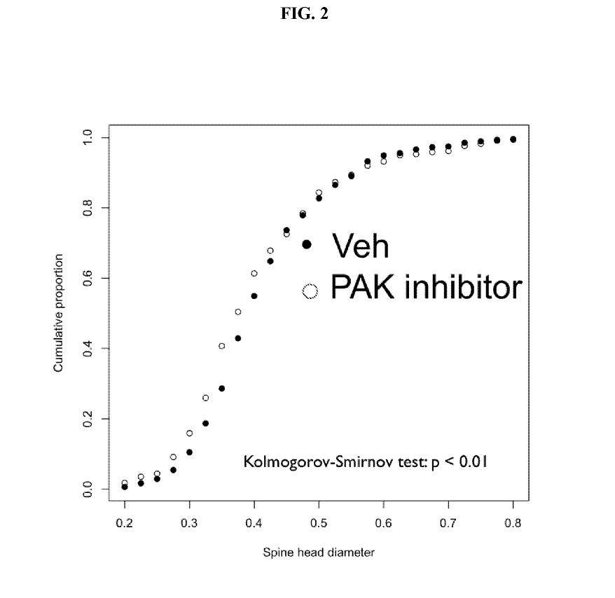

FIG. 3 describes modulation of dendritic spine length by a small molecule PAK inhibitor.

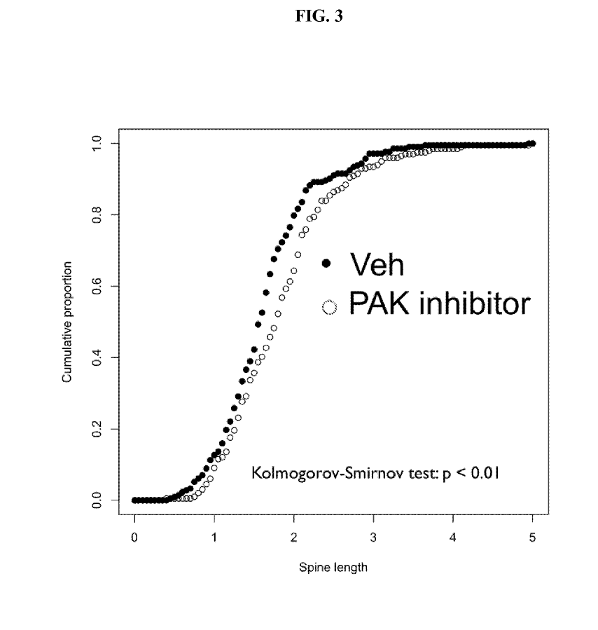

### DETAILED DESCRIPTION OF THE INVENTION

Provided herein are methods for treatment of Fragile X syndrome by administration of inhibitors of certain p21 activated kinases to individuals in need thereof. Such kinase inhibitors are inhibitors of one or more of PAK1, PAK2, PAK3, PAK4, PAK5 or PAK6 kinases. In certain embodiments, the individual has been diagnosed with or is suspected of suffering from Fragile X syndrome. In some instances, provided herein are methods for treating Fragile X syndrome characterized by abnormal dendritic spine morphology and/or spine density and/or spine length and/or spine thickness comprising inhibiting PAK activity by administration of a therapeutically effective amount of a PAK inhibitor to an individual diagnosed with or suspected of suffering from Fragile X syndrome.

Some CNS disorders are characterized by abnormal dendritic spine morphology, spine size, spine plasticity and/or spine density as described in a number of studies referred to herein. PAK kinase activity has been implicated in spine morphogenesis, maturation, and maintenance. See, e.g., Kreis et al (2007), J Biol Chem, 282(29):21497-21506; Hayashi et al (2007), Proc Natl Acad Sci USA., 104(27):11489-11494, Hayashi et al (2004), Neuron, 42(5):773-787; Penzes et al (2003), Neuron, 37:263-274. In some embodiments, inhibition or partial inhibition of one or more PAKs normalizes aberrant dendritic spine morphology and/or synaptic function.

In some instances, Fragile X syndrome is associated with abnormal dendritic spine morphology, spine size, spine plasticity, spine motility, spine density and/or abnormal synaptic function. In some instances, activation of one or more of PAK1, PAK2, PAK3, PAK4, PAK5 and/or PAK6 kinases is implicated in defective spine morphogenesis, maturation, and maintenance. Described herein are methods for suppressing or reducing PAK activity (e.g., by administering a PAK inhibitor for rescue of defects in spine morphology, size, plasticity spine motility and/or density) associated with Fragile X syndrome as described herein. Accordingly, in some embodiments, the methods described herein are used to treat an individual suffering from Fragile X syndrome associated with abnormal dendritic spine density, spine size, spine plasticity, spine morphology, spine plasticity, or spine motility.

In some embodiments, any inhibitor of one or more p21-activated kinases described herein reverses or partially reverses defects in dendritic spine morphology and/or dendritic spine density and/or synaptic function that are associated with Fragile X syndrome. In some embodiments, modulation of dendritic spine morphology and/or dendritic spine density and/or synaptic function alleviates or reverses cognitive impairment and/or negative behavioral symptoms (e.g., social withdrawal, anhedonia or the like) associated with Fragile X such as psychiatric conditions. In some embodiments, modulation of dendritic spine morphology and/or dendritic spine density and/or synaptic function halts or delays progression of cognitive impairment and/or loss of bodily functions associated with Fragile X syndrome.

In some instances, cellular changes in brain cells contribute to pathogenesis of Fragile X syndrome. In some instances, abnormal dendritic spine density in the brain contributes to the pathogenesis of Fragile X syndrome. In some instances, abnormal dendritic spine morphology contributes to the pathogenesis of Fragile X syndrome. In some instances, an abnormal pruning of dendritic spines or synapses during puberty contributes to the pathogenesis of Fragile X syndrome. In some instances, abnormal synaptic function contributes to the pathogenesis of Fragile X syndrome. In some instances, activation of one or more PAKs is associated with abnormal dendritic spine density and/or dendritic morphology and/or synaptic function and contributes to the pathogenesis of Fragile X syndrome. In some instances, modulation of PAK activity (e.g., attenuation, inhibition or partial inhibition of PAK activity) reverses or reduces abnormal dendritic spine morphology and/or dendritic spine density and/or synaptic function. In certain embodiments, modulation of activity of one or more Group I PAKs (one or more of PAK1, PAK2 and/or PAK3) reverses or reduces abnormal dendritic spine morphology and/or dendritic spine density and/or synaptic function associated with Fragile X syndrome.

### Dendritic Spines

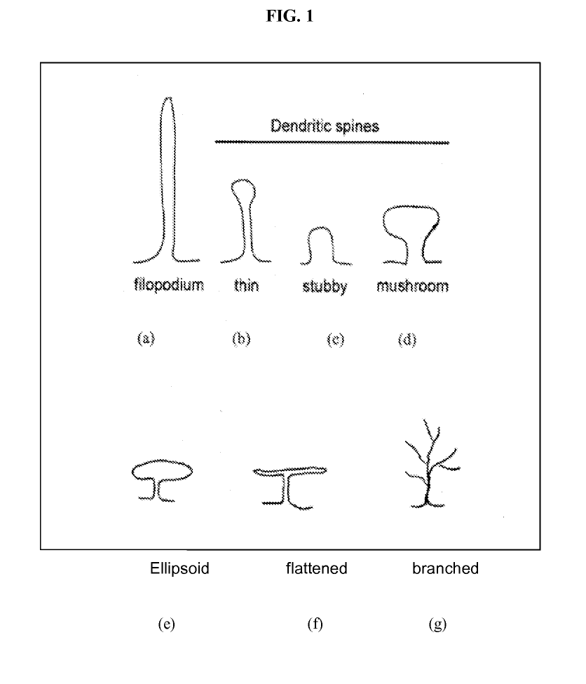

A dendritic spine is a small membranous protrusion from a neuron's dendrite that serves as a specialized structure for the formation, maintenance, and/or function of synapses. Dendritic spines vary in size and shape. In some instances, spines have a bulbous head (the spine head) of varying shape, and a thin neck that connects the head of the spine to the shaft of the dendrite. In some instances, spine numbers and shape are regulated by physiological and pathological events. In some instances, a dendritic spine head is a site of synaptic contact. In some instances, a dendritic spine shaft is a site of synaptic contact. FIG. 1 shows examples of different shapes of dendritic spines. Dendritic spines are “plastic.” In other words, spines are dynamic and continually change in shape, volume, and number in a highly regulated process. In some instances, spines change in shape, volume, length, thickness or number in a few hours. In some instances, spines change in shape, volume, length, thickness or number occurs within a few minutes. In some instances, spines change in shape, volume, length, thickness or number occurs in response to synaptic transmission and/or induction of synaptic plasticity. By way of example, dendritic spines are headless (filopodia as shown, for example, in FIG. 1a), thin (for example, as shown in FIG. 1b), stubby (for example as shown in FIG. 1c), mushroom-shaped (have door-knob heads with thick necks, for example as shown in FIG. 1d), ellipsoid (have prolate spheroid heads with thin necks, for example as shown in FIG. 1e), flattened (flattened heads with thin neck, for example as shown in FIG. 1f) or branched (for example as shown in FIG. 1g).

In some instances, mature spines have variably-shaped bulbous tips or heads, ˜0.5-2 μm in diameter, connected to a parent dendrite by thin stalks 0.1-1 μm long. In some instances, an immature dendritic spine is filopodia-like, with a length of 1.5-4 μm and no detectable spine head. In some instances, spine density ranges from 1 to 10 spines per micrometer length of dendrite, and varies with maturational stage of the spine and/or the neuronal cell. In some instances, dendritic spine density ranges from 1 to 40 spines per 10 micrometer in medium spiny neurons.

In some instances, the shape of the dendritic spine head determines synpatic function. Defects in dendritic spine morphology and/or function have been described as associated with Fragile X syndrome. As an example, neurons from patients with Fragile X mental retardation show a significant increase in the overall density of dendritic spines, together with an increase in the proportion of “immature”, filopodia-like spines and a corresponding reduction of “mature”, mushrooms-shaped spines (Irvin et al, Cerebral Cortex, 2000; 10:1038-1044). In many cases, the dendritic spine defects found in samples from human brains have been recapitulated in rodent models of the disease and correlated to defective synapse function and/or plasticity. In some instances, dendritic spines with larger spine head diameter form more stable synapses compared with dendritic spines with smaller head diameter. In some instances, a mushroom-shaped spine head is associated with normal or partially normal synaptic function. In some instances, a mushroom-shaped spine is a healthier spine (e.g., having normal or partially normal synapses) compared to a spine with a reduced spine head size, spine head volume and/or spine head diameter. In some instances, inhibition or partial inhibition of PAK activity results in an increase in spine head diameter and/or spine head volume and/or reduction of spine length, thereby normalizing or partially normalizing synaptic function in individuals suffering or suspected of suffering from Fragile X syndrome.

p21-Activated Kinases (PAKs)

The PAKs constitute a family of serine-threonine kinases that is composed of “conventional”, or Group I PAKs, that includes PAK1, PAK2, and PAK3, and “non-conventional”, or Group II PAKs, that includes PAK4, PAK5, and PAK6. See, e.g., Zhao et al. (2005), Biochem J, 386:201-214. These kinases function downstream of the small GTPases Rac and/or Cdc42 to regulate multiple cellular functions, including dendritic morphogenesis and maintenance (see, e.g., Ethell et al (2005), Prog in Neurobiol, 75:161-205; Penzes et al (2003), Neuron, 37:263-274), motility, morphogenesis, angiogenesis, and apoptosis, (see, e.g., Bokoch et al., 2003, Annu. Rev. Biochem., 72:743; and Hofmann et al., 2004, J. Cell Sci., 117:4343;). GTP-bound Rac and/or Cdc42 bind to inactive PAK, releasing steric constraints imposed by a PAK autoinhibitory domain and/or permitting PAK phosphorylation and/or activation. Numerous phosphorylation sites have been identified that serve as markers for activated PAK.

In some instances, upstream effectors of PAK include, but are not limited to, TrkB receptors; NMDA receptors; adenosine receptors; estrogen receptors; integrins, EphB receptors; CDK5, FMRP; Rho-family GTPases, including Cdc42, Rac (including but not limited to Rac1 and Rac2), Chp, TC10, and Wrnch-1; guanine nucleotide exchange factors (“GEFs”), such as but not limited to GEFT, α-p-21-activated kinase interacting exchange factor (αPIX), Kalirin-7, and Tiam1; G protein-coupled receptor kinase-interacting protein 1 (GIT1), and sphingosine.

In some instances, downstream effectors of PAK include, but are not limited to, substrates of PAK kinase, such as Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), Ras, Raf, Mek, p47phox, BAD, caspase 3, estrogen and/or progesterone receptors, RhoGEF, GEF-H1, NET1, Gaz, phosphoglycerate mutase-B, RhoGDI, prolactin, p41Arc, cortactin and/or Aurora-A (See, e.g., Bokoch et al., 2003, Annu. Rev. Biochem., 72:743; and Hofmann et al., 2004, J. Cell Sci., 117:4343). Other substances that bind to PAK in cells include CIB; sphingolipids; lysophosphatidic acid, G-protein β and/or γ subunits; PIX/COOL; GIT/PKL; Nef; Paxillin; NESH; 5H3-containing proteins (e.g. Nck and/or Grb2); kinases (e.g. Akt, PDK1, PI 3-kinase/p85, Cdk5, Cdc2, Src kinases, Abl, and/or protein kinase A (PKA)); and/or phosphatases (e.g. phosphatase PP2A, POPX1, and/or POPX2).

### PAK Inhibitors

Described herein are PAK inhibitors that treat one or more symptoms associated with Fragile X syndrome. Also described herein are pharmaceutical compositions comprising a PAK inhibitor (e.g., a PAK inhibitor compound described herein) for reversing or reducing one or more of cognitive impairment and/or dementia and/or negative symptoms and/or positive symptoms associated with Fragile X syndrome. Also described herein are pharmaceutical compositions comprising a PAK inhibitor (e.g., a PAK inhibitor compound described herein) for halting or delaying the progression of cognitive impairment and/or dementia and/or negative symptoms and/or positive symptoms associated with Fragile X syndrome. Described herein is the use of a PAK inhibitor for manufacture of a medicament for treatment of one or more symptoms of Fragile X syndrome.

In some embodiments, the PAK inhibitor is a Group I PAK inhibitor that inhibits, for example, one or more Group I PAK polypeptides, for example, PAK1, PAK2, and/or PAK3. In some embodiments, the PAK inhibitor is a PAK1 inhibitor. In some embodiments, the PAK inhibitor is a PAK2 inhibitor. In some embodiments, the PAK inhibitor is a PAK3 inhibitor. In some embodiments, the PAK inhibitor is a mixed PAK1/PAK3 inhibitor. In some embodiments, the PAK inhibitor is a mixed PAK1/PAK2 inhibitor. In some embodiments, the PAK inhibitor is a mixed PAK1/PAK4 inhibitor. In some embodiments, the PAK inhibitor is a mixed PAK1/PAK2/PAK4 inhibitor. In some embodiments, the PAK inhibitor is a mixed PAK1/PAK2/PAK3/PAK4 inhibitor. In some embodiments, the PAK inhibitor inhibits all three Group I PAK isoforms (PAK1, 2 and PAK3) with equal or similar potency. In some embodiments, the PAK inhibitor is a Group II PAK inhibitor that inhibits one or more Group II PAK polypeptides, for example PAK4, PAK5, and/or PAK6. In some embodiments, the PAK inhibitor is a PAK4 inhibitor. In some embodiments, the PAK inhibitor is a PAK5 inhibitor. In some embodiments, the PAK inhibitor is a PAK6 inhibitor.

In certain embodiments, a PAK inhibitor described herein reduces or inhibits the activity of one or more of PAK1, PAK2, PAK3, and/or PAK4 while not affecting the activity of PAK5 and PAK6. In some embodiments, a PAK inhibitor described herein reduces or inhibits the activity of one or more of PAK1, PAK2 and/or PAK3 while not affecting the activity of PAK4, PAK5 and/or PAK6. In some embodiments, a PAK inhibitor described herein reduces or inhibits the activity of one or more of PAK1, PAK2, PAK3, and/or one or more of PAK4, PAK5 and/or PAK6. In some embodiments, a PAK inhibitor described herein is a substantially complete inhibitor of one or more PAKs. As used herein, “substantially complete inhibition” means, for example, >95% inhibition of one or more targeted PAKs. In other embodiments, “substantially complete inhibition” means, for example, >90% inhibition of one or more targeted PAKs. In some other embodiments, “substantially complete inhibition” means, for example, >80% inhibition of one or more targeted PAKs. In some embodiments, a PAK inhibitor described herein is a partial inhibitor of one or more PAKs. As used herein, “partial inhibition” means, for example, between about 40% to about 60% inhibition of one or more targeted PAKs. In other embodiments, “partial inhibition” means, for example, between about 50% to about 70% inhibition of one or more targeted PAKs. As used herein, where a PAK inhibitor substantially inhibits or partially inhibits the activity of a certain PAK isoform while not affecting the activity of another isoform, it means, for example, less than about 10% inhibition of the non-affected isoform when the isoform is contacted with the same concentration of the PAK inhibitor as the other substantially inhibited or partially inhibited isoforms. In other instances, where a PAK inhibitor substantially inhibits or partially inhibits the activity of a certain PAK isoform while not affecting the activity of another isoform, it means, for example, less than about 5% inhibition of the non-affected isoform when the isoform is contacted with the same concentration of the PAK inhibitor as the other substantially inhibited or partially inhibited isoforms. In yet other instances, where a PAK inhibitor substantially inhibits or partially inhibits the activity of a certain PAK isoform while not affecting the activity of another isoform, it means, for example, less than about 1% inhibition of the non-affected isoform when the isoform is contacted with the same concentration of the PAK inhibitor as the other substantially inhibited or partially inhibited isoforms.

### Compounds of the Present Disclosure

Provided herein, in certain embodiments, are compounds having the structure of Formula I or pharmaceutically acceptable salt or N-oxide thereof:

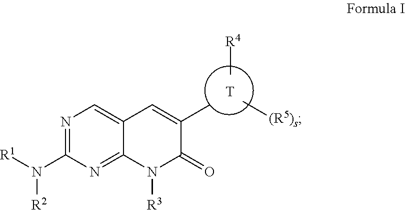

wherein:
ring T is an aryl or heteroaryl ring;
R¹ is H, or substituted or unsubstituted alkyl;
R² is alkyl substituted with —OH, —OMe, —SH, —SMe, or halogen;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted heteroaryl attached to ring T via a carbon atom of R⁴, or substituted or unsubstituted heterocycloalkyl attached to ring T via a carbon atom of R⁴;
each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

In one embodiment is a compound of Formula I wherein ring T is aryl. In a refinement, aryl is phenyl. In another refinement, aryl is naphthalene.

In one embodiment is a compound of Formula I wherein ring T is selected from pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl. In some embodiments, ring T is pyrrolyl. In some embodiments, ring T is furanyl. In some embodiments, ring T is thiophenyl. In some embodiments, ring T is pyrazolyl. In some embodiments, ring T is imidazolyl. In some embodiments, ring T is isoxazolyl. In some embodiments, ring T is oxazolyl. In some embodiments, ring T is isothiazolyl. In some embodiments, ring T is thiazolyl. In some embodiments, ring T is 1,2,3-triazolyl. In some embodiments, ring T is 1,3,4-triazolyl. In some embodiments, ring T is 1-oxa-2,3-diazolyl. In some embodiments, ring T is 1-oxa-2,4-diazolyl. In some embodiments, ring T is 1-oxa-2,5-diazolyl. In some embodiments, ring T is 1-oxa-3,4-diazolyl. In some embodiments, ring T is 1-thia-2,3-diazolyl. In some embodiments, ring T is 1-thia-2,4-diazolyl. In some embodiments, ring T is 1-thia-2,5-diazolyl. In some embodiments, ring T is 1-thia-3,4-diazolyl. In some embodiments, ring T is tetrazolyl. In some embodiments, ring T is pyridinyl. In some embodiments, ring T is pyridazinyl. In some embodiments, ring T is pyrimidinyl. In some embodiments, ring T is pyrazinyl. In some embodiments, ring T is triazinyl. In some embodiments, ring T is indolyl. In some embodiments, ring T is benzofuranyl. In some embodiments, ring T is benzimidazolyl. In some embodiments, ring T is indazolyl. In some embodiments, ring T is pyrrolopyridinyl. In some embodiments, ring T is imidazopyridinyl.

In a further embodiment is a compound of Formula I, wherein R⁴ is a substituted or unsubstituted C-linked heterocycloalkyl. In a further embodiment, the C-linked heterocycloalkyl is pyrrolidinyl, tetrahydrofuranyl, piperidinyl, tetrahydropyranyl, tetrahydrothiopyranyl, morpholinyl, or piperazinyl. In some embodiments, the C-linked heterocycloalkyl is pyrrolidinyl. In some embodiments, the C-linked heterocycloalkyl is tetrahydrofuranyl. In some embodiments, the C-linked heterocycloalkyl is piperidinyl. In some embodiments, the C-linked heterocycloalkyl is tetrahydropyranyl. In some embodiments, the C-linked heterocycloalkyl is tetrahydrothiopyranyl. In some embodiments, the C-linked heterocycloalkyl is morpholinyl. In some embodiments, the C-linked heterocycloalkyl is piperazinyl. In a further embodiment, the C-linked heterocycloalkyl is substituted with at least one C₁-C₆alkyl or halogen. In another embodiment, the C₁-C₆alkyl is methyl, ethyl, or n-propyl.

In one embodiment is a compound of Formula I, wherein R⁴ is a substituted or unsubstituted C-linked heteroaryl. In one embodiment, R⁴ is selected from a C-linked pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl. In some embodiments, R⁴ is a C-linked pyrrolyl. In some embodiments, R⁴ is a C-linked furanyl. In some embodiments, R⁴ is a C-linked thiophenyl. In some embodiments, R⁴ is a C-linked pyrazolyl. In some embodiments, R⁴ is a C-linked imidazolyl. In some embodiments, R⁴ is a C-linked isoxazolyl. In some embodiments, R⁴ is a C-linked oxazolyl. In some embodiments, R⁴ is a C-linked isothiazolyl. In some embodiments, R⁴ is a C-linked thiazolyl. In some embodiments, R⁴ is a C-linked 1,2,3-triazolyl. In some embodiments, R⁴ is a C-linked 1,3,4-triazolyl. In some embodiments, R⁴ is a C-linked 1-oxa-2,3-diazolyl. In some embodiments, R⁴ is a C-linked 1-oxa-2,4-diazolyl. In some embodiments, R⁴ is a C-linked 1-oxa-2,5-diazolyl. In some embodiments, R⁴ is a C-linked 1-oxa-3,4-diazolyl. In some embodiments, R⁴ is a C-linked 1-thia-2,3-diazolyl. In some embodiments, R⁴ is a C-linked 1-thia-2,4-diazolyl. In some embodiments, R⁴ is a C-linked 1-thia-2,5-diazolyl. In some embodiments, R⁴ is a C-linked 1-thia-3,4-diazolyl. In some embodiments, R⁴ is a C-linked tetrazolyl. In some embodiments, R⁴ is a C-linked pyridinyl. In some embodiments, R⁴ is a C-linked pyridazinyl. In some embodiments, R⁴ is a C-linked pyrimidinyl. In some embodiments, R⁴ is a C-linked pyrazinyl. In some embodiments, R⁴ is a C-linked triazinyl. In some embodiments, R⁴ is a C-linked indolyl. In some embodiments, R⁴ is a C-linked benzofuranyl. In some embodiments, R⁴ is a C-linked benzimidazolyl. In some embodiments, R⁴ is a C-linked indazolyl. In some embodiments, R⁴ is a C-linked pyrrolopyridinyl. In some embodiments, R⁴ is a C-linked imidazopyridinyl.

In yet another embodiment is a compound of Formula I, wherein R⁴ is a C-linked heteroaryl substituted with at least one group selected from halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, the C-linked heteroaryl is substituted with C₁-C₆alkyl. In another embodiment, C₁-C₆alkyl is methyl, ethyl, n-propyl, iso-propyl, n-butyl, iso-butyl, or tert-butyl. In a further embodiment, the C-linked heteroaryl is substituted with methyl. In another embodiment, the C-linked heteroaryl is substituted with ethyl. In a further embodiment, the C-linked heteroaryl is substituted with n-propyl or iso-propyl.

In another embodiment is a compound of Formula I having the structure of Formula Ia:

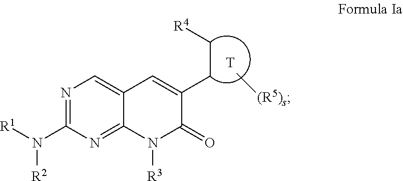

wherein ring T, R¹, R², R³, R⁴, R⁵, and s are described previously.

In another embodiment is a compound of Formula I having the structure of Formula Ib:

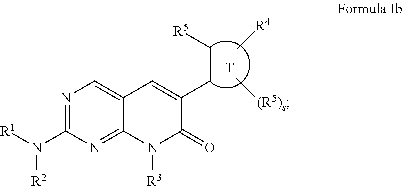

wherein ring T, R¹, R², R³, R⁴, R⁵ are described previously and s is 0-3.

In another embodiment are compounds having the structure of Formula Ic or pharmaceutically acceptable salt or N-oxide thereof:

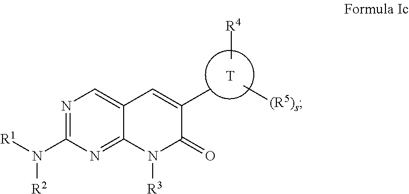

wherein:
ring T is an aryl or heteroaryl ring;
R¹ is H, or substituted or unsubstituted alkyl;
R² is alkyl substituted with —OH, —OMe, —SH, —SMe, or halogen;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted cycloalkyl, substituted or unsubstituted aryl, substituted or unsubstituted heteroaryl attached to ring T via a carbon atom of R⁴, or substituted or unsubstituted heterocycloalkyl attached to ring T via a carbon atom of R⁴;
each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

In one embodiment is a compound of Formula Ic wherein ring T is aryl. In a refinement, aryl is phenyl. In another refinement, aryl is naphthalene.

In one embodiment is a compound of Formula Ic wherein ring T is selected from pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl. In some embodiments, ring T is pyrrolyl. In some embodiments, ring T is furanyl. In some embodiments, ring T is thiophenyl. In some embodiments, ring T is pyrazolyl. In some embodiments, ring T is imidazolyl. In some embodiments, ring T is isoxazolyl. In some embodiments, ring T is oxazolyl. In some embodiments, ring T is isothiazolyl. In some embodiments, ring T is thiazolyl. In some embodiments, ring T is 1,2,3-triazolyl. In some embodiments, ring T is 1,3,4-triazolyl. In some embodiments, ring T is 1-oxa-2,3-diazolyl. In some embodiments, ring T is 1-oxa-2,4-diazolyl. In some embodiments, ring T is 1-oxa-2,5-diazolyl. In some embodiments, ring T is 1-oxa-3,4-diazolyl. In some embodiments, ring T is 1-thia-2,3-diazolyl. In some embodiments, ring T is 1-thia-2,4-diazolyl. In some embodiments, ring T is 1-thia-2,5-diazolyl. In some embodiments, ring T is 1-thia-3,4-diazolyl. In some embodiments, ring T is tetrazolyl. In some embodiments, ring T is pyridinyl. In some embodiments, ring T is pyridazinyl. In some embodiments, ring T is pyrimidinyl. In some embodiments, ring T is pyrazinyl. In some embodiments, ring T is triazinyl. In some embodiments, ring T is indolyl. In some embodiments, ring T is benzofuranyl. In some embodiments, ring T is benzimidazolyl. In some embodiments, ring T is indazolyl. In some embodiments, ring T is pyrrolopyridinyl. In some embodiments, ring T is imidazopyridinyl.

In a further embodiment is a compound of Formula Ic, wherein R⁴ is a substituted or unsubstituted C-linked heterocycloalkyl. In a further embodiment, the C-linked heterocycloalkyl is pyrrolidinyl, tetrahydrofuranyl, piperidinyl, tetrahydropyranyl, tetrahydrothiopyranyl, morpholinyl, or piperazinyl. In some embodiments, the C-linked heterocycloalkyl is pyrrolidinyl. In some embodiments, the C-linked heterocycloalkyl is tetrahydrofuranyl. In some embodiments, the C-linked heterocycloalkyl is piperidinyl. In some embodiments, the C-linked heterocycloalkyl is tetrahydropyranyl. In some embodiments, the C-linked heterocycloalkyl is tetrahydrothiopyranyl. In some embodiments, the C-linked heterocycloalkyl is morpholinyl. In some embodiments, the C-linked heterocycloalkyl is piperazinyl. In a further embodiment, the C-linked heterocycloalkyl is substituted with at least one C₁-C₆alkyl or halogen. In another embodiment, the C₁-C₆alkyl is methyl, ethyl, or n-propyl.

In one embodiment is a compound of Formula Ic, wherein R⁴ is a substituted or unsubstituted C-linked heteroaryl. In one embodiment, R⁴ is selected from a C-linked pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl. In some embodiments, R⁴ is a C-linked pyrrolyl. In some embodiments, R⁴ is a C-linked furanyl. In some embodiments, R⁴ is a C-linked thiophenyl. In some embodiments, R⁴ is a C-linked pyrazolyl. In some embodiments, R⁴ is a C-linked imidazolyl. In some embodiments, R⁴ is a C-linked isoxazolyl. In some embodiments, R⁴ is a C-linked oxazolyl. In some embodiments, R⁴ is a C-linked isothiazolyl. In some embodiments, R⁴ is a C-linked thiazolyl. In some embodiments, R⁴ is a C-linked 1,2,3-triazolyl. In some embodiments, R⁴ is a C-linked 1,3,4-triazolyl. In some embodiments, R⁴ is a C-linked 1-oxa-2,3-diazolyl. In some embodiments, R⁴ is a C-linked 1-oxa-2,4-diazolyl. In some embodiments, R⁴ is a C-linked 1-oxa-2,5-diazolyl. In some embodiments, R⁴ is a C-linked 1-oxa-3,4-diazolyl. In some embodiments, R⁴ is a C-linked 1-thia-2,3-diazolyl. In some embodiments, R⁴ is a C-linked 1-thia-2,4-diazolyl. In some embodiments, R⁴ is a C-linked 1-thia-2,5-diazolyl. In some embodiments, R⁴ is a C-linked 1-thia-3,4-diazolyl. In some embodiments, R⁴ is a C-linked tetrazolyl. In some embodiments, R⁴ is a C-linked pyridinyl. In some embodiments, R⁴ is a C-linked pyridazinyl. In some embodiments, R⁴ is a C-linked pyrimidinyl. In some embodiments, R⁴ is a C-linked pyrazinyl. In some embodiments, R⁴ is a C-linked triazinyl. In some embodiments, R⁴ is a C-linked indolyl. In some embodiments, R⁴ is a C-linked benzofuranyl. In some embodiments, R⁴ is a C-linked benzimidazolyl. In some embodiments, R⁴ is a C-linked indazolyl. In some embodiments, R⁴ is a C-linked pyrrolopyridinyl. In some embodiments, R⁴ is a C-linked imidazopyridinyl.

In yet another embodiment is a compound of Formula Ic, wherein R⁴ is a C-linked heteroaryl substituted with at least one group selected from halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, the C-linked heteroaryl is substituted with C₁-C₆alkyl. In another embodiment, C₁-C₆alkyl is methyl, ethyl, n-propyl, iso-propyl, n-butyl, iso-butyl, or tert-butyl. In a further embodiment, the C-linked heteroaryl is substituted with methyl. In another embodiment, the C-linked heteroaryl is substituted with ethyl. In a further embodiment, the C-linked heteroaryl is substituted with n-propyl or iso-propyl.

In another embodiment is a compound of Ic wherein R⁴ is a substituted or unsubstituted cycloalkyl. In a further embodiment, cycloalkyl is selected from cyclopropyl, cyclobutyl, cyclopentyl, or cyclohexyl. In a further embodiment, R⁴ is cyclopentyl. In another embodiment, R⁴ is cyclohexyl.

In another embodiment is a compound of Ic wherein R⁴ is a substituted or unsubstituted aryl. In another embodiment is a compound of Ic wherein R⁴ is a substituted or unsubstituted phenyl.

In another embodiment, are compounds having the structure of Formula II or pharmaceutically acceptable salt or N-oxide thereof:

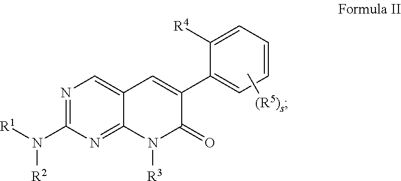

wherein:
R¹ is H, or substituted or unsubstituted alkyl;
R² is alkyl substituted with —OH, —OMe, —SH, —SMe, or halogen;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted heteroaryl attached to the phenyl ring via a carbon atom of R⁴, or substituted or unsubstituted heterocycloalkyl attached to the phenyl ring via a carbon atom of R⁴;
each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

In a further embodiment is a compound of Formula II, wherein R⁴ is a substituted or unsubstituted C-linked heterocycloalkyl. In a further embodiment, the C-linked heterocycloalkyl is pyrrolidinyl, tetrahydrofuranyl, piperidinyl, tetrahydropyranyl, tetrahydrothiopyranyl, morpholinyl, or piperazinyl. In some embodiments, the C-linked heterocycloalkyl is pyrrolidinyl. In some embodiments, the C-linked heterocycloalkyl is tetrahydrofuranyl. In some embodiments, the C-linked heterocycloalkyl is piperidinyl. In some embodiments, the C-linked heterocycloalkyl is tetrahydropyranyl. In some embodiments, the C-linked heterocycloalkyl is tetrahydrothiopyranyl. In some embodiments, the C-linked heterocycloalkyl is morpholinyl. In some embodiments, the C-linked heterocycloalkyl is piperazinyl. In a further embodiment, the C-linked heterocycloalkyl is substituted with at least one C₁-C₆alkyl or halogen. In another embodiment, the C₁-C₆alkyl is methyl, ethyl, or n-propyl.

In one embodiment is a compound of Formula II, wherein R⁴ is a substituted or unsubstituted C-linked heteroaryl. In one embodiment, R⁴ is selected from a C-linked pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl. In some embodiments, R⁴ is a C-linked pyrrolyl. In some embodiments, R⁴ is a C-linked furanyl. In some embodiments, R⁴ is a C-linked thiophenyl. In some embodiments, R⁴ is a C-linked pyrazolyl. In some embodiments, R⁴ is a C-linked imidazolyl. In some embodiments, R⁴ is a C-linked isoxazolyl. In some embodiments, R⁴ is a C-linked oxazolyl. In some embodiments, R⁴ is a C-linked isothiazolyl. In some embodiments, R⁴ is a C-linked thiazolyl. In some embodiments, R⁴ is a C-linked 1,2,3-triazolyl. In some embodiments, R⁴ is a C-linked 1,3,4-triazolyl. In some embodiments, R⁴ is a C-linked 1-oxa-2,3-diazolyl. In some embodiments, R⁴ is a C-linked 1-oxa-2,4-diazolyl. In some embodiments, R⁴ is a C-linked 1-oxa-2,5-diazolyl. In some embodiments, R⁴ is a C-linked 1-oxa-3,4-diazolyl. In some embodiments, R⁴ is a C-linked 1-thia-2,3-diazolyl. In some embodiments, R⁴ is a C-linked 1-thia-2,4-diazolyl. In some embodiments, R⁴ is a C-linked 1-thia-2,5-diazolyl. In some embodiments, R⁴ is a C-linked 1-thia-3,4-diazolyl. In some embodiments, R⁴ is a C-linked tetrazolyl. In some embodiments, R⁴ is a C-linked pyridinyl. In some embodiments, R⁴ is a C-linked pyridazinyl. In some embodiments, R⁴ is a C-linked pyrimidinyl. In some embodiments, R⁴ is a C-linked pyrazinyl. In some embodiments, R⁴ is a C-linked triazinyl. In some embodiments, R⁴ is a C-linked indolyl. In some embodiments, R⁴ is a C-linked benzofuranyl. In some embodiments, R⁴ is a C-linked benzimidazolyl. In some embodiments, R⁴ is a C-linked indazolyl. In some embodiments, R⁴ is a C-linked pyrrolopyridinyl. In some embodiments, R⁴ is a C-linked imidazopyridinyl.

In yet another embodiment is a compound of Formula II, wherein R⁴ is a C-linked heteroaryl substituted with at least one group selected from halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, the C-linked heteroaryl is substituted with C₁-C₆alkyl. In another embodiment, C₁-C₆alkyl is methyl, ethyl, n-propyl, iso-propyl, n-butyl, iso-butyl, or tert-butyl. In a further embodiment, the C-linked heteroaryl is substituted with methyl. In another embodiment, the C-linked heteroaryl is substituted with ethyl. In a further embodiment, the C-linked heteroaryl is substituted with n-propyl or iso-propyl.

In another embodiment, are compounds having the structure of Formula III or pharmaceutically acceptable salt or N-oxide thereof:

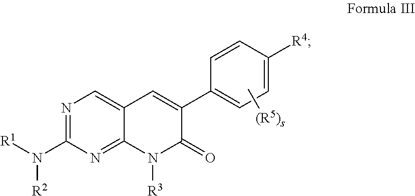

wherein:
R¹ is H, or substituted or unsubstituted alkyl;
R² is alkyl substituted with —OH, —OMe, —SH, —SMe, or halogen;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted heteroaryl attached to the phenyl ring via a carbon atom of R⁴, or substituted or unsubstituted heterocycloalkyl attached to the phenyl ring via a carbon atom of R⁴;
each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

In a further embodiment is a compound of Formula III, wherein R⁴ is a substituted or unsubstituted C-linked heterocycloalkyl. In a further embodiment, the C-linked heterocycloalkyl is pyrrolidinyl, tetrahydrofuranyl, piperidinyl, tetrahydropyranyl, tetrahydrothiopyranyl, morpholinyl, or piperazinyl. In some embodiments, the C-linked heterocycloalkyl is pyrrolidinyl. In some embodiments, the C-linked heterocycloalkyl is tetrahydrofuranyl. In some embodiments, the C-linked heterocycloalkyl is piperidinyl. In some embodiments, the C-linked heterocycloalkyl is tetrahydropyranyl. In some embodiments, the C-linked heterocycloalkyl is tetrahydrothiopyranyl. In some embodiments, the C-linked heterocycloalkyl is morpholinyl. In some embodiments, the C-linked heterocycloalkyl is piperazinyl. In a further embodiment, the C-linked heterocycloalkyl is substituted with at least one C₁-C₆alkyl or halogen. In another embodiment, the C₁-C₆alkyl is methyl, ethyl, or n-propyl.

In one embodiment is a compound of Formula III, wherein R⁴ is a substituted or unsubstituted C-linked heteroaryl. In one embodiment, R⁴ is selected from a C-linked pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl. In some embodiments, R⁴ is a C-linked pyrrolyl. In some embodiments, R⁴ is a C-linked furanyl. In some embodiments, R⁴ is a C-linked thiophenyl. In some embodiments, R⁴ is a C-linked pyrazolyl. In some embodiments, R⁴ is a C-linked imidazolyl. In some embodiments, R⁴ is a C-linked isoxazolyl. In some embodiments, R⁴ is a C-linked oxazolyl. In some embodiments, R⁴ is a C-linked isothiazolyl. In some embodiments, R⁴ is a C-linked thiazolyl. In some embodiments, R⁴ is a C-linked 1,2,3-triazolyl. In some embodiments, R⁴ is a C-linked 1,3,4-triazolyl. In some embodiments, R⁴ is a C-linked 1-oxa-2,3-diazolyl. In some embodiments, R⁴ is a C-linked 1-oxa-2,4-diazolyl. In some embodiments, R⁴ is a C-linked 1-oxa-2,5-diazolyl. In some embodiments, R⁴ is a C-linked 1-oxa-3,4-diazolyl. In some embodiments, R⁴ is a C-linked 1-thia-2,3-diazolyl. In some embodiments, R⁴ is a C-linked 1-thia-2,4-diazolyl. In some embodiments, R⁴ is a C-linked 1-thia-2,5-diazolyl. In some embodiments, R⁴ is a C-linked 1-thia-3,4-diazolyl. In some embodiments, R⁴ is a C-linked tetrazolyl. In some embodiments, R⁴ is a C-linked pyridinyl. In some embodiments, R⁴ is a C-linked pyridazinyl. In some embodiments, R⁴ is a C-linked pyrimidinyl. In some embodiments, R⁴ is a C-linked pyrazinyl. In some embodiments, R⁴ is a C-linked triazinyl. In some embodiments, R⁴ is a C-linked indolyl. In some embodiments, R⁴ is a C-linked benzofuranyl. In some embodiments, R⁴ is a C-linked benzimidazolyl. In some embodiments, R⁴ is a C-linked indazolyl. In some embodiments, R⁴ is a C-linked pyrrolopyridinyl. In some embodiments, R⁴ is a C-linked imidazopyridinyl.

In yet another embodiment is a compound of Formula III, wherein R⁴ is a C-linked heteroaryl substituted with at least one group selected from halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, the C-linked heteroaryl is substituted with C₁-C₆alkyl. In another embodiment, C₁-C₆alkyl is methyl, ethyl, n-propyl, iso-propyl, n-butyl, iso-butyl, or tert-butyl. In a further embodiment, the C-linked heteroaryl is substituted with methyl. In another embodiment, the C-linked heteroaryl is substituted with ethyl. In a further embodiment, the C-linked heteroaryl is substituted with n-propyl or iso-propyl.

In another embodiment is a compound of Formula III having the structure of Formula IIIa:

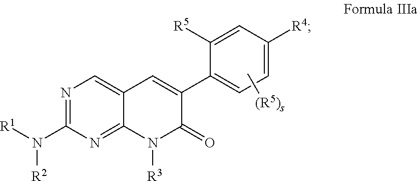

wherein R¹, R², R³, R⁴, R⁵ are described previously and s is 0-3.

In another embodiment is a compound of Formula III having the structure of Formula Mb:

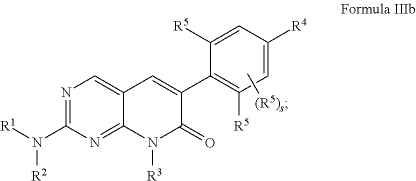

wherein R¹, R², R³, R⁴, R⁵ are described previously and s is 0-2.

In another embodiment, are compounds having the structure of Formula IV or pharmaceutically acceptable salt or N-oxide thereof:

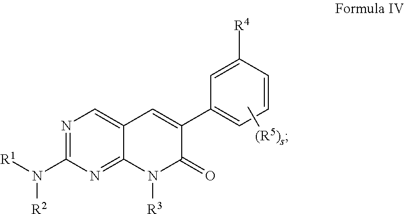

wherein:
R¹ is H, or substituted or unsubstituted alkyl;
R² is alkyl substituted with —OH, —OMe, —SH, —SMe, or halogen;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted 6-membered monocyclic heteroaryl ring attached to the phenyl ring via a carbon atom of R⁴, substituted or unsubstituted bicyclic heteroaryl ring attached to the phenyl ring via a carbon atom of R⁴, or substituted or unsubstituted heterocycloalkyl attached to the phenyl ring via a carbon atom of R⁴;
each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

In a further embodiment is a compound of Formula IV, wherein R⁴ is a substituted or unsubstituted C-linked heterocycloalkyl. In a further embodiment, the C-linked heterocycloalkyl is pyrrolidinyl, tetrahydrofuranyl, piperidinyl, tetrahydropyranyl, tetrahydrothiopyranyl, morpholinyl, or piperazinyl. In some embodiments, the C-linked heterocycloalkyl is pyrrolidinyl. In some embodiments, the C-linked heterocycloalkyl is tetrahydrofuranyl. In some embodiments, the C-linked heterocycloalkyl is piperidinyl. In some embodiments, the C-linked heterocycloalkyl is tetrahydropyranyl. In some embodiments, the C-linked heterocycloalkyl is tetrahydrothiopyranyl. In some embodiments, the C-linked heterocycloalkyl is morpholinyl. In some embodiments, the C-linked heterocycloalkyl is piperazinyl. In a further embodiment, the C-linked heterocycloalkyl is substituted with at least one C₁-C₆alkyl or halogen. In another embodiment, the C₁-C₆alkyl is methyl, ethyl, or n-propyl.

In another embodiment is a compound of Formula IV, wherein R⁴ is a substituted or unsubstituted C-linked 6-membered monocyclic heteroaryl ring. In some embodiments, R⁴ is selected from a C-linked pyridine, pyridazinyl, pyrimidinyl, pyrazinyl, and triazinyl. In some embodiments, R⁴ is a C-linked pyridinyl. In some embodiments, R⁴ is a C-linked pyridazinyl. In some embodiments, R⁴ is a C-linked pyrimidinyl. In some embodiments, R⁴ is a C-linked pyrazinyl. In some embodiments, R⁴ is a C-linked triazinyl.

In another embodiment is a compound of Formula IV, wherein R⁴ is a substituted or unsubstituted C-linked bicyclic heteroaryl ring. In some embodiments, R⁴ is selected from a C-linked indolyl, benzofuranyl, benzimidazolyl, indazolyl, pyrrolopyridinyl, and imidazopyridinyl. In some embodiments, R⁴ is a C-linked indolyl. In some embodiments, R⁴ is a C-linked benzofuranyl. In some embodiments, R⁴ is a C-linked benzimidazolyl. In some embodiments, R⁴ is a C-linked indazolyl. In some embodiments, R⁴ is a C-linked pyrrolopyridinyl. In some embodiments, R⁴ is a C-linked imidazopyridinyl.

In another embodiment is a compound of Formula IV, wherein R⁴ is a C-linked 6-membered monocyclic heteroaryl ring substituted with at least one group selected from halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, the C-linked heteroaryl is substituted with C₁-C₆alkyl. In another embodiment, C₁-C₆alkyl is methyl, ethyl, n-propyl, iso-propyl, n-butyl, iso-butyl, or tert-butyl. In a further embodiment, the C-linked heteroaryl is substituted with methyl. In another embodiment, the C-linked heteroaryl is substituted with ethyl. In a further embodiment, the C-linked heteroaryl is substituted with n-propyl or iso-propyl.

In yet another embodiment is a compound of Formula IV, wherein R⁴ is a C-linked bicyclic heteroaryl ring substituted with at least one group selected from halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R¹⁰, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, the C-linked heteroaryl is substituted with C₁-C₆alkyl. In another embodiment, C₁-C₆alkyl is methyl, ethyl, n-propyl, iso-propyl, n-butyl, iso-butyl, or tert-butyl. In a further embodiment, the C-linked heteroaryl is substituted with methyl. In another embodiment, the C-linked heteroaryl is substituted with ethyl. In a further embodiment, the C-linked heteroaryl is substituted with n-propyl or iso-propyl.

In further embodiments of any of the aforementioned embodiments, each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl. In a further embodiment, each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, or substituted or unsubstituted alkoxy. In yet a further embodiment, each R⁵ is independently halogen, —N(R¹⁰)₂, or substituted or unsubstituted alkyl. In some embodiments, R⁵ is halogen. In some embodiments, R⁵ is fluoro. In some embodiments, R⁵ is chloro. In some embodiments, R⁵ is —N(R¹⁰)₂. In some embodiments, R⁵ is dimethylamino. In some embodiments, R⁵ is substituted or unsubstituted alkyl. In some embodiments, R⁵ is methyl. In some embodiments, R⁵ is ethyl. In some embodiments, R⁵ is propyl. In some embodiments, R⁵ is isopropyl.

In further embodiments of any of the aforementioned embodiments, s is 0. In a further embodiment of any of the aforementioned embodiments, s is 1. In a further embodiment of any of the aforementioned embodiments, s is 2.

In further embodiments of any of the aforementioned embodiments, R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl. In a further embodiment, R³ is H. In a further embodiment, R³ is substituted or unsubstituted alkoxy or a substituted or unsubstituted amino. In a further embodiment, R³ is substituted or unsubstituted alkyl or a substituted or unsubstituted heteroalkyl. In a further embodiment, R³ is substituted or unsubstituted cycloalkyl or a substituted or unsubstituted heterocycloalkyl. In a further embodiment, R³ is substituted or unsubstituted cycloalkylalkyl or a substituted or unsubstituted heterocycloalkylalkyl. In a further embodiment, R³ is substituted or unsubstituted aryl or a substituted or unsubstituted heteroaryl. In a further embodiment, R³ is substituted or unsubstituted aralkyl or a substituted or unsubstituted heteroarylalkyl. In a further embodiment, R³ is substituted or unsubstituted alkyl. In a further embodiment, R³ is methyl. In a further embodiment, R³ is ethyl. In a further embodiment, R³ is propyl. In a further embodiment, R³ is isopropyl. In a further embodiment, R³ is substituted or unsubstituted alkoxy. In a further embodiment, R³ is substituted or unsubstituted methoxy. In a further embodiment, R³ is substituted or unsubstituted ethoxy. In a further embodiment, R³ is substituted or unsubstituted amino. In a further embodiment, R³ is substituted or unsubstituted heteroalkyl. In a further embodiment, R³ is substituted or unsubstituted heterocycloalkyl. In a further embodiment, R³ is pyrrolidinyl, tetrahydrofuranyl, piperidinyl, tetrahydropyranyl, tetrahydrothiopyranyl, morpholinyl, or piperazinyl. In a further embodiment, R³ is substituted or unsubstituted cycloalkyl. In a further embodiment, R³ is cyclopropyl, cyclobutyl, cyclopentyl, cyclohexyl or cycloheptyl. In a further embodiment, R³ is substituted or unsubstituted cycloalkylalkyl. In a further embodiment, R³ is substituted or unsubstituted heterocycloalkylalkyl. In a further embodiment, R³ is substituted or unsubstituted aryl. In a further embodiment, R³ is substituted or unsubstituted phenyl. In a further embodiment, R³ is substituted or unsubstituted aralkyl. In a further embodiment, R³ is substituted or unsubstituted heteroaryl. In a further embodiment, R³ is pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, or imidazopyridinyl. In a further embodiment, R³ is substituted or unsubstituted heteroarylalkyl.

In a further embodiment of any of the aforementioned embodiments, R² is C₁-C₄alkyl substituted with hydroxy or C1-C₄alkyl substituted with methoxy. In a refinement, R² is —CH₂CH₂OH. In another refinement, R² is —CH₂CH₂OCH₃. In another refinement, R² is —CH₂CH₂CH₂OH. In another refinement, R² is —CH₂CH₂CH₂O CH₃. In another refinement, R² is —CH₂C(CH₃)₂OH.

In another further embodiment of the aforementioned embodiments, R² is —CH(CH₂CH₂OH)₂.

In a further embodiment of any of the aforementioned embodiments, R¹ is H. In a further embodiment of any of the aforementioned embodiments, R¹ is substituted or unsubstituted alkyl. In a further embodiment, R¹ is methyl. In a further embodiment, R¹ is ethyl. In a further embodiment, R¹ is propyl. In a further embodiment, R¹ is isopropyl.

In a further aspect is a compound having the structure:

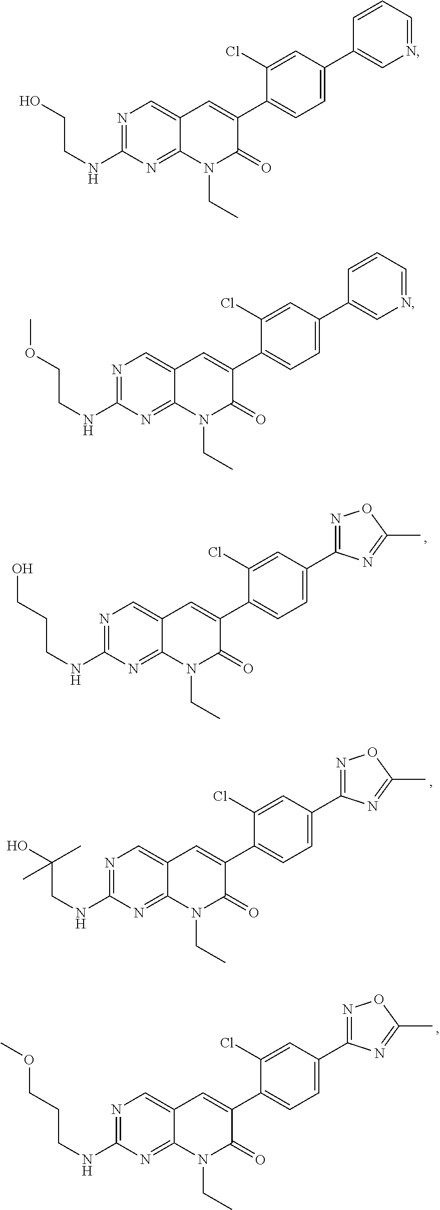

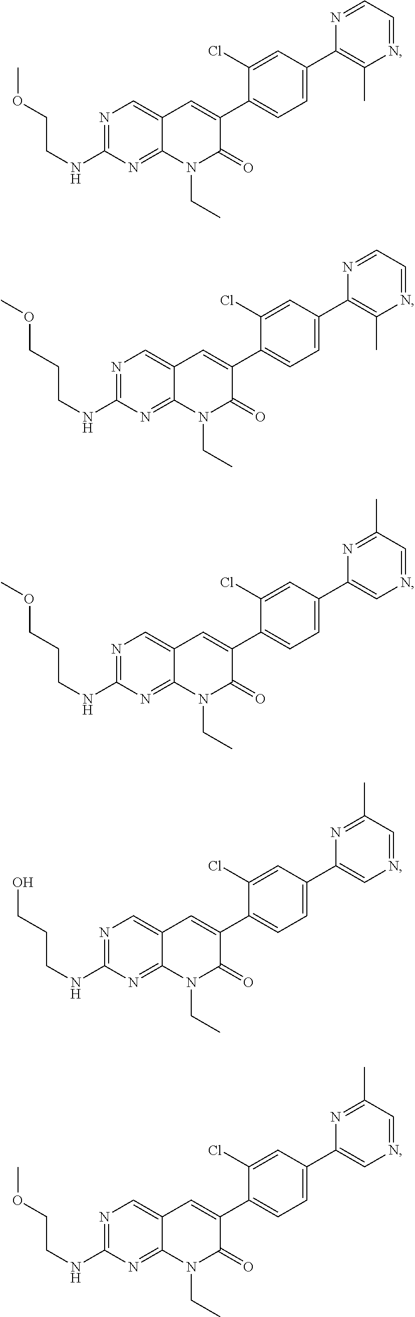 [SMILES: CCn1c(=O)c(-c2ccc(-c3nccnc3C)cc2Cl)cc2cnc(NCCOC)nc21 ; CCn1c(=O)c(-c2ccc(-c3nccnc3C)cc2Cl)cc2cnc(NCCCOC)nc21 ; CCn1c(=O)c(-c2ccc(-c3cncc(C)n3)cc2Cl)cc2cnc(NCCCOC)nc21 ; CCn1c(=O)c(-c2ccc(-c3cncc(C)n3)cc2Cl)cc2cnc(NCCCO)nc21 ; CCn1c(=O)c(-c2ccc(-c3cncc(C)n3)cc2Cl)cc2cnc(NCCOC)nc21]

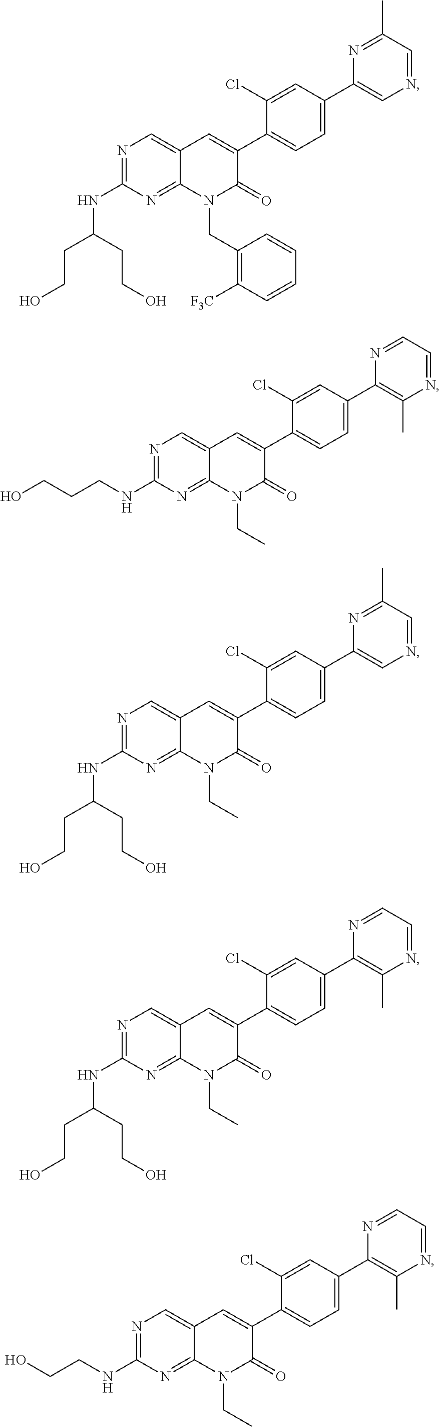 [SMILES: Cc1cncc(-c2ccc(-c3cc4cnc(NC(CCO)CCO)nc4n(Cc4ccccc4C(F)(F)F)c3=O)c(Cl)c2)n1 ; CCn1c(=O)c(-c2ccc(-c3nccnc3C)cc2Cl)cc2cnc(NCCCO)nc21 ; CCn1c(=O)c(-c2ccc(-c3cncc(C)n3)cc2Cl)cc2cnc(NC(CCO)CCO)nc21 ; CCn1c(=O)c(-c2ccc(-c3nccnc3C)cc2Cl)cc2cnc(NC(CCO)CCO)nc21 ; CCn1c(=O)c(-c2ccc(-c3nccnc3C)cc2Cl)cc2cnc(NCCO)nc21]

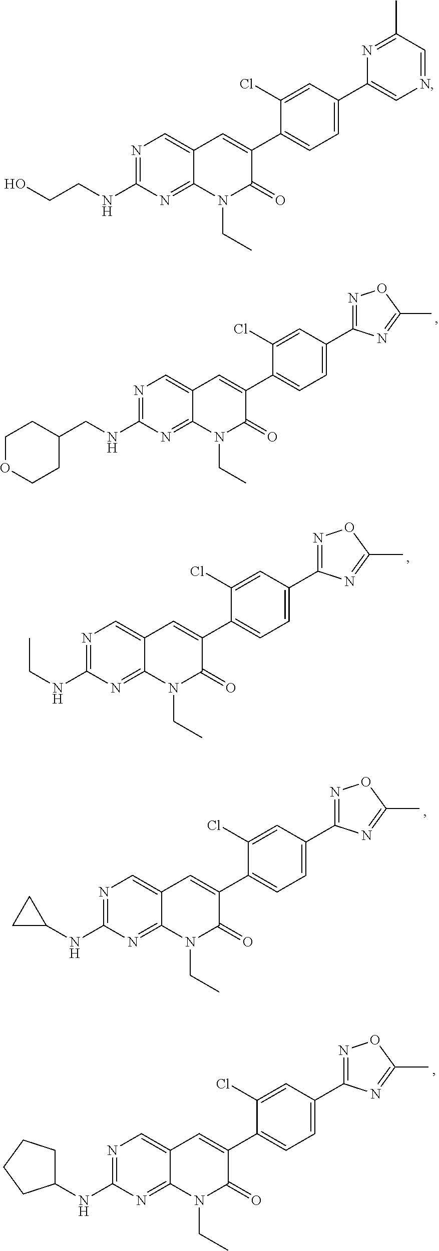 [SMILES: CCn1c(=O)c(-c2ccc(-c3cncc(C)n3)cc2Cl)cc2cnc(NCCO)nc21 ; CCn1c(=O)c(-c2ccc(-c3noc(C)n3)cc2Cl)cc2cnc(NCC3CCOCC3)nc21 ; CCNc1ncc2cc(-c3ccc(-c4noc(C)n4)cc3Cl)c(=O)n(CC)c2n1 ; CCn1c(=O)c(-c2ccc(-c3noc(C)n3)cc2Cl)cc2cnc(NC3CC3)nc21 ; CCn1c(=O)c(-c2ccc(-c3noc(C)n3)cc2Cl)cc2cnc(NC3CCCC3)nc21]

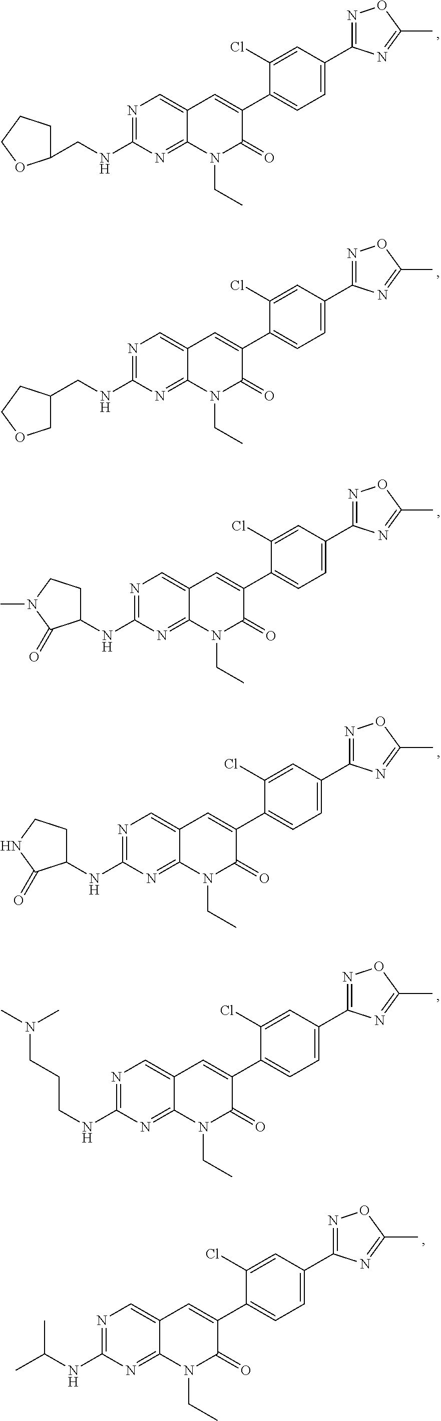

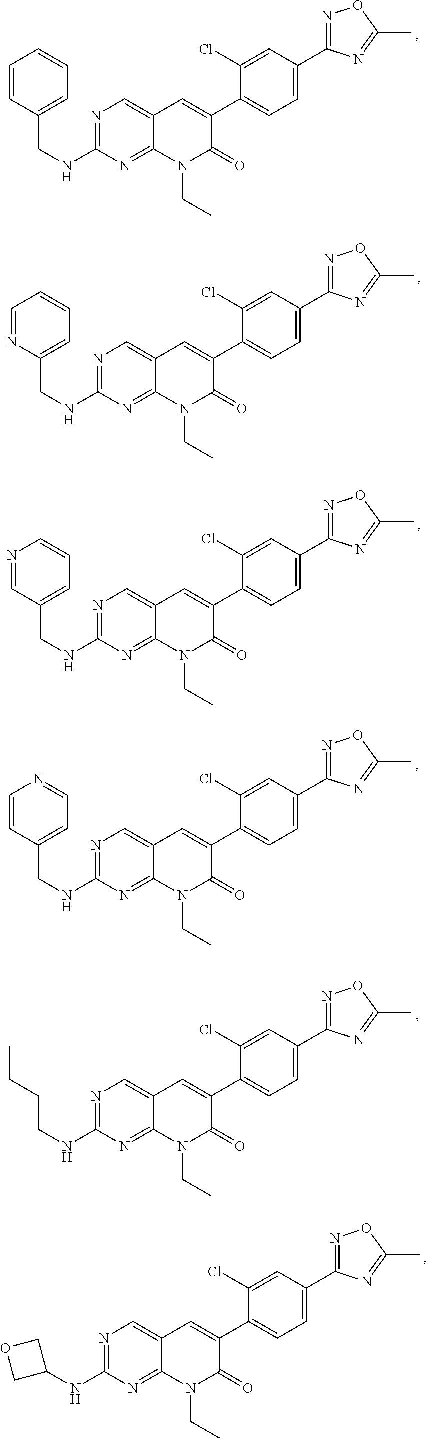

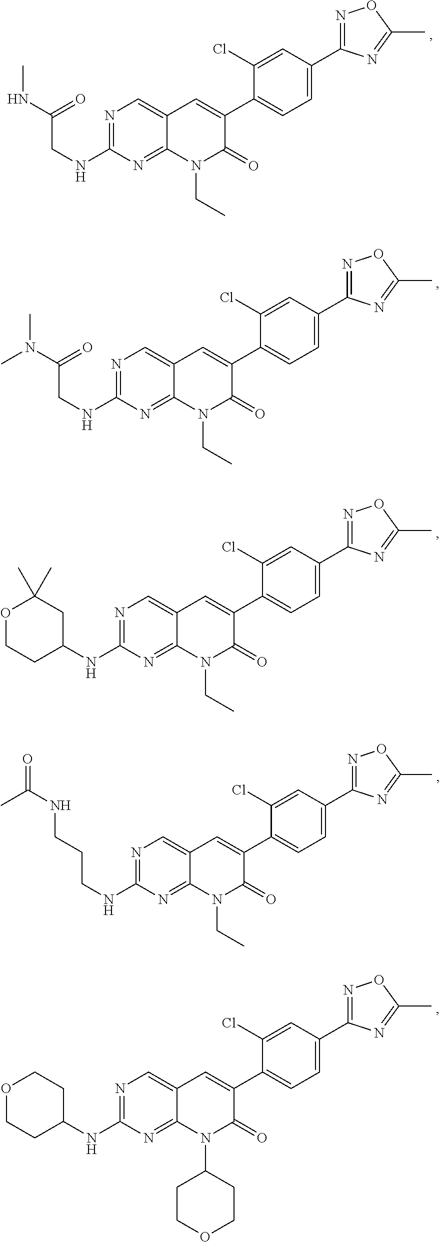

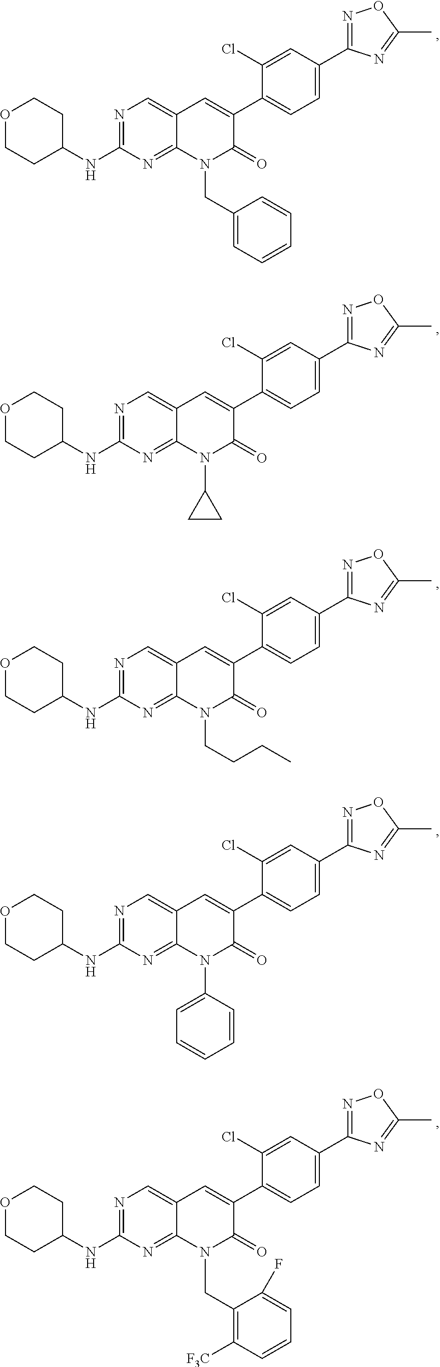 [SMILES: Cc1nc(-c2ccc(-c3cc4cnc(NC5CCOCC5)nc4n(Cc4ccccc4)c3=O)c(Cl)c2)no1 ; Cc1nc(-c2ccc(-c3cc4cnc(NC5CCOCC5)nc4n(C4CC4)c3=O)c(Cl)c2)no1 ; CCCCn1c(=O)c(-c2ccc(-c3noc(C)n3)cc2Cl)cc2cnc(NC3CCOCC3)nc21 ; Cc1nc(-c2ccc(-c3cc4cnc(NC5CCOCC5)nc4n(-c4ccccc4)c3=O)c(Cl)c2)no1 ; Cc1nc(-c2ccc(-c3cc4cnc(NC5CCOCC5)nc4n(Cc4c(F)cccc4C(F)(F)F)c3=O)c(Cl)c2)no1]

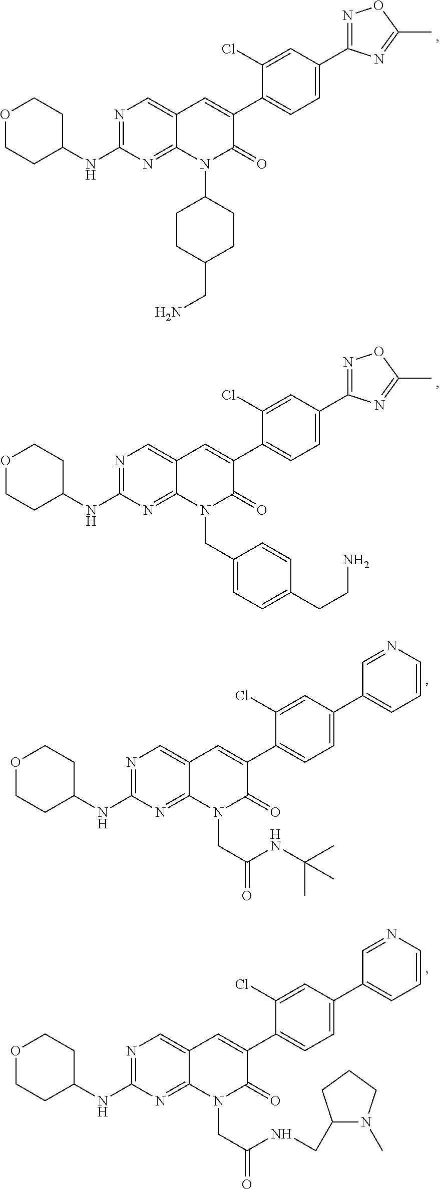 [SMILES: Cc1nc(-c2ccc(-c3cc4cnc(NC5CCOCC5)nc4n(C4CCC(CN)CC4)c3=O)c(Cl)c2)no1 ; Cc1nc(-c2ccc(-c3cc4cnc(NC5CCOCC5)nc4n(Cc4ccc(CCN)cc4)c3=O)c(Cl)c2)no1 ; CC(C)(C)NC(=O)Cn1c(=O)c(-c2ccc(-c3cccnc3)cc2Cl)cc2cnc(NC3CCOCC3)nc21 ; CN1CCCC1CNC(=O)Cn1c(=O)c(-c2ccc(-c3cccnc3)cc2Cl)cc2cnc(NC3CCOCC3)nc21]

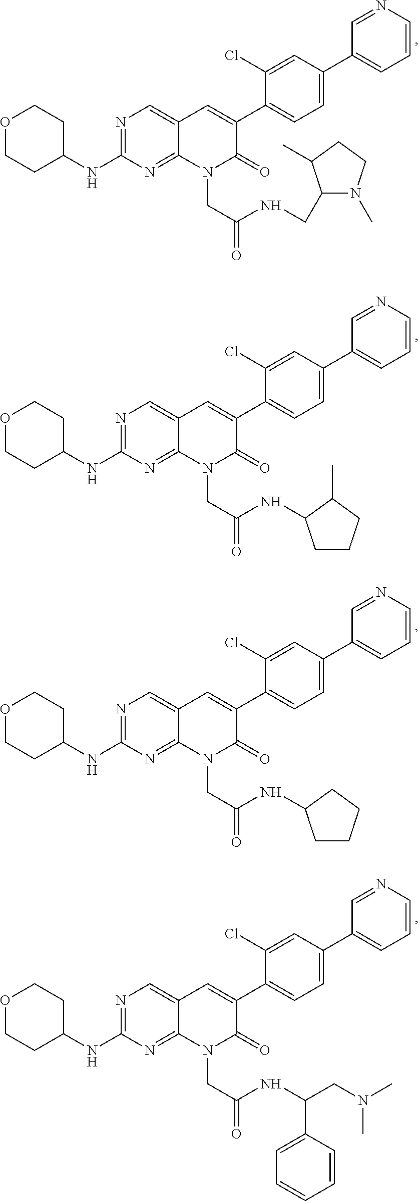 [SMILES: CC1CCN(C)C1CNC(=O)Cn1c(=O)c(-c2ccc(-c3cccnc3)cc2Cl)cc2cnc(NC3CCOCC3)nc21 ; CC1CCCC1NC(=O)Cn1c(=O)c(-c2ccc(-c3cccnc3)cc2Cl)cc2cnc(NC3CCOCC3)nc21 ; O=C(Cn1c(=O)c(-c2ccc(-c3cccnc3)cc2Cl)cc2cnc(NC3CCOCC3)nc21)NC1CCCC1 ; CN(C)CC(NC(=O)Cn1c(=O)c(-c2ccc(-c3cccnc3)cc2Cl)cc2cnc(NC3CCOCC3)nc21)c1ccccc1]

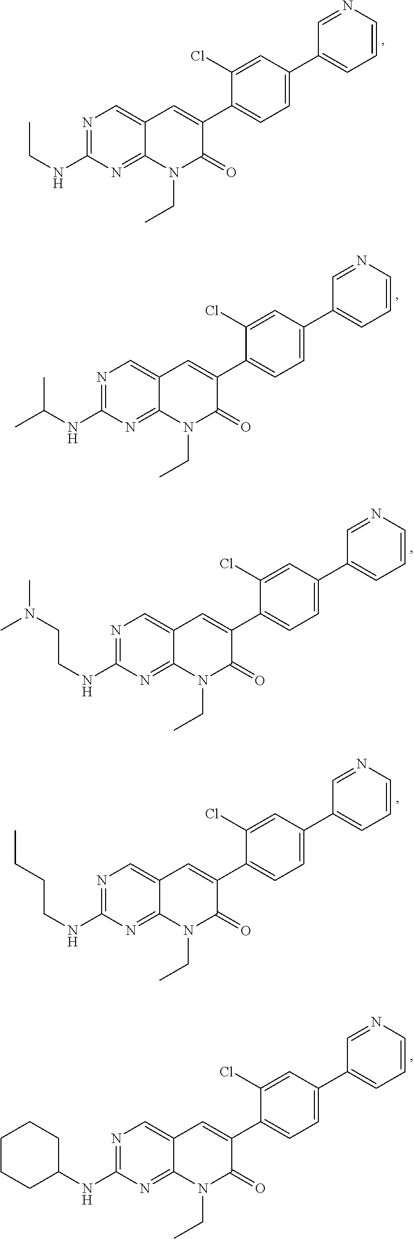 [SMILES: CCNc1ncc2cc(-c3ccc(-c4cccnc4)cc3Cl)c(=O)n(CC)c2n1 ; CCn1c(=O)c(-c2ccc(-c3cccnc3)cc2Cl)cc2cnc(NC(C)C)nc21 ; CCn1c(=O)c(-c2ccc(-c3cccnc3)cc2Cl)cc2cnc(NCCN(C)C)nc21 ; CCCCNc1ncc2cc(-c3ccc(-c4cccnc4)cc3Cl)c(=O)n(CC)c2n1 ; CCn1c(=O)c(-c2ccc(-c3cccnc3)cc2Cl)cc2cnc(NC3CCCCC3)nc21]

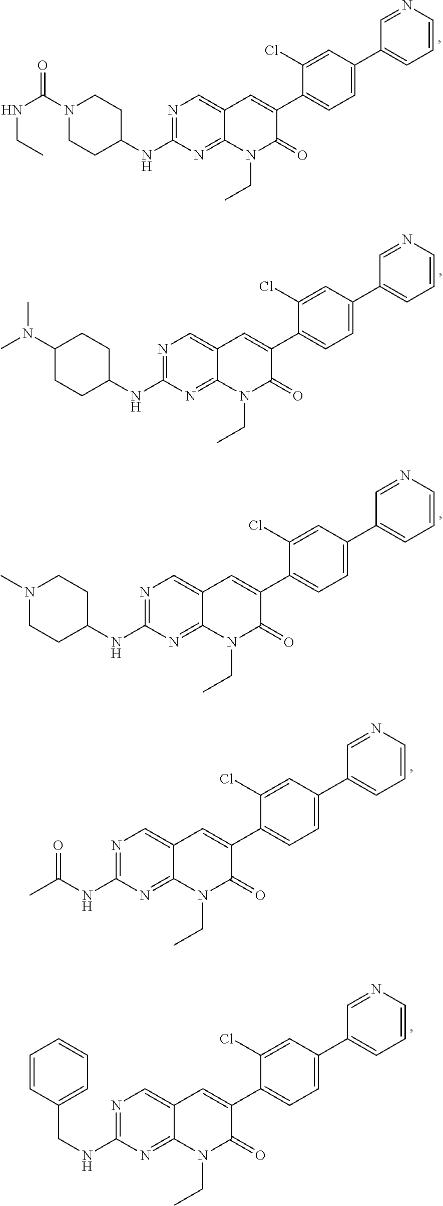 [SMILES: CCNC(=O)N1CCC(Nc2ncc3cc(-c4ccc(-c5cccnc5)cc4Cl)c(=O)n(CC)c3n2)CC1 ; CCn1c(=O)c(-c2ccc(-c3cccnc3)cc2Cl)cc2cnc(NC3CCC(N(C)C)CC3)nc21 ; CCn1c(=O)c(-c2ccc(-c3cccnc3)cc2Cl)cc2cnc(NC3CCN(C)CC3)nc21 ; CCn1c(=O)c(-c2ccc(-c3cccnc3)cc2Cl)cc2cnc(NC(C)=O)nc21 ; CCn1c(=O)c(-c2ccc(-c3cccnc3)cc2Cl)cc2cnc(NCc3ccccc3)nc21]

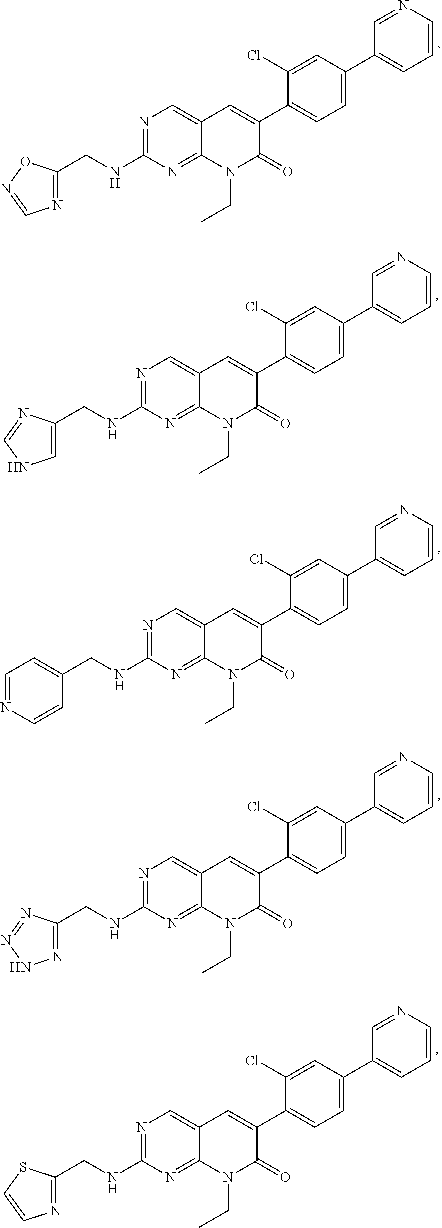 [SMILES: CCn1c(=O)c(-c2ccc(-c3cccnc3)cc2Cl)cc2cnc(NCc3ncno3)nc21 ; CCn1c(=O)c(-c2ccc(-c3cccnc3)cc2Cl)cc2cnc(NCc3c[nH]cn3)nc21 ; CCn1c(=O)c(-c2ccc(-c3cccnc3)cc2Cl)cc2cnc(NCc3ccncc3)nc21 ; CCn1c(=O)c(-c2ccc(-c3cccnc3)cc2Cl)cc2cnc(NCc3nn[nH]n3)nc21 ; CCn1c(=O)c(-c2ccc(-c3cccnc3)cc2Cl)cc2cnc(NCc3nccs3)nc21]

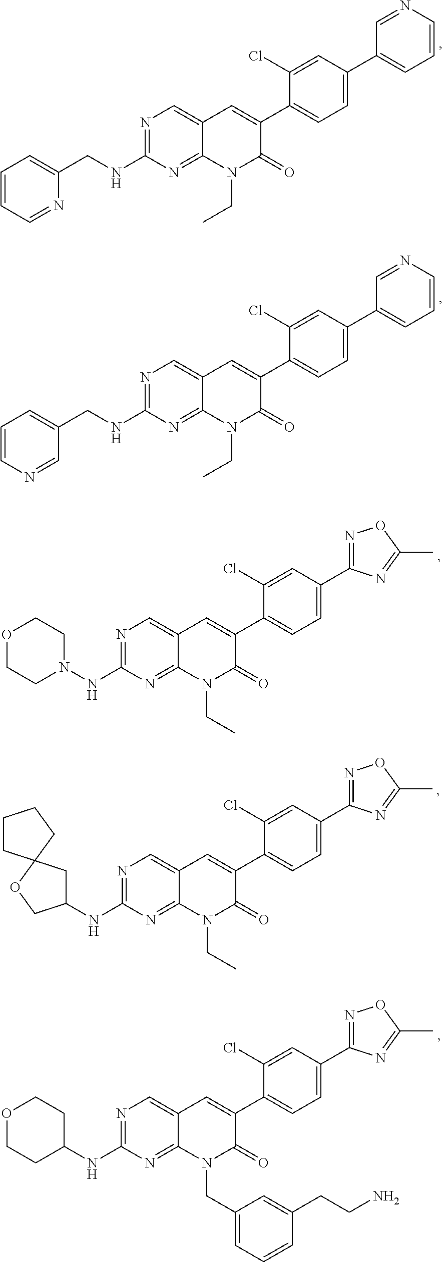 [SMILES: CCn1c(=O)c(-c2ccc(-c3cccnc3)cc2Cl)cc2cnc(NCc3ccccn3)nc21 ; CCn1c(=O)c(-c2ccc(-c3cccnc3)cc2Cl)cc2cnc(NCc3cccnc3)nc21 ; CCn1c(=O)c(-c2ccc(-c3noc(C)n3)cc2Cl)cc2cnc(NN3CCOCC3)nc21 ; CCn1c(=O)c(-c2ccc(-c3noc(C)n3)cc2Cl)cc2cnc(NC3COC4(CCCC4)C3)nc21 ; Cc1nc(-c2ccc(-c3cc4cnc(NC5CCOCC5)nc4n(Cc4cccc(CCN)c4)c3=O)c(Cl)c2)no1]

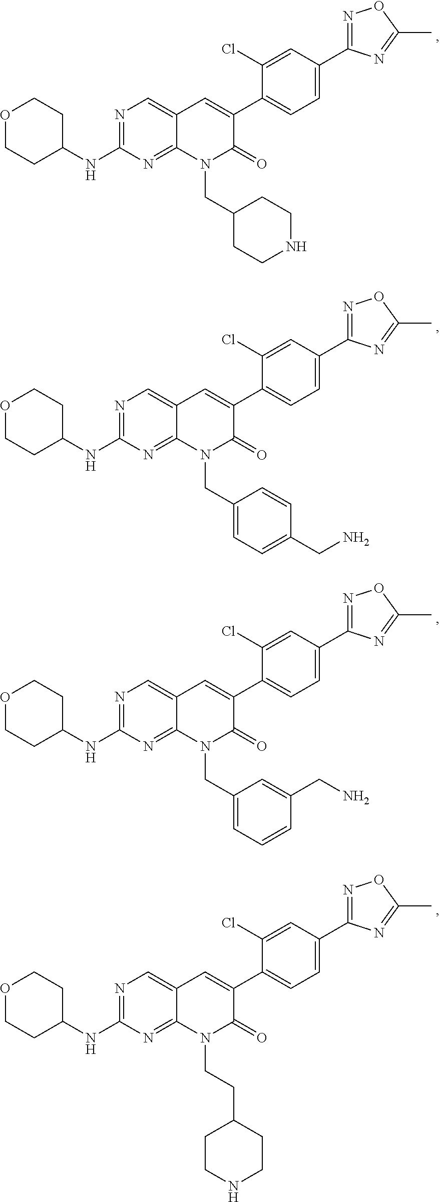 [SMILES: Cc1nc(-c2ccc(-c3cc4cnc(NC5CCOCC5)nc4n(CC4CCNCC4)c3=O)c(Cl)c2)no1 ; Cc1nc(-c2ccc(-c3cc4cnc(NC5CCOCC5)nc4n(Cc4ccc(CN)cc4)c3=O)c(Cl)c2)no1 ; Cc1nc(-c2ccc(-c3cc4cnc(NC5CCOCC5)nc4n(Cc4cccc(CN)c4)c3=O)c(Cl)c2)no1 ; Cc1nc(-c2ccc(-c3cc4cnc(NC5CCOCC5)nc4n(CCC4CCNCC4)c3=O)c(Cl)c2)no1]

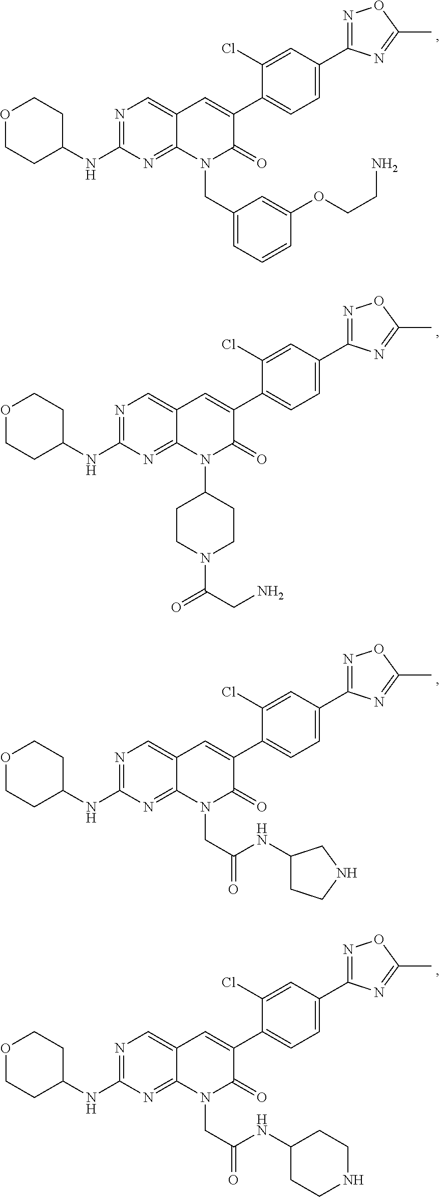 [SMILES: Cc1nc(-c2ccc(-c3cc4cnc(NC5CCOCC5)nc4n(Cc4cccc(OCCN)c4)c3=O)c(Cl)c2)no1 ; Cc1nc(-c2ccc(-c3cc4cnc(NC5CCOCC5)nc4n(C4CCN(C(=O)CN)CC4)c3=O)c(Cl)c2)no1 ; Cc1nc(-c2ccc(-c3cc4cnc(NC5CCOCC5)nc4n(CC(=O)NC4CCNC4)c3=O)c(Cl)c2)no1 ; Cc1nc(-c2ccc(-c3cc4cnc(NC5CCOCC5)nc4n(CC(=O)NC4CCNCC4)c3=O)c(Cl)c2)no1]

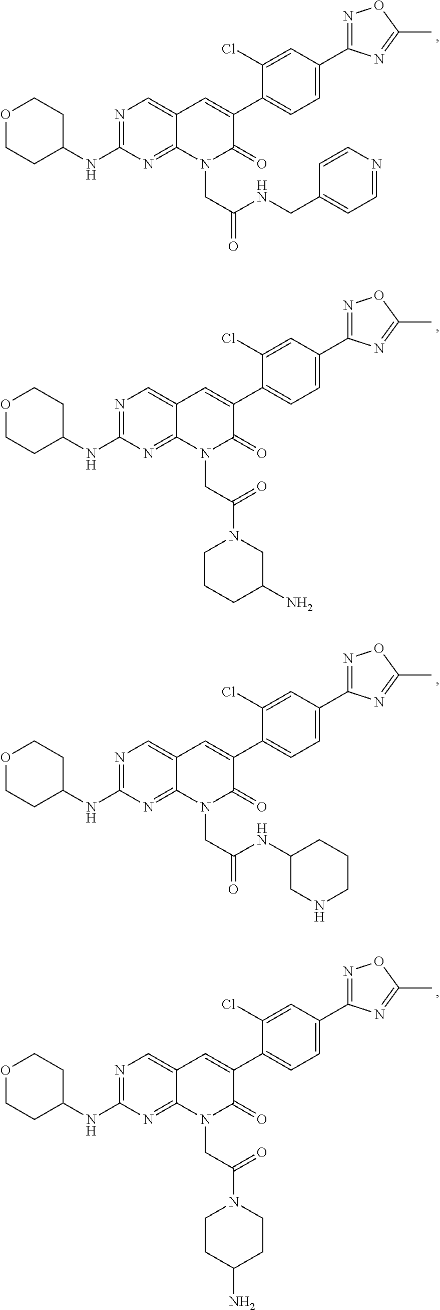 [SMILES: Cc1nc(-c2ccc(-c3cc4cnc(NC5CCOCC5)nc4n(CC(=O)NCc4ccncc4)c3=O)c(Cl)c2)no1 ; Cc1nc(-c2ccc(-c3cc4cnc(NC5CCOCC5)nc4n(CC(=O)N4CCCC(N)C4)c3=O)c(Cl)c2)no1 ; Cc1nc(-c2ccc(-c3cc4cnc(NC5CCOCC5)nc4n(CC(=O)NC4CCCNC4)c3=O)c(Cl)c2)no1 ; Cc1nc(-c2ccc(-c3cc4cnc(NC5CCOCC5)nc4n(CC(=O)N4CCC(N)CC4)c3=O)c(Cl)c2)no1]

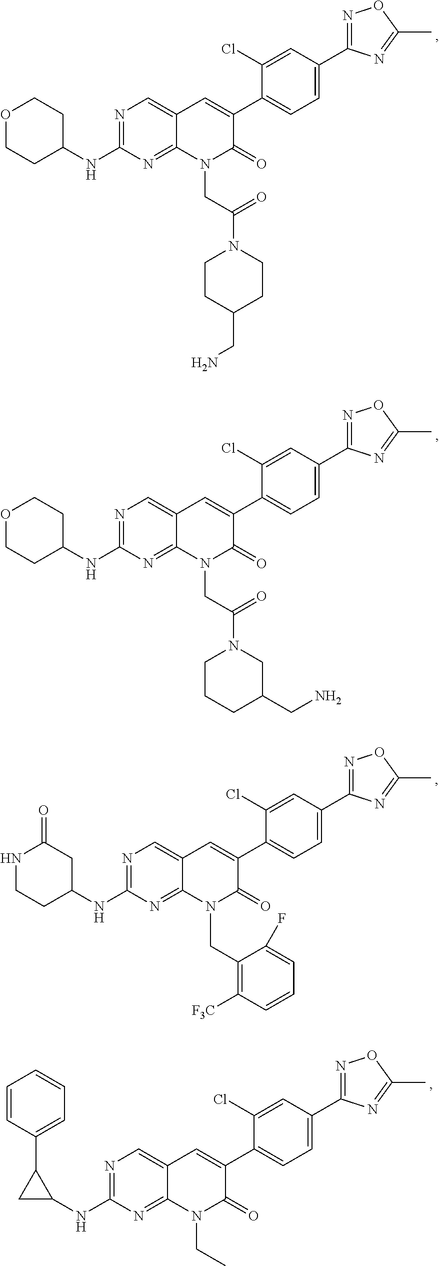 [SMILES: Cc1nc(-c2ccc(-c3cc4cnc(NC5CCOCC5)nc4n(CC(=O)N4CCC(CN)CC4)c3=O)c(Cl)c2)no1 ; Cc1nc(-c2ccc(-c3cc4cnc(NC5CCOCC5)nc4n(CC(=O)N4CCCC(CN)C4)c3=O)c(Cl)c2)no1 ; Cc1nc(-c2ccc(-c3cc4cnc(NC5CCNC(=O)C5)nc4n(Cc4c(F)cccc4C(F)(F)F)c3=O)c(Cl)c2)no1 ; CCn1c(=O)c(-c2ccc(-c3noc(C)n3)cc2Cl)cc2cnc(NC3CC3c3ccccc3)nc21]

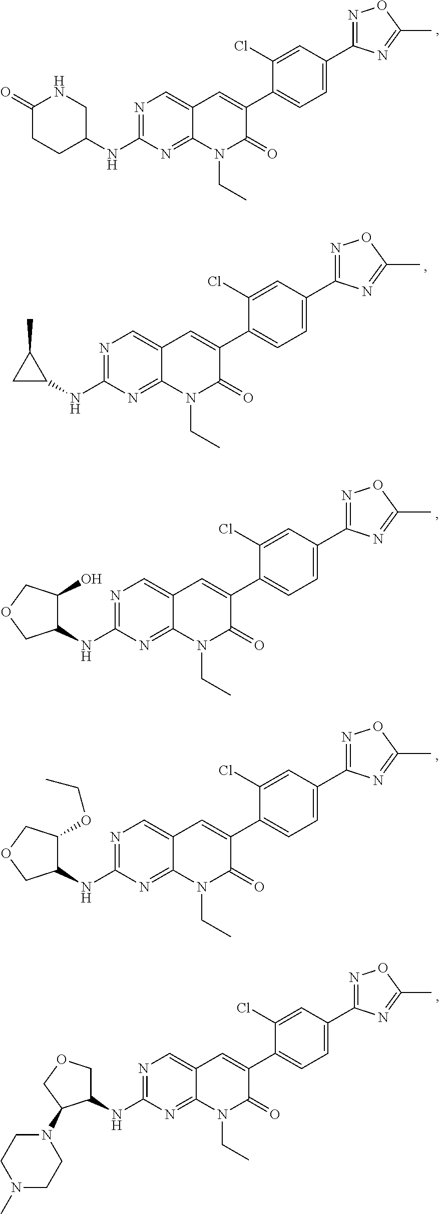

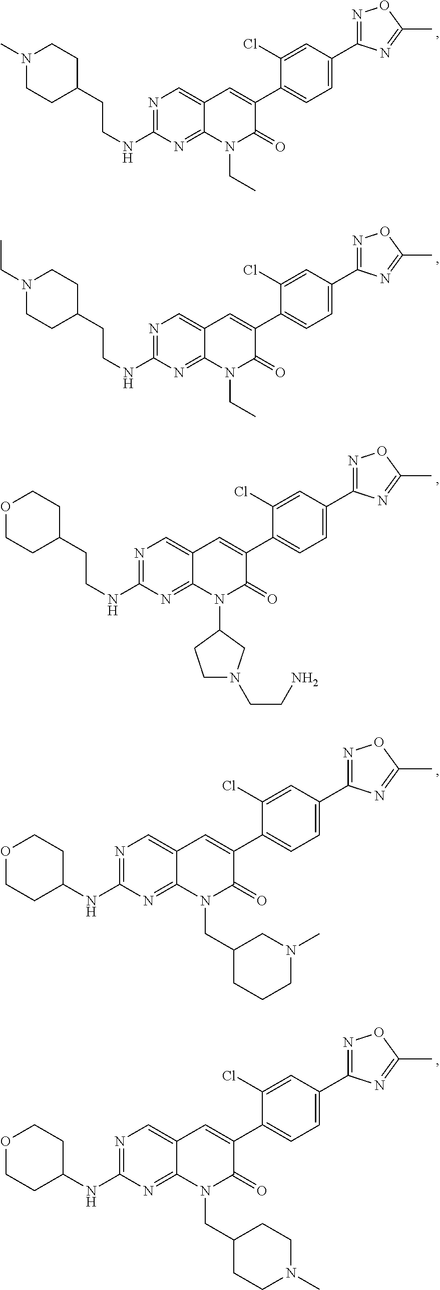

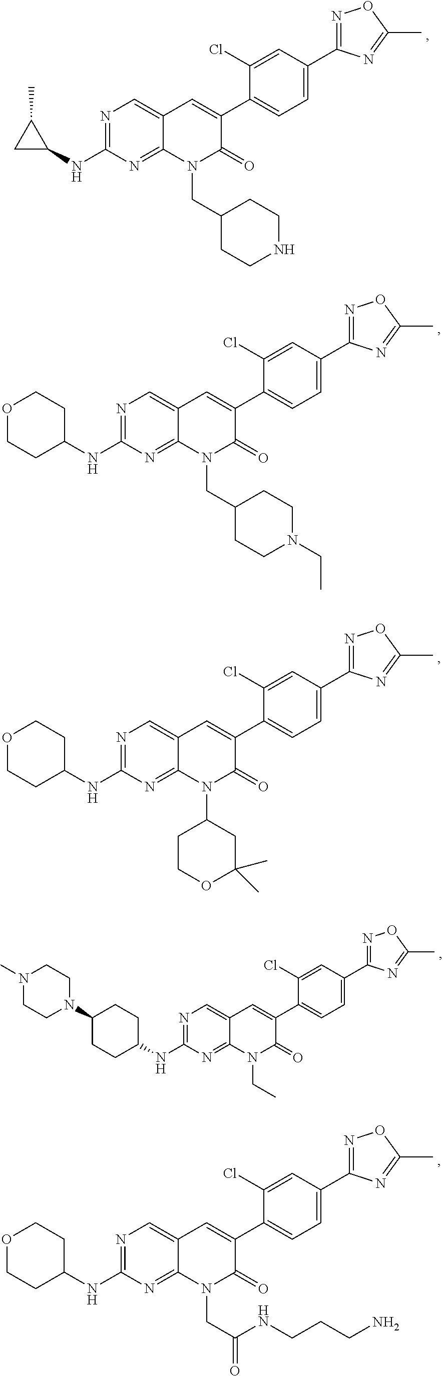

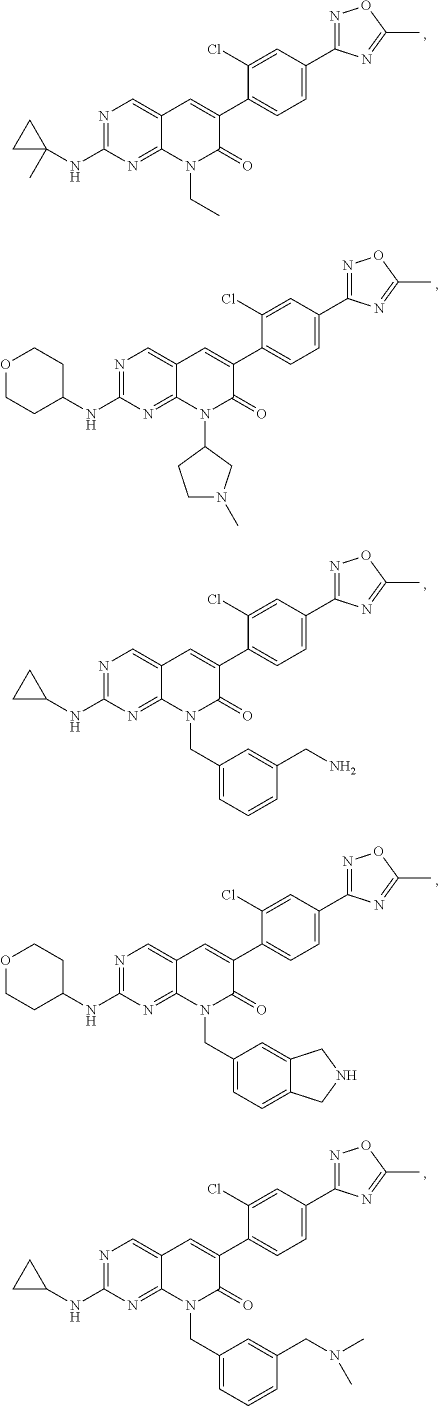 [SMILES: CCn1c(=O)c(-c2ccc(-c3noc(C)n3)cc2Cl)cc2cnc(NC3(C)CC3)nc21 ; Cc1nc(-c2ccc(-c3cc4cnc(NC5CCOCC5)nc4n(C4CCN(C)C4)c3=O)c(Cl)c2)no1 ; Cc1nc(-c2ccc(-c3cc4cnc(NC5CC5)nc4n(Cc4cccc(CN)c4)c3=O)c(Cl)c2)no1 ; Cc1nc(-c2ccc(-c3cc4cnc(NC5CCOCC5)nc4n(Cc4ccc5c(c4)CNC5)c3=O)c(Cl)c2)no1 ; Cc1nc(-c2ccc(-c3cc4cnc(NC5CC5)nc4n(Cc4cccc(CN(C)C)c4)c3=O)c(Cl)c2)no1]

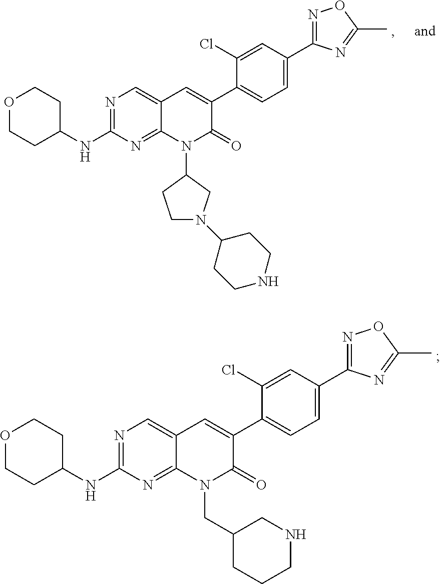 [SMILES: Cc1nc(-c2ccc(-c3cc4cnc(NC5CCOCC5)nc4n(C4CCN(C5CCNCC5)C4)c3=O)c(Cl)c2)no1 ; Cc1nc(-c2ccc(-c3cc4cnc(NC5CCOCC5)nc4n(CC4CCCNC4)c3=O)c(Cl)c2)no1]

or a pharmaceutically acceptable salt or N-oxide thereof.

In certain embodiments, compounds described herein have one or more chiral centers. As such, all stereoisomers are envisioned herein. In various embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieve in any suitable manner, including by way of non-limiting example, by resolution of the racemic form by recrystallization techniques, by synthesis from optically-active starting materials, by chiral synthesis, or by chromatographic separation using a chiral stationary phase. In some embodiments, mixtures of one or more isomer is utilized as the therapeutic compound described herein. In certain embodiments, compounds described herein contains one or more chiral centers. These compounds are prepared by any means, including enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, chromatography, and the like.

In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-tolunenesulfonate, mesylate and the like. Furthermore, pharmaceutically acceptable salts include, by way of non-limiting example, alkaline earth metal salts (e.g., calcium or magnesium), alkali metal salts (e.g., sodium-dependent or potassium), ammonium salts and the like.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, ³⁵S or the like. In some embodiments, isotopically-labeled compounds are useful in drug and/or substrate tissue distribution studies. In some embodiments, substitution with heavier isotopes such as deuterium affords certain therapeutic advantages resulting from greater metabolic stability (for example, increased in vivo half-life or reduced dosage requirements). In some embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference for such disclosure). General methods for the preparation of compound as described herein are modified by the use of appropriate reagents and conditions, for the introduction of the various moieties found in the formula as provided herein. As a guide the following synthetic methods are utilized.

Compounds described herein are synthesized using any suitable procedures starting from compounds that are available from commercial sources, or are prepared using procedures described herein.

Formation of Covalent Linkages by Reaction of an Electrophile with a Nucleophile

The compounds described herein are modified using various electrophiles and/or nucleophiles to form new functional groups or substituents. Table A entitled “Examples of Covalent Linkages and Precursors Thereof” lists selected non-limiting examples of covalent linkages and precursor functional groups which yield the covalent linkages. Table A is used as guidance toward the variety of electrophiles and nucleophiles combinations available that provide covalent linkages. Precursor functional groups are shown as electrophilic groups and nucleophilic groups.

TABLE A
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

### Use of Protecting Groups

In the reactions described, it is necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. In some embodiments it is contemplated that each protective group be removable by a different means. Protective groups that are cleaved under totally disparate reaction conditions fulfill the requirement of differential removal.

In some embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl in the presence of amines blocked with acid labile groups such as t-butyl carbamate or with carbamates that are both acid and base stable but hydrolytically removable.

In some embodiments carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a Pd⁰-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups are selected from:

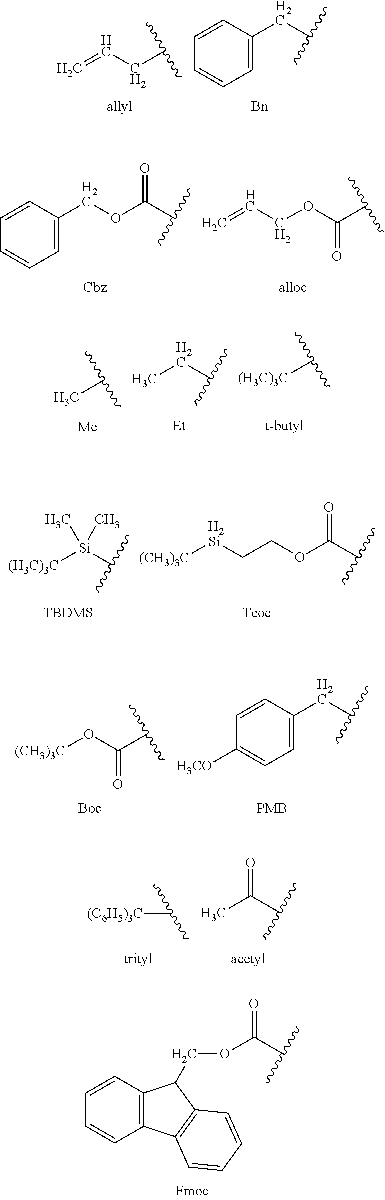

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### CERTAIN DEFINITIONS

As used herein the term “Treatment”, “treat”, or “treating” includes achieving a therapeutic benefit and/or a prophylactic benefit. Therapeutic benefit is meant to include eradication or amelioration of the underlying disorder or condition being treated. For example, in an individual with Huntington's disease, therapeutic benefit includes alleviation or partial and/or complete halting of the progression of the disease, or partial or complete reversal of the disease. Also, a therapeutic benefit is achieved with the eradication or amelioration of one or more of the physiological or psychological symptoms associated with the underlying condition such that an improvement is observed in the patient, notwithstanding the fact that the patient is still affected by the condition. For example, in an individual suffering from epilepsy, therapeutic benefit includes alleviation or partial and/or complete halting of seizures, or reduction in frequency of seizures. A prophylactic benefit of treatment includes prevention of a condition, retarding the progress of a condition, or decreasing the likelihood of occurrence of a condition. As used herein, “treat”, “treating” or “treatment” includes prophylaxis.

As used herein, the phrase “abnormal spine size” refers to dendritic spine volumes or dendritic spine surface areas (e.g., volumes or surface areas of the spine heads and/or spine necks) associated with Fragile X syndrome that deviate significantly relative to spine volumes or surface areas in the same brain region (e.g., the CA1 region, the prefrontal cortex) in a normal individual (e.g., a mouse, rat, or human) of the same age; such abnormalities are determined as appropriate, by methods including, e.g., tissue samples, relevant animal models, post-mortem analyses, or other model systems.

The phrase “defective spine morphology” or “abnormal spine morphology” or “aberrant spine morphology” refers to abnormal dendritic spine shapes, volumes, surface areas, length, width (e.g., diameter of the neck), spine head diameter, spine head volume, spine head surface area, spine density, ratio of mature to immature spines, ratio of spine volume to spine length, or the like that is associated with Fragile X syndrome relative to the dendritic spine shapes, volumes, surface areas, length, width (e.g., diameter of the neck), spine density, ratio of mature to immature spines, ratio of spine volume to spine length, or the like observed in the same brain region in a normal individual (e.g., a mouse, rat, or human) of the same age; such abnormalities or defects are determined as appropriate, by methods including, e.g., tissue samples, relevant animal models, post-mortem analyses, or other model systems.

The phrase “abnormal spine function” or “defective spine function” or “aberrant spine function” refers to a defect of dendritic spines to undergo stimulus-dependent morphological or functional changes (e.g., following activation of AMPA and/or NMDA receptors, LTP, LTD, etc) associated with Fragile X syndrome as compared to dendritic spines in the same brain region in a normal individual of the same age. The “defect” in spine function includes, e.g., a reduction in dendritic spine plasticity, (e.g., an abnormally small change in dendritic spine morphology or actin re-arrangement in the dendritic spine), or an excess level of dendritic plasticity, (e.g., an abnormally large change in dendritic spine morphology or actin re-arrangement in the dendritic spine). Such abnormalities or defects are determined as appropriate, by methods including, e.g., tissue samples, relevant animal models, post-mortem analyses, or other model systems.

The phrase “abnormal spine motility” refers to a significant low or high movement of dendritic spines associated with Fragile X syndrome as compared to dendritic spines in the same brain region in a normal individual of the same age. Any defect in spine morphology (e.g., spine length, density or the like) or synaptic plasticity or synaptic function (e.g., LTP, LTD or the like) or spine motility occurs in any region of the brain, including, for example, the frontal cortex, the hippocampus, the amygdala, the CA1 region, the prefrontal cortex or the like. Such abnormalities or defects are determined as appropriate, by methods including, e.g., tissue samples, relevant animal models, post-mortem analyses, or other model systems.

As used herein, the phrase “biologically active” refers to a characteristic of any substance that has activity in a biological system and/or organism. For instance, a substance that, when administered to an organism, has a biological effect on that organism is considered to be biologically active. In particular embodiments, where a protein or polypeptide is biologically active, a portion of that protein or polypeptide that shares at least one biological activity of the protein or polypeptide is typically referred to as a “biologically active” portion.

As used herein, the term “effective amount” is an amount, which when administered systemically, is sufficient to effect beneficial or desired results, such as beneficial or desired clinical results, or enhanced cognition, memory, mood, or other desired effects. An effective amount is also an amount that produces a prophylactic effect, e.g., an amount that delays, reduces, or eliminates the appearance of a pathological or undesired condition associated with Fragile X syndrome. An effective amount is optionally administered in one or more administrations. In terms of treatment, an “effective amount” of a composition described herein is an amount that is sufficient to palliate, alleviate, ameliorate, stabilize, reverse or slow the progression of Fragile X syndrome. An “effective amount” includes any PAK inhibitor used alone or in conjunction with one or more agents used to treat a disease or disorder. An “effective amount” of a therapeutic agent as described herein will be determined by a patient's attending physician or other medical care provider. Factors which influence what a therapeutically effective amount will be include, the absorption profile (e.g., its rate of uptake into the brain) of the PAK inhibitor, time elapsed since the initiation of disease, and the age, physical condition, existence of other disease states, and nutritional status of an individual being treated. Additionally, other medication the patient is receiving, e.g., antidepressant drugs used in combination with a PAK inhibitor, will typically affect the determination of the therapeutically effective amount of the therapeutic agent to be administered.

As used herein, the term “inhibitor” refers to a molecule which is capable of inhibiting (including partially inhibiting or allosteric inhibition) one or more of the biological activities of a target molecule, e.g., a p21-activated kinase Inhibitors, for example, act by reducing or suppressing the activity of a target molecule and/or reducing or suppressing signal transduction. In some embodiments, a PAK inhibitor described herein causes substantially complete inhibition of one or more PAKs. In some embodiments, the phrase “partial inhibitor” refers to a molecule which can induce a partial response for example, by partially reducing or suppressing the activity of a target molecule and/or partially reducing or suppressing signal transduction. In some instances, a partial inhibitor mimics the spatial arrangement, electronic properties, or some other physicochemical and/or biological property of the inhibitor. In some instances, in the presence of elevated levels of an inhibitor, a partial inhibitor competes with the inhibitor for occupancy of the target molecule and provides a reduction in efficacy, relative to the inhibitor alone. In some embodiments, a PAK inhibitor described herein is a partial inhibitor of one or more PAKs. In some embodiments, a PAK inhibitor described herein is an allosteric modulator of PAK. In some embodiments, a PAK inhibitor described herein blocks the p21 binding domain of PAK. In some embodiments, a PAK inhibitor described herein blocks the ATP binding site of PAK. In some embodiments, a PAK inhibitor is a “Type II” kinase inhibitor. In some embodiment a PAK inhibitor stabilizes PAK in its inactive conformation. In some embodiments, a PAK inhibitor stabilizes the “DFG-out” conformation of PAK.

In some embodiments, PAK inhibitors reduce, abolish, and/or remove the binding between PAK and at least one of its natural binding partners (e.g., Cdc42 or Rac). In some instances, binding between PAK and at least one of its natural binding partners is stronger in the absence of a PAK inhibitor (by e.g., 90%, 80%, 70%, 60%, 50%, 40%, 30% or 20%) than in the presence of a PAK inhibitor. Alternatively or additionally, PAK inhibitors inhibit the phosphotransferase activity of PAK, e.g., by binding directly to the catalytic site or by altering the conformation of PAK such that the catalytic site becomes inaccessible to substrates. In some embodiments, PAK inhibitors inhibit the ability of PAK to phosphorylate at least one of its target substrates, e.g., LIM kinase 1 (LIMK1), myosin light chain kinase (MLCK), cortactin; or itself. PAK inhibitors include inorganic and/or organic compounds.

In some embodiments, PAK inhibitors described herein increase dendritic spine length. In some embodiments, PAK inhibitors described herein decrease dendritic spine length. In some embodiments, PAK inhibitors described herein increase dendritic neck diameter. In some embodiments, PAK inhibitors described herein decrease dendritic neck diameter. In some embodiments, PAK inhibitors described herein increase dendritic spine head diameter. In some embodiments, PAK inhibitors described herein decrease dendritic spine head diameter. In some embodiments, PAK inhibitors described herein increase dendritic spine head volume. In some embodiments, PAK inhibitors described herein decrease dendritic spine head volume. In some embodiments, PAK inhibitors described herein increase dendritic spine surface area. In some embodiments, PAK inhibitors described herein decrease dendritic spine surface area. In some embodiments, PAK inhibitors described herein increase dendritic spine density. In some embodiments, PAK inhibitors described herein decrease dendritic spine density. In some embodiments, PAK inhibitors described herein increase the number of mushroom shaped spines. In some embodiments, PAK inhibitors described herein decrease the number of mushroom shaped spines.

In some embodiments, a PAK inhibitor suitable for the methods described herein is a direct PAK inhibitor. In some embodiments, a PAK inhibitor suitable for the methods described herein is an indirect PAK inhibitor. In some embodiments, a PAK inhibitor suitable for the methods described herein decreases PAK activity relative to a basal level of PAK activity by about 1.1 fold to about 100 fold, e.g., to about 1.2 fold, 1.5 fold, 1.6 fold, 1.7 fold, 2.0 fold, 3.0 fold, 5.0 fold, 6.0 fold, 7.0 fold, 8.5 fold, 9.7 fold, 10 fold, 12 fold, 14 fold, 15 fold, 20 fold, 30 fold, 40 fold, 50 fold, 60 fold, 70 fold, 90 fold, 95 fold, or by any other amount from about 1.1 fold to about 100 fold relative to basal PAK activity. In some embodiments, the PAK inhibitor is a reversible PAK inhibitor. In other embodiments, the PAK inhibitor is an irreversible PAK inhibitor. Direct PAK inhibitors are optionally used for the manufacture of a medicament for treating Fragile X syndrome.

In some embodiments, a PAK inhibitor used for the methods described herein has in vitro ED₅₀ for PAK activation of less than 100 μM (e.g., less than 10 μM, less than 5 μM, less than 4 μM, less than 3 μM, less than 1 μM, less than 0.8 μM, less than 0.6 μM, less than 0.5 μM, less than 0.4 μM, less than 0.3 μM, less than less than 0.2 μM, less than 0.1 μM, less than 0.08 μM, less than 0.06 μM, less than 0.05 μM, less than 0.04 μM, less than 0.03 μM, less than less than 0.02 μM, less than 0.01 μM, less than 0.0099 μM, less than 0.0098 μM, less than 0.0097 μM, less than 0.0096 μM, less than 0.0095 μM, less than 0.0094 μM, less than 0.0093 μM, less than 0.00092 μM, or less than 0.0090 μM).

In some embodiments, a PAK inhibitor used for the methods described herein has in vitro ED₅₀ for PAK activation of less than 100 μM (e.g., less than 10 μM, less than 5 μM, less than 4 μM, less than 3 μM, less than 1 μM, less than 0.8 μM, less than 0.6 μM, less than 0.5 μM, less than 0.4 μM, less than 0.3 μM, less than less than 0.2 μM, less than 0.1 μM, less than 0.08 μM, less than 0.06 μM, less than 0.05 μM, less than 0.04 μM, less than 0.03 μM, less than less than 0.02 μM, less than 0.01 μM, less than 0.0099 μM, less than 0.0098 μM, less than 0.0097 μM, less than 0.0096 μM, less than 0.0095 μM, less than 0.0094 μM, less than 0.0093 μM, less than 0.00092 μM, or less than 0.0090 μM).

As used herein, synaptic function refers to synaptic transmission and/or synaptic plasticity, including stabilization of synaptic plasticity. As used herein, “defect in synaptic plasticity” or “aberrant synaptic plasticity” refers to abnormal synaptic plasticity following stimulation of that synapse. In some embodiments, a defect in synaptic plasticity is a decrease in LTP. In some embodiments, a defect in synaptic plasticity is an increase in LTD. In some embodiments, a defect in synaptic plasticity is erratic (e.g., fluctuating, randomly increasing or decreasing) synaptic plasticity. In some instances, measures of synaptic plasticity are LTP and/or LTD (induced, for example, by theta-burst stimulation, high-frequency stimulation for LTP, low-frequency (e.g., e.g., 1 Hz) stimulation for LTD) and LTP and/or LTD after stabilization. In some embodiments, stabilization of LTP and/or LTD occurs in any region of the brain including the frontal cortex, the hippocampus, the prefrontal cortex, the amygdala or any combination thereof.

As used herein “stabilization of synaptic plasticity” refers to stable LTP or LTD following induction (e.g., by theta-burst stimulation, high-frequency stimulation for LTP, low-frequency (e.g., e.g., 1 Hz) stimulation for LTD).

“Aberrant stabilization of synaptic transmission” (for example, aberrant stabilization of LTP or LTD), refers to failure to establish a stable baseline of synaptic transmission following an induction paradigm (e.g., by theta-burst stimulation, high-frequency stimulation for LTP, low-frequency (e.g., 1 Hz) stimulation for LTD) or an extended period of vulnerability to disruption by pharmacological or electrophysiological means

As used herein “synaptic transmission” or “baseline synaptic transmission” refers to the EPSP and/or IPSP amplitude and frequency, neuronal excitability or population spike thresholds of a normal individual (e.g., an individual not suffering from Fragile X syndrome) or that predicted for an animal model for a normal individual. As used herein “aberrant synaptic transmission” or “defective synaptic transmission” refers to any deviation in synaptic transmission compared to synaptic transmission of a normal individual or that predicted for an animal model for a normal individual. In some embodiments, an individual suffering from Fragile X syndrome has a defect in baseline synaptic transmission that is a decrease in baseline synaptic transmission compared to the baseline synaptic transmission in a normal individual or that predicted for an animal model for a normal individual. In some embodiments, an individual suffering from Fragile X syndrome has a defect in baseline synaptic transmission that is an increase in baseline synaptic transmission compared to the baseline synaptic transmission in a normal individual or that predicted for an animal model for a normal individual.

As used herein “sensorimotor gating” is assessed, for example, by measuring prepulse inhibition (PPI) and/or habituation of the human startle response. In some embodiments, a defect in sensorimotor gating is a deficit in sensorimotor gating. In some embodiments, a defect in sensorimotor gating is an enhancement of sensorimotor gating.

As used herein, “normalization of aberrant synaptic plasticity” refers to a change in aberrant synaptic plasticity in an individual suffering from, suspected of having, or pre-disposed to Fragile X syndrome to a level of synaptic plasticity that is substantially the same as the synaptic plasticity of a normal individual or to that predicted from an animal model for a normal individual. As used herein, substantially the same means, for example, about 90% to about 110% of the measured synaptic plasticity in a normal individual or to that predicted from an animal model for a normal individual. In other embodiments, substantially the same means, for example, about 80% to about 120% of the measured synaptic plasticity in a normal individual or to that predicted from an animal model for a normal individual. In yet other embodiments, substantially the same means, for example, about 70% to about 130% of the synaptic plasticity in a normal individual or to that predicted from an animal model for a normal individual. As used herein, “partial normalization of aberrant synaptic plasticity” refers to any change in aberrant synaptic plasticity in an individual suffering from, suspected of having, or pre-disposed to Fragile X syndrome that trends towards synaptic plasticity of a normal individual or to that predicted from an animal model for a normal individual. As used herein “partially normalized synaptic plasticity” or “partially normal synaptic plasticity” is, for example, ± about 25%, ± about 35%, ± about 45%, ± about 55%, ± about 65%, or ± about 75% of the synaptic plasticity of a normal individual or to that predicted from an animal model for a normal individual. In some embodiments, normalization or partial normalization of aberrant synaptic plasticity in an individual suffering from, suspected of having, or pre-disposed to Fragile X syndrome is lowering of aberrant synaptic plasticity where the aberrant synaptic plasticity is higher than the synaptic plasticity of a normal individual or to that predicted from an animal model for a normal individual. In some embodiments, normalization or partial normalization of aberrant synaptic plasticity in an individual suffering from, suspected of having, or pre-disposed to Fragile X syndrome is an increase in aberrant synaptic plasticity where the aberrant synaptic plasticity is lower than the synaptic plasticity of a normal individual or to that predicted from an animal model for a normal individual. In some embodiments, normalization or partial normalization of synaptic plasticity in an individual suffering from, suspected of having, or pre-disposed to Fragile X syndrome is a change from an erratic (e.g., fluctuating, randomly increasing or decreasing) synaptic plasticity to a normal (e.g. stable) or partially normal (e.g., less fluctuating) synaptic plasticity compared to the synaptic plasticity of a normal individual or to that predicted from an animal model for a normal individual. In some embodiments, normalization or partial normalization of synaptic plasticity in an individual suffering from, suspected of having, or pre-disposed to Fragile X syndrome is a change from a non-stabilizing synaptic plasticity to a normal (e.g., stable) or partially normal (e.g., partially stable) synaptic plasticity compared to the synaptic plasticity of a normal individual or to that predicted from an animal model for a normal individual.

As used herein, “normalization of aberrant baseline synaptic transmission” refers to a change in aberrant baseline synaptic transmission in an individual suffering from, suspected of having, or pre-disposed to Fragile X syndrome to a level of baseline synaptic transmission that is substantially the same as the baseline synaptic transmission of a normal individual or to that predicted from an animal model for a normal individual. As used herein, substantially the same means, for example, about 90% to about 110% of the measured baseline synaptic transmission in a normal individual or to that predicted from an animal model for a normal individual. In other embodiments, substantially the same means, for example, about 80% to about 120% of the measured baseline synaptic transmission in a normal individual or to that predicted from an animal model for a normal individual. In yet other embodiments, substantially the same means, for example, about 70% to about 130% of the measured baseline synaptic transmission in a normal individual or to that predicted from an animal model for a normal individual. As used herein, “partial normalization of aberrant baseline synaptic transmission” refers to any change in aberrant baseline synaptic transmission in an individual suffering from, suspected of having, or pre-disposed to Fragile X syndrome that trends towards baseline synaptic transmission of a normal individual or to that predicted from an animal model for a normal individual. As used herein “partially normalized baseline synaptic transmission” or “partially normal baseline synaptic transmission” is, for example, ± about 25%, ± about 35%, ± about 45%, ± about 55%, ± about 65%, or ± about 75% of the measured baseline synaptic transmission of a normal individual or to that predicted from an animal model for a normal individual. In some embodiments, normalization or partial normalization of aberrant baseline synaptic transmission in an individual suffering from, suspected of having, or pre-disposed to Fragile X syndrome is lowering of aberrant baseline synaptic transmission where the aberrant baseline synaptic transmission is higher than the baseline synaptic transmission of a normal individual or to that predicted from an animal model for a normal individual. In some embodiments, normalization or partial normalization of aberrant baseline synaptic transmission in an individual suffering from, suspected of having, or pre-disposed to Fragile X syndrome is an increase in aberrant baseline synaptic transmission where the aberrant baseline synaptic transmission is lower than the baseline synaptic transmission of a normal individual or to that predicted from an animal model for a normal individual. In some embodiments, normalization or partial normalization of baseline synaptic transmission in an individual suffering from, suspected of having, or pre-disposed to Fragile X syndrome is a change from an erratic (e.g., fluctuating, randomly increasing or decreasing) baseline synaptic transmission to a normal (e.g. stable) or partially normal (e.g., less fluctuating) baseline synaptic transmission compared to the baseline synaptic transmission of a normal individual or to that predicted from an animal model for a normal individual. In some embodiments, normalization or partial normalization of aberrant baseline synaptic transmission in an individual suffering from, suspected of having, or pre-disposed to Fragile X syndrome is a change from a non-stabilizing baseline synaptic transmission to a normal (e.g., stable) or partially normal (e.g., partially stable) baseline synaptic transmission compared to the baseline synaptic transmission of a normal individual or to that predicted from an animal model for a normal individual.

As used herein, “normalization of aberrant synaptic function” refers to a change in aberrant synaptic function in an individual suffering from, suspected of having, or pre-disposed to Fragile X syndrome to a level of synaptic function that is substantially the same as the synaptic function of a normal individual or to that predicted from an animal model for a normal individual. As used herein, substantially the same means, for example, about 90% to about 110% of the synaptic function in a normal individual or to that predicted from an animal model for a normal individual. In other embodiments, substantially the same means, for example, about 80% to about 120% of the synaptic function in a normal individual or to that predicted from an animal model for a normal individual. In yet other embodiments, substantially the same means, for example, about 70% to about 130% of the synaptic function in a normal individual or to that predicted from an animal model for a normal individual. As used herein, “partial normalization of aberrant synaptic function” refers to any change in aberrant synaptic function in an individual suffering from, suspected of having, or pre-disposed to Fragile X syndrome that trends towards synaptic function of a normal individual or to that predicted from an animal model for a normal individual. As used herein “partially normalized synaptic function” or “partially normal synaptic function” is, for example, ± about 25%, ± about 35%, ± about 45%, ± about 55%, ± about 65%, or ± about 75% of the measured synaptic function of a normal individual or to that predicted from an animal model for a normal individual. In some embodiments, normalization or partial normalization of aberrant synaptic function in an individual suffering from, suspected of having, or pre-disposed to Fragile X syndrome is lowering of aberrant synaptic function where the aberrant synaptic function is higher than the synaptic function of a normal individual or to that predicted from an animal model for a normal individual. In some embodiments, normalization or partial normalization of aberrant synaptic function in an individual suffering from, suspected of having, or pre-disposed to Fragile X syndrome is an increase in aberrant synaptic function where the aberrant synaptic function is lower than the synaptic function of a normal individual or to that predicted from an animal model for a normal individual. In some embodiments, normalization or partial normalization of synaptic function in an individual suffering from, suspected of having, or pre-disposed to Fragile X syndrome is a change from an erratic (e.g., fluctuating, randomly increasing or decreasing) synaptic function to a normal (e.g. stable) or partially normal (e.g., less fluctuating) synaptic function compared to the synaptic function of a normal individual or to that predicted from an animal model for a normal individual. In some embodiments, normalization or partial normalization of aberrant synaptic function in an individual suffering from, suspected of having, or pre-disposed to Fragile X syndrome is a change from a non-stabilizing synaptic function to a normal (e.g., stable) or partially normal (e.g., partially stable) synaptic function compared to the synaptic function of a normal individual or to that predicted from an animal model for a normal individual.

As used herein, “normalization of aberrant long term potentiation (LTP)” refers to a change in aberrant LTP in an individual suffering from, suspected of having, or pre-disposed to Fragile X syndrome to a level of LTP that is substantially the same as the LTP of a normal individual or to that predicted from an animal model for a normal individual. As used herein, substantially the same means, for example, about 90% to about 110% of the LTP in a normal individual or to that predicted from an animal model for a normal individual. In other embodiments, substantially the same means, for example, about 80% to about 120% of the LTP in a normal individual or to that predicted from an animal model for a normal individual. In yet other embodiments, substantially the same means, for example, about 70% to about 130% of the LTP in a normal individual or to that predicted from an animal model for a normal individual. As used herein, “partial normalization of aberrant LTP” refers to any change in aberrant LTP in an individual suffering from, suspected of having, or pre-disposed to Fragile X syndrome that trends towards LTP of a normal individual or to that predicted from an animal model for a normal individual. As used herein “partially normalized LTP” or “partially normal LTP” is, for example, ± about 25%, ± about 35%, ± about 45%, ± about 55%, ± about 65%, or ± about 75% of the measured LTP of a normal individual or to that predicted from an animal model for a normal individual. In some embodiments, normalization or partial normalization of aberrant LTP in an individual suffering from, suspected of having, or pre-disposed to Fragile X syndrome is lowering of aberrant LTP where the aberrant LTP is higher than the LTP of a normal individual or to that predicted from an animal model for a normal individual. In some embodiments, normalization or partial normalization of aberrant LTP in an individual suffering from, suspected of having, or pre-disposed to Fragile X syndrome is an increase in aberrant LTP where the aberrant LTP is lower than the LTP of a normal individual or to that predicted from an animal model for a normal individual. In some embodiments, normalization or partial normalization of LTP in an individual suffering from, suspected of having, or pre-disposed to Fragile X syndrome is a change from an erratic (e.g., fluctuating, randomly increasing or decreasing) LTP to a normal (e.g. stable) or partially normal (e.g., less fluctuating) LTP compared to the LTP of a normal individual or to that predicted from an animal model for a normal individual. In some embodiments, normalization or partial normalization of aberrant LTP in an individual suffering from, suspected of having, or pre-disposed to Fragile X syndrome is a change from a non-stabilizing LTP to a normal (e.g., stable) or partially normal (e.g., partially stable) LTP compared to the LTP of a normal individual or to that predicted from an animal model for a normal individual.

As used herein, “normalization of aberrant long term depression (LTD)” refers to a change in aberrant LTD in an individual suffering from, suspected of having, or pre-disposed to Fragile X syndrome to a level of LTD that is substantially the same as the LTD of a normal individual or to that predicted from an animal model for a normal individual. As used herein, substantially the same means, for example, about 90% to about 110% of the LTD in a normal individual or to that predicted from an animal model for a normal individual. In other embodiments, substantially the same means, for example, about 80% to about 120% of the LTD in a normal individual or to that predicted from an animal model for a normal individual. In yet other embodiments, substantially the same means, for example, about 70% to about 130% of the LTD in a normal individual or to that predicted from an animal model for a normal individual. As used herein, “partial normalization of aberrant LTD” refers to any change in aberrant LTD in an individual suffering from, suspected of having, or pre-disposed to Fragile X syndrome that trends towards LTD of a normal individual or to that predicted from an animal model for a normal individual. As used herein “partially normalized LTD” or “partially normal LTD” is, for example, ± about 25%, ± about 35%, ± about 45%, ± about 55%, ± about 65%, or ± about 75% of the measured LTD of a normal individual or to that predicted from an animal model for a normal individual. In some embodiments, normalization or partial normalization of aberrant LTD in an individual suffering from, suspected of having, or pre-disposed to Fragile X syndrome is lowering of aberrant LTD where the aberrant LTD is higher than the LTD of a normal individual or to that predicted from an animal model for a normal individual. In some embodiments, normalization or partial normalization of aberrant LTD in an individual suffering from, suspected of having, or pre-disposed to Fragile X syndrome is an increase in aberrant LTD where the aberrant LTD is lower than the LTD of a normal individual or to that predicted from an animal model for a normal individual. In some embodiments, normalization or partial normalization of LTD in an individual suffering from, suspected of having, or pre-disposed to Fragile X syndrome is a change from an erratic (e.g., fluctuating, randomly increasing or decreasing) LTD to a normal (e.g. stable) or partially normal (e.g., less fluctuating) LTD compared to the LTD of a normal individual or to that predicted from an animal model for a normal individual. In some embodiments, normalization or partial normalization of aberrant LTD in an individual suffering from, suspected of having, or pre-disposed to Fragile X syndrome is a change from a non-stabilizing LTD to a normal (e.g., stable) or partially normal (e.g., partially stable) LTD compared to the LTD of a normal individual or to that predicted from an animal model for a normal individual.

As used herein, “normalization of aberrant sensorimotor gating” refers to a change in aberrant sensorimotor gating in an individual suffering from, suspected of having, or pre-disposed to Fragile X syndrome to a level of sensorimotor gating that is substantially the same as the sensorimotor gating of a normal individual or to that predicted from an animal model for a normal individual. As used herein, substantially the same means, for example, about 90% to about 110% of the sensorimotor gating in a normal individual or to that predicted from an animal model for a normal individual. In other embodiments, substantially the same means, for example, about 80% to about 120% of the sensorimotor gating in a normal individual or to that predicted from an animal model for a normal individual. In yet other embodiments, substantially the same means, for example, about 70% to about 130% of the sensorimotor gating in a normal individual or to that predicted from an animal model for a normal individual. As used herein, “partial normalization of aberrant sensorimotor gating” refers to any change in aberrant sensorimotor gating in an individual suffering from, suspected of having, or pre-disposed to Fragile X syndrome that trends towards sensorimotor gating of a normal individual or to that predicted from an animal model for a normal individual. As used herein “partially normalized sensorimotor gating” or “partially normal sensorimotor gating” is, for example, ± about 25%, ± about 35%, ± about 45%, ± about 55%, ± about 65%, or ± about 75% of the measured sensorimotor gating of a normal individual or to that predicted from an animal model for a normal individual. In some embodiments, normalization or partial normalization of aberrant sensorimotor gating in an individual suffering from, suspected of having, or pre-disposed to Fragile X syndrome is lowering of aberrant sensorimotor gating where the aberrant sensorimotor gating is higher than the sensorimotor gating of a normal individual or to that predicted from an animal model for a normal individual. In some embodiments, normalization or partial normalization of aberrant sensorimotor gating in an individual suffering from, suspected of having, or pre-disposed to Fragile X syndrome is an increase in aberrant sensorimotor gating where the aberrant sensorimotor gating is lower than the sensorimotor gating of a normal individual or to that predicted from an animal model for a normal individual. In some embodiments, normalization or partial normalization of sensorimotor gating in an individual suffering from, suspected of having, or pre-disposed to Fragile X syndrome is a change from an erratic (e.g., fluctuating, randomly increasing or decreasing) sensorimotor gating to a normal (e.g. stable) or partially normal (e.g., less fluctuating) sensorimotor gating compared to the sensorimotor gating of a normal individual or to that predicted from an animal model for a normal individual. In some embodiments, normalization or partial normalization of aberrant sensorimotor gating in an individual suffering from, suspected of having, or pre-disposed to Fragile X syndrome is a change from a non-stabilizing sensorimotor gating to a normal (e.g., stable) or partially normal (e.g., partially stable) sensorimotor gating compared to the sensorimotor gating of a normal individual or to that predicted from an animal model for a normal individual.

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; (4) post-translational modification of a polypeptide or protein.

As used herein the term “PAK polypeptide” or “PAK protein” or “PAK” refers to a protein that belongs in the family of p21-activated serine/threonine protein kinases. These include mammalian isoforms of PAK, e.g., the Group I PAK proteins (sometimes referred to as Group A PAK proteins), including PAK1, PAK2, PAK3, as well as the Group II PAK proteins (sometimes referred to as Group B PAK proteins), including PAK4, PAK5, and/or PAK6 Also included as PAK polypeptides or PAK proteins are lower eukaryotic isoforms, such as the yeast Ste20 (Leberter et al., 1992, EMBO J., 11:4805; incorporated herein by reference) and/or the Dictyostelium single-headed myosin I heavy chain kinases (Wu et al., 1996, J. Biol. Chem., 271:31787; incorporated herein by reference). Representative examples of PAK amino acid sequences include, but are not limited to, human PAK1 (GenBank Accession Number AAA65441), human PAK2 (GenBank Accession Number AAA65442), human PAK3 (GenBank Accession Number AAC36097), human PAK 4 (GenBank Accession Numbers NP_{—}005875 and CAA09820), human PAK5 (GenBank Accession Numbers CAC18720 and BAA94194), human PAK6 (GenBank Accession Numbers NP 064553 and AAF82800), human PAK7 (GenBank Accession Number Q9P286), C. elegans PAK (GenBank Accession Number BAA11844), D. melanogaster PAK (GenBank Accession Number AAC47094), and rat PAK1 (GenBank Accession Number AAB95646). In some embodiments, a PAK polypeptide comprises an amino acid sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers AAA65441, AAA65442, AAC36097, NP_{—}005875, CAA09820, CAC18720, BAA94194, NP_{—}064553, AAF82800, Q9P286, BAA11844, AAC47094, and/or AAB95646. In some embodiments, a Group I PAK polypeptide comprises an amino acid sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers AAA65441, AAA65442, and/or AAC36097.

Representative examples of PAK genes encoding PAK proteins include, but are not limited to, human PAK1 (GenBank Accession Number U24152), human PAK2 (GenBank Accession Number U24153), human PAK3 (GenBank Accession Number AF068864), human PAK4 (GenBank Accession Number AJ011855), human PAK5 (GenBank Accession Number AB040812), and human PAK6 (GenBank Accession Number AF276893). In some embodiments, a PAK gene comprises a nucleotide sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers U24152, U24153, AF068864, AJ011855, AB040812, and/or AF276893. In some embodiments, a Group I PAK gene comprises a nucleotide sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers U24152, U24153, and/or AF068864.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent homology between the two sequences is a function of the number of identical positions shared by the sequences (i.e., % identity=# of identical positions/total # of positions (e.g., overlapping positions)×100). In one embodiment the two sequences are the same length.

To determine percent homology between two sequences, the algorithm of Karlin and Altschul (1990) Proc. Natl. Acad. Sci. USA 87:2264-2268, modified as in Karlin and Altschul (1993) Proc. Natl. Acad. Sci. USA 90:5873-5877 is used. Such an algorithm is incorporated into the NBLAST and XBLAST programs of Altschul, et al. (1990) J. Mol. Biol. 215:403-410. BLAST nucleotide searches are performed with the NBLAST program, score=100, wordlength=12 to obtain nucleotide sequences homologous to a nucleic acid molecules described or disclose herein. BLAST protein searches are performed with the XBLAST program, score=50, wordlength=3. To obtain gapped alignments for comparison purposes, Gapped BLAST is utilized as described in Altschul et al. (1997) Nucleic Acids Res. 25:3389-3402. When utilizing BLAST and Gapped BLAST programs, the default parameters of the respective programs (e.g., XBLAST and NBLAST) are used. See the website of the National Center for Biotechnology Information for further details (on the world wide web at ncbi.nlm.nih.gov). Proteins suitable for use in the methods described herein also includes proteins having between 1 to 15 amino acid changes, e.g., 1, 2, 3, 4, 5, 6, 7, 8, 9, 10, 11, 12, 13, 14, or 15 amino acid substitutions, deletions, or additions, compared to the amino acid sequence of any protein PAK inhibitor described herein. In other embodiments, the altered amino acid sequence is at least 75% identical, e.g., 77%, 80%, 82%, 85%, 88%, 90%, 92%, 95%, 97%, 98%, 99%, or 100% identical to the amino acid sequence of any protein PAK inhibitor described herein. Such sequence-variant proteins are suitable for the methods described herein as long as the altered amino acid sequence retains sufficient biological activity to be functional in the compositions and methods described herein. Where amino acid substitutions are made, the substitutions should be conservative amino acid substitutions. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff et al (1992), Proc. Natl Acad. Sci. USA, 89:10915-10919). Accordingly, the BLOSUM62 substitution frequencies are used to define conservative amino acid substitutions that may be introduced into the amino acid sequences described or described herein. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

As used herein, the term “PAK activity,” unless otherwise specified, includes, but is not limited to, at least one of PAK protein-protein interactions, PAK phosphotransferase activity (intermolecular or intermolecular), translocation, etc of one or more PAK isoforms.

As used herein, a “PAK inhibitor” refers to any molecule, compound, or composition that directly or indirectly decreases the PAK activity. In some embodiments, PAK inhibitors inhibit, decrease, and/or abolish the level of a PAK mRNA and/or protein or the half-life of PAK mRNA and/or protein, such inhibitors are referred to as “clearance agents”. In some embodiments, a PAK inhibitor is a PAK antagonist that inhibits, decreases, and/or abolishes an activity of PAK. In some embodiments, a PAK inhibitor also disrupts, inhibits, or abolishes the interaction between PAK and its natural binding partners (e.g., a substrate for a PAK kinase, a Rac protein, a cdc42 protein, LIM kinase) or a protein that is a binding partner of PAK in a pathological condition, as measured using standard methods. In some embodiments, the PAK inhibitor is a Group I PAK inhibitor that inhibits, for example, one or more Group I PAK polypeptides, for example, PAK1, PAK2, and/or PAK3. In some embodiments, the PAK inhibitor is a PAK1 inhibitor. In some embodiments, the PAK inhibitor is a PAK2 inhibitor. In some embodiments, the PAK inhibitor is a PAK3 inhibitor. In some embodiments, the PAK inhibitor is a mixed PAK1/PAK3 inhibitor. In some embodiments, the PAK inhibitor inhibits all three Group I PAK isoforms (PAK1, PAK2 and PAK3) with equal or similar potency. In some embodiments, the PAK inhibitor is a Group II PAK inhibitor that inhibits one or more Group II PAK polypeptides, for example PAK4, PAK5, and/or PAK6. In some embodiments, the PAK inhibitor is a PAK4 inhibitor. In some embodiments, the PAK inhibitor is a PAK5 inhibitor. In some embodiments, the PAK inhibitor is a PAK6 inhibitor. In some embodiments, the PAK inhibitor is a PAK7 inhibitor. As used herein, a PAK5 polypeptide is substantially homologous to a PAK7 polypeptide.

In some embodiments, PAK inhibitors reduce, abolish, and/or remove the binding between PAK and at least one of its natural binding partners (e.g., Cdc42 or Rac). In some instances, binding between PAK and at least one of its natural binding partners is stronger in the absence of a PAK inhibitor (by e.g., 90%, 80%, 70%, 60%, 50%, 40%, 30% or 20%) than in the presence of a PAK inhibitor. In some embodiments, PAK inhibitors prevent, reduce, or abolish binding between PAK and a protein that abnormally accumulates or aggregates in cells or tissue in a disease state. In some instances, binding between PAK and at least one of the proteins that aggregates or accumulates in a cell or tissue is stronger in the absence of a PAK inhibitor (by e.g., 90%, 80%, 70%, 60%, 50%, 40%, 30% or 20%) than in the presence of an inhibitor.

An “individual” or an “individual,” as used herein, is a mammal. In some embodiments, an individual is an animal, for example, a rat, a mouse, a dog or a monkey. In some embodiments, an individual is a human patient. In some embodiments an “individual” or an “individual” is a human. In some embodiments, an individual suffers from Fragile X syndrome or is suspected to be suffering from Fragile X syndrome or is pre-disposed to Fragile X syndrome.

In some embodiments, a pharmacological composition comprising a PAK inhibitor is “administered peripherally” or “peripherally administered.” As used herein, these terms refer to any form of administration of an agent, e.g., a therapeutic agent, to an individual that is not direct administration to the CNS, i.e., that brings the agent in contact with the non-brain side of the blood-brain barrier. “Peripheral administration,” as used herein, includes intravenous, intra-arterial, subcutaneous, intramuscular, intraperitoneal, transdermal, by inhalation, transbuccal, intranasal, rectal, oral, parenteral, sublingual, or trans-nasal. In some embodiments, a PAK inhibitor is administered by an intracerebral route.

The terms “polypeptide,” and “protein” are used interchangeably herein to refer to a polymer of amino acid residues. That is, a description directed to a polypeptide applies equally to a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “isolated” and “purified” refer to a material that is substantially or essentially removed from or concentrated in its natural environment. For example, an isolated nucleic acid is one that is separated from the nucleic acids that normally flank it or other nucleic acids or components (proteins, lipids, etc.) in a sample. In another example, a polypeptide is purified if it is substantially removed from or concentrated in its natural environment. Methods for purification and isolation of nucleic acids and proteins are documented methodologies.

The term “optionally substituted” or “substituted” means that the referenced group substituted with one or more additional group(s). In certain embodiments, the one or more additional group(s) are individually and independently selected from amide, ester, alkyl, cycloalkyl, heteroalkyl, aryl, heteroaryl, heteroalicyclic, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, amido.

An “alkyl” group refers to an aliphatic hydrocarbon group. Reference to an alkyl group includes “saturated alkyl” and/or “unsaturated alkyl”. The alkyl group, whether saturated or unsaturated, includes branched, straight chain, or cyclic groups. By way of example only, alkyl includes methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. A “lower alkyl” is a C₁-C₆ alkyl. A “heteroalkyl” group substitutes any one of the carbons of the alkyl group with a heteroatom having the appropriate number of hydrogen atoms attached (e.g., a CH₂ group to an NH group or an O group).

An “alkoxy” group refers to a (alkyl)O— group, where alkyl is as defined herein.

The term “alkylamine” refers to the —N(alkyl)ₓH_{y} group, wherein alkyl is as defined herein and x and y are selected from the group x=1, y=1 and x=2, y=0. When x=2, the alkyl groups, taken together with the nitrogen to which they are attached, optionally form a cyclic ring system.

An “amide” is a chemical group with formula —C(═O)NRR′, where R and R′ is independently selected from hydrogen, alkyl, cycloalkyl, aryl, heteroaryl (bonded through a ring carbon) and heteroalicyclic (bonded through a ring carbon); or where R and R′ together with the nitrogen they attached form a heteroalicyclic.

“Amido” refers to a RC(═O)NR′—, where R and R′ is independently selected from the group consisting of alkyl, cycloalkyl, aryl, heteroaryl and heteroalicyclic.

The term “ester” refers to a chemical group with formula —C(═O)OR, where R is selected from the group consisting of alkyl, cycloalkyl, aryl, heteroaryl and heteroalicyclic.

“Alkoyloxy” refers to a RC(═O)O— group, where R is selected from the group consisting of alkyl, cycloalkyl, aryl, heteroaryl and heteroalicyclic.

“Alkoyl” refers to a RC(═O)— group, where R is selected from the group consisting of alkyl, cycloalkyl, aryl, heteroaryl and heteroalicyclic.

A “cyano” group refers to a —CN group.

An “isocyanato” group refers to a —NCO group.

A “thiocyanato” group refers to a —CNS group.

An “isothiocyanato” group refers to a —NCS group.

As used herein, the term “aryl” refers to an aromatic ring wherein each of the atoms forming the ring is a carbon atom. Aryl rings described herein include rings having five, six, seven, eight, nine, or more than nine carbon atoms. Aryl groups are optionally substituted. Examples of aryl groups include, but are not limited to phenyl, and naphthalenyl.

The term “cycloalkyl” refers to a monocyclic or polycyclic non-aromatic radical, wherein each of the atoms forming the ring (i.e. skeletal atoms) is a carbon atom. In various embodiments, cycloalkyls are saturated, or partially unsaturated. In some embodiments, cycloalkyls are fused with an aromatic ring. Cycloalkyl groups include groups having from 3 to 10 ring atoms. Illustrative examples of cycloalkyl groups include, but are not limited to, the following moieties:

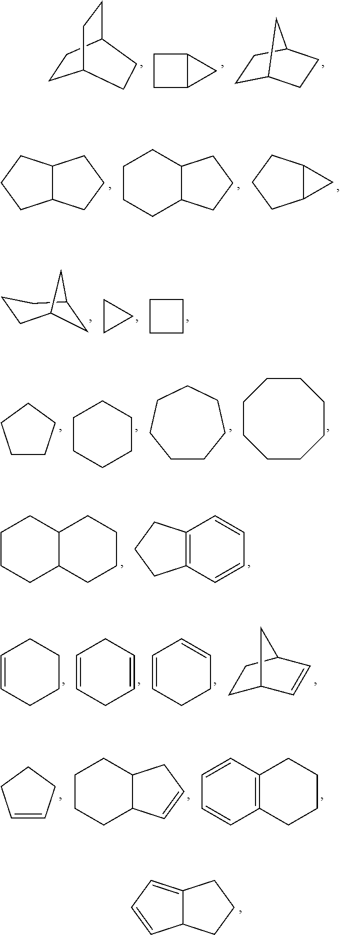

and the like. Monocyclic cycloalkyls include, but are not limited to, cyclopropyl, cyclobutyl, cyclopentyl, cyclohexyl, cycloheptyl, and cyclooctyl. Dicylclic cycloalkyls include, but are not limited to tetrahydronaphthyl, indanyl, tetrahydropentalene or the like. Polycyclic cycloalkyls include adamantane, norbornane or the like. The term cycloalkyl includes “unsaturated nonaromatic carbocyclyl” or “nonaromatic unsaturated carbocyclyl” groups both of which refer to a nonaromatic carbocycle, as defined herein, that contains at least one carbon carbon double bond or one carbon carbon triple bond.

The term “heterocyclo” refers to heteroaromatic and heteroalicyclic groups containing one to four ring heteroatoms each selected from O, S and N. In certain instances, each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In certain embodiments, heteroaryl groups are optionally substituted. In certain embodiments, heteroaryl groups are monocyclic or polycyclic. Examples of monocyclic heteroaryl groups include and are not limited to:

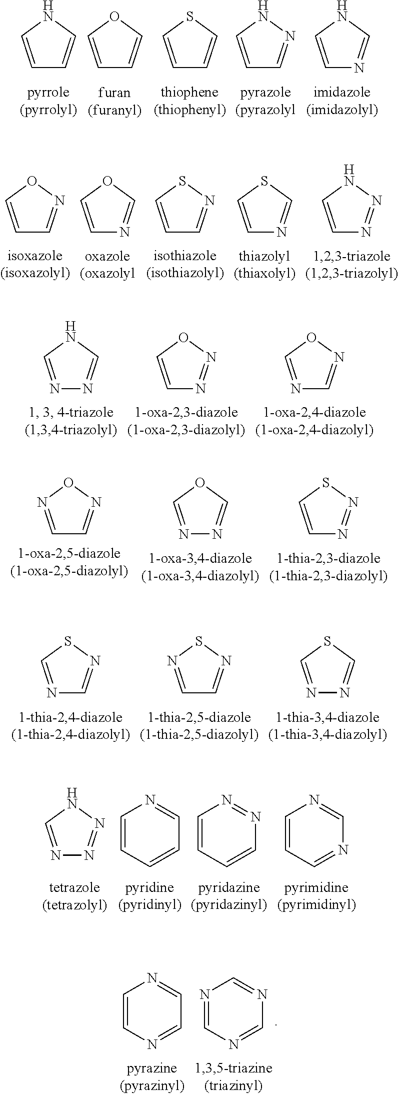

Examples of bicyclic heteroaryl groups include and are not limited to:

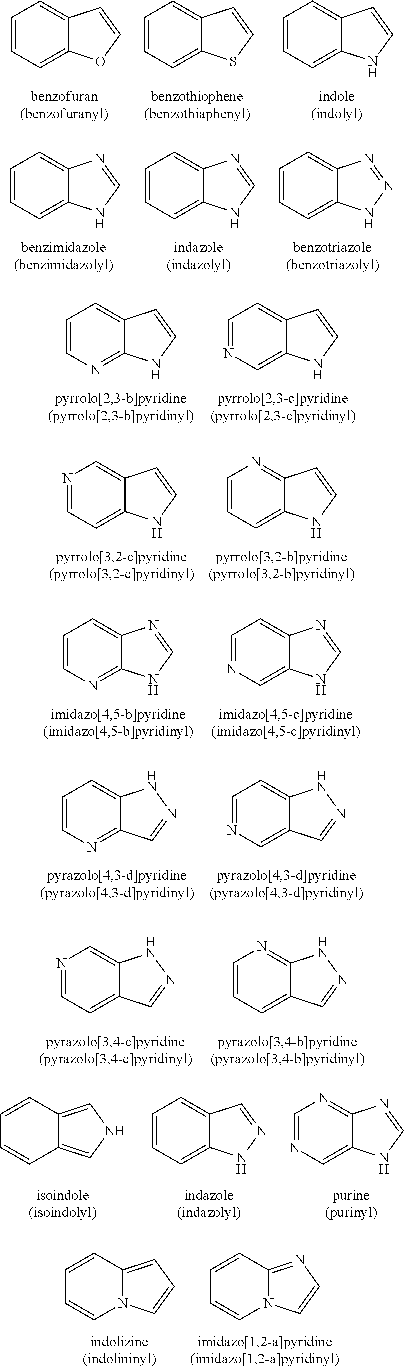

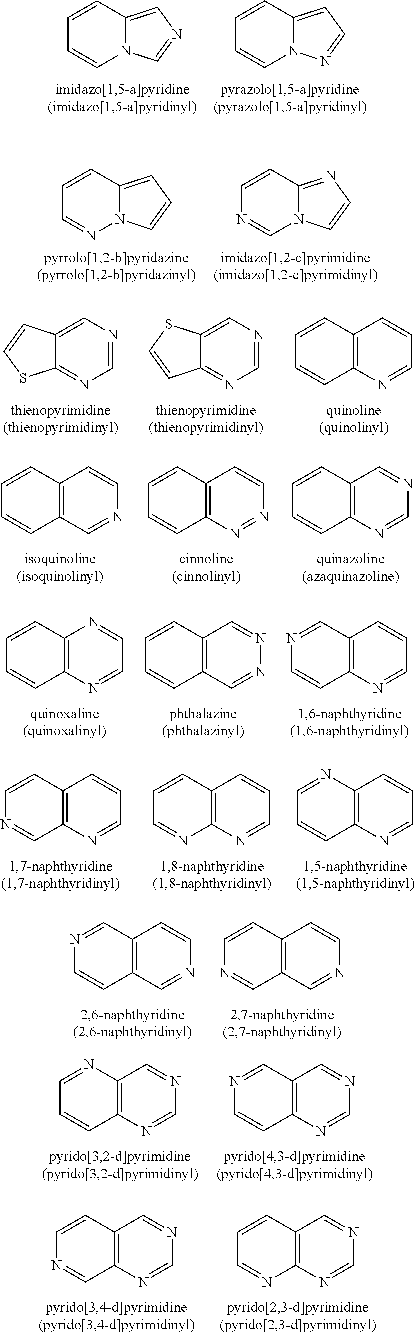

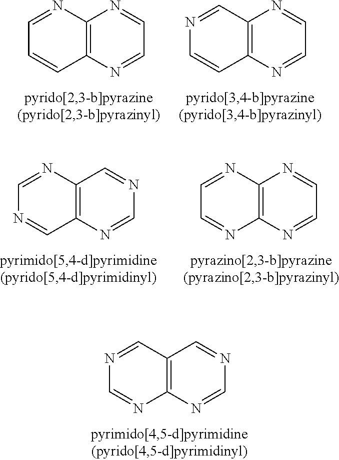

or the like.

A “heteroalicyclic” group or “heterocycloalkyl” group refers to a cycloalkyl group, wherein at least one skeletal ring atom is a heteroatom selected from nitrogen, oxygen and sulfur. In some embodiments, the radicals are fused with an aryl or heteroaryl. Example of saturated heterocyloalkyl groups include

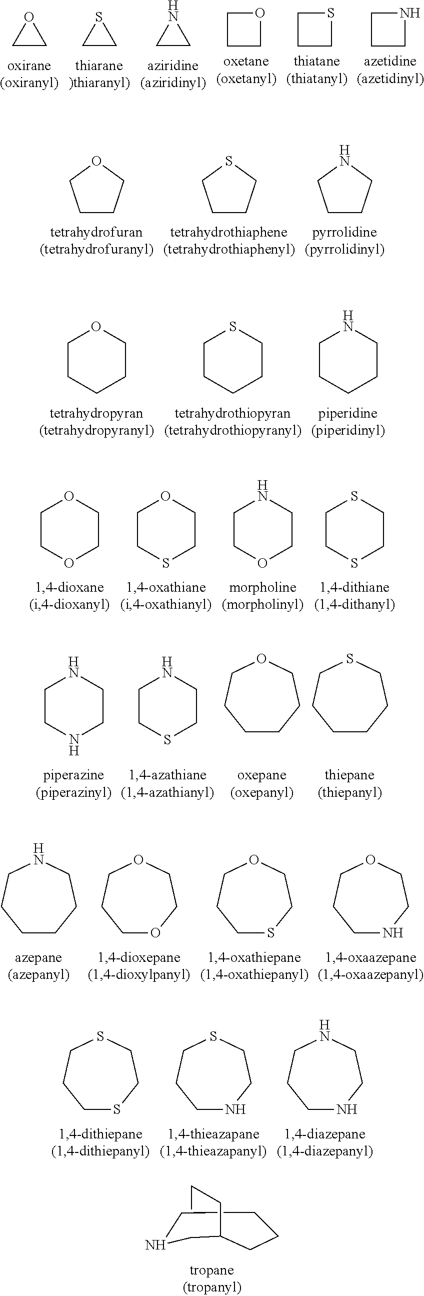

Examples of partially unsaturated heterocycloalkyl groups include

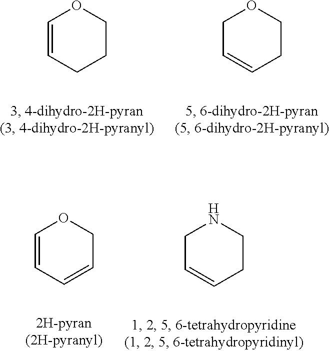 [SMILES: C1=COCCC1 ; C1=CCOCC1 ; C1=CCOC=C1 ; C1=CCNCC1]

Other illustrative examples of heterocycloalkyl groups, also referred to as non-aromatic heterocycles, include:

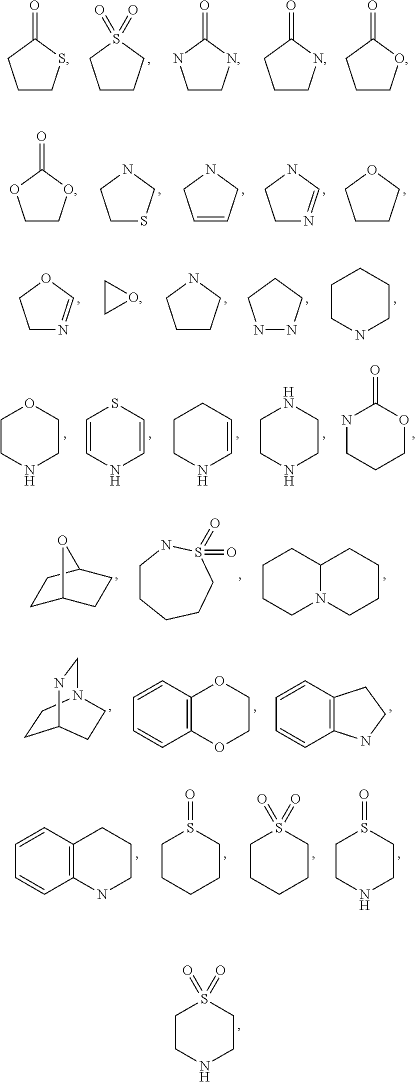

or the like.

The term heteroalicyclic also includes all ring forms of the carbohydrates, including but not limited to the monosaccharides, the disaccharides and the oligosaccharides.

The term “halo” or, alternatively, “halogen” means fluoro, chloro, bromo and iodo.

The terms “haloalkyl,” and “haloalkoxy” include alkyl and alkoxy structures that are substituted with one or more halogens. In embodiments, where more than one halogen is included in the group, the halogens are the same or they are different. The terms “fluoroalkyl” and “fluoroalkoxy” include haloalkyl and haloalkoxy groups, respectively, in which the halo is fluorine.

The term “heteroalkyl” include optionally substituted alkyl radicals which have one or more skeletal chain atoms selected from an atom other than carbon, e.g., oxygen, nitrogen, sulfur, phosphorus, silicon, or combinations thereof. When the heteroatom(s) is oxygen or sulfur, the heteroatom(s) is placed at any interior position other than immediately next to the carbon atom at the end of the skeletal chain. Otherwise, the heteroatom(s) is placed at any interior position of the skeletal chain. Examples of heteroalkyl include, but are not limited to, —CH₂—O—CH₂—CH₃, —CH₂—CH₂—O—CH₂—CH₃, —CH₂—O—CH₂—CH₂—CH₃, —CH₂—CH₂—O—CH₂—CH₂—CH₃, —CH₂—NH—CH₃, —CH₂—CH₂—NH—CH₂—CH₃, —CH₂—N(CH₃)—CH₂—CH₃, —CH₂—CH₂—NH—CH₂—CH₂—CH₃, —CH₂—CH₂—N(CH₃)₂, —CH₂—CH₂—CH₂—N(CH₃)₂, —CH₂—S—CH₂—CH₃, —CH₂—CH₂—S(O)—CH₂—CH₃, —CH₂—CH₂—S(O)₂—CH₂—CH₃, —CH₂—CH₂—O—CH₂—CH₂—NH₂, —CH₂—CH₂—O—CH₂—CH₂—N(CH₃)₂, —CH₂—CH₂—CH₂—O—CH₂—CH₂—CH₂—NH₂, —CH₂—CH₂—CH₂—O—CH₂—CH₂—CH₂—N(CH₃)₂, and —Si(CH₃)₃. In some embodiments, up to two heteroatoms are consecutive, such as, by way of example, —CH₂—NH—O—CH₂—CH₃ and —CH₂—O—Si—CH₂—CH₃. When the heteroatom(s) is oxygen or sulfur and is placed immediately next to the carbon atom at the end of the skeletal chain, such as in —CH₂—O—CH₃, —CH₂—CH₂—O—CH₃, —CH₂—CH₂—S—CH₃, and —CH₂—S—CH₃, the group is not characterized as a heteroalkyl. Instead, such groups are characterized as alkyls substituted with methoxy or thiomethoxy in the present disclosure.

### Synthesis of Compounds

In some embodiments, compounds of Formula I-IV and A-D are synthesized according to procedures described in Scheme 1.

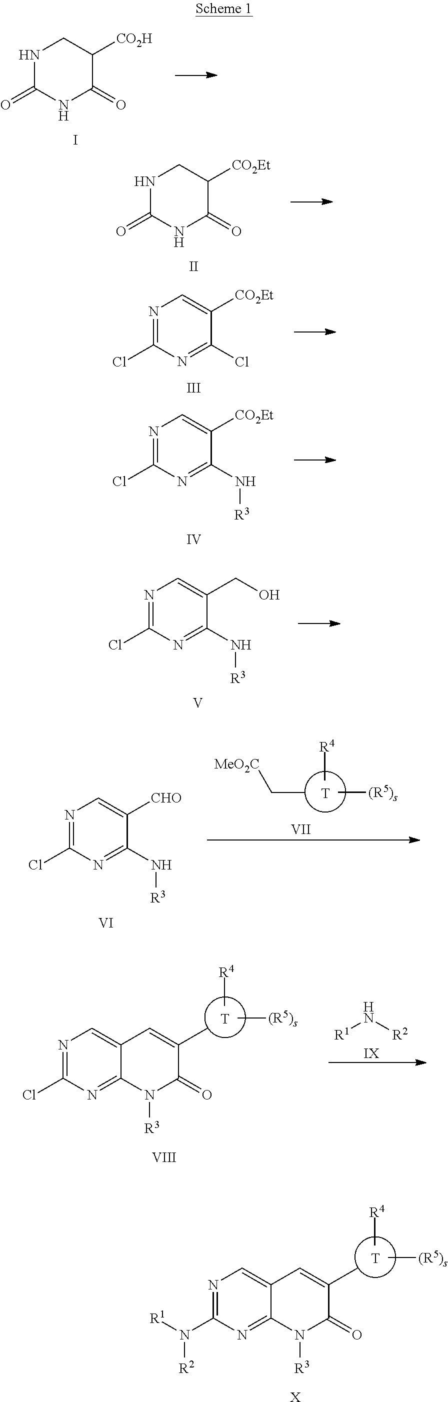

Generally, compounds of Formula X described herein are synthesized by conversion of I to its ethyl ester derivative II, followed by dichloropyrimidine formation to III. Substitution of the chlorine with an amine containing R³ forms the substituted compound IV. Reduction to alcohol V, followed by oxidation to the aldehyde, provides the substrate VI that undergoes condensation and intramolecular cyclization with the functionalized T ring VII to form VIII. Finally, chlorine displacement with the appropriate NR¹R² yields the target molecules X.

### Method of Treating Fragile X Syndrome of the Present Disclosure

In addition to the compounds described herein under “Compounds of the Present Disclosure,” other compounds, such as compounds of Formula I-IV in which R₂ is unsubstituted alkyl or alkyl substituted with substituted or unsubstituted amino, amido, nitro, arylthio, alkylsulfoxide, arylsulfoxide, alkylsulfone, arylsulfone, aryloxy, alkoloxo, amide, ester, alkoyl, cyano, aryl, or heteroaryl; substituted or unsubstituted alkoxy; substituted or unsubstituted aralkoxy; substituted or unsubstituted heteroalkyl; substituted or unsubstituted cycloalkyl; substituted or unsubstituted cycloalkylalkyl; substituted or unsubstituted heterocycloalkyl; substituted or unsubstituted heterocycloalkylalkyl; spiro-cycloakyl-heterocycloalkyl; -alkylene-S(═O)R⁹; -alkylene-S(═O)₂R⁹; or —S(═O)₂R⁹ described in the concurrently filed PCT application (Docket No. 36367-724.601), are also suitable for the method of treating Fragile X syndrome described herein. Although those compounds (disclosed in the concurrently filed PCT application) are not a part of the present disclosure directed to chemical compounds, they are part of the present disclosure directed to method of treating Fragile X syndrome.

Provided herein, in some embodiments, are methods for treating Fragile X syndrome, wherein the method comprises administering to an individual in need thereof a therapeutically effective amount of a compound having the structure of Formula A, Formula B, or Formula C, or a pharmaceutically acceptable salt or N-oxide thereof:

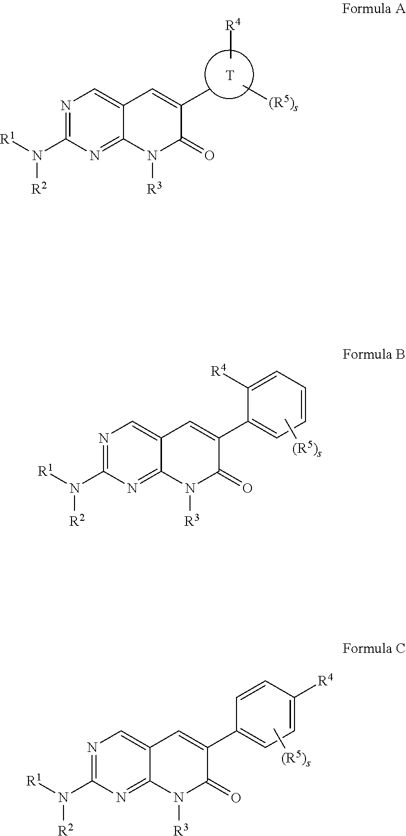

wherein:
ring T is a heteroaryl ring;
R¹ is H, or substituted or unsubstituted alkyl;
R² is substituted or unsubstituted alkyl, substituted or unsubstituted alkoxy, substituted or unsubstituted aralkoxy, substituted or unsubstituted heteroalkyl, substituted or unsubstituted cycloalkyl, substituted or unsubstituted cycloalkylalkyl, substituted or unsubstituted heterocycloalkyl, substituted or unsubstituted heterocycloalkylalkyl, spiro-cycloakyl-heterocycloalkyl, -alkylene-S(═O)R⁹, -alkylene-S(═O)₂R⁹, —S(═O)₂R⁹;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted heteroaryl attached to ring T or the phenyl ring via a carbon atom of R⁴, or substituted or unsubstituted heterocycloalkyl attached to ring T or the phenyl ring via a carbon atom of R⁴;
each R⁵ is independently halogen, —CN, —NO₂, —OH, —OCF₃, —OCH₂F, —OCF₂H, —CF₃, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and s is 0-4.

In some embodiments of the method of treating Fragile X syndrome, the compound has the structure of Formula A or pharmaceutically acceptable salt or N-oxide thereof:

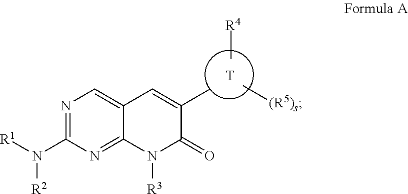

wherein:
ring T is a heteroaryl ring;
R¹ is H, or substituted or unsubstituted alkyl;
R² is substituted or unsubstituted alkyl, substituted or unsubstituted alkoxy, substituted or unsubstituted aralkoxy, substituted or unsubstituted heteroalkyl, substituted or unsubstituted cycloalkyl, substituted or unsubstituted cycloalkylalkyl, substituted or unsubstituted heterocycloalkyl, substituted or unsubstituted heterocycloalkylalkyl, spiro-cycloakyl-heterocycloalkyl, -alkylene-S(═O)R⁹, -alkylene-S(═O)₂R⁹, —S(═O)₂R⁹;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted heteroaryl attached to ring T via a carbon atom of R⁴, or substituted or unsubstituted heterocycloalkyl attached to ring T via a carbon atom of R⁴;
each R⁵ is independently halogen, —CN, —NO₂, —OH, —OCF₃, —OCH₂F, —OCF₂H, —CF₃, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

In one embodiment, ring T of Formula A is selected from pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl. In some embodiments, ring T is pyrrolyl. In some embodiments, ring T is furanyl. In some embodiments, ring T is thiophenyl. In some embodiments, ring T is pyrazolyl. In some embodiments, ring T is imidazolyl. In some embodiments, ring T is isoxazolyl. In some embodiments, ring T is oxazolyl. In some embodiments, ring T is isothiazolyl. In some embodiments, ring T is thiazolyl. In some embodiments, ring T is 1,2,3-triazolyl. In some embodiments, ring T is 1,3,4-triazolyl. In some embodiments, ring T is 1-oxa-2,3-diazolyl. In some embodiments, ring T is 1-oxa-2,4-diazolyl. In some embodiments, ring T is 1-oxa-2,5-diazolyl. In some embodiments, ring T is 1-oxa-3,4-diazolyl. In some embodiments, ring T is 1-thia-2,3-diazolyl. In some embodiments, ring T is 1-thia-2,4-diazolyl. In some embodiments, ring T is 1-thia-2,5-diazolyl. In some embodiments, ring T is 1-thia-3,4-diazolyl. In some embodiments, ring T is tetrazolyl. In some embodiments, ring T is pyridinyl. In some embodiments, ring T is pyridazinyl. In some embodiments, ring T is pyrimidinyl. In some embodiments, ring T is pyrazinyl. In some embodiments, ring T is triazinyl. In some embodiments, ring T is indolyl. In some embodiments, ring T is benzofuranyl. In some embodiments, ring T is benzimidazolyl. In some embodiments, ring T is indazolyl. In some embodiments, ring T is pyrrolopyridinyl. In some embodiments, ring T is imidazopyridinyl.

In another embodiment, R⁴ in Formula A is a substituted or unsubstituted C-linked heterocycloalkyl. In a further embodiment, the C-linked heterocycloalkyl is pyrrolidinyl, tetrahydrofuranyl, piperidinyl, tetrahydropyranyl, tetrahydrothiopyranyl, morpholinyl, or piperazinyl. In some embodiments, the C-linked heterocycloalkyl is pyrrolidinyl. In some embodiments, the C-linked heterocycloalkyl is tetrahydrofuranyl. In some embodiments, the C-linked heterocycloalkyl is piperidinyl. In some embodiments, the C-linked heterocycloalkyl is tetrahydropyranyl. In some embodiments, the C-linked heterocycloalkyl is tetrahydrothiopyranyl. In some embodiments, the C-linked heterocycloalkyl is morpholinyl. In some embodiments, the C-linked heterocycloalkyl is piperazinyl. In a further embodiment, the C-linked heterocycloalkyl is substituted with at least one C₁-C₆alkyl or halogen. In another embodiment, the C₁-C₆alkyl is methyl, ethyl, or n-propyl.

In one embodiment, R⁴ in Formula A is a substituted or unsubstituted C-linked heteroaryl. In one embodiment, R⁴ is selected from a C-linked pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl. In some embodiments, R⁴ is a C-linked pyrrolyl. In some embodiments, R⁴ is a C-linked furanyl. In some embodiments, R⁴ is a C-linked thiophenyl. In some embodiments, R⁴ is a C-linked pyrazolyl. In some embodiments, R⁴ is a C-linked imidazolyl. In some embodiments, R⁴ is a C-linked isoxazolyl. In some embodiments, R⁴ is a C-linked oxazolyl. In some embodiments, R⁴ is a C-linked isothiazolyl. In some embodiments, R⁴ is a C-linked thiazolyl. In some embodiments, R⁴ is a C-linked 1,2,3-triazolyl. In some embodiments, R⁴ is a C-linked 1,3,4-triazolyl. In some embodiments, R⁴ is a C-linked 1-oxa-2,3-diazolyl. In some embodiments, R⁴ is a C-linked 1-oxa-2,4-diazolyl. In some embodiments, R⁴ is a C-linked 1-oxa-2,5-diazolyl. In some embodiments, R⁴ is a C-linked 1-oxa-3,4-diazolyl. In some embodiments, R⁴ is a C-linked 1-thia-2,3-diazolyl. In some embodiments, R⁴ is a C-linked 1-thia-2,4-diazolyl. In some embodiments, R⁴ is a C-linked 1-thia-2,5-diazolyl. In some embodiments, R⁴ is a C-linked 1-thia-3,4-diazolyl. In some embodiments, R⁴ is a C-linked tetrazolyl. In some embodiments, R⁴ is a C-linked pyridinyl. In some embodiments, R⁴ is a C-linked pyridazinyl. In some embodiments, R⁴ is a C-linked pyrimidinyl. In some embodiments, R⁴ is a C-linked pyrazinyl. In some embodiments, R⁴ is a C-linked triazinyl. In some embodiments, R⁴ is a C-linked indolyl. In some embodiments, R⁴ is a C-linked benzofuranyl. In some embodiments, R⁴ is a C-linked benzimidazolyl. In some embodiments, R⁴ is a C-linked indazolyl. In some embodiments, R⁴ is a C-linked pyrrolopyridinyl. In some embodiments, R⁴ is a C-linked imidazopyridinyl.

In another embodiment, R⁴ in Formula A is a C-linked heteroaryl substituted with at least one group selected from halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, the C-linked heteroaryl is substituted with C₁-C₆alkyl. In another embodiment, C₁-C₆alkyl is methyl, ethyl, n-propyl, iso-propyl, n-butyl, iso-butyl, or tert-butyl. In a further embodiment, the C-linked heteroaryl is substituted with methyl. In another embodiment, the C-linked heteroaryl is substituted with ethyl. In a further embodiment, the C-linked heteroaryl is substituted with n-propyl or iso-propyl.

In another embodiment, the compound of Formula A has the structure of Formula A1:

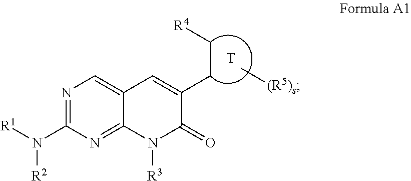

wherein ring T, R¹, R², R³, R⁴, R⁵, and s are described previously.

In another embodiment, the compound of Formula A has the structure of Formula A2:

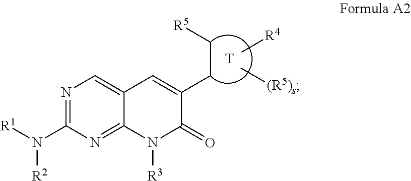

wherein ring T, R¹, R², R³, R⁴, R⁵ are described previously and s is 0-3.

In another embodiment, the compound of Formula A has the structure of Formula A3:

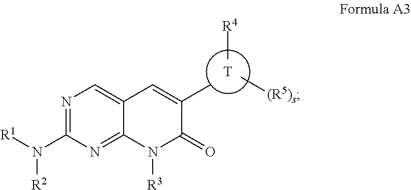

wherein:
ring T is an aryl or heteroaryl ring;
R¹ is H, or substituted or unsubstituted alkyl;
R² is substituted or unsubstituted alkyl, substituted or unsubstituted alkoxy, substituted or unsubstituted aralkoxy, substituted or unsubstituted heteroalkyl, substituted or unsubstituted cycloalkylalkyl, substituted or unsubstituted heterocycloalkyl, substituted or unsubstituted heterocycloalkylalkyl, substituted or unsubstituted aralkyl, substituted or unsubstituted heteroarylalkyl, spiro-cycloakyl-heterocycloalkyl, -alkylene-S(═O)R⁹, -alkylene-S(═O)₂R⁹, —S(═O)₂R⁹;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted cycloalkyl, substituted or unsubstituted aryl, substituted or unsubstituted heteroaryl attached to ring T via a carbon atom of R⁴, or substituted or unsubstituted heterocycloalkyl attached to ring T via a carbon atom of R⁴;
each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

In one embodiment, ring T in Formula A3 is selected from pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl. In some embodiments, ring T is pyrrolyl. In some embodiments, ring T is furanyl. In some embodiments, ring T is thiophenyl. In some embodiments, ring T is pyrazolyl. In some embodiments, ring T is imidazolyl. In some embodiments, ring T is isoxazolyl. In some embodiments, ring T is oxazolyl. In some embodiments, ring T is isothiazolyl. In some embodiments, ring T is thiazolyl. In some embodiments, ring T is 1,2,3-triazolyl. In some embodiments, ring T is 1,3,4-triazolyl. In some embodiments, ring T is 1-oxa-2,3-diazolyl. In some embodiments, ring T is 1-oxa-2,4-diazolyl. In some embodiments, ring T is 1-oxa-2,5-diazolyl. In some embodiments, ring T is 1-oxa-3,4-diazolyl. In some embodiments, ring T is 1-thia-2,3-diazolyl. In some embodiments, ring T is 1-thia-2,4-diazolyl. In some embodiments, ring T is 1-thia-2,5-diazolyl. In some embodiments, ring T is 1-thia-3,4-diazolyl. In some embodiments, ring T is tetrazolyl. In some embodiments, ring T is pyridinyl. In some embodiments, ring T is pyridazinyl. In some embodiments, ring T is pyrimidinyl. In some embodiments, ring T is pyrazinyl. In some embodiments, ring T is triazinyl. In some embodiments, ring T is indolyl. In some embodiments, ring T is benzofuranyl. In some embodiments, ring T is benzimidazolyl. In some embodiments, ring T is indazolyl. In some embodiments, ring T is pyrrolopyridinyl. In some embodiments, ring T is imidazopyridinyl.

In another embodiment, R⁴ in Formula A3 is a substituted or unsubstituted C-linked heterocycloalkyl. In a further embodiment, the C-linked heterocycloalkyl is pyrrolidinyl, tetrahydrofuranyl, piperidinyl, tetrahydropyranyl, tetrahydrothiopyranyl, morpholinyl, or piperazinyl. In some embodiments, the C-linked heterocycloalkyl is pyrrolidinyl. In some embodiments, the C-linked heterocycloalkyl is tetrahydrofuranyl. In some embodiments, the C-linked heterocycloalkyl is piperidinyl. In some embodiments, the C-linked heterocycloalkyl is tetrahydropyranyl. In some embodiments, the C-linked heterocycloalkyl is tetrahydrothiopyranyl. In some embodiments, the C-linked heterocycloalkyl is morpholinyl. In some embodiments, the C-linked heterocycloalkyl is piperazinyl. In a further embodiment, the C-linked heterocycloalkyl is substituted with at least one C₁-C₆alkyl or halogen. In another embodiment, the C₁-C₆alkyl is methyl, ethyl, or n-propyl.

In another embodiment, R⁴ in Formula A3 is a substituted or unsubstituted C-linked heteroaryl. In one embodiment, R⁴ is selected from a C-linked pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl. In some embodiments, R⁴ is a C-linked pyrrolyl. In some embodiments, R⁴ is a C-linked furanyl. In some embodiments, R⁴ is a C-linked thiophenyl. In some embodiments, R⁴ is a C-linked pyrazolyl. In some embodiments, R⁴ is a C-linked imidazolyl. In some embodiments, R⁴ is a C-linked isoxazolyl. In some embodiments, R⁴ is a C-linked oxazolyl. In some embodiments, R⁴ is a C-linked isothiazolyl. In some embodiments, R⁴ is a C-linked thiazolyl. In some embodiments, R⁴ is a C-linked 1,2,3-triazolyl. In some embodiments, R⁴ is a C-linked 1,3,4-triazolyl. In some embodiments, R⁴ is a C-linked 1-oxa-2,3-diazolyl. In some embodiments, R⁴ is a C-linked 1-oxa-2,4-diazolyl. In some embodiments, R⁴ is a C-linked 1-oxa-2,5-diazolyl. In some embodiments, R⁴ is a C-linked 1-oxa-3,4-diazolyl. In some embodiments, R⁴ is a C-linked 1-thia-2,3-diazolyl. In some embodiments, R⁴ is a C-linked 1-thia-2,4-diazolyl. In some embodiments, R⁴ is a C-linked 1-thia-2,5-diazolyl. In some embodiments, R⁴ is a C-linked 1-thia-3,4-diazolyl. In some embodiments, R⁴ is a C-linked tetrazolyl. In some embodiments, R⁴ is a C-linked pyridinyl. In some embodiments, R⁴ is a C-linked pyridazinyl. In some embodiments, R⁴ is a C-linked pyrimidinyl. In some embodiments, R⁴ is a C-linked pyrazinyl. In some embodiments, R⁴ is a C-linked triazinyl. In some embodiments, R⁴ is a C-linked indolyl. In some embodiments, R⁴ is a C-linked benzofuranyl. In some embodiments, R⁴ is a C-linked benzimidazolyl. In some embodiments, R⁴ is a C-linked indazolyl. In some embodiments, R⁴ is a C-linked pyrrolopyridinyl. In some embodiments, R⁴ is a C-linked imidazopyridinyl.

In another embodiment, wherein R⁴ in Formula A3 is a C-linked heteroaryl substituted with at least one group selected from halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, the C-linked heteroaryl is substituted with C₁-C₆alkyl. In another embodiment, C₁-C₆alkyl is methyl, ethyl, n-propyl, iso-propyl, n-butyl, iso-butyl, or tert-butyl. In a further embodiment, the C-linked heteroaryl is substituted with methyl. In another embodiment, the C-linked heteroaryl is substituted with ethyl. In a further embodiment, the C-linked heteroaryl is substituted with n-propyl or iso-propyl.

In another embodiment, R⁴ in Formula A3 is a substituted or unsubstituted cycloalkyl. In a further embodiment, cycloalkyl is selected from cyclopropyl, cyclobutyl, cyclopentyl, or cyclohexyl. In a further embodiment, R⁴ is cyclopentyl. In another embodiment, R⁴ is cyclohexyl.

In another embodiment, R⁴ in Formula A3 is a substituted or unsubstituted aryl. In another embodiment, R⁴ in Formula A3 is a substituted or unsubstituted phenyl.

In some embodiments of the method of treating Fragile X syndrome, the compound has the structure of Formula B or pharmaceutically acceptable salt or N-oxide thereof:

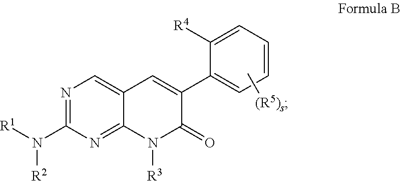

wherein:
R¹ is H, or substituted or unsubstituted alkyl;
R² is substituted or unsubstituted alkyl, substituted or unsubstituted alkoxy, substituted or unsubstituted aralkoxy, substituted or unsubstituted heteroalkyl, substituted or unsubstituted cycloalkyl, substituted or unsubstituted cycloalkylalkyl, substituted or unsubstituted heterocycloalkyl, substituted or unsubstituted heterocycloalkylalkyl, spiro-cycloakyl-heterocycloalkyl, -alkylene-S(═O)R⁹, -alkylene-S(═O)₂R⁹, —S(═O)₂R⁹;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted heteroaryl attached to the phenyl ring via a carbon atom of R⁴, or substituted or unsubstituted heterocycloalkyl attached to the phenyl ring via a carbon atom of R⁴;
each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl; each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

In one embodiment, R⁴ in Formula B is a substituted or unsubstituted C-linked heterocycloalkyl. In a further embodiment, the C-linked heterocycloalkyl is pyrrolidinyl, tetrahydrofuranyl, piperidinyl, tetrahydropyranyl, tetrahydrothiopyranyl, morpholinyl, or piperazinyl. In some embodiments, the C-linked heterocycloalkyl is pyrrolidinyl. In some embodiments, the C-linked heterocycloalkyl is tetrahydrofuranyl. In some embodiments, the C-linked heterocycloalkyl is piperidinyl. In some embodiments, the C-linked heterocycloalkyl is tetrahydropyranyl. In some embodiments, the C-linked heterocycloalkyl is tetrahydrothiopyranyl. In some embodiments, the C-linked heterocycloalkyl is morpholinyl. In some embodiments, the C-linked heterocycloalkyl is piperazinyl. In a further embodiment, the C-linked heterocycloalkyl is substituted with at least one C₁-C₆alkyl or halogen. In another embodiment, the C₁-C₆alkyl is methyl, ethyl, or n-propyl.

In another embodiment, R⁴ in Formula B is a substituted or unsubstituted C-linked heteroaryl. In one embodiment, R⁴ is selected from a C-linked pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl. In some embodiments, R⁴ is a C-linked pyrrolyl. In some embodiments, R⁴ is a C-linked furanyl. In some embodiments, R⁴ is a C-linked thiophenyl. In some embodiments, R⁴ is a C-linked pyrazolyl. In some embodiments, R⁴ is a C-linked imidazolyl. In some embodiments, R⁴ is a C-linked isoxazolyl. In some embodiments, R⁴ is a C-linked oxazolyl. In some embodiments, R⁴ is a C-linked isothiazolyl. In some embodiments, R⁴ is a C-linked thiazolyl. In some embodiments, R⁴ is a C-linked 1,2,3-triazolyl. In some embodiments, R⁴ is a C-linked 1,3,4-triazolyl. In some embodiments, R⁴ is a C-linked 1-oxa-2,3-diazolyl. In some embodiments, R⁴ is a C-linked 1-oxa-2,4-diazolyl. In some embodiments, R⁴ is a C-linked 1-oxa-2,5-diazolyl. In some embodiments, R⁴ is a C-linked 1-oxa-3,4-diazolyl. In some embodiments, R⁴ is a C-linked 1-thia-2,3-diazolyl. In some embodiments, R⁴ is a C-linked 1-thia-2,4-diazolyl. In some embodiments, R⁴ is a C-linked 1-thia-2,5-diazolyl. In some embodiments, R⁴ is a C-linked 1-thia-3,4-diazolyl. In some embodiments, R⁴ is a C-linked tetrazolyl. In some embodiments, R⁴ is a C-linked pyridinyl. In some embodiments, R⁴ is a C-linked pyridazinyl. In some embodiments, R⁴ is a C-linked pyrimidinyl. In some embodiments, R⁴ is a C-linked pyrazinyl. In some embodiments, R⁴ is a C-linked triazinyl. In some embodiments, R⁴ is a C-linked indolyl. In some embodiments, R⁴ is a C-linked benzofuranyl. In some embodiments, R⁴ is a C-linked benzimidazolyl. In some embodiments, R⁴ is a C-linked indazolyl. In some embodiments, R⁴ is a C-linked pyrrolopyridinyl. In some embodiments, R⁴ is a C-linked imidazopyridinyl.

In another embodiment, R⁴ in Formula B is a C-linked heteroaryl substituted with at least one group selected from halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, the C-linked heteroaryl is substituted with C₁-C₆alkyl. In another embodiment, C₁-C₆alkyl is methyl, ethyl, n-propyl, iso-propyl, n-butyl, iso-butyl, or tert-butyl. In a further embodiment, the C-linked heteroaryl is substituted with methyl. In another embodiment, the C-linked heteroaryl is substituted with ethyl. In a further embodiment, the C-linked heteroaryl is substituted with n-propyl or iso-propyl.

In some embodiments of the method of treating Fragile X syndrome, the compound has the structure of Formula C or pharmaceutically acceptable salt or N-oxide thereof:

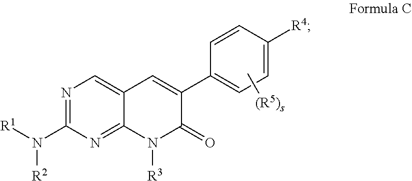

wherein:
R¹ is H, or substituted or unsubstituted alkyl;
R² is substituted or unsubstituted alkyl, substituted or unsubstituted alkoxy, substituted or unsubstituted aralkoxy, substituted or unsubstituted heteroalkyl, substituted or unsubstituted cycloalkyl, substituted or unsubstituted cycloalkylalkyl, substituted or unsubstituted heterocycloalkyl, substituted or unsubstituted heterocycloalkylalkyl, spiro-cycloakyl-heterocycloalkyl, -alkylene-S(═O)R⁹, -alkylene-S(═O)₂R⁹, —S(═O)₂R⁹;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted heteroaryl attached to the phenyl ring via a carbon atom of R⁴, or substituted or unsubstituted heterocycloalkyl attached to the phenyl ring via a carbon atom of R⁴;
each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

In one embodiment, R⁴ in Formula C is a substituted or unsubstituted C-linked heterocycloalkyl. In a further embodiment, the C-linked heterocycloalkyl is pyrrolidinyl, tetrahydrofuranyl, piperidinyl, tetrahydropyranyl, tetrahydrothiopyranyl, morpholinyl, or piperazinyl. In some embodiments, the C-linked heterocycloalkyl is pyrrolidinyl. In some embodiments, the C-linked heterocycloalkyl is tetrahydrofuranyl. In some embodiments, the C-linked heterocycloalkyl is piperidinyl. In some embodiments, the C-linked heterocycloalkyl is tetrahydropyranyl. In some embodiments, the C-linked heterocycloalkyl is tetrahydrothiopyranyl. In some embodiments, the C-linked heterocycloalkyl is morpholinyl. In some embodiments, the C-linked heterocycloalkyl is piperazinyl. In a further embodiment, the C-linked heterocycloalkyl is substituted with at least one C₁-C₆alkyl or halogen. In another embodiment, the C₁-C₆alkyl is methyl, ethyl, or n-propyl.

In another embodiment, R⁴ in Formula C is a substituted or unsubstituted C-linked heteroaryl. In one embodiment, R⁴ is selected from a C-linked pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl. In some embodiments, R⁴ is a C-linked pyrrolyl. In some embodiments, R⁴ is a C-linked furanyl. In some embodiments, R⁴ is a C-linked thiophenyl. In some embodiments, R⁴ is a C-linked pyrazolyl. In some embodiments, R⁴ is a C-linked imidazolyl. In some embodiments, R⁴ is a C-linked isoxazolyl. In some embodiments, R⁴ is a C-linked oxazolyl. In some embodiments, R⁴ is a C-linked isothiazolyl. In some embodiments, R⁴ is a C-linked thiazolyl. In some embodiments, R⁴ is a C-linked 1,2,3-triazolyl. In some embodiments, R⁴ is a C-linked 1,3,4-triazolyl. In some embodiments, R⁴ is a C-linked 1-oxa-2,3-diazolyl. In some embodiments, R⁴ is a C-linked 1-oxa-2,4-diazolyl. In some embodiments, R⁴ is a C-linked 1-oxa-2,5-diazolyl. In some embodiments, R⁴ is a C-linked 1-oxa-3,4-diazolyl. In some embodiments, R⁴ is a C-linked 1-thia-2,3-diazolyl. In some embodiments, R⁴ is a C-linked 1-thia-2,4-diazolyl. In some embodiments, R⁴ is a C-linked 1-thia-2,5-diazolyl. In some embodiments, R⁴ is a C-linked 1-thia-3,4-diazolyl. In some embodiments, R⁴ is a C-linked tetrazolyl. In some embodiments, R⁴ is a C-linked pyridinyl. In some embodiments, R⁴ is a C-linked pyridazinyl. In some embodiments, R⁴ is a C-linked pyrimidinyl. In some embodiments, R⁴ is a C-linked pyrazinyl. In some embodiments, R⁴ is a C-linked triazinyl. In some embodiments, R⁴ is a C-linked indolyl. In some embodiments, R⁴ is a C-linked benzofuranyl. In some embodiments, R⁴ is a C-linked benzimidazolyl. In some embodiments, R⁴ is a C-linked indazolyl. In some embodiments, R⁴ is a C-linked pyrrolopyridinyl. In some embodiments, R⁴ is a C-linked imidazopyridinyl.

In another embodiment, R⁴ in Formula C is a C-linked heteroaryl substituted with at least one group selected from halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, the C-linked heteroaryl is substituted with C₁-C₆alkyl. In another embodiment, C₁-C₆alkyl is methyl, ethyl, n-propyl, iso-propyl, n-butyl, iso-butyl, or tert-butyl. In a further embodiment, the C-linked heteroaryl is substituted with methyl. In another embodiment, the C-linked heteroaryl is substituted with ethyl. In a further embodiment, the C-linked heteroaryl is substituted with n-propyl or iso-propyl.

In another embodiment, the compound of Formula C has the structure of Formula C1:

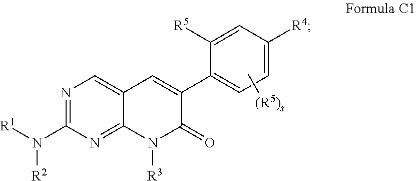

wherein R¹, R², R³, R⁴, R⁵ are described previously and s is 0-3.

In another embodiment, the compound of Formula C has the structure of Formula C2:

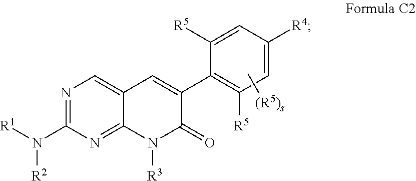

wherein R¹, R², R³, R⁴, R⁵ are described previously and s is 0-2.

In some embodiments of the method of treating Fragile X syndrome, the compound has the structure of Formula D or pharmaceutically acceptable salt or N-oxide thereof:

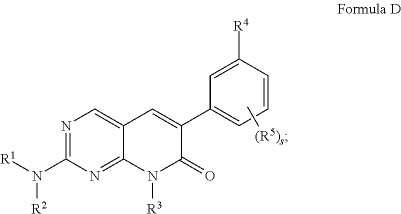

wherein:
R¹ is H, or substituted or unsubstituted alkyl;
R² is substituted or unsubstituted alkyl, substituted or unsubstituted alkoxy, substituted or unsubstituted aralkoxy, substituted or unsubstituted heteroalkyl, substituted or unsubstituted cycloalkyl, substituted or unsubstituted cycloalkylalkyl, substituted or unsubstituted heterocycloalkyl, substituted or unsubstituted heterocycloalkylalkyl, spiro-cycloakyl-heterocycloalkyl, -alkylene-S(═O)R⁹, -alkylene-S(═O)₂R⁹, —S(═O)₂R⁹;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted 6-membered monocyclic heteroaryl ring attached to the phenyl ring via a carbon atom of R⁴, substituted or unsubstituted bicyclic heteroaryl ring attached to the phenyl ring via a carbon atom of R⁴, or substituted or unsubstituted heterocycloalkyl attached to the phenyl ring via a carbon atom of R⁴;
each R⁵ is independently halogen, —CN, —NO₂, —OH, —OCF₃, —OCH₂F, —OCF₂H, —CF₃, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

In one embodiment, R⁴ in Formula D is a substituted or unsubstituted C-linked heterocycloalkyl. In a further embodiment, the C-linked heterocycloalkyl is pyrrolidinyl, tetrahydrofuranyl, piperidinyl, tetrahydropyranyl, tetrahydrothiopyranyl, morpholinyl, or piperazinyl. In some embodiments, the C-linked heterocycloalkyl is pyrrolidinyl. In some embodiments, the C-linked heterocycloalkyl is tetrahydrofuranyl. In some embodiments, the C-linked heterocycloalkyl is piperidinyl. In some embodiments, the C-linked heterocycloalkyl is tetrahydropyranyl. In some embodiments, the C-linked heterocycloalkyl is tetrahydrothiopyranyl. In some embodiments, the C-linked heterocycloalkyl is morpholinyl. In some embodiments, the C-linked heterocycloalkyl is piperazinyl. In a further embodiment, the C-linked heterocycloalkyl is substituted with at least one C₁-C₆alkyl or halogen. In another embodiment, the C₁-C₆alkyl is methyl, ethyl, or n-propyl.

In another embodiment, R⁴ in Formula D is a substituted or unsubstituted C-linked 6-membered monocyclic heteroaryl ring. In some embodiments, R⁴ is selected from a C-linked pyridine, pyridazinyl, pyrimidinyl, pyrazinyl, and triazinyl. In some embodiments, R⁴ is a C-linked pyridinyl. In some embodiments, R⁴ is a C-linked pyridazinyl. In some embodiments, R⁴ is a C-linked pyrimidinyl. In some embodiments, R⁴ is a C-linked pyrazinyl. In some embodiments, R⁴ is a C-linked triazinyl.

In another embodiment, R⁴ in Formula D is a substituted or unsubstituted C-linked bicyclic heteroaryl ring. In some embodiments, R⁴ is selected from a C-linked indolyl, benzofuranyl, benzimidazolyl, indazolyl, pyrrolopyridinyl, and imidazopyridinyl. In some embodiments, R⁴ is a C-linked indolyl. In some embodiments, R⁴ is a C-linked benzofuranyl. In some embodiments, R⁴ is a C-linked benzimidazolyl. In some embodiments, R⁴ is a C-linked indazolyl. In some embodiments, R⁴ is a C-linked pyrrolopyridinyl. In some embodiments, R⁴ is a C-linked imidazopyridinyl.

In another embodiment, R⁴ in Formula D is a C-linked 6-membered monocyclic heteroaryl ring substituted with at least one group selected from halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, the C-linked heteroaryl is substituted with C₁-C₆alkyl. In another embodiment, C₁-C₆alkyl is methyl, ethyl, n-propyl, iso-propyl, n-butyl, iso-butyl, or tert-butyl. In a further embodiment, the C-linked heteroaryl is substituted with methyl. In another embodiment, the C-linked heteroaryl is substituted with ethyl. In a further embodiment, the C-linked heteroaryl is substituted with n-propyl or iso-propyl.

In another embodiment, R⁴ in Formula D is a C-linked bicyclic heteroaryl ring substituted with at least one group selected from halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, the C-linked heteroaryl is substituted with C₁-C₆alkyl. In another embodiment, C₁-C₆alkyl is methyl, ethyl, n-propyl, iso-propyl, n-butyl, iso-butyl, or tert-butyl. In a further embodiment, the C-linked heteroaryl is substituted with methyl. In another embodiment, the C-linked heteroaryl is substituted with ethyl. In a further embodiment, the C-linked heteroaryl is substituted with n-propyl or iso-propyl.

In some embodiments of the method for treating Fragile X syndrome, the compound having the structure of Formula A is selected from:

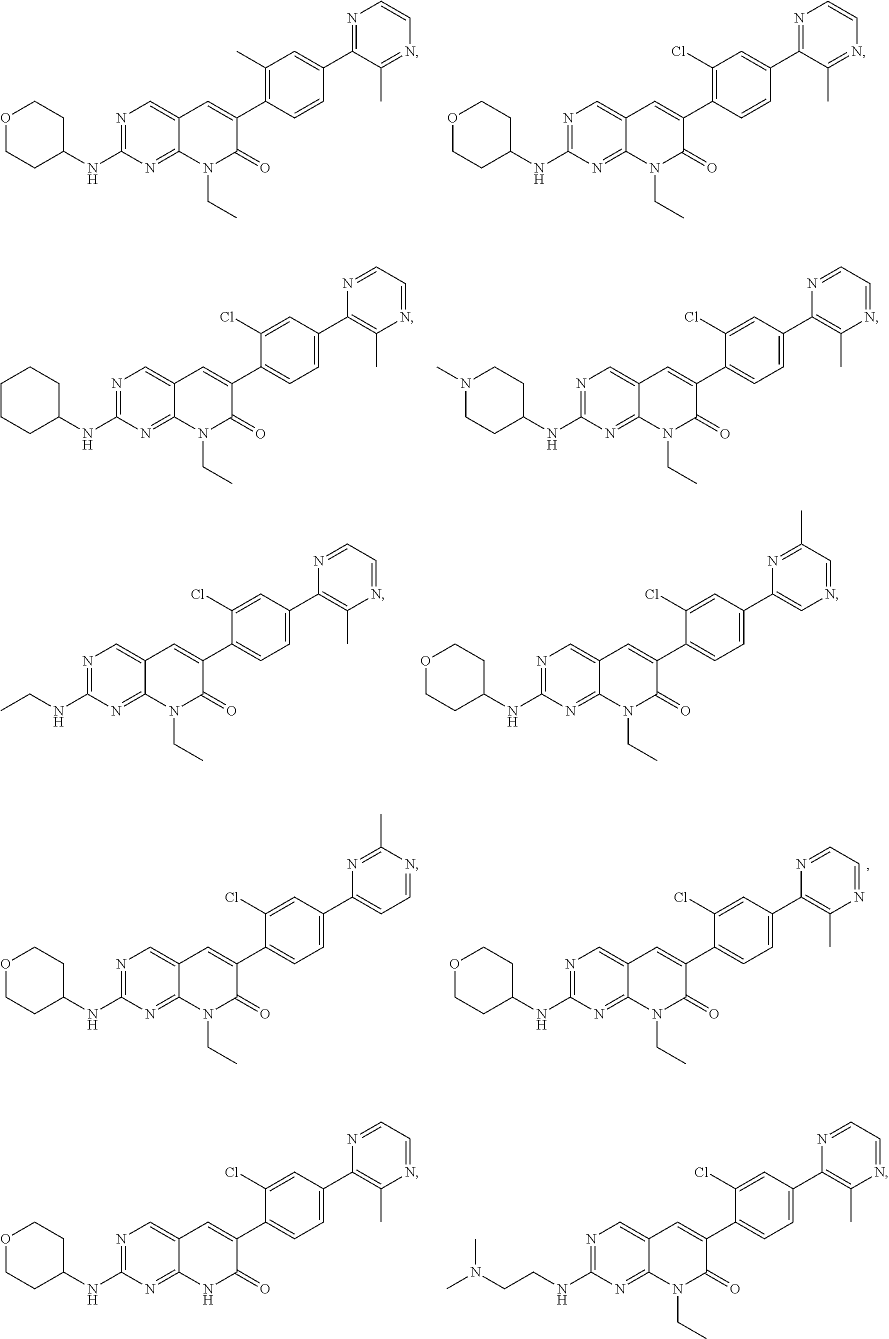

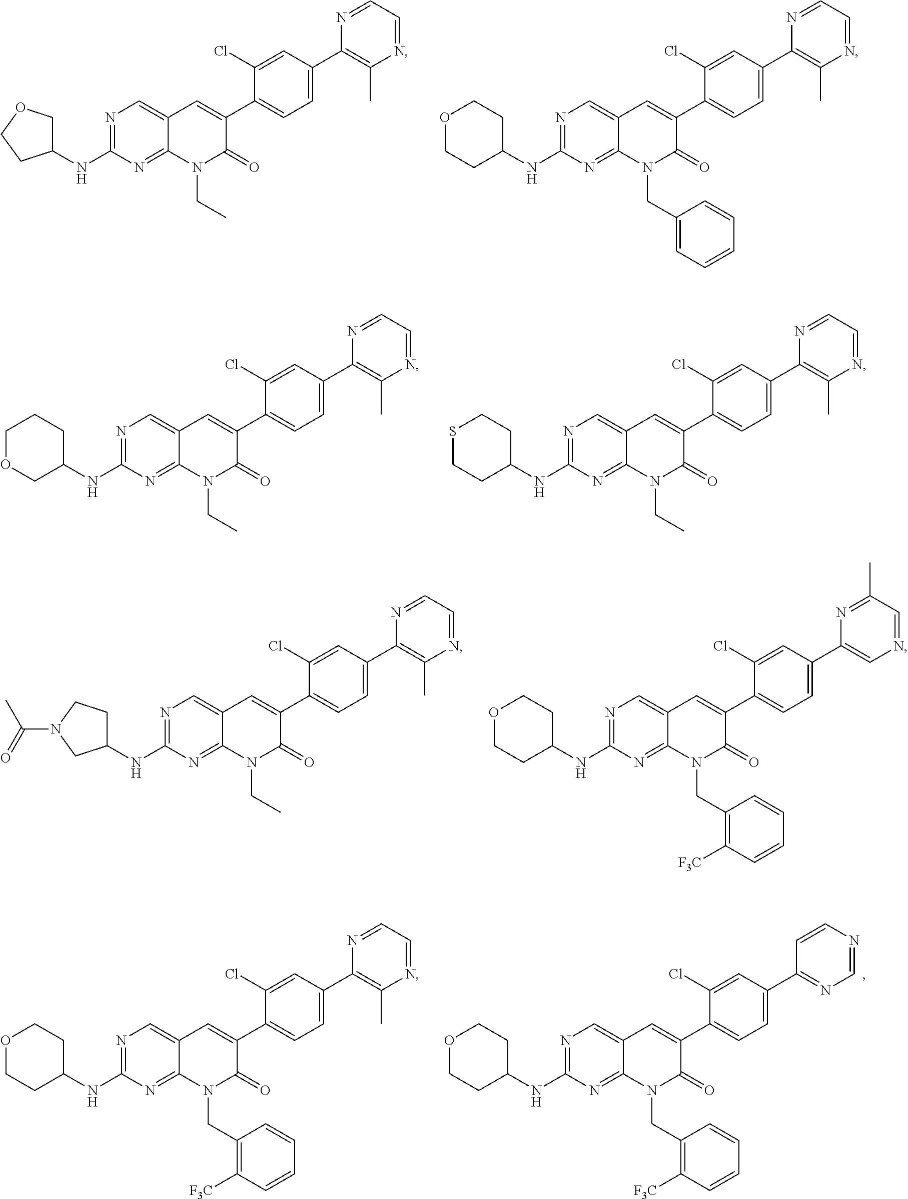

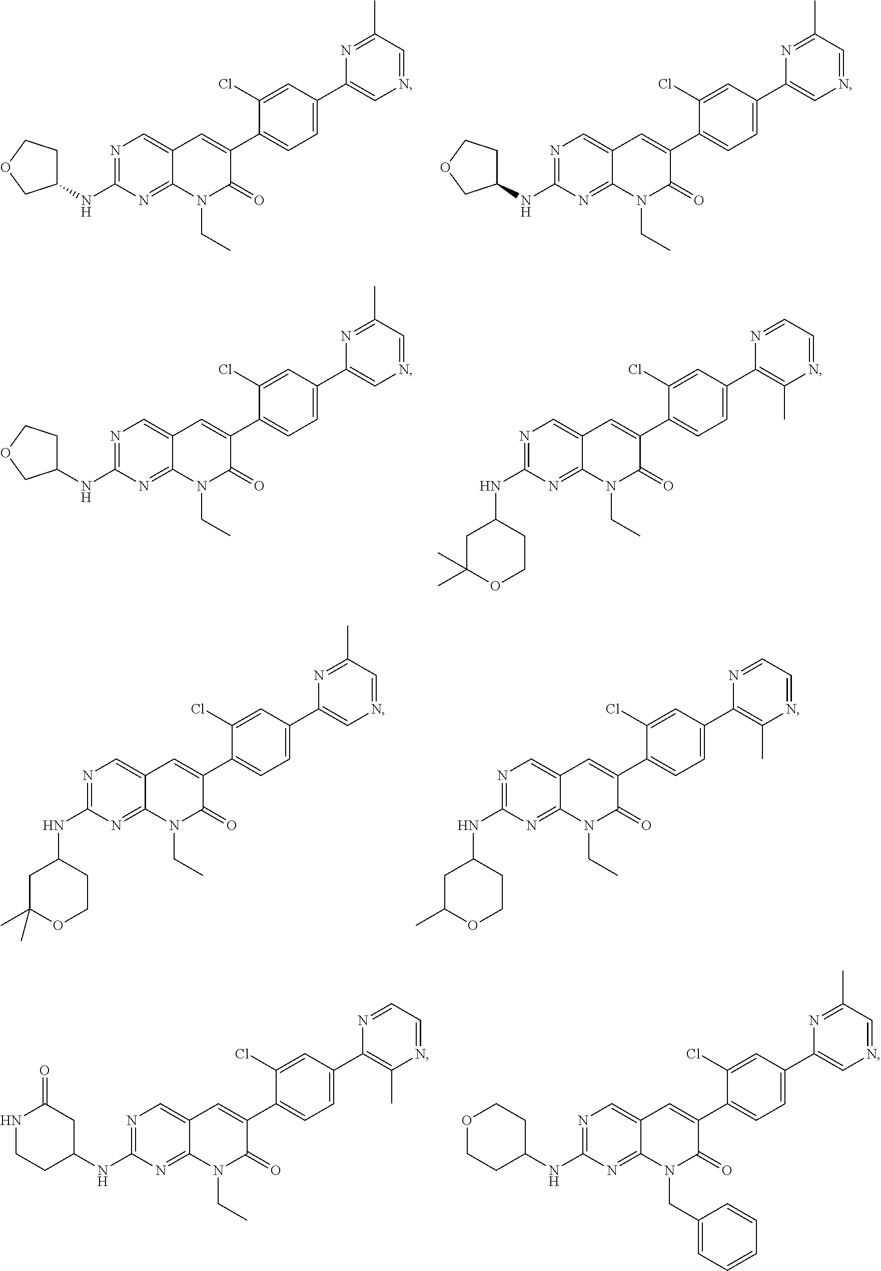

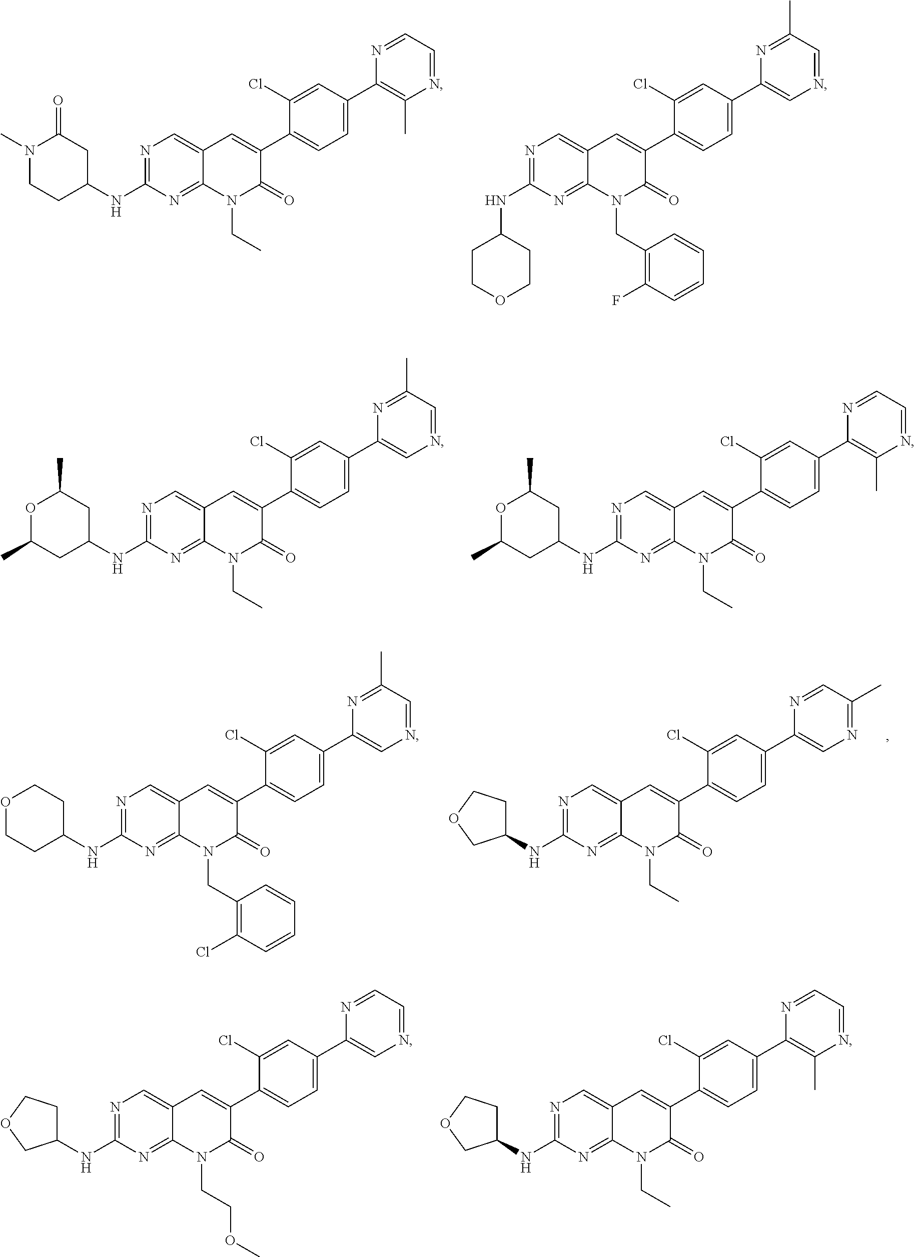

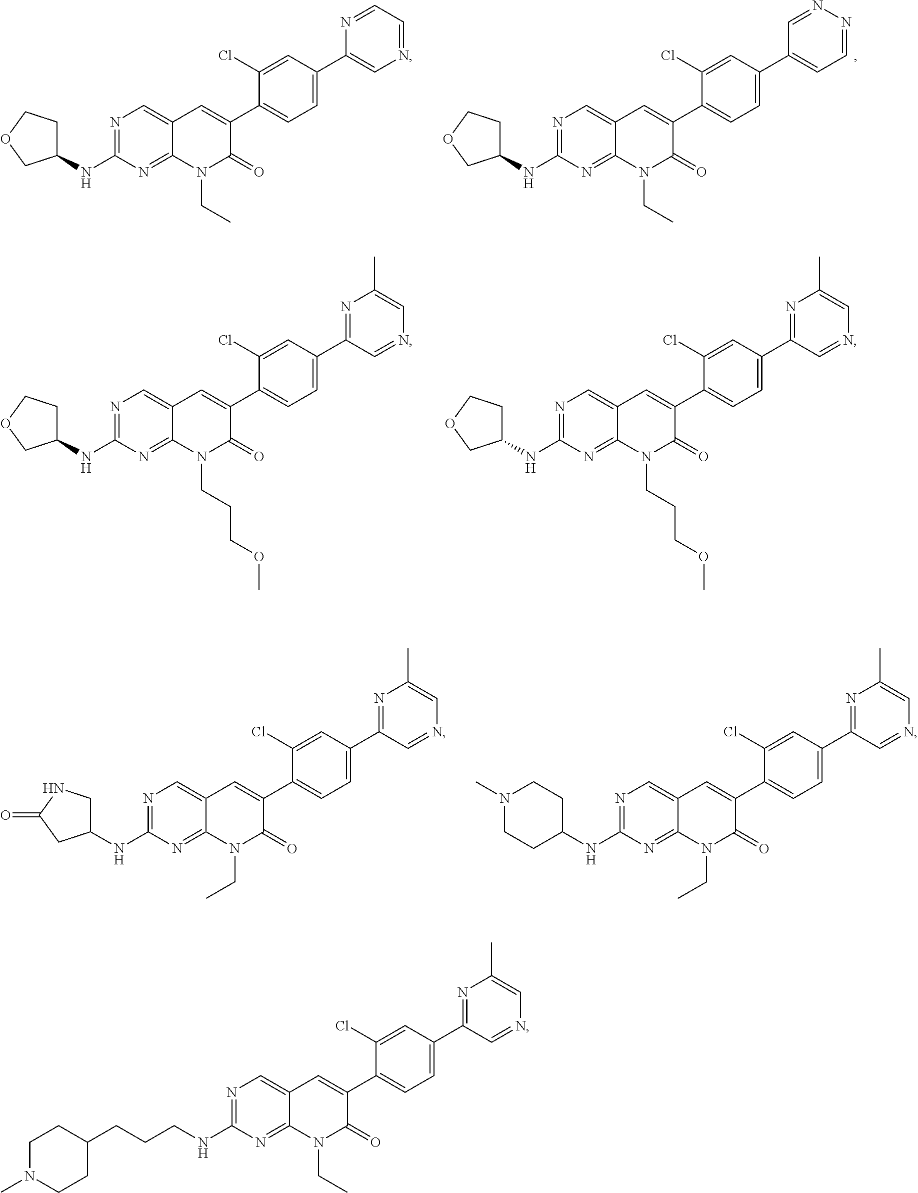

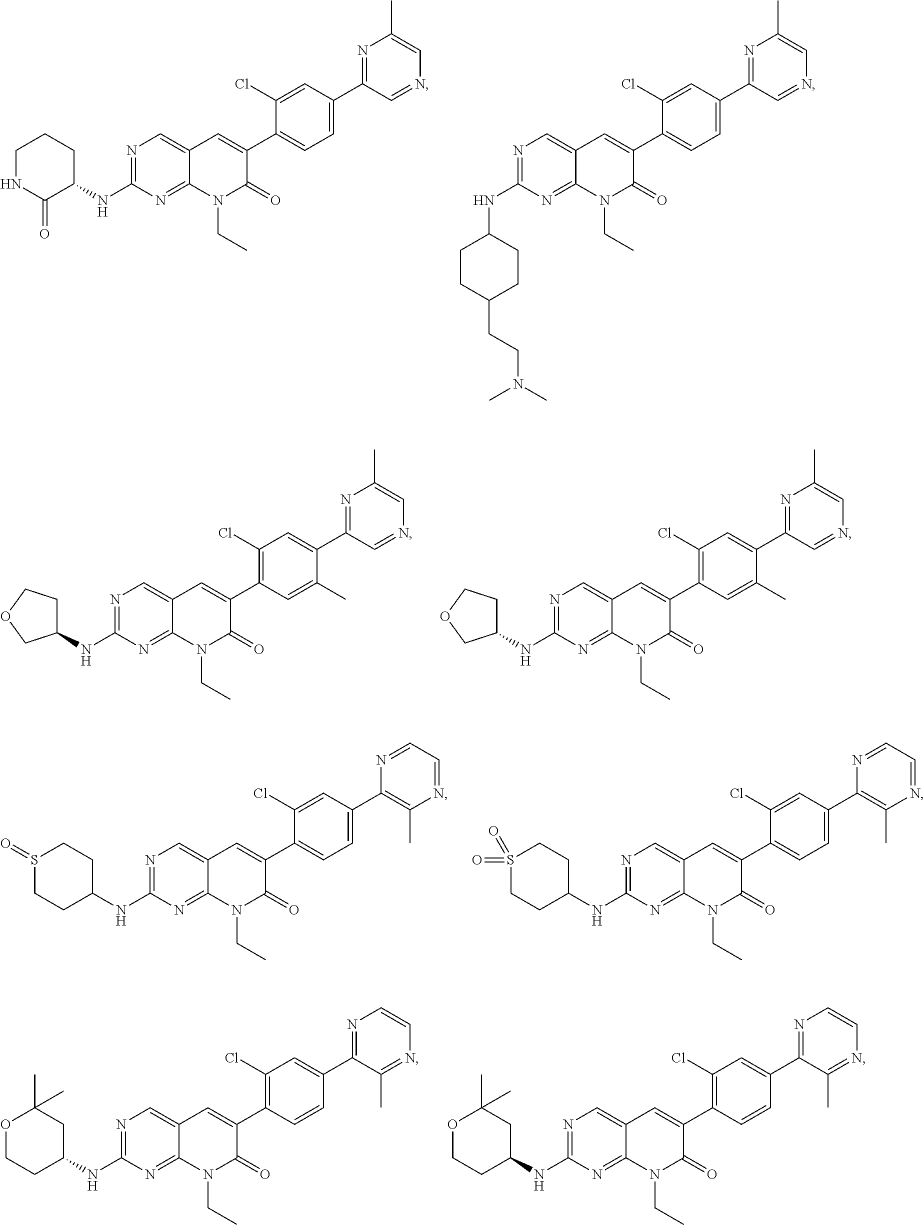

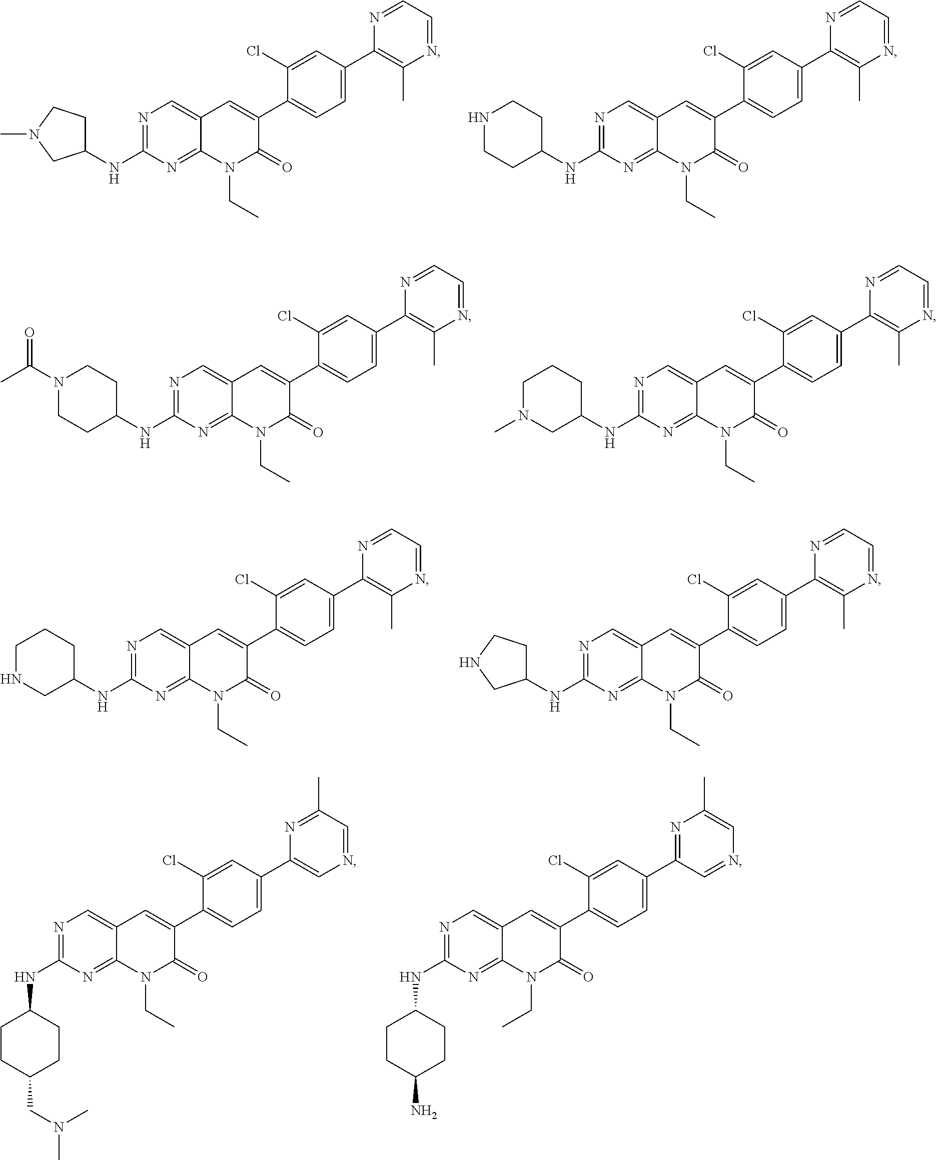

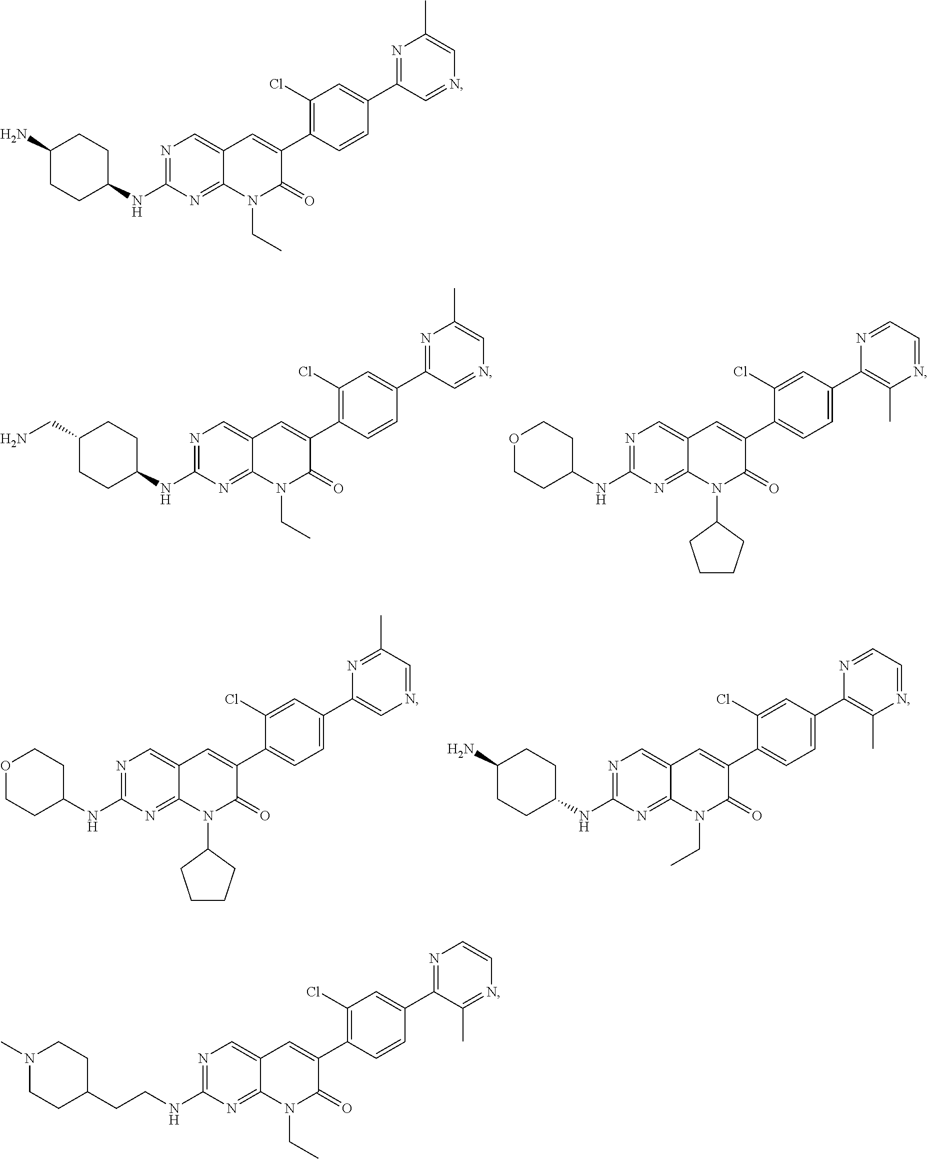 [SMILES: CCn1c(=O)c(-c2ccc(-c3cncc(C)n3)cc2Cl)cc2cnc(N[C@H]3CC[C@@H](N)CC3)nc21 ; CCn1c(=O)c(-c2ccc(-c3cncc(C)n3)cc2Cl)cc2cnc(N[C@H]3CC[C@H](CN)CC3)nc21 ; Cc1nccnc1-c1ccc(-c2cc3cnc(NC4CCOCC4)nc3n(C3CCCC3)c2=O)c(Cl)c1 ; Cc1cncc(-c2ccc(-c3cc4cnc(NC5CCOCC5)nc4n(C4CCCC4)c3=O)c(Cl)c2)n1 ; CCn1c(=O)c(-c2ccc(-c3nccnc3C)cc2Cl)cc2cnc(N[C@H]3CC[C@H](N)CC3)nc21 ; CCn1c(=O)c(-c2ccc(-c3nccnc3C)cc2Cl)cc2cnc(NCCC3CCN(C)CC3)nc21]

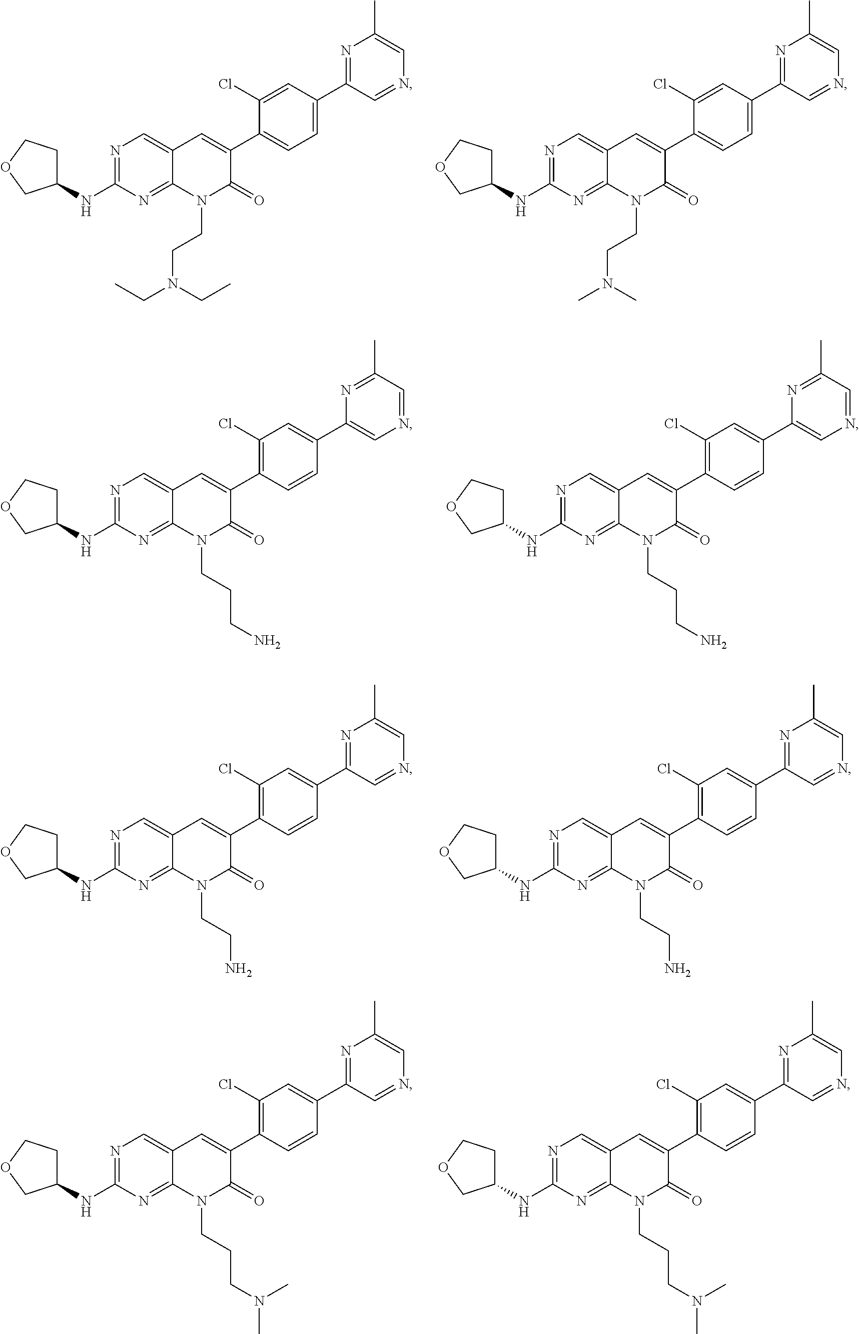

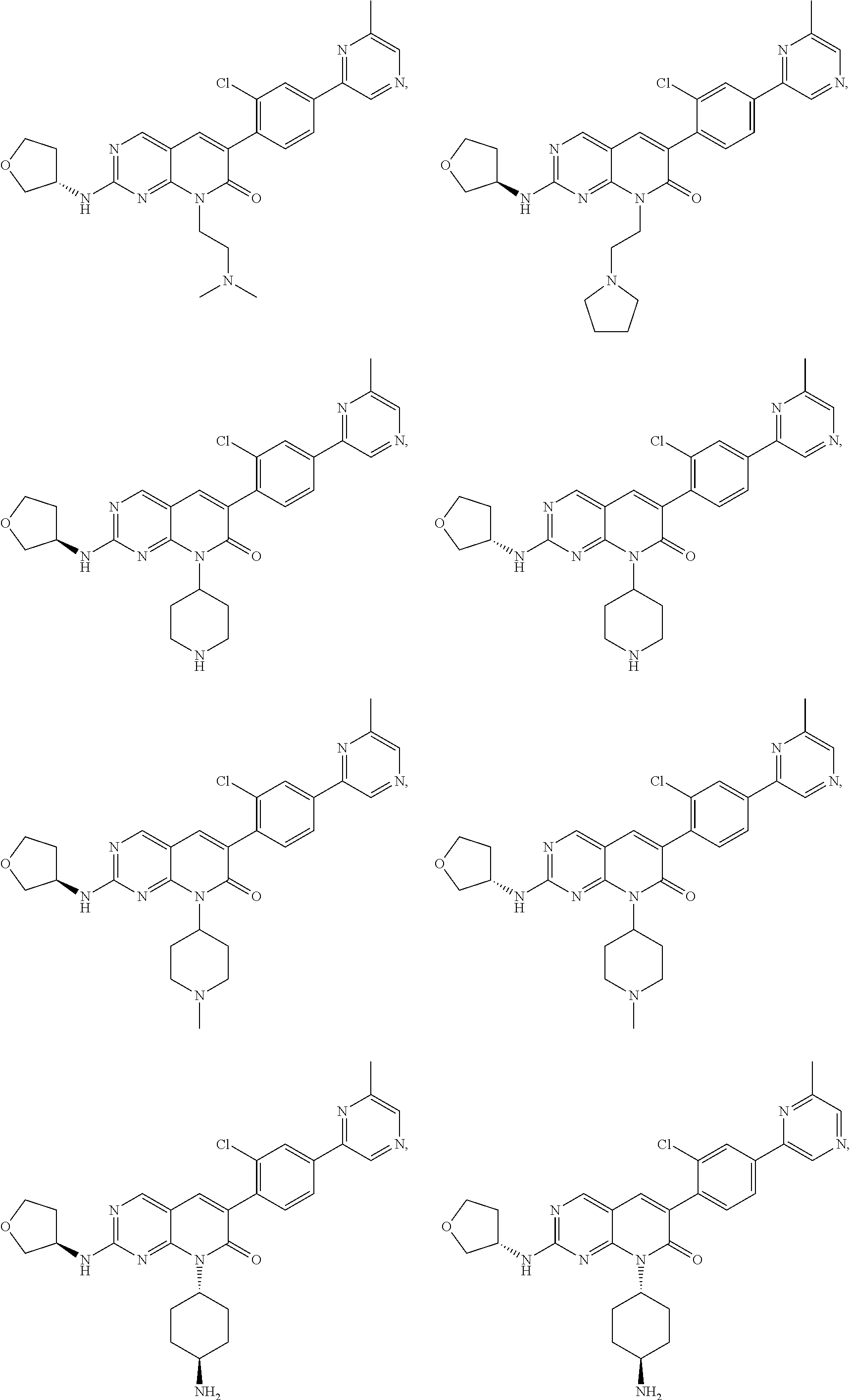

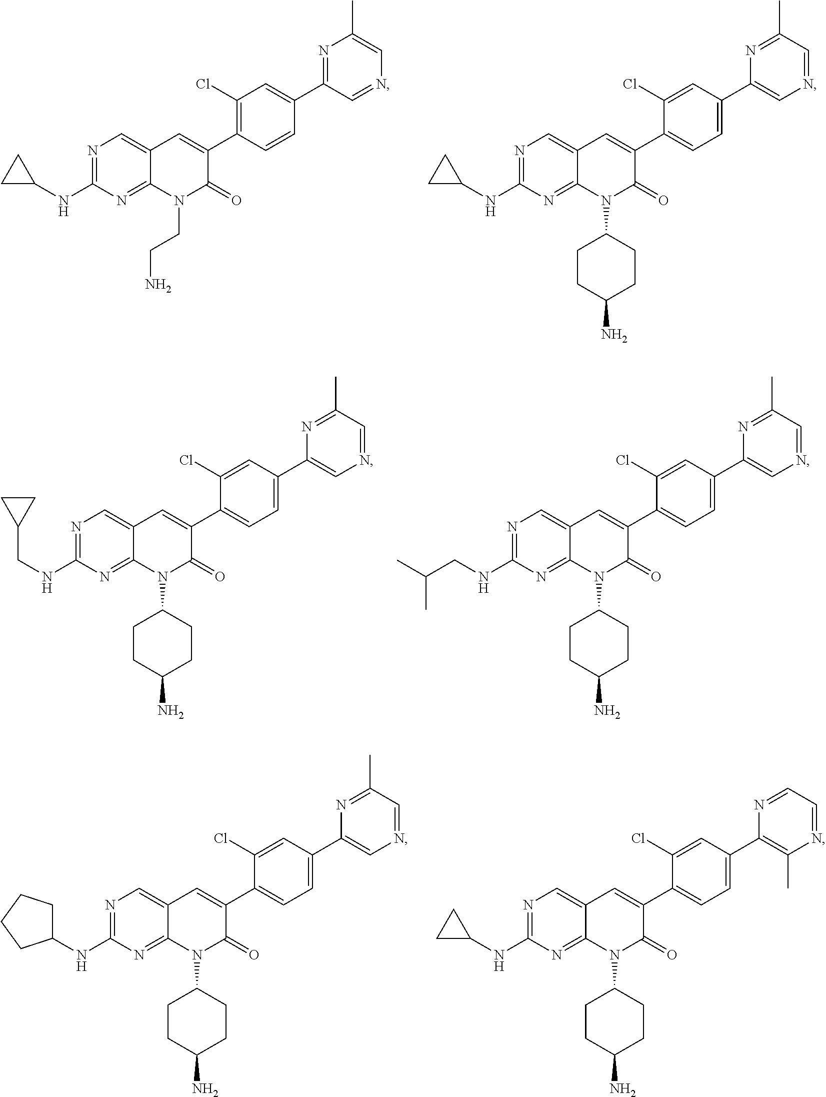 [SMILES: Cc1cncc(-c2ccc(-c3cc4cnc(NC5CC5)nc4n(CCN)c3=O)c(Cl)c2)n1 ; Cc1cncc(-c2ccc(-c3cc4cnc(NC5CC5)nc4n([C@H]4CC[C@H](N)CC4)c3=O)c(Cl)c2)n1 ; Cc1cncc(-c2ccc(-c3cc4cnc(NCC5CC5)nc4n([C@H]4CC[C@H](N)CC4)c3=O)c(Cl)c2)n1 ; Cc1cncc(-c2ccc(-c3cc4cnc(NCC(C)C)nc4n([C@H]4CC[C@H](N)CC4)c3=O)c(Cl)c2)n1 ; Cc1cncc(-c2ccc(-c3cc4cnc(NC5CCCC5)nc4n([C@H]4CC[C@H](N)CC4)c3=O)c(Cl)c2)n1 ; Cc1nccnc1-c1ccc(-c2cc3cnc(NC4CC4)nc3n([C@H]3CC[C@H](N)CC3)c2=O)c(Cl)c1]

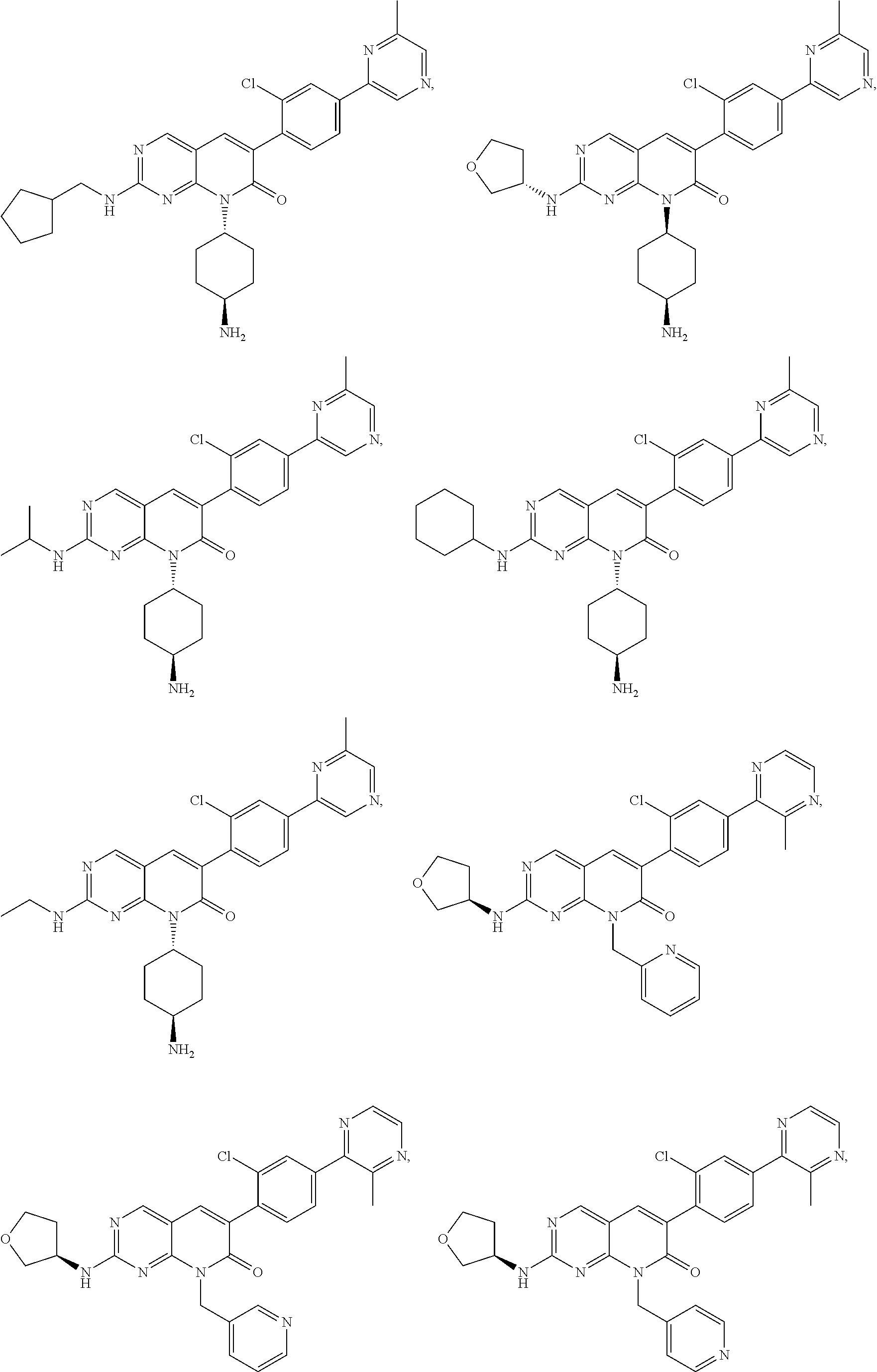

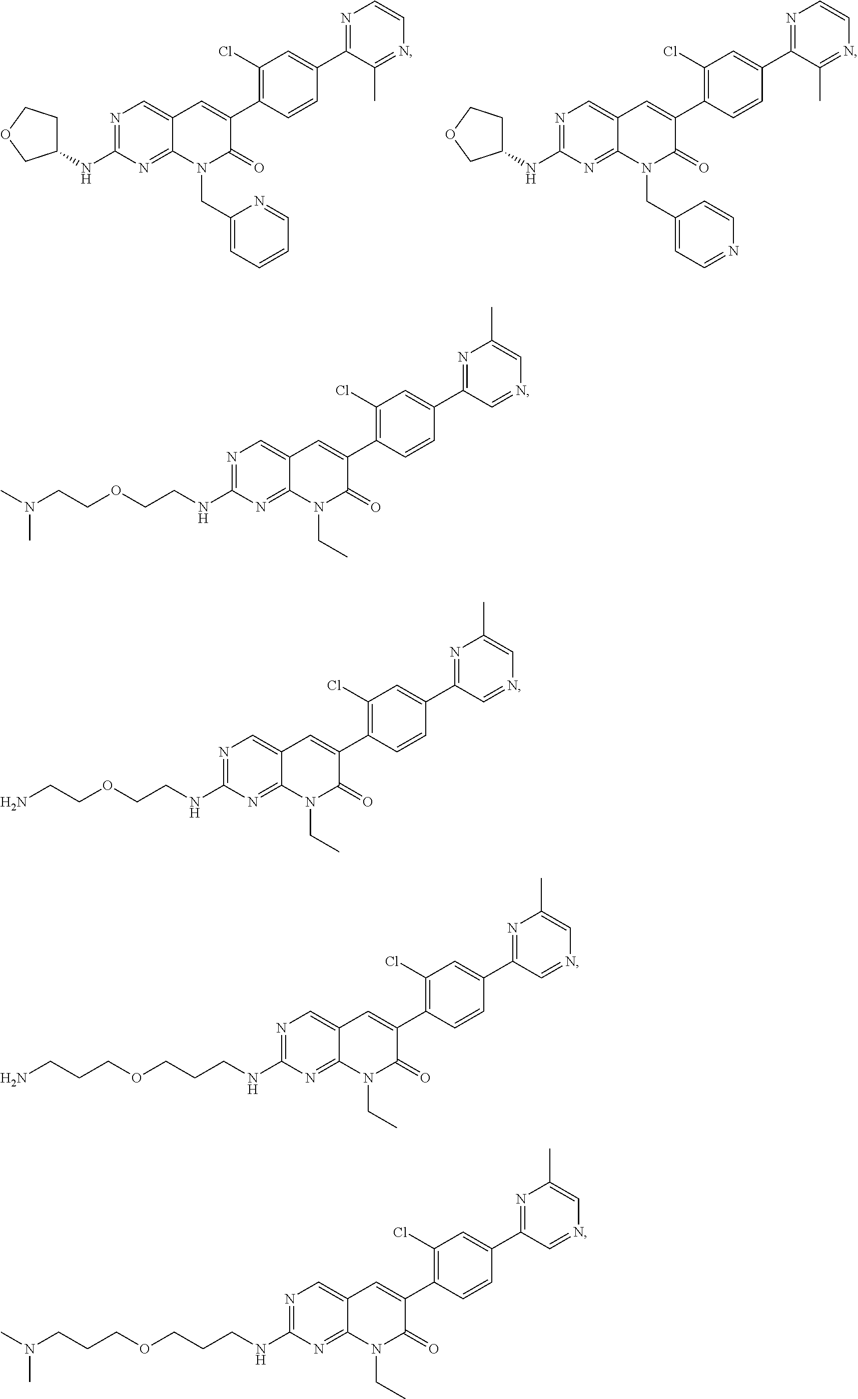 [SMILES: Cc1nccnc1-c1ccc(-c2cc3cnc(N[C@H]4CCOC4)nc3n(Cc3ccccn3)c2=O)c(Cl)c1 ; Cc1nccnc1-c1ccc(-c2cc3cnc(N[C@H]4CCOC4)nc3n(Cc3ccncc3)c2=O)c(Cl)c1 ; CCn1c(=O)c(-c2ccc(-c3cncc(C)n3)cc2Cl)cc2cnc(NCCOCCN(C)C)nc21 ; CCn1c(=O)c(-c2ccc(-c3cncc(C)n3)cc2Cl)cc2cnc(NCCOCCN)nc21 ; CCn1c(=O)c(-c2ccc(-c3cncc(C)n3)cc2Cl)cc2cnc(NCCCOCCCN)nc21 ; CCn1c(=O)c(-c2ccc(-c3cncc(C)n3)cc2Cl)cc2cnc(NCCCOCCCN(C)C)nc21]

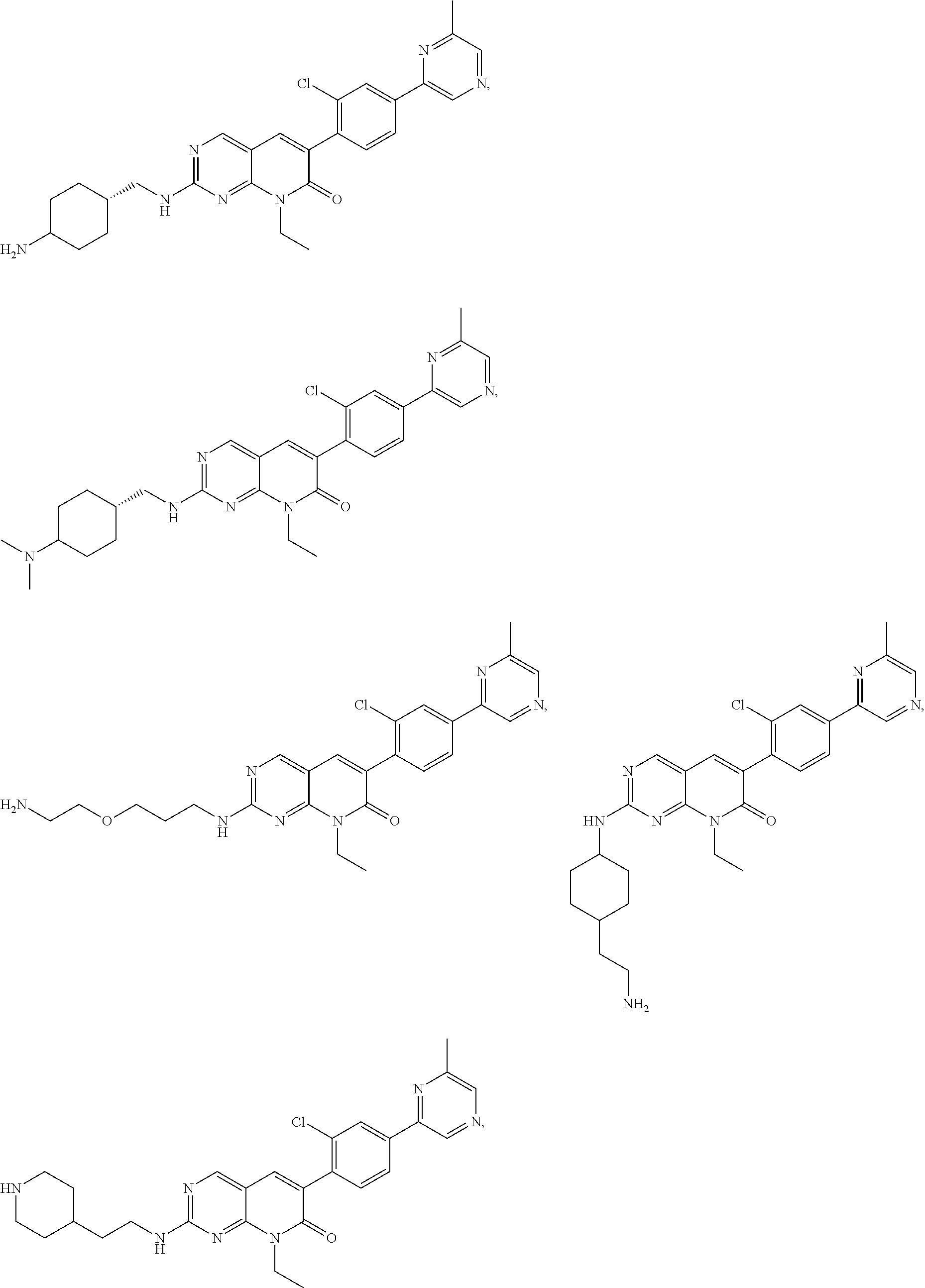

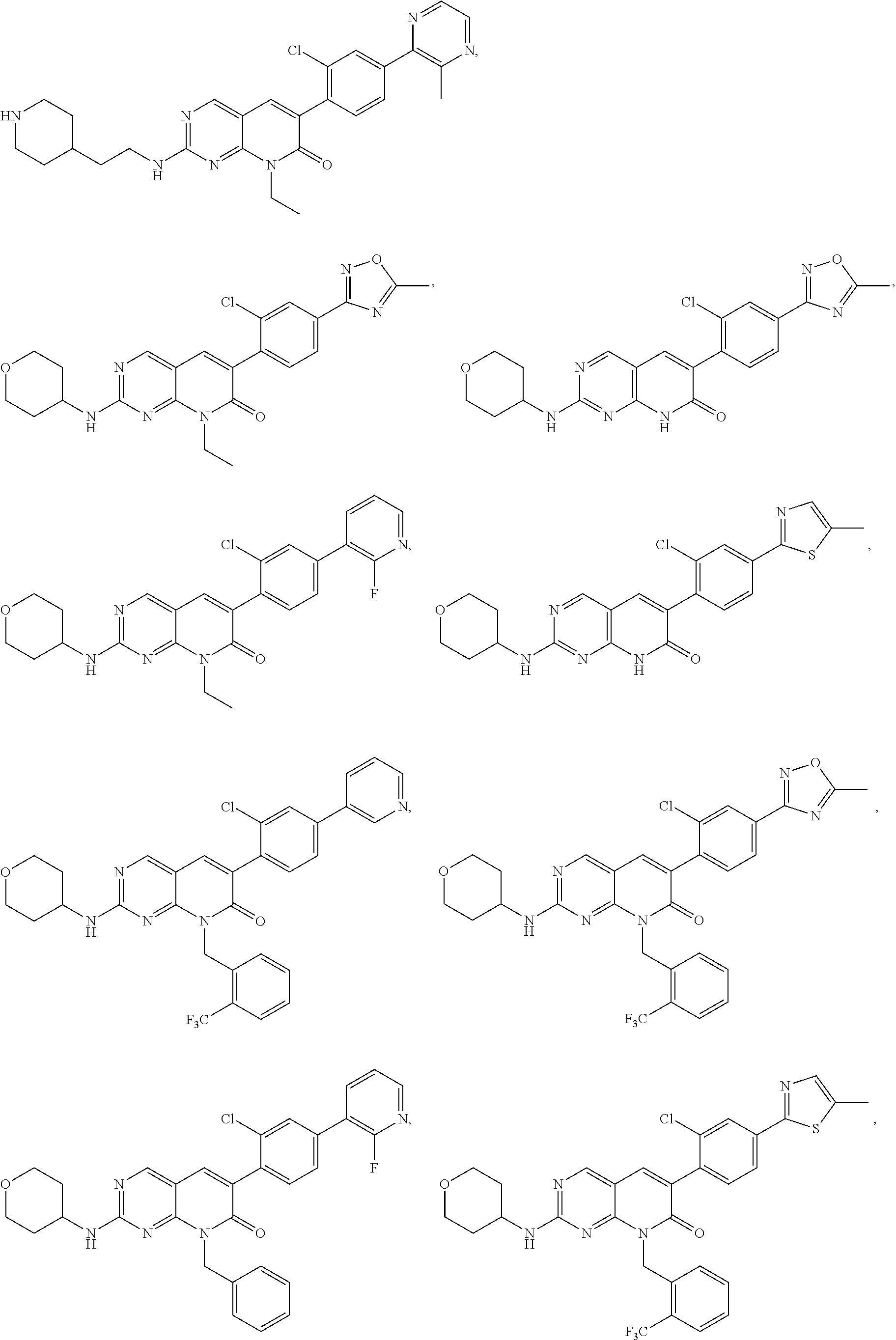

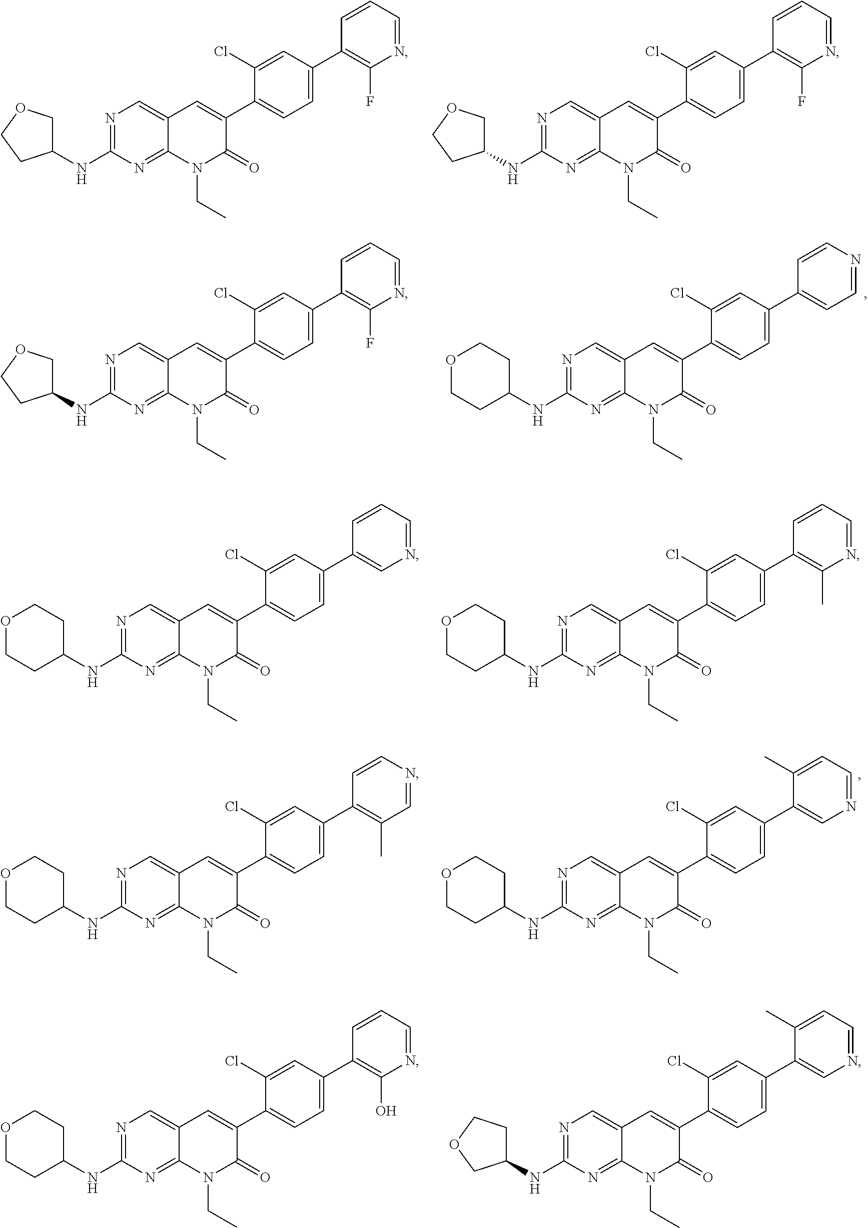

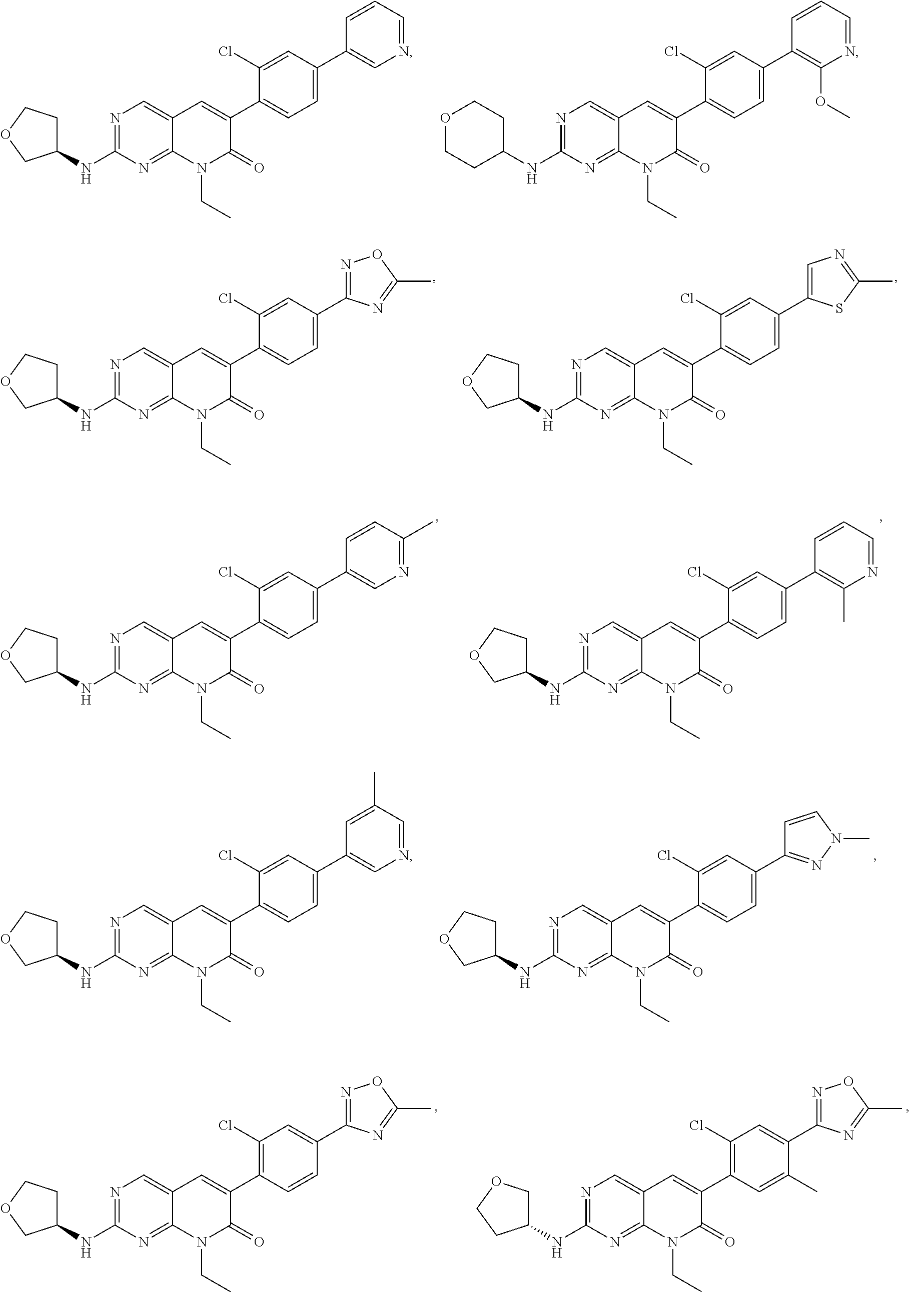

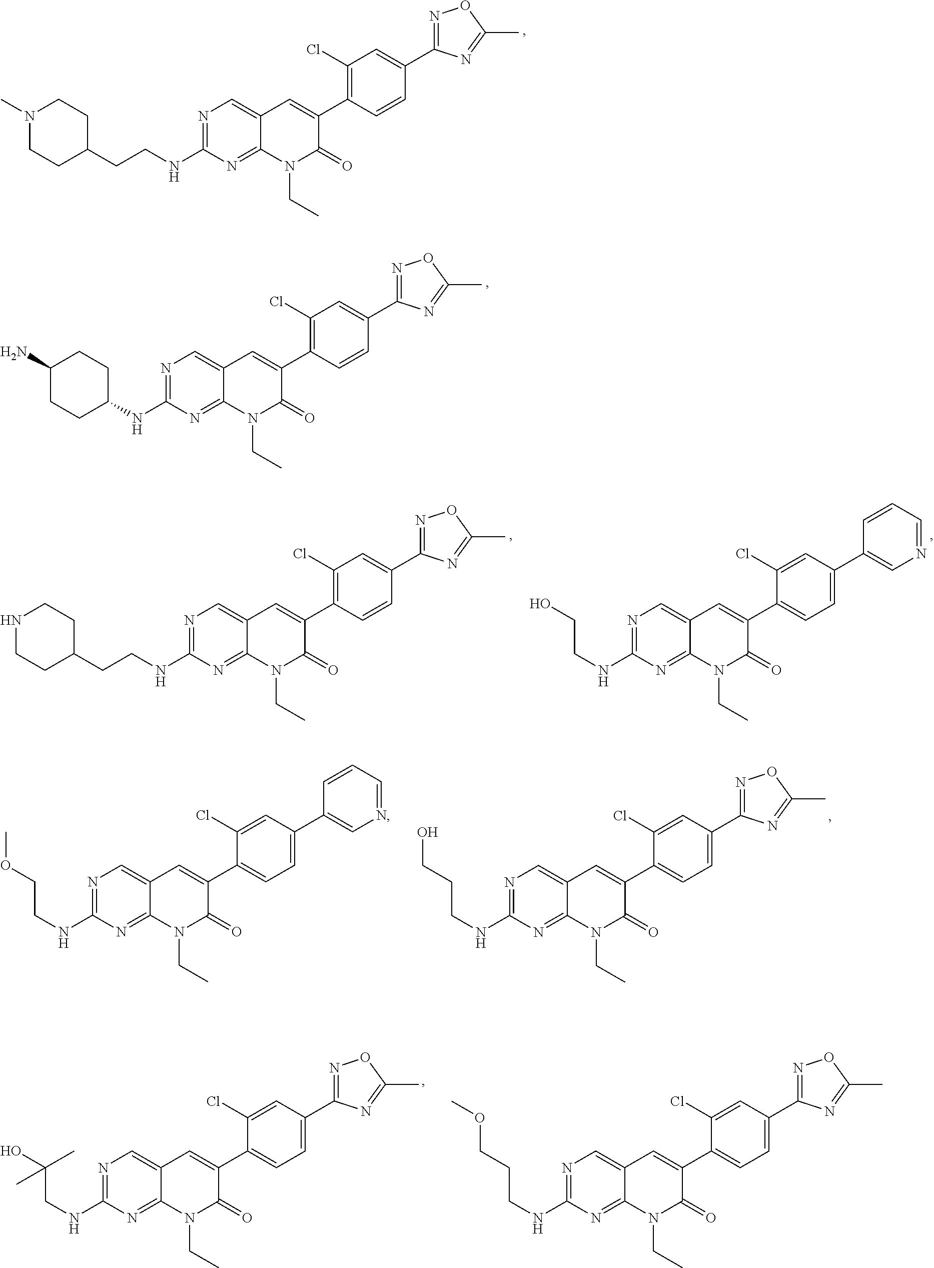

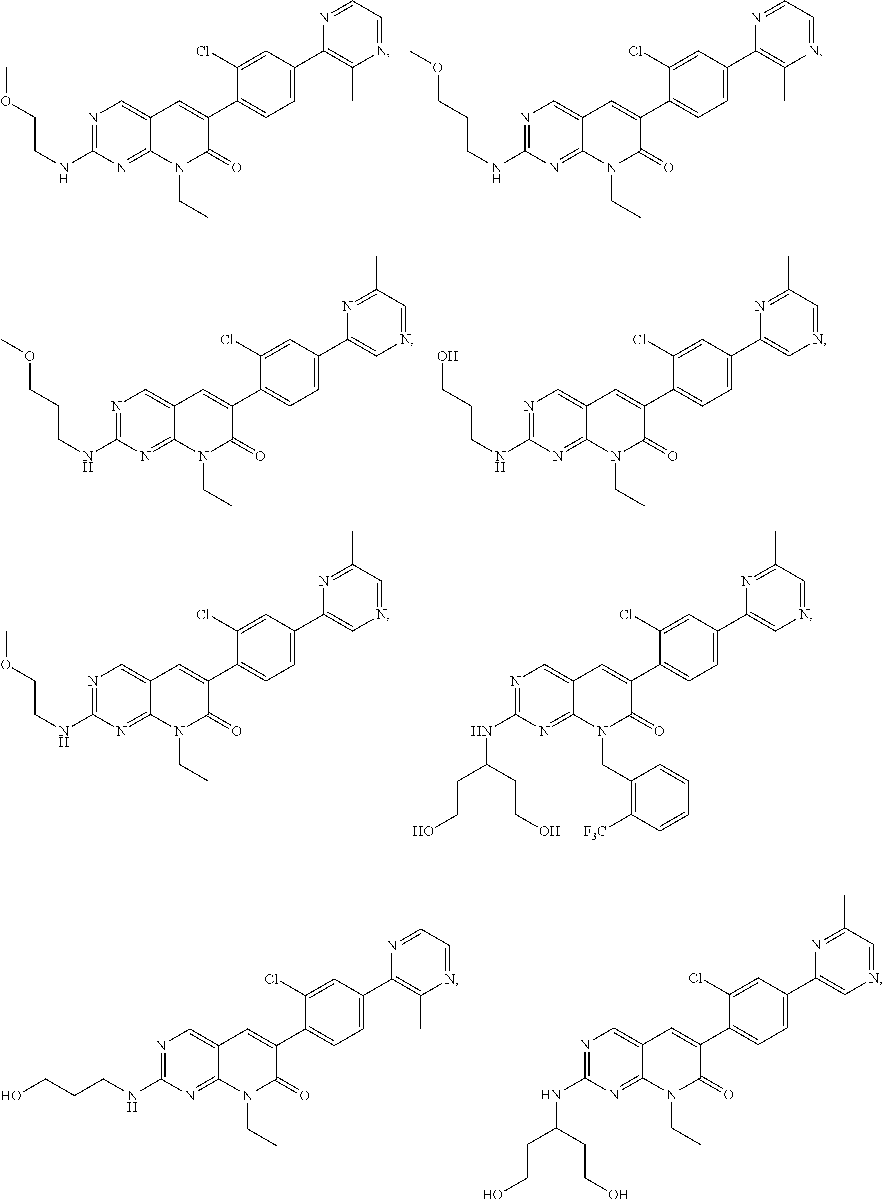

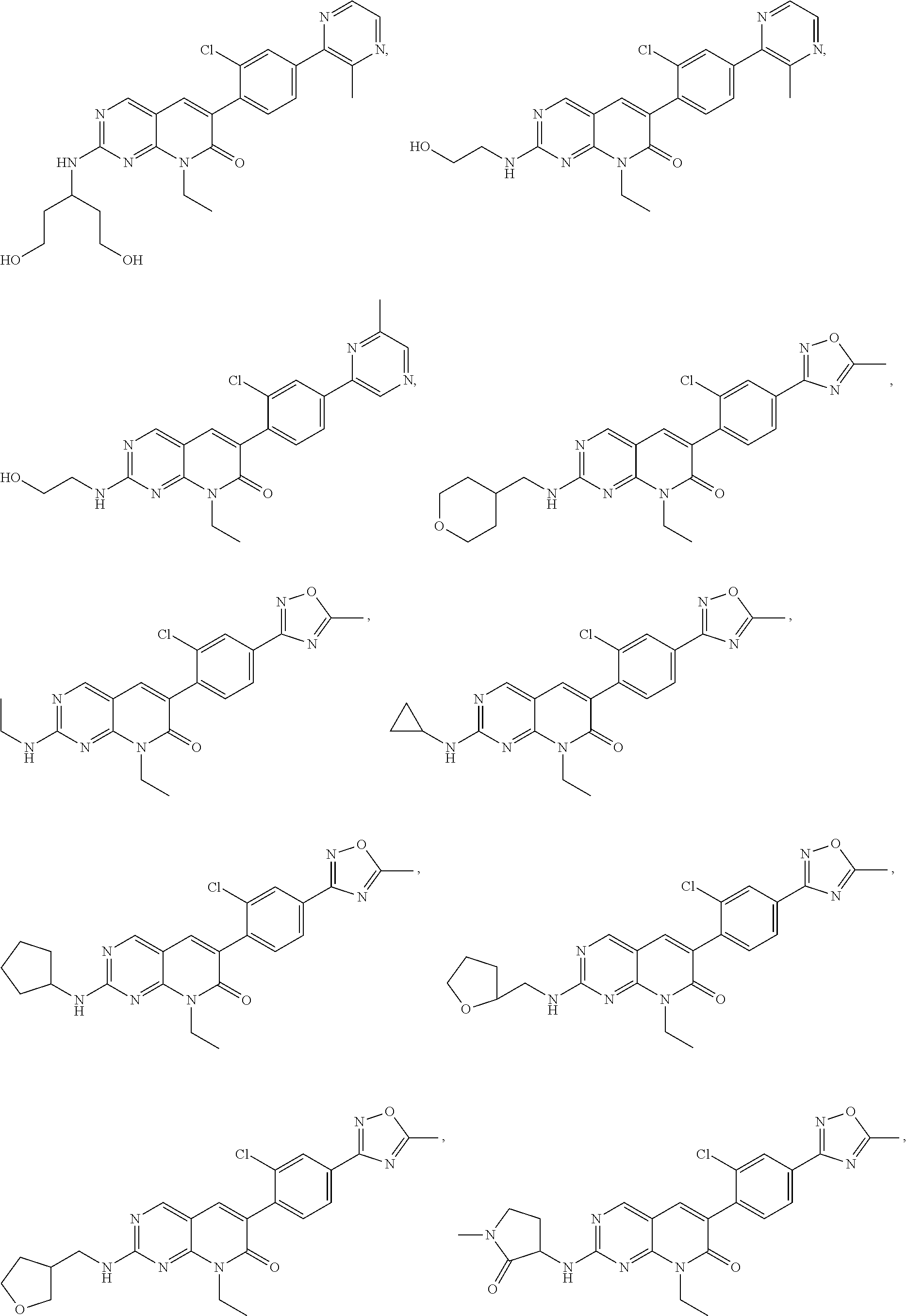

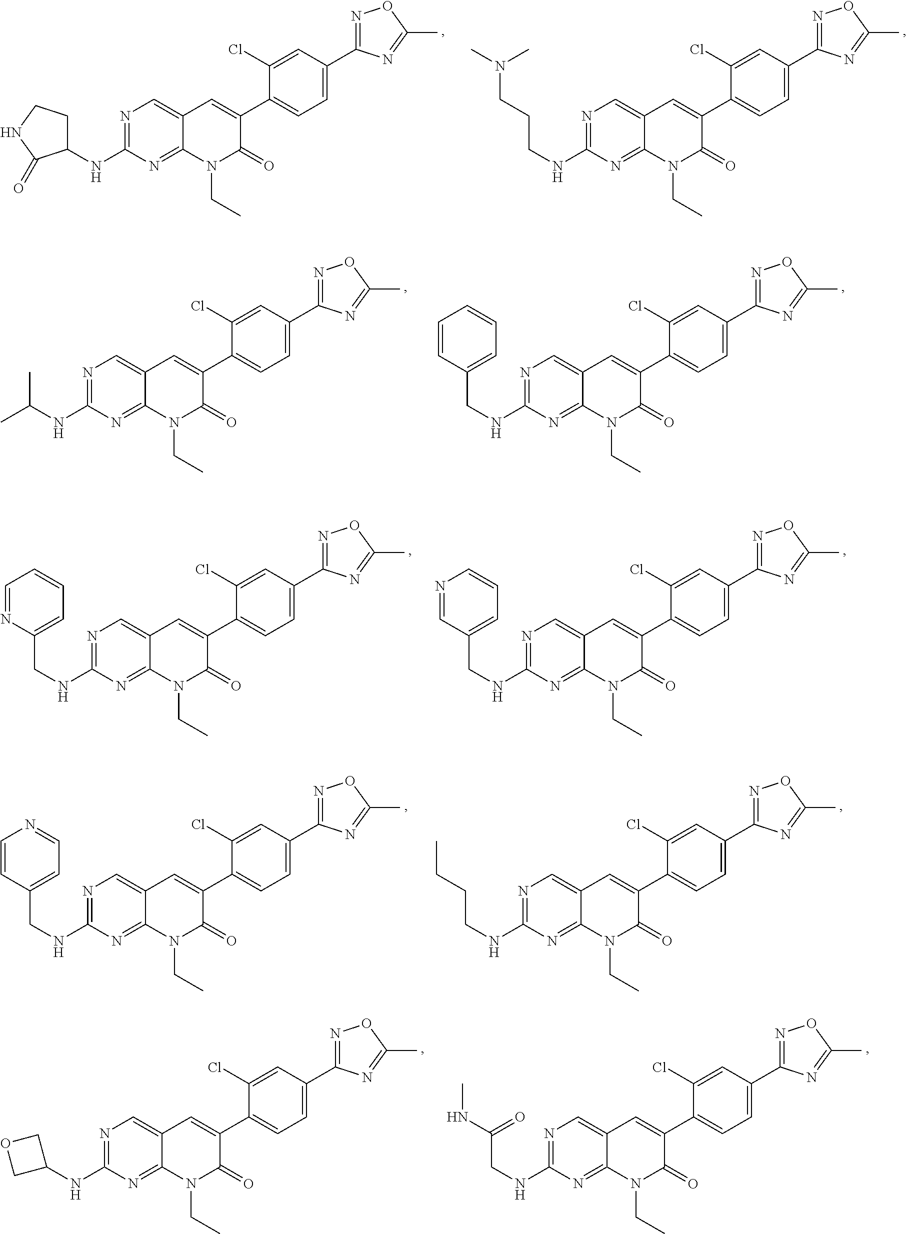

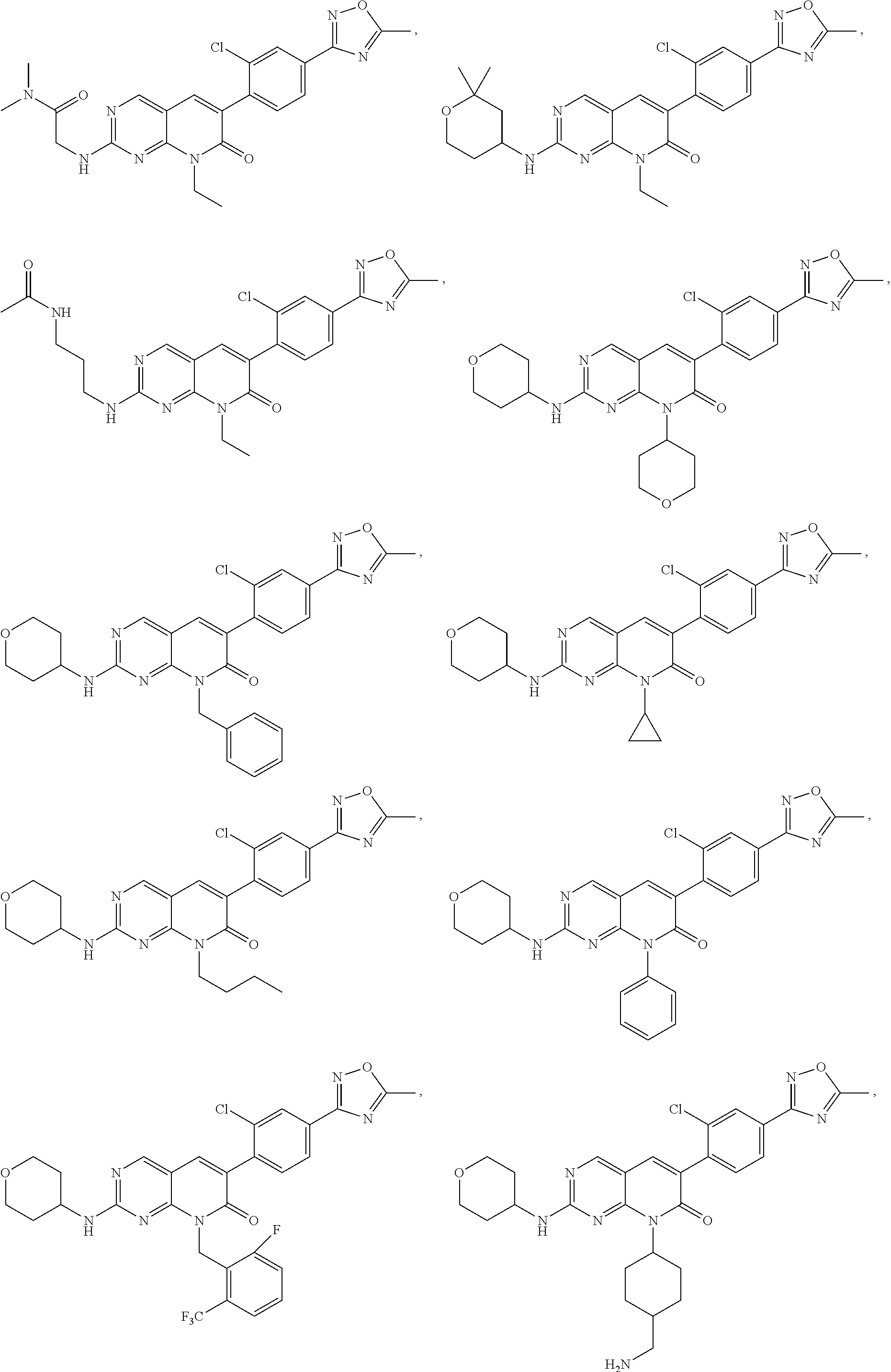

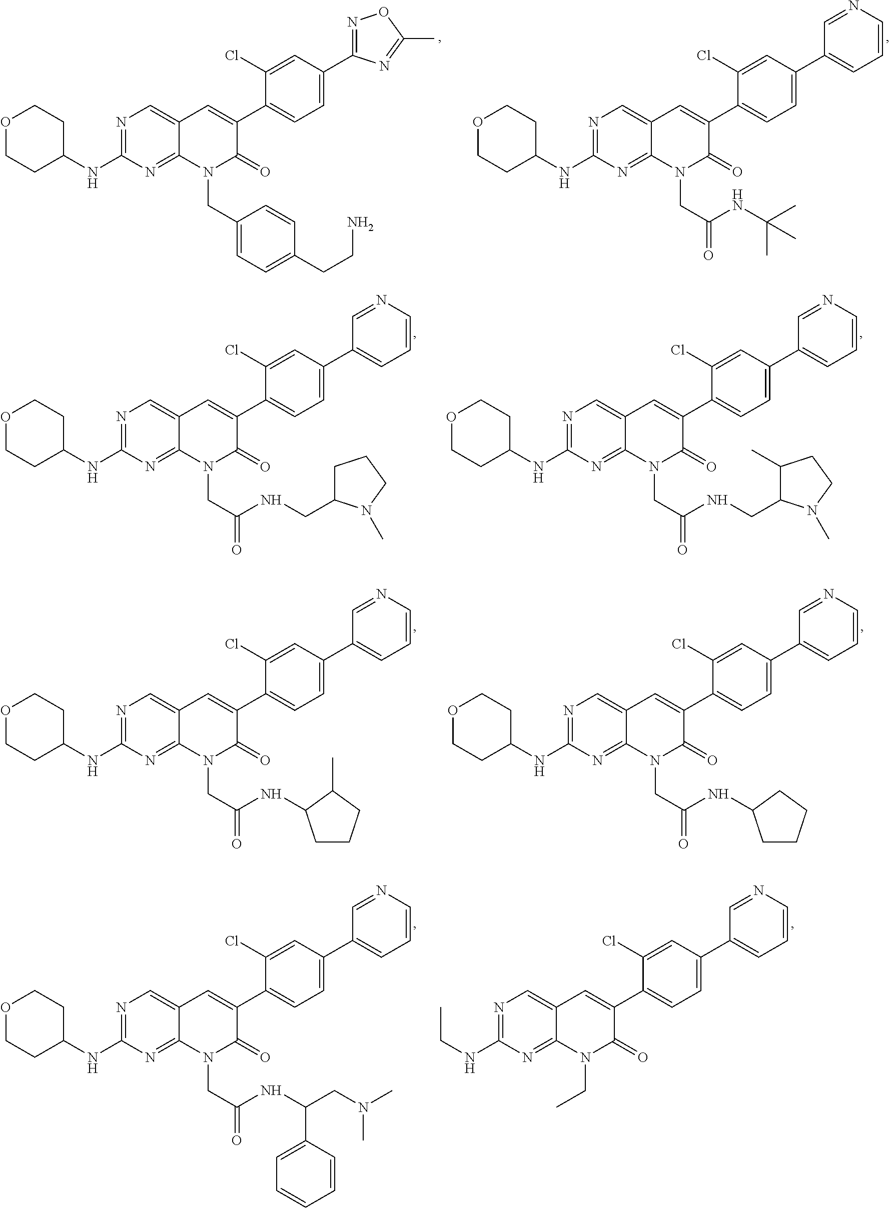

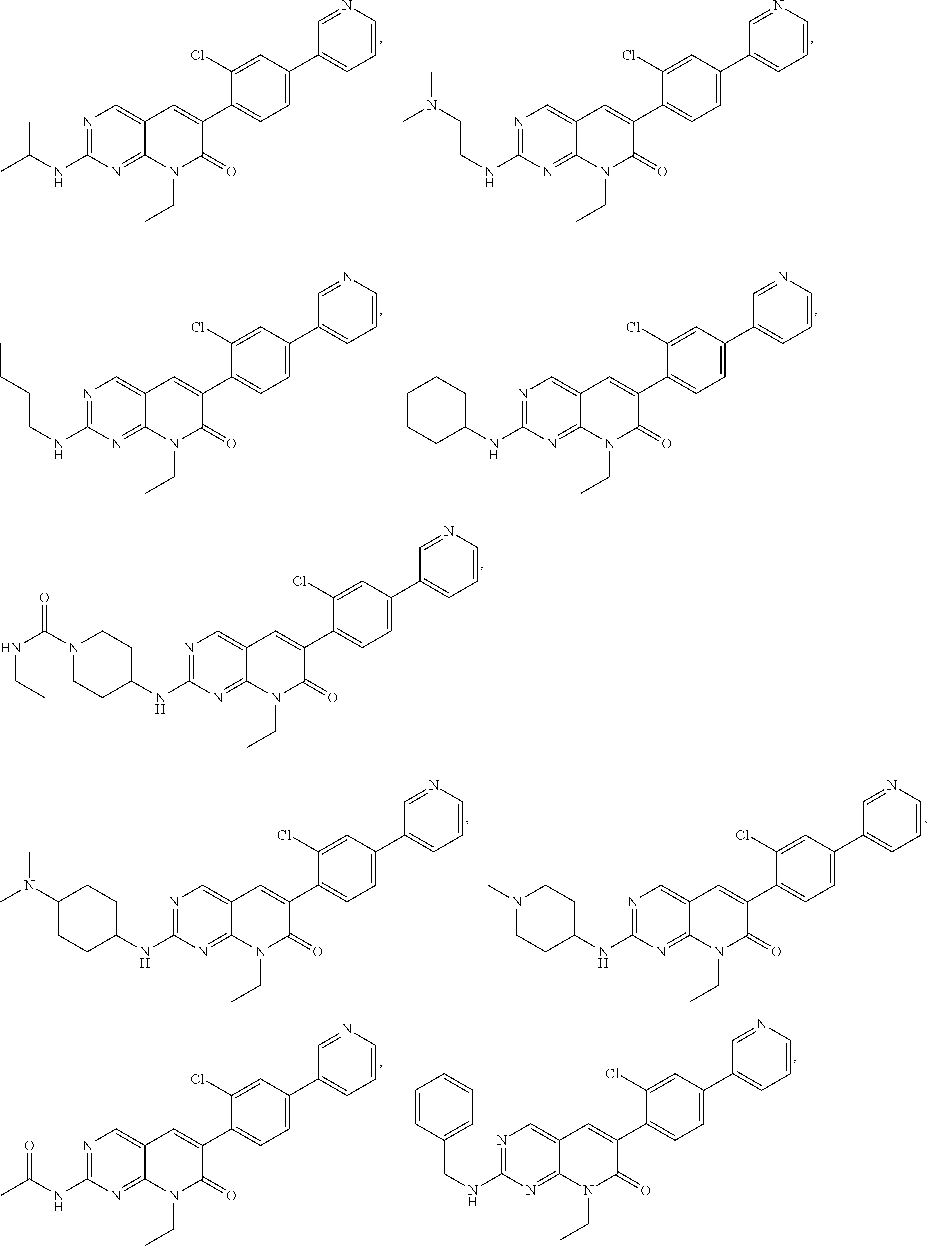

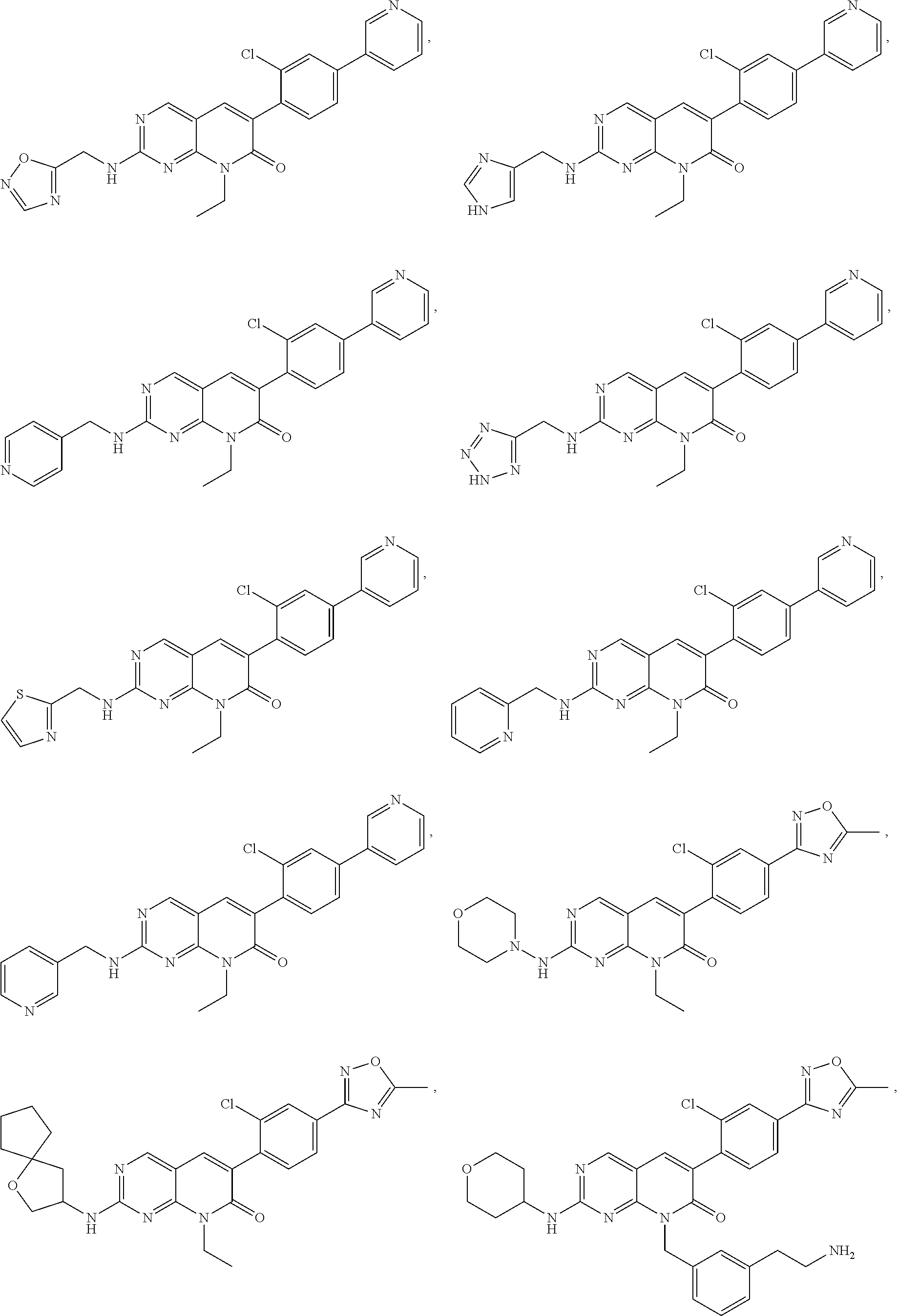

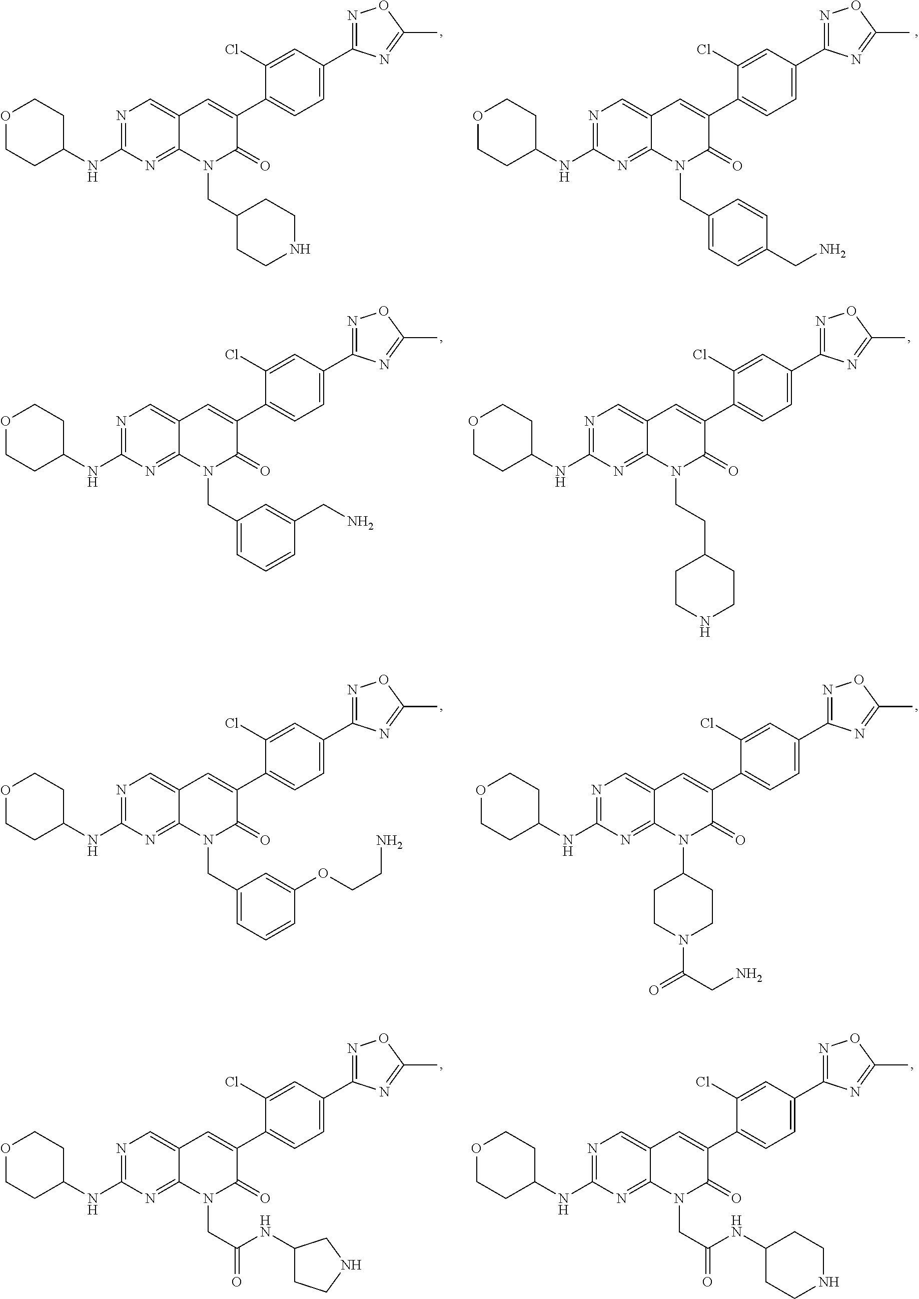

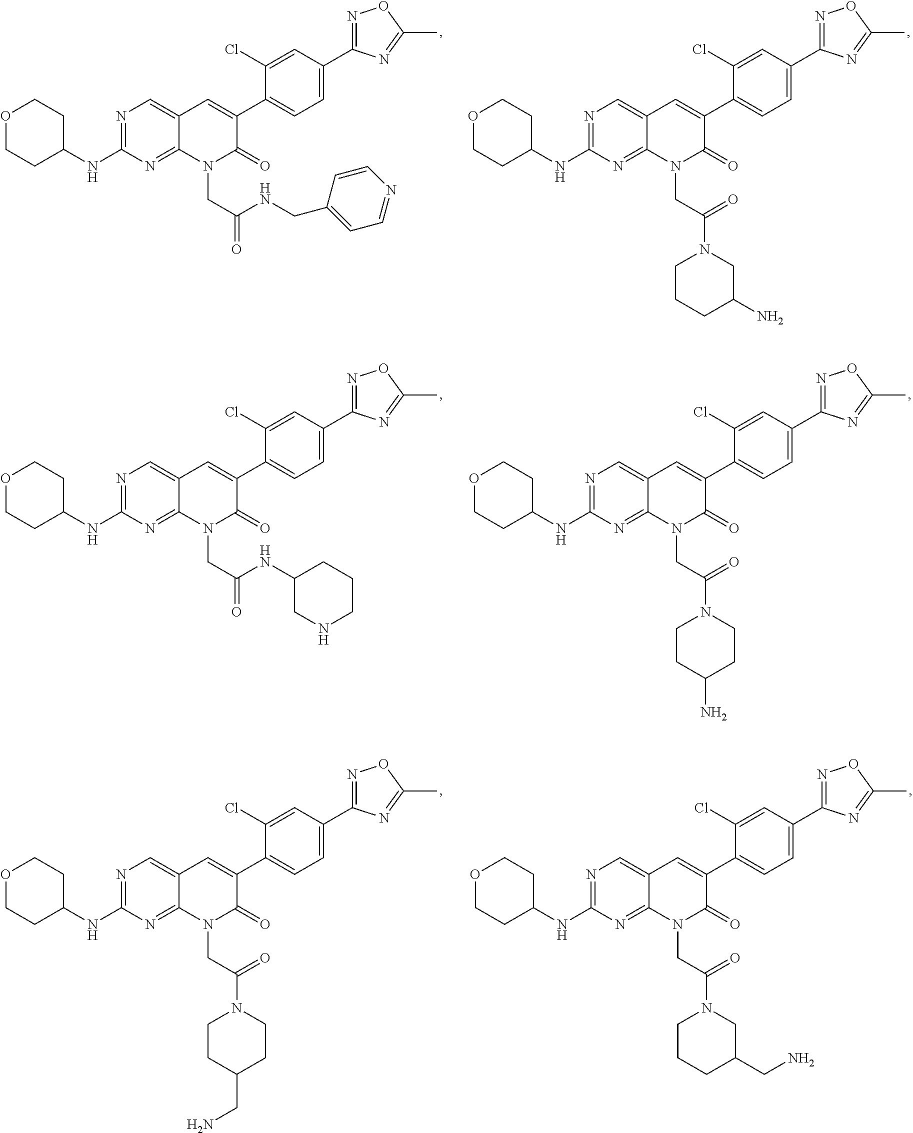 [SMILES: Cc1nc(-c2ccc(-c3cc4cnc(NC5CCOCC5)nc4n(CC(=O)NCc4ccncc4)c3=O)c(Cl)c2)no1 ; Cc1nc(-c2ccc(-c3cc4cnc(NC5CCOCC5)nc4n(CC(=O)N4CCCC(N)C4)c3=O)c(Cl)c2)no1 ; Cc1nc(-c2ccc(-c3cc4cnc(NC5CCOCC5)nc4n(CC(=O)NC4CCCNC4)c3=O)c(Cl)c2)no1 ; Cc1nc(-c2ccc(-c3cc4cnc(NC5CCOCC5)nc4n(CC(=O)N4CCC(N)CC4)c3=O)c(Cl)c2)no1 ; Cc1nc(-c2ccc(-c3cc4cnc(NC5CCOCC5)nc4n(CC(=O)N4CCC(CN)CC4)c3=O)c(Cl)c2)no1 ; Cc1nc(-c2ccc(-c3cc4cnc(NC5CCOCC5)nc4n(CC(=O)N4CCCC(CN)C4)c3=O)c(Cl)c2)no1]

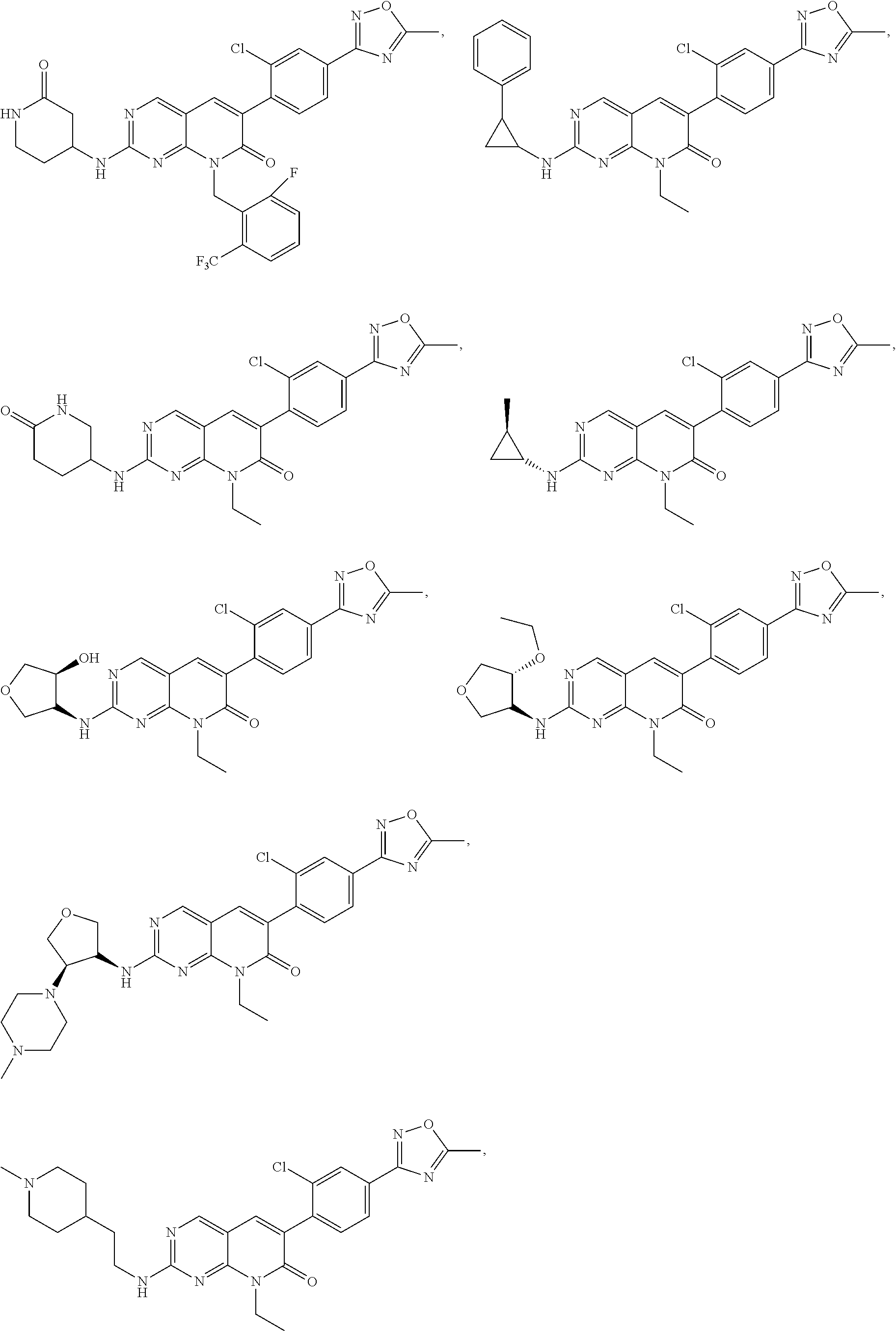

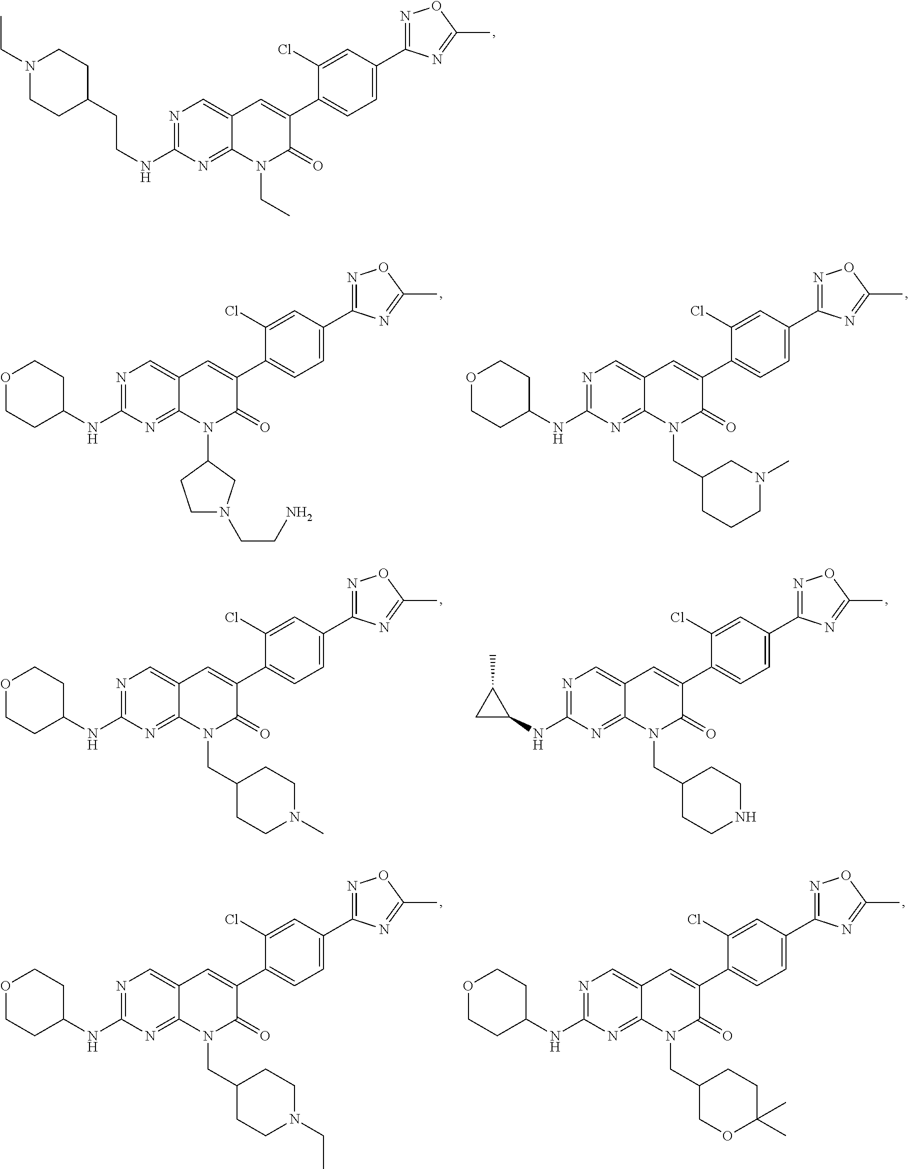

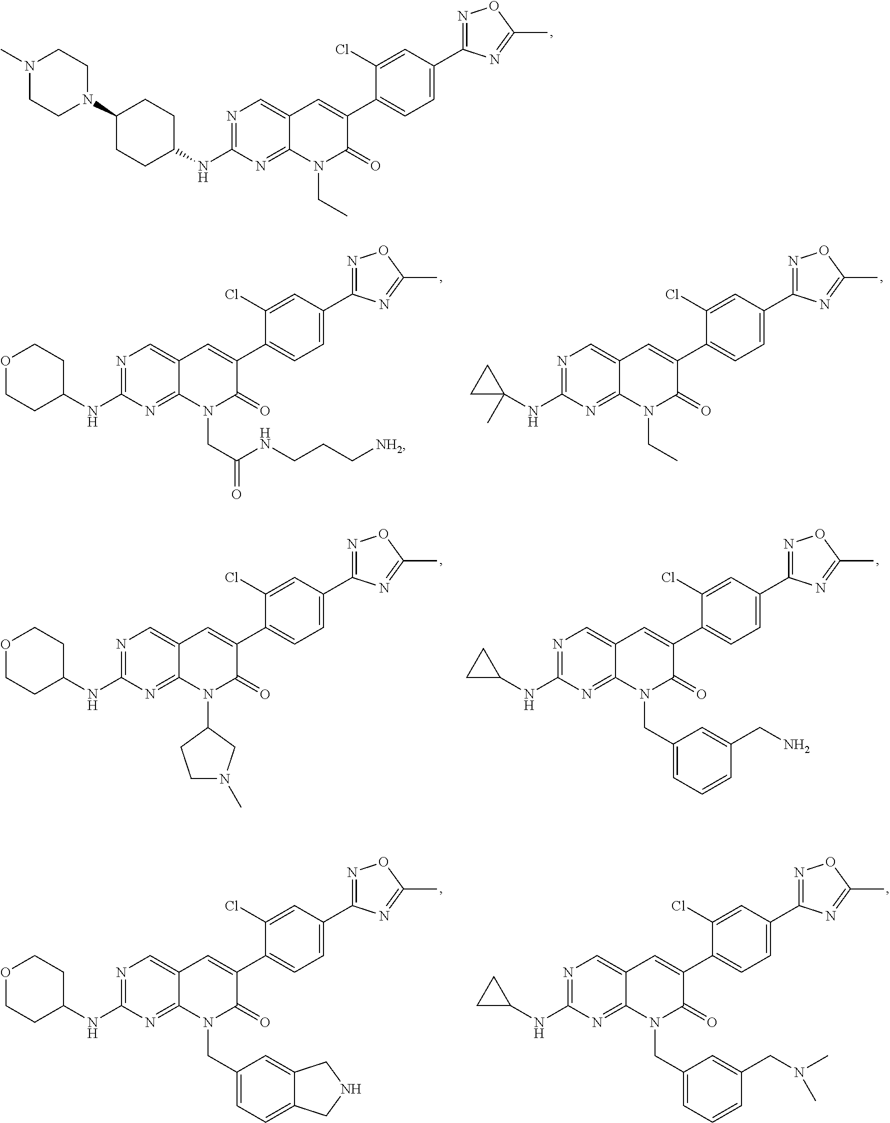

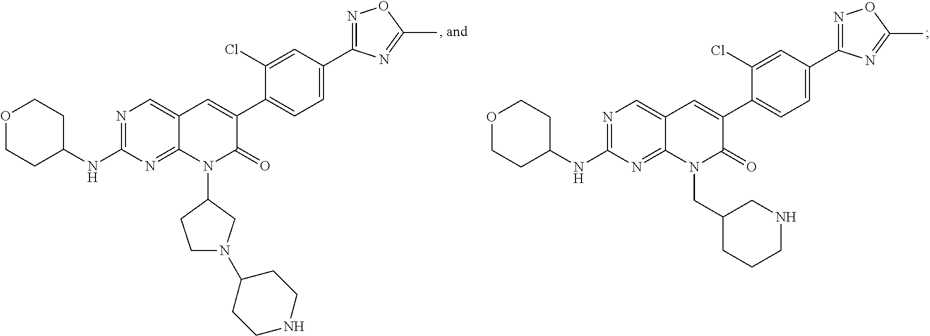 [SMILES: Cc1nc(-c2ccc(-c3cc4cnc(NC5CCOCC5)nc4n(C4CCN(C5CCNCC5)C4)c3=O)c(Cl)c2)no1 ; Cc1nc(-c2ccc(-c3cc4cnc(NC5CCOCC5)nc4n(CC4CCCNC4)c3=O)c(Cl)c2)no1]

or a pharmaceutically acceptable salt or N-oxide thereof

Provided herein are methods for treating Fragile X syndrome comprising administration of a therapeutically effective amount of a p21-activated kinase inhibitor (e.g., a compound of Formula I-IV and A-D) to an individual in need thereof. In some embodiments of the methods provided herein, administration of a p21-activated kinase inhibitor alleviates or reverses one or more behavioral symptoms (e.g., social withdrawal, depersonalization, loss of appetite, loss of hygiene, delusions, hallucinations, depression, blunted affect, avolition, anhedonia, alogia, the sense of being controlled by outside forces or the like) of Fragile X syndrome. In some embodiments of the methods provided herein, administration of a p21-activated kinase inhibitor (e.g., a compound of Formula I-IV and A-D) alleviates or reverses one or more negative symptoms and/or cognition impairment associated with Fragile X syndrome.

Also provided herein are methods for modulation of dendritic spine morphology and/or synaptic function comprising administering to an individual in need thereof (e.g., Fragile X syndrome) a therapeutically effective amount of a PAK inhibitor (e.g., a compound of Formula I-IV and A-D). In some embodiments, modulation of dendritic spine morphology and/or synaptic function alleviates or reverses negative symptoms and/or cognitive impairment associated with Fragile X syndrome. In some embodiments, modulation of dendritic spine morphology and/or synaptic function halts or delays further deterioration of symptoms associated with Fragile X syndrome. In some embodiments, modulation of dendritic spine morphology and/or synaptic function stabilizes or reverses symptoms of Fragile X syndrome. In some embodiments of the methods provided herein, administration of a p21-activated kinase inhibitor halts or delays progressive loss of memory and/or cognition associated with Fragile X syndrome.

Provided herein are methods for modulation of synaptic function or synaptic plasticity comprising administering to an individual in need thereof (e.g., an individual suffering from or suspected of having Fragile X syndrome described herein) a therapeutically effective amount of a PAK inhibitor (e.g., a compound of Formula I-IV and A-D). Modulation of synaptic function or plasticity includes, for example, alleviation or reversal of defects in LTP, LTD or the like.

Defects in LTP include, for example, an increase in LTP or a decrease in LTP in any region of the brain in an individual suffering from or suspected of having Fragile X syndrome. Defects in LTD include for example a decrease in LTD or an increase in LTD in any region of the brain (e.g., the temporal lobe, parietal lobe, the frontal cortex, the cingulate gyrus, the prefrontal cortex, the cortex, or the hippocampus or any other region in the brain or a combination thereof) in an individual suffering from or suspected of having Fragile X syndrome.

In some embodiments of the methods, administration of a PAK inhibitor (e.g., a compound of Formula I-IV and A-D) modulates synaptic function (e.g., synaptic transmission and/or plasticity) by increasing long term potentiation (LTP) in an individual suffering from or suspected of having Fragile X syndrome. In some embodiments of the methods described herein, administration of a PAK inhibitor (e.g., a compound of Formula I-IV and A-D) to an individual in need thereof modulates synaptic function (e.g., synaptic transmission and/or plasticity) by increasing long term potentiation (LTP) in the prefrontal cortex, or the cortex, or the hippocampus or any other region in the brain or a combination thereof. In some embodiments of the methods described herein, administration of a PAK inhibitor modulates synaptic function (e.g., synaptic transmission and/or plasticity) by decreasing long term depression (LTD) in an individual suffering from or suspected of having Fragile X syndrome. In some embodiments of the methods described herein, administration of a PAK inhibitor to an individual in need thereof modulates synaptic function (e.g., synaptic transmission and/or plasticity) by decreasing long term depression (LTD) in the temporal lobe, parietal lobe, the frontal cortex, the cingulate gyrus, the prefrontal cortex, the cortex, or the hippocampus or any other region in the brain or a combination thereof.

In some embodiments of the methods described herein, administration of a PAK inhibitor reverses defects in synaptic function (i.e. synaptic transmission and/or synaptic plasticity, induced by soluble Abeta dimers or oligomers. In some embodiments of the methods described herein, administration of a PAK inhibitor reverses defects in synaptic function (i.e. synaptic transmission and/or synaptic plasticity, induced by insoluble Abeta oligomers and/or Abeta-containing plaques.

Provided herein are methods for stabilization of synaptic plasticity comprising administering to an individual in need thereof (e.g., an individual suffering from or suspected of having Fragile X syndrome) a therapeutically effective amount of a PAK inhibitor (e.g., a compound of Formula I-IV and A-D). In some embodiments of the methods described herein, administration of a PAK inhibitor stabilizes LTP or LTD following induction (e.g., by theta-burst stimulation, high-frequency stimulation for LTP, low-frequency (e.g., 1 Hz) stimulation for LTD).

Provided herein are methods for stabilization of synaptic transmission comprising administering to an individual in need thereof (e.g., an individual suffering from or suspected of having Fragile X syndrome) a therapeutically effective amount of a PAK inhibitor (e.g., a compound of Formula I-IV and A-D). In some embodiments of the methods described herein, administration of a PAK inhibitor stabilizes LTP or LTD following induction (e.g., by theta-burst stimulation, high-frequency stimulation for LTP, low-frequency (e.g., 1 Hz) stimulation for LTD).

Also provided herein are methods for alleviation or reversal of cortical hypofrontality during performance of a cognitive task comprising administering to an individual in need thereof (e.g., an individual suffering from or suspected of having Fragile X syndrome) a therapeutically effective amount of a PAK inhibitor (e.g., a compound of Formula I-IV and A-D). In some embodiments of the methods described herein, administration of a PAK inhibitor to an individual suffering from or suspected of having Fragile X syndrome alleviates deficits in the frontal cortex, for example deficits in frontal cortical activation, during the performance of a cognitive task (e.g., a Wisconsin Card Sort test, Mini-Mental State Examination (MMSE), MATRICS cognitive battery, BACS score, Alzheimer's disease Assessment Scale—Cognitive Subscale (ADAS-Cog), Alzheimer's disease Assessment Scale—Behavioral Subscale (ADAS-Behav), Hopkins Verbal Learning Test-Revised or the like) and improves cognition scores of the individual.

Provided herein are methods for reversing abnormalities in dendritic spine morphology or synaptic function that are caused by mutations in high-risk genes (e.g. mutations in Amyloid Precursor Protein (APP), mutations in presenilin 1 and 2, the epsilon4 allele, the 91 bp allele in the telomeric region of 12q, Apolipoprotein E-4 (APOE4) gene, SORL1 gene, reelin gene, DISC1 gene, or any other high-risk allele) comprising administering to an individual in need thereof a therapeutically effective amount of a PAK inhibitor (e.g., a compound of Formula I-IV and A-D). In some embodiments of the methods described herein, prophylactic administration of a PAK inhibitor to an individual at a high risk for developing Fragile X syndrome reverses abnormalities in dendritic spine morphology and/or synaptic function and prevents development of Fragile X syndrome.

Provided herein are methods for stabilizing, reducing or reversing abnormalities in dendritic spine morphology or synaptic function that are caused by increased activation of PAK at the synapse, comprising administration of a therapeutically effective amount of a PAK inhibitor (e.g., a compound of Formula I-IV and A-D) to an individual in need thereof (e.g., an individual suffering from or suspected of having Fragile X syndrome). In some embodiments of the methods described herein, increased activation of PAK at the synapse is caused by Abeta. In some instances, increased activation of PAK at the synapse is caused by redistribution of PAK from the cytosol to the synapse. In some embodiments of the methods described herein, administration of a therapeutically effective amount of a PAK inhibitor (e.g., a compound of Formula I-IV and A-D) to an individual in need thereof (e.g., an individual suffering from or suspected of having Fragile X syndrome) reduces or prevents redistribution of PAK from the cytosol to the synapse in neurons, thereby stabilizing, reducing or reversing abnormalities in dendritic spine morphology or synaptic function that are caused by increased activation of PAK at the synapse.

Provided herein are methods for delaying the onset of Fragile X syndrome comprising administering to an individual in need thereof (e.g., an individual with a high-risk allele for a NC) a therapeutically effective amount of a PAK inhibitor (e.g., a compound of Formula I-IV and A-D). Provided herein are methods for delaying the loss of dendritic spine density comprising administering to an individual in need thereof (e.g., an individual with a high-risk allele for Fragile X syndrome) a therapeutically effective amount of a PAK inhibitor. Provided herein are methods for modulation of spine density, shape, spine length, spine head volume, or spine neck diameter or the like comprising administering to an individual in need thereof (e.g., an individual suffering from or suspected of having Fragile X syndrome) a therapeutically effective amount of a PAK inhibitor (e.g., a compound of Formula I-IV and A-D). Provided herein are methods of modulating the ratio of mature dendritic spines to immature dendritic spines comprising administering to an individual in need thereof (e.g., an individual suffering from or suspected of having Fragile X syndrome) a therapeutically effective amount of a PAK inhibitor. Provided herein are methods of modulating the ratio of dendritic spines head volume to dendritic spines length comprising administering to an individual in need thereof (e.g., an individual suffering from or suspected of having Fragile X syndrome) a therapeutically effective amount of a PAK inhibitor (e.g., a compound of Formula I-IV and A-D).

In some embodiments of the methods described herein, administration of a PAK inhibitor (e.g., a maintenance dose of a PAK inhibitor) reduces the incidence of recurrence of one or more symptoms or pathologies in an individual (e.g., recurrence of psychotic episodes, epileptic seizures or the like). In some embodiments of the methods described herein, administration of a PAK inhibitor causes substantially complete inhibition of PAK and restores dendritic spine morphology and/or synaptic function to normal levels. In some embodiments of the methods described herein, administration of a PAK inhibitor causes partial inhibition of PAK and restores dendritic spine morphology and/or synaptic function to normal levels.

Provided herein are methods for stabilizing, reducing or reversing neuronal withering and/or atrophy or nervous tissue and/or degeneration of nervous tissue that is associated with Fragile X syndrome. In some embodiments of the methods described herein, administration of a PAK inhibitor to an individual suffering from or suspected of having Fragile X syndrome stabilizes, alleviates or reverses neuronal withering and/or atrophy and/or degeneration in the temporal lobe, parietal lobe, the frontal cortex, the cingulate gyrus or the like. In some embodiments of the methods described herein, administration of a PAK inhibitor to an individual suffering from or suspected of having Fragile X syndrome stabilizes, reduces or reverses deficits in memory and/or cognition and/or control of bodily functions.

In some instances, Fragile X syndrome is associated with a decrease in dendritic spine density. In some embodiments of the methods described herein, administration of a PAK inhibitor increases dendritic spine density. In some instances, Fragile X syndrome is associated with an increase in dendritic spine length. In some embodiments of the methods described herein, administration of a PAK inhibitor decreases dendritic spine length. In some instances, Fragile X syndrome is associated with a decrease in dendritic spine neck diameter. In some embodiments of the methods described herein, administration of a PAK inhibitor increases dendritic spine neck diameter. In some instances, Fragile X syndrome is associated with a decrease in dendritic spine head diameter and/or dendritic spine head surface area and/or dendritic spine head volume. In some embodiments of the methods described herein, administration of a PAK inhibitor increases dendritic spine head diameter and/or dendritic spine head volume and/or dendritic spine head surface area.

In some instances, Fragile X syndrome is associated with an increase in immature spines and a decrease in mature spines. In some embodiments of the methods described herein, administration of a PAK inhibitor modulates the ratio of immature spines to mature spines. In some instances, Fragile X syndrome is associated with an increase in stubby spines and a decrease in mushroom-shaped spines. In some embodiments of the methods described herein, administration of a PAK inhibitor modulates the ratio of stubby spines to mushroom-shaped spines.

In some embodiments of the methods described herein, administration of a PAK inhibitor modulates a spine:head ratio, e.g., ratio of the volume of the spine to the volume of the head, ratio of the length of a spine to the head diameter of the spine, ratio of the surface area of a spine to the surface area of the head of a spine, or the like, compared to a spine:head ratio in the absence of a PAK inhibitor. In certain embodiments, a PAK inhibitor suitable for the methods described herein modulates the volume of the spine head, the width of the spine head, the surface area of the spine head, the length of the spine shaft, the diameter of the spine shaft, or a combination thereof. In some embodiments, provided herein is a method of modulating the volume of a spine head, the width of a spine head, the surface area of a spine head, the length of a spine shaft, the diameter of a spine shaft, or a combination thereof, by contacting a neuron comprising the dendritic spine with an effective amount of a PAK inhibitor described herein. In specific embodiments, the neuron is contacted with the PAK inhibitor in vivo.

### Other Agents

In some embodiments, one or more PAK inhibitors are used in combination with one or more agents that modulate dendritic spine morphology or synaptic function. Examples of agents that modulate dendritic spine morphology include minocycline, trophic factors (e.g., brain derived neutrophic factor, glial cell-derived neurtrophic factor), or anesthetics that modulate spine motility, or the like. In some embodiments, one or more PAK inhibitors are used in combination with one or more agents that modulate cognition. In some embodiments, a second therapeutic agent is a nootropic agent that enhances cognition. Examples of nootropic agents include and are not limited to piracetam, pramiracetam, oxiracetam, and aniracetam.

### Blood Brain Barrier Facilitators

In some instances, a PAK inhibitor is optionally administered in combination with a blood brain barrier facilitator. In certain embodiments, an agent that facilitates the transport of a PAK inhibitor is covalently attached to the PAK inhibitor. In some instances, PAK inhibitors described herein are modified by covalent attachment to a lipophilic carrier or co-formulation with a lipophilic carrier. In some embodiments, a PAK inhibitor is covalently attached to a lipophilic carrier, such as e.g., DHA, or a fatty acid. In some embodiments, a PAK inhibitor is covalently attached to artificial low density lipoprotein particles. In some instances, carrier systems facilitate the passage of PAK inhibitors described herein across the blood-brain barrier and include but are not limited to, the use of a dihydropyridine pyridinium salt carrier redox system for delivery of drug species across the blood brain barrier. In some instances a PAK inhibitor described herein is coupled to a lipophilic phosphonate derivative. In certain instances, PAK inhibitors described herein are conjugated to PEG-oligomers/polymers or aprotinin derivatives and analogs. In some instances, an increase in influx of a PAK inhibitor described herein across the blood brain barrier is achieved by modifying A PAK inhibitor described herein (e.g., by reducing or increasing the number of charged groups on the compound) and enhancing affinity for a blood brain barrier transporter. In certain instances, a PAK inhibitor is co-administered with an agent that reduces or inhibits efflux across the blood brain barrier, e.g. an inhibitor of P-glycoprotein pump (PGP) mediated efflux (e.g., cyclosporin, SCH66336 (Ionafarnib, Schering)).

In some embodiments, compounds of Formula I-IV and A-D are optionally administered in combination with, e.g., compounds described in U.S. Pat. Nos. 5,863,532, 6,191,169, 6,248,549, and 6,498,163; U.S. Patent Applications 200200045564, 20020086390, 20020106690, 20020142325, 20030124107, 20030166623, 20040091992, 20040102623, 20040208880, 200500203114, 20050037965, 20050080002, and 20050233965, 20060088897; EP Patent Publication 1492871; PCT patent publication WO 9902701; PCT patent publication WO 2008/047307; Kumar et al., (2006), Nat. Rev. Cancer, 6:459; and Eswaran et al., (2007), Structure, 15:201-213, all of which are incorporated herein by reference for disclosure of kinase inhibitors and/or PAK inhibitors described therein.

In some embodiments, compounds of Formula I-IV and A-D are optionally administered in combination with compounds including and not limited to BMS-387032; SNS-032; CHI4-258; TKI-258; EKB-569; JNJ-7706621; PKC-412; staurosporine; SU-14813; sunitinib; N-(3-chloro-4-fluoro-phenyl)-7-methoxy-6-(3-morpholin-4-ylpropoxy)quinazolin-4-amine (gefitinib), VX-680; MK-0457; combinations thereof or salts, prodrugs thereof.

In some embodiments, compounds of Formula I-IV and A-D are optionally administered in combination with a polypeptide comprising an amino acid sequence about 80% to about 100% identical, e.g., 85%, 90%, 92%, 93%, 95%, 96%, 97%, 98%, 99%, or any other percent from about 80% to about 100% identical the following amino acid sequence:
HTIHVGFDAVTGEFTGMPEQWARLLQTSNITKSEQKKNPQAVLDVLEFYNSKKTSNSQ KYMSFTDKS

The above sequence corresponds to the PAK autoinhibitory domain (PAD) polypeptide amino acids 83-149 of PAK1 polypeptide as described in, e.g., Zhao et al (1998). In some embodiments, the PAK inhibitor is a fusion protein comprising the above-described PAD amino acid sequence. In some embodiments, in order to facilitate cell penetration the fusion polypeptide (e.g., N-terminal or C-terminal) further comprises a polybasic protein transduction domain (PTD) amino acid sequence, e.g.: RKKRRQRR; YARAAARQARA; THRLPRRRRRR; or GGRRARRRRRR.

In some embodiments, in order to enhance uptake into the brain, the fusion polypeptide further comprises a human insulin receptor antibody as described in U.S. patent application Ser. No. 11/245,546.

In some embodiments, compounds of Formula I-IV and A-D are optionally administered in combination with a peptide inhibitor comprising a sequence at least 60% to 100%, e.g., 65%, 70%, 75%, 80%, 85%, 90%, 92%, 93%, 95%, 96%, 97%, 98%, 99%, or any other percent from about 60% to about 100% identical the following amino acid sequence: PPVIAPREHTKSVYTRS as described in, e.g., Zhao et al (2006), Nat Neurosci, 9(2):234-242. In some embodiments, the peptide sequence further comprises a PTD amino acid sequence as described above.

In some embodiments, compounds of Formula I-IV and A-D are optionally administered in combination with a polypeptide comprising an amino acid sequence at least 80% to 100%, e.g., 85%, 90%, 92%, 93%, 95%, 96%, 97%, 98%, 99%, or any other percent from about 80% to about 100% identical to the FMRP1 protein (GenBank Accession No. Q06787), where the polypeptide is able to bind with a PAK (for example, PAK1, PAK2, PAK3, PAK4, PAK5and/or PAK6). In some embodiments compounds of Formula I-IV and A-D are optionally administered in combination with a polypeptide comprising an amino acid sequence at least 80% to 100%, e.g., 85%, 90%, 92%, 93%, 95%, 96%, 97%, 98%, 99%, or any other percent from about 80% to about 100% identical to the FMRP1 protein (GenBank Accession No. Q06787), where the polypeptide is able to bind with a Group I PAK, such as, for example PAK1 (see, e.g., Hayashi et al (2007), Proc Natl Acad Sci USA, 104(27):11489-11494. In some embodiments, compounds of Formula I-IV and A-D are optionally administered in combination with a polypeptide comprising a fragment of human FMRP1 protein with an amino acid sequence at least 80% to 100%, e.g., 85%, 90%, 92%, 93%, 95%, 96%, 97%, 98%, 99%, or any other percent from about 80% to about 100% identical to the sequence of amino acids 207-425 of the human FMRP1 protein (i.e., comprising the KH1 and KH2 domains), where the polypeptide is able to bind to PAK1.

In some embodiments, compounds of Formula I-IV and A-D are optionally administered in combination with a polypeptide comprising an amino acid sequence at least 80% to 100%, e.g., 85%, 90%, 92%, 93%, 95%, 96%, 97%, 98%, 99%, or any other percent from about 80% to about 100% identical to at least five, at least ten at least twenty, at least thirty, at least forty, at least fifty, at least sixty, at least seventy, at least eighty, at least ninety contiguous amino acids of the huntingtin (htt) protein (GenBank Accession No. NP 002102, gi 90903231), where the polypeptide is able to bind to a Group 1 PAK (for example, PAK1, PAK2, and/or PAK3). In some embodiments, compounds of Formula I-IV and A-D are optionally administered in combination with a polypeptide comprising an amino acid sequence at least 80% to 100%, e.g., 85%, 90%, 92%, 93%, 95%, 96%, 97%, 98%, 99%, or any other percent from about 80% to about 100% identical to at least a portion of the huntingtin (htt) protein (GenBank Accession No. NP 002102, gi 90903231), where the polypeptide is able to bind to PAK1. In some embodiments, compounds of Formula I-IV and A-D are optionally administered in combination with a polypeptide comprising a fragment of human huntingtin protein with an amino acid sequence at least 80% to 100%, e.g., 85%, 90%, 92%, 93%, 95%, 96%, 97%, 98%, 99%, or any other percent from about 80% to about 100% identical to a sequence of at least five, at least ten, at least twenty, at least thirty, at least forty, at least fifty, at least sixty, at least seventy, at least eighty, at least ninety, or at least 100 contiguous amino acids of the human huntingtin protein that is outside of the sequence encoded by exon 1 of the htt gene (i.e., a fragment that does not contain poly glutamate domains), where the polypeptide binds a PAK. In some embodiments, compounds of Formula I-IV and A-D are optionally administered in combination with a polypeptide comprising a fragment of human huntingtin protein with an amino acid sequence at least 80% identical to a sequence of the human huntingtin protein that is outside of the sequence encoded by exon 1 of the htt gene (i.e., a fragment that does not contain poly glutamate domains), where the polypeptide binds PAK1.

### Upstream Regulators of p21 Activated Kinases

In certain embodiments, compounds of Formula I-IV and A-D are optionally administered in combination with an indirect PAK modulator (e.g., an indirect PAK inhibitor) that affects the activity of a molecule that acts in a signaling pathway upstream of PAK (upstream regulators of PAK). Upstream effectors of PAK include, but are not limited to: TrkB receptors; NMDA receptors; EphB receptors; adenosine receptors; estrogen receptors; integrins; FMRP; Rho-family GTPases, including Cdc42, Rac (including but not limited to Rac1 and Rac2), CDK5, PI3 kinases, NCK, PDK1, EKT, GRB2, Chp, TC10, Tcl, and Wrch-1; guanine nucleotide exchange factors (“GEFs”), such as but not limited to GEFT, members of the Dbl family of GEFs, p21-activated kinase interacting exchange factor (PIX), DEF6, Zizimin 1, Vav1, Vav2, Dbs, members of the DOCK180 family, Kalirin-7, and Tiam1; G protein-coupled receptor kinase-interacting protein 1 (GIT1), CIB1, filamin A, Etk/Bmx, and sphingosine.

Modulators of NMDA receptor include, but are not limited to, 1-aminoadamantane, dextromethorphan, dextrorphan, ibogaine, ketamine, nitrous oxide, phencyclidine, riluzole, tiletamine, memantine, neramexane, dizocilpine, aptiganel, remacimide, 7-chlorokynurenate, DCKA (5,7-dichlorokynurenic acid), kynurenic acid, 1-aminocyclopropanecarboxylic acid (ACPC), AP7 (2-amino-7-phosphonoheptanoic acid), APV (R-2-amino-5-phosphonopentanoate), CPPene (3-[(R)-2-carboxypiperazin-4-yl]-prop-2-enyl-1-phosphonic acid); (+)-(1S,2S)-1-(4-hydroxy-phenyl)-2-(4-hydroxy-4-phenylpiperidino)-1-pro-panol; (1S,2S)-1-(4-hydroxy-3-methoxyphenyl)-2-(4-hydroxy-4-phenylpiperidino)-1-propanol; (3R,4S)-3-(4-(4-fluorophenyl)-4-hydroxypiperidin-1-yl-)-chroman-4,7-diol; (1R*,2R*)-1-(4-hydroxy-3-methylphenyl)-2-(4-(4-fluoro-phenyl)-4-hydroxypiperidin-1-yl)-propan-1-ol-mesylate; and/or combinations thereof

Modulators of estrogen receptors include, and are not limited to, PPT (4,4′,4″-(4-Propyl-[1H]-1,3,5-triyl)trisphenol); SKF-82958 (6-chloro-7,8-dihydroxy-3-allyl-1-phenyl-2,3,4,5-tetrahydro-1H-3-benzazepine); estrogen; estradiol; estradiol derivatives, including but not limited to 17-13 estradiol, estrone, estriol, ERβ-131, phytoestrogen, MK 101 (bioNovo); VG-1010 (bioNovo); DPN (diarylpropiolitrile); ERB-041; WAY-202196; WAY-214156; genistein; estrogen; estradiol; estradiol derivatives, including but not limited to 17-β estradiol, estrone, estriol, benzopyrans and triazolo-tetrahydrofluorenones, disclosed in U.S. Pat. No. 7,279,499, and Parker et al., Bioorg. & Med. Chem. Ltrs. 16: 4652-4656 (2006), each of which is incorporated herein by reference for such disclosure.

Modulators of TrkB include by way of example, neutorophic factors including BDNF and GDNF. Modulators of EphB include XL647 (Exelixis), EphB modulator compounds described in WO/2006081418 and US Appl. Pub. No. 20080300245, incorporated herein by reference for such disclosure, or the like.

Modulators of integrins include by way of example, ATN-161, PF-04605412, MEDI-522, Volociximab, natalizumab, Volociximab, Ro 27-2771, Ro 27-2441, etaracizumab, CNTO-95, JSM6427, cilengitide, R411 (Roche), EMD 121974, integrin antagonist compounds described in J. Med. Chem., 2002, 45 (16), pp 3451-3457, incorporated herein by reference for such disclosure, or the like.

Adenosine receptor modulators include, by way of example, theophylline, 8-Cyclopentyl-1,3-dimethylxanthine (CPX), 8-Cyclopentyl-1,3-dipropylxanthine (DPCPX), 8-Phenyl-1,3-dipropylxanthine, PSB 36, istradefylline, SCH-58261, SCH-442,416, ZM-241,385, CVT-6883, MRS-1706, MRS-1754, PSB-603, PSB-0788, PSB-1115, MRS-1191, MRS-1220, MRS-1334, MRS-1523, MRS-3777, MRE3008F20, PSB-10, PSB-11, VUF-5574, N6-Cyclopentyladenosine, CCPA, 2′-MeCCPA, GR 79236, SDZ WAG 99, ATL-146e, CGS-21680, Regadenoson, 5′-N-ethylcarboxamidoadenosine, BAY 60-6583, LUF-5835, LUF-5845, 2-(1-Hexynyl)-N-methyladenosine, CF-101 (IB-MECA), 2-C1-IB-MECA, CP-532,903, MRS-3558, Rosuvastatin, KW-3902, SLV320, mefloquine, regadenoson, or the like.

In some embodiments, compounds reducing PAK levels decrease PAK transcription or translation or reduce RNA or protein levels. In some embodiments, a compound that decreases PAK levels is an upstream effector of PAK. In some embodiments, exogenous expression of the activated forms of the Rho family GTPases Chp and cdc42 in cells leads to increased activation of PAK while at the same time increasing turnover of the PAK protein, significantly lowering its level in the cell (Hubsman et al. (2007) Biochem. J. 404: 487-497). PAK clearance agents include agents that increase expression of one or more Rho family GTPases and/or one or more guanine nucleotide exchange factors (GEFs) that regulate the activity of Rho family GTPases, in which overexpression of a Rho family GTPase and/or a GEF results in lower levels of PAK protein in cells. PAK clearance agents also include agonists of Rho family GTPases, as well as agonists of GTP exchange factors that activate Rho family GTPases, such as but not limited to agonists of GEFs of the Dbl family that activate Rho family GTPases.

Overexpression of a Rho family GTPase is optionally by means of introducing a nucleic acid expression construct into the cells or by administering a compound that induces transcription of the endogenous gene encoding the GTPase. In some embodiments, the Rho family GTPase is Rac (e.g., Rac1, Rac2, or Rac3), cdc42, Chp, TC10, Tcl, or Wrnch-1. For example, a Rho family GTPase includes Rac1, Rac2, Rac3, or cdc42. A gene introduced into cells that encodes a Rho family GTPase optionally encodes a mutant form of the gene, for example, a more active form (for example, a constitutively active form, Hubsman et al. (2007) Biochem. J. 404: 487-497). In some embodiments, a PAK clearance agent is, for example, a nucleic acid encoding a Rho family GTPase, in which the Rho family GTPase is expressed from a constitutive or inducible promoter. PAK levels in some embodiments are reduced by a compound that directly or indirectly enhances expression of an endogenous gene encoding a Rho family GTPase.

In some embodiments, compounds of Formula I-IV and A-D are optionally administered in combination with a PAK clearance agent.

In some embodiments, compounds of Formula I-IV and A-D are optionally administered in combination with a compound that directly or indirectly decreases the activation or activity of the upstream effectors of PAK. For example, in some embodiments a compound that inhibits the GTPase activity of the small Rho-family GTPases such as Rac and cdc42 thereby reduce the activation of PAK kinase. In some embodiments, the compound that decreases PAK activation is by secramine that inhibits cdc42 activation, binding to membranes and GTP in the cell (Pelish et al. (2005) Nat. Chem. Biol. 2: 39-46). In some embodiments, PAK activation is decreased by EHT 1864, a small molecule that inhibits Rac1, Rac1b, Rac2 and Rac3 function by preventing binding to guanine nucleotide association and engagement with downstream effectors (Shutes et al. (2007) J. Biol. Chem. 49: 35666-35678). In some embodiments, PAK activation is also decreased by the NSC23766 small molecule that binds directly to Rac1 and prevents its activation by Rac-specific RhoGEFs (Gao et al. (2004) Proc. Natl. Acad. Sci. U.S.A. 101: 7618-7623). In some embodiments, PAK activation is also decreased by the 16 kDa fragment of prolactin (16 k PRL), generated from the cleavage of the 23 kDa prolactin hormone by matrix metalloproteases and cathepsin D in various tissues and cell types. 16 k PRL down-regulates the Ras-Tiam1-Rac1-Pak1 signaling pathway by reducing Rac1 activation in response to cell stimuli such as wounding (Lee et al. (2007) Cancer Res 67:11045-11053). In some embodiments, PAK activation is decreased by inhibition of NMDA and/or AMPA receptors. Examples of modulators of AMPA receptors include and are not limited to ketamine, MK801, CNQX (6-cyano-7-nitroquinoxaline-2,3-dione); NBQX (2,3-dihydroxy-6-nitro-7-sulfamoylbenzo[f]quinoxaline-2,3-dione); DNQX (6,7-dinitroquinoxaline-2,3-dione); kynurenic acid; 2,3-dihydroxy-6-nitro-7-sulfamoylbenzo-[f]quinoxaline; PCP or the like. In some embodiments, PAK activation is decreased by inhibition of TrkB activation. In some embodiments, PAK activation is decreased by inhibition of BDNF activation of TrkB. In some embodiments, compounds of Formula I-IV and A-D are optionally administered in combination with an antibody to BDNF. In some embodiments, PAK activation is decreased by inhibition of TrkB receptors; NMDA receptors; EphB receptors; adenosine receptors; estrogen receptors; integrins; Rho-family GTPases, including Cdc42, Rac (including but not limited to Rac1 and Rac2), CDK5, PI3 kinases, NCK, PDK1, EKT, GRB2, Chp, TC10, Tcl, and Wrch-1; guanine nucleotide exchange factors (“GEFs”), such as but not limited to GEFT, members of the Dbl family of GEFs, p21-activated kinase interacting exchange factor (PIX), DEF6, Zizimin 1, Vav1, Vav2, Dbs, members of the DOCK180 family, Kalirin-7, and Tiam1; G protein-coupled receptor kinase-interacting protein 1 (GIT1), CIB1, filamin A, Etk/Bmx, and/or binding to FMRP and/or sphingosine.

In some embodiments, compounds of Formula I-IV and A-D are optionally administered in combination with a compound that decreases PAK levels in the cell, e.g., a compound that directly or indirectly increases the activity of a guanine exchange factor (GEF) that promotes the active state of a Rho family GTPase, such as an agonist of a GEF that activates a Rho family GTPase, such as but not limited to, Rac or cdc42. Activation of GEFs is also effected by compounds that activate TrkB, NMDA, or EphB receptors.

In some embodiments, a PAK clearance agent is a nucleic acid encoding a GEF that activates a Rho family GTPase, in which the GEF is expressed from a constitutive or inducible promoter. In some embodiments, a guanine nucleotide exchange factor (GEF), such as but not limited to a GEF that activates a Rho family GTPase is overexpressed in cells to increase the activation level of one or more Rho family GTPases and thereby lower the level of PAK in cells. GEFs include, for example, members of the Dbl family of GTPases, such as but not limited to, GEFT, PIX (e.g., alphaPIX, betaPIX), DEF6, Zizimin 1, Vav1, Vav2, Dbs, members of the DOCK180 family, hPEM-2, F1100018, kalirin, Tiam1, STEF, DOCK2, DOCK6, DOCK7, DOCK9, Asf, EhGEF3, or GEF-1. In some embodiments, PAK levels are also reduced by a compound that directly or indirectly enhances expression of an endogenous gene encoding a GEF. A GEF expressed from a nucleic acid construct introduced into cells is in some embodiments a mutant GEF, for example a mutant having enhanced activity with respect to wild type.

The clearance agent is optionally a bacterial toxin such as Salmonella typhinmurium toxin SpoE that acts as a GEF to promote cdc42 nucleotide exchange (Buchwald et al. (2002) EMBO J. 21: 3286-3295; Schlumberger et al. (2003) J. Biological Chem. 278: 27149-27159). Toxins such as SopE, fragments thereof, or peptides or polypeptides having an amino acid sequence at least 80% to 100%, e.g., 85%, 90%, 92%, 93%, 95%, 96%, 97%, 98%, 99%, or any other percent from about 80% to about 100% identical to a sequence of at least five, at least ten, at least twenty, at least thirty, at least forty, at least fifty, at least sixty, at least seventy, at least eighty, at least ninety, or at least 100 contiguous amino acids of the toxin are also optionally used as downregulators of PAK activity. The toxin is optionally produced in cells from nucleic acid constructs introduced into cells.

### Modulators of Upstream Regulators of PAKs

In some embodiments, compounds of Formula I-IV and A-D are optionally administered in combination with a modulator of an upstream regulator of PAKs. In some embodiments, a modulator of an upstream regulator of PAKs is an indirect inhibitor of PAK. In certain instances, a modulator of an upstream regulator of PAKs is a modulator of PDK1. In some instances, a modulator of PDK1 reduces of inhibits the activity of PDK1. In some instances a PDK1 inhibitor is an antisense compound (e.g., any PDK1 inhibitor described in U.S. Pat. No. 6,124,272, which PDK1 inhibitor is incorporated herein by reference). In some instances, a PDK1 inhibitor is a compound described in e.g., U.S. Pat. Nos. 7,344,870, and 7,041,687, which PDK1 inhibitors are incorporated herein by reference. In some embodiments, an indirect inhibitor of PAK is a modulator of a PI3 kinase. In some instances a modulator of a PI3 kinase is a PI3 kinase inhibitor. In some instances, a PI3 kinase inhibitor is an antisense compound (e.g., any PI3 kinase inhibitor described in WO 2001/018023, which PI3 kinase inhibitors are incorporated herein by reference). In some instances, an inhibitor of a PI3 kinase is 3-morpholino-5-phenylnaphthalen-1(4H)-one (LY294002), or a peptide based covalent conjugate of LY294002, (e.g., SF1126, Semaphore pharmaceuticals). In certain embodiments, an indirect inhibitor of PAK is a modulator of Cdc42. In certain embodiments, a modulator of Cdc42 is an inhibitor of Cdc42. In certain embodiments, a Cdc42 inhibitor is an antisense compound (e.g., any Cdc42 inhibitor described in U.S. Pat. No. 6,410,323, which Cdc42 inhibitors are incorporated herein by reference). In some instances, an indirect inhibitor of PAK is a modulator of GRB2. In some instances, a modulator of GRB2 is an inhibitor of GRB2. In some instances a GRB2 inhibitor is a GRb2 inhibitor described in e.g., U.S. Pat. No. 7,229,960, which GRB2 inhibitor is incorporated by reference herein. In certain embodiments, an indirect inhibitor of PAK is a modulator of NCK. In certain embodiments, an indirect inhibitor of PAK is a modulator of ETK. In some instances, a modulator of ETK is an inhibitor of ETK. In some instances an ETK inhibitor is a compound e.g., α-Cyano-(3,5-di-t-butyl-4-hydroxy)thiocinnamide (AG 879).

In some embodiments, indirect PAK inhibitors act by decreasing transcription and/or translation of PAK. An indirect PAK inhibitor in some embodiments decreases transcription and/or translation of a PAK. For example, in some embodiments, modulation of PAK transcription or translation occurs through the administration of specific or non-specific inhibitors of PAK transcription or translation. In some embodiments, proteins or non-protein factors that bind the upstream region of the PAK gene or the 5′ UTR of a PAK mRNA are assayed for their affect on transcription or translation using transcription and translation assays (see, for example, Baker, et al. (2003) J. Biol. Chem. 278: 17876-17884; Jiang et al. (2006) J. Chromatography A 1133: 83-94; Novoa et al. (1997) Biochemistry 36: 7802-7809; Brandi et al. (2007) Methods Enzymol. 431: 229-267). PAK inhibitors include DNA or RNA binding proteins or factors that reduce the level of transcription or translation or modified versions thereof. In other embodiments, compounds of Formula I-IV and A-D are optionally administered in combination with an agent that is a modified form (e.g., mutant form or chemically modified form) of a protein or other compound that positively regulates transcription or translation of PAK, in which the modified form reduces transcription or translation of PAK. In yet other embodiments, a transcription or translation inhibitor is an antagonist of a protein or compound that positively regulates transcription or translation of PAK, or is an agonist of a protein that represses transcription or translation.

Regions of a gene other than those upstream of the transcriptional start site and regions of an mRNA other than the 5′ UTR (such as but not limited to regions 3′ of the gene or in the 3′ UTR of an mRNA, or regions within intron sequences of either a gene or mRNA) also include sequences to which effectors of transcription, translation, mRNA processing, mRNA transport, and mRNA stability bind. In some embodiments, compounds of Formula I-IV and A-D are optionally administered in combination with a clearance agent comprising a polypeptide having homology to an endogenous protein that affects mRNA processing, transport, or stability, or is an antagonist or agonist of one or more proteins that affect mRNA processing, transport, or turnover, such that the inhibitor reduces the expression of PAK protein by interfering with PAK mRNA transport or processing, or by reducing the half-life of PAK mRNA. A PAK clearance agents in some embodiments interferes with transport or processing of a PAK mRNA, or by reducing the half-life of a PAK mRNA.

For example, PAK clearance agents decrease RNA and/or protein half-life of a PAK isoform, for example, by directly affecting mRNA and/or protein stability. In certain embodiments, PAK clearance agents cause PAK mRNA and/or protein to be more accessible and/or susceptible to nucleases, proteases, and/or the proteasome. In some embodiments, compounds of Formula I-IV and A-D are optionally administered in combination with agents that decrease the processing of PAK mRNA thereby reducing PAK activity. For example, PAK clearance agents function at the level of pre-mRNA splicing, 5′ end formation (e.g. capping), 3′ end processing (e.g. cleavage and/or polyadenylation), nuclear export, and/or association with the translational machinery and/or ribosomes in the cytoplasm. In some embodiments, PAK clearance agents cause a decrease in the level of PAK mRNA and/or protein, the half-life of PAK mRNA and/or protein by at least about 5%, at least about 10%, at least about 20%, at least about 30%, at least about 40%, at least about 50%, at least about 60%, at least about 80%, at least about 90%, at least about 95%, or substantially 100%.

In some embodiments, the clearance agent comprises one or more RNAi or antisense oligonucleotides directed against one or more PAK isoform RNAs. In some embodiments, compounds of Formula I-IV and A-D are optionally administered in combination with agent that comprise one or more ribozymes directed against one or more PAK isoform RNAs. The design, synthesis, and use of RNAi constructs, antisense oligonucleotides, and ribozymes are found, for example, in Dykxhoorn et al. (2003) Nat. Rev. Mol. Cell. Biol. 4: 457-467; Hannon et al. (2004) Nature 431: 371-378; Sarver et al. (1990) Science 247:1222-1225; Been et al. (1986) Cell 47:207-216). In some embodiments, nucleic acid constructs that induce triple helical structures are also introduced into cells to inhibit transcription of the PAK gene (Helene (1991) Anticancer Drug Des. 6:569-584).

For example, a clearance agent is in some embodiments an RNAi molecule or a nucleic acid construct that produces an RNAi molecule. An RNAi molecule comprises a double-stranded RNA of at least about seventeen bases having a 2-3 nucleotide single-stranded overhangs on each end of the double-stranded structure, in which one strand of the double-stranded RNA is substantially complementary to the target PAK RNA molecule whose downregulation is desired. “Substantially complementary” means that one or more nucleotides within the double-stranded region are not complementary to the opposite strand nucleotide(s). Tolerance of mismatches is optionally assessed for individual RNAi structures based on their ability to downregulate the target RNA or protein. In some embodiments, RNAi is introduced into the cells as one or more short hairpin RNAs (“shRNAs”) or as one or more DNA constructs that are transcribed to produce one or more shRNAs, in which the shRNAs are processed within the cell to produce one or more RNAi molecules.

Nucleic acid constructs for the expression of siRNA, shRNA, antisense RNA, ribozymes, or nucleic acids for generating triple helical structures are optionally introduced as RNA molecules or as recombinant DNA constructs. DNA constructs for reducing gene expression are optionally designed so that the desired RNA molecules are expressed in the cell from a promoter that is transcriptionally active in mammalian cells, such as, for example, the SV40 promoter, the human cytomegalovirus immediate-early promoter (CMV promoter), or the pol III and/or pol II promoter using known methods. For some purposes, it is desirable to use viral or plasmid-based nucleic acid constructs. Viral constructs include but are not limited to retroviral constructs, lentiviral constructs, or based on a pox virus, a herpes simplex virus, an adenovirus, or an adeno-associated virus (AAV).

In other embodiments, compounds of Formula I-IV and A-D are optionally administered in combination with a polypeptide that decreases the activity of PAK. Protein and peptide inhibitors of PAK are optionally based on natural substrates of PAK, e.g., Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), cortactin, cofilin, Ras, Raf, Mek, p47(phox), BAD, caspase 3, estrogen and/or progesterone receptors, NET1, Gaz, phosphoglycerate mutase-B, RhoGDI, prolactin, p41Arc, cortactin and/or Aurora-A. In some embodiments, compounds of Formula I-IV and A-D are optionally administered in combination with an agent that is based on a sequence of PAK itself, for example, the autoinhibitory domain in the N-terminal portion of the PAK protein that binds the catalytic domain of a partner PAK molecule when the PAK molecule is in its homodimeric state (Zhao et al. (1998) Mol. Cell Biol. 18:2153-2163; Knaus et al. (1998) J. Biol. Chem. 273: 21512-21518; Hofman et al. (2004) J. Cell Sci. 117: 4343-4354). In some embodiments, polypeptide inhibitors of PAK comprise peptide mimetics, in which the peptide has binding characteristics similar to a natural binding partner or substrate of PAK.

In some embodiments, provided herein are compounds that downregulate PAK protein level. In some embodiments, the compounds described herein activate or increase the activity of an upstream regulator or downstream target of PAK. In some embodiments, compounds described herein downregulate protein level of a PAK. In some instances compounds described herein reduce at least one of the symptoms related Fragile X syndrome by reducing the amount of PAK in a cell. In some embodiments a compound that decreases PAK protein levels in cells also decreases the activity of PAK in the cells. In some embodiments a compound that decreases PAK protein levels does not have a substantial impact on PAK activity in cells. In some embodiments a compound that increases PAK activity in cells decreases PAK protein levels in the cells.

In some embodiments, a compound that decreases the amount of PAK protein in cells decreases transcription and/or translation of PAK or increases the turnover rate of PAK mRNA or protein by modulating the activity of an upstream effector or downstream regulator of PAK. In some embodiments, PAK expression or PAK levels are influenced by feedback regulation based on the conformation, chemical modification, binding status, or activity of PAK itself. In some embodiments, PAK expression or PAK levels are influenced by feedback regulation based on the conformation, chemical modification, binding status, or activity of molecules directly or indirectly acted on by PAK signaling pathways. As used herein “binding status” refers to any or a combination of whether PAK, an upstream regulator of PAK, or a downstream effector of PAK is in a monomeric state or in an oligomeric complex with itself, or whether it is bound to other polypeptides or molecules. For example, a downstream target of PAK, when phosphorylated by PAK, in some embodiments directly or indirectly downregulates PAK expression or decrease the half-life of PAK mRNA or protein. Downstream targets of PAK include but are not limited to: Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), Ras, Raf, Mek, p47^{phox}, BAD, caspase 3, estrogen and/or progesterone receptors, NET1, Gaz, phosphoglycerate mutase-B, RhoGDI, prolactin, p41^{Arc}, cortactin and/or Aurora-A. Downregulators of PAK levels include downstream targets of PAK or fragments thereof in a phosphorylated state and downstream targets of PAK or fragments thereof in a hyperphosphorylated state.

A fragment of a downstream target of PAK includes any fragment with an amino acid sequence at least 80% to 100%, e.g., 85%, 90%, 92%, 93%, 95%, 96%, 97%, 98%, 99%, or any other percent from about 80% to about 100% identical to a sequence of at least five, at least ten, at least twenty, at least thirty, at least forty, at least fifty, at least sixty, at least seventy, at least eighty, at least ninety, or at least 100 contiguous amino acids of the downstream regulator, in which the fragment of the downstream target of PAK is able to downregulate PAK mRNA or protein expression or increase turnover of PAK mRNA or protein. In some embodiments, the fragment of a downstream regulator of PAK comprises a sequence that includes a phosphorylation site recognized by PAK, in which the site is phosphorylated.

In some embodiments, compounds of Formula I-IV and A-D are optionally administered in combination with a compound that decreases the level of PAK including a peptide, polypeptide, or small molecule that inhibits dephosphorylation of a downstream target of PAK, such that phosphorylation of the downstream target remains at a level that leads to downregulation of PAK levels.

In some embodiments, PAK activity is reduced or inhibited via activation and/or inhibition of an upstream regulator and/or downstream target of PAK. In some embodiments, the protein expression of a PAK is downregulated. In some embodiments, the amount of PAK in a cell is decreased. In some embodiments a compound that decreases PAK protein levels in cells also decreases the activity of PAK in the cells. In some embodiments a compound that decreases PAK protein levels does not decrease PAK activity in cells. In some embodiments a compound that increases PAK activity in cells decreases PAK protein levels in the cells.

In some instances, compounds of Formula I-IV and A-D are optionally administered in combination with a polypeptide that is delivered to one or more brain regions of an individual by administration of a viral expression vector, e.g., an AAV vector, a lentiviral vector, an adenoviral vector, or a HSV vector. A number of viral vectors for delivery of therapeutic proteins are described in, e.g., U.S. Pat. Nos. 7,244,423, 6,780,409, 5,661,033. In some embodiments, the PAK inhibitor polypeptide to be expressed is under the control of an inducible promoter (e.g., a promoter containing a tet-operator). Inducible viral expression vectors include, for example, those described in U.S. Pat. No. 6,953,575. Inducible expression of a PAK inhibitor polypeptide allows for tightly controlled and reversible increases of PAK inhibitor polypeptide expression by varying the dose of an inducing agent (e.g., tetracycline) administered to an individual.

### Examples of Pharmaceutical Compositions and Methods of Administration

Provided herein, in certain embodiments, are compositions comprising a therapeutically effective amount of any compound described herein (e.g., a compound of Formula I-IV).

Pharmaceutical compositions are formulated using one or more physiologically acceptable carriers including excipients and auxiliaries which facilitate processing of the active compounds into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

Provided herein are pharmaceutical compositions that include one or more PAK inhibitors and a pharmaceutically acceptable diluent(s), excipient(s), or carrier(s). In addition, the PAK inhibitor is optionally administered as pharmaceutical compositions in which it is mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

A pharmaceutical composition, as used herein, refers to a mixture of a PAK inhibitor with other chemical components, such as carriers, stabilizers, diluents, dispersing agents, suspending agents, thickening agents, and/or excipients. The pharmaceutical composition facilitates administration of the PAK inhibitor to an organism. In practicing the methods of treatment or use provided herein, therapeutically effective amounts of a PAK inhibitor are administered in a pharmaceutical composition to a mammal having a condition, disease, or disorder to be treated. Preferably, the mammal is a human A therapeutically effective amount varies depending on the severity and stage of the condition, the age and relative health of an individual, the potency of the PAK inhibitor used and other factors. The PAK inhibitor is optionally used singly or in combination with one or more therapeutic agents as components of mixtures.

The pharmaceutical formulations described herein are optionally administered to an individual by multiple administration routes, including but not limited to, oral, parenteral (e.g., intravenous, subcutaneous, intramuscular), intranasal, buccal, topical, rectal, or transdermal administration routes. By way of example only, Example 26a is describes a parenteral formulation, Example 26f describes a rectal formulation. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions will include at least one PAK inhibitor, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these PAK inhibitors having the same type of activity. In some situations, PAK inhibitors exist as tautomers. All tautomers are included within the scope of the compounds presented herein. Additionally, the PAK inhibitor exists in unsolvated as well as solvated forms with pharmaceutically acceptable solvents such as water, ethanol, and the like. The solvated forms of the PAK inhibitors presented herein are also considered to be disclosed herein.

“Carrier materials” include any commonly used excipients in pharmaceutics and should be selected on the basis of compatibility with compounds disclosed herein, such as, a PAK inhibitor, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

Moreover, the pharmaceutical compositions described herein, which include a PAK inhibitor, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, a formulation comprising a PAK inhibitor is a solid drug dispersion. A solid dispersion is a dispersion of one or more active ingredients in an inert carrier or matrix at solid state prepared by the melting (or fusion), solvent, or melting-solvent methods (Chiou and Riegelman, Journal of Pharmaceutical Sciences, 60, 1281 (1971)). The dispersion of one or more active agents in a solid diluent is achieved without mechanical mixing. Solid dispersions are also called solid-state dispersions. In some embodiments, any compound described herein (e.g., a compound of Formula I-IV and A-D is formulated as a spray dried dispersion (SDD). An SDD is a single phase amorphous molecular dispersion of a drug in a polymer matrix. It is a solid solution prepared by dissolving the drug and a polymer in a solvent (e.g., acetone, methanol or the like) and spray drying the solution. The solvent rapidly evaporates from droplets which rapidly solidifies the polymer and drug mixture trapping the drug in amorphous form as an amorphous molecular dispersion. In some embodiments, such amorphous dispersions are filled in capsules and/or constituted into oral powders for reconstitution. Solubility of an SDD comprising a drug is higher than the solubility of a crystalline form of a drug or a non-SDD amorphous form of a drug. In some embodiments of the methods described herein, PAK inhibitors are administered as SDDs constituted into appropriate dosage forms described herein.

Pharmaceutical preparations for oral use are optionally obtained by mixing one or more solid excipient with a PAK inhibitor, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate.

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active compound doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. By way of example, Example 26b describes a solid dosage formulation that is a capsule. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of a PAK inhibitor are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including a PAK inhibitor, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

The pharmaceutical solid oral dosage forms including formulations described herein, which include a PAK inhibitor, are optionally further formulated to provide a controlled release of the PAK inhibitor. Controlled release refers to the release of the PAK inhibitor from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to an individual over an extended period of time according to a predetermined profile. Such release rates provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In other embodiments, the formulations described herein, which include a PAK inhibitor, are delivered using a pulsatile dosage form. A pulsatile dosage form is capable of providing one or more immediate release pulses at predetermined time points after a controlled lag time or at specific sites. Pulsatile dosage forms including the formulations described herein, which include a PAK inhibitor, are optionally administered using a variety of pulsatile formulations that include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the PAK inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the PAK inhibitor is optionally in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of the PAK inhibitor and a suitable powder base such as lactose or starch. By way of example, Example 26e describes an inhalation formulation.

Buccal formulations that include a PAK inhibitor include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period, wherein the delivery of the PAK inhibitor, is provided essentially throughout. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner By way of example, Examples 26c and 26d describe sublingual formulations.

Transdermal formulations of a PAK inhibitor are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144. By way of example, Example 26g describes a topical formulation.

The transdermal formulations described herein include at least three components: (1) a formulation of a PAK inhibitor; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration of a PAK inhibitor employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery of the PAK inhibitor is optionally accomplished by means of iontophoretic patches and the like. Additionally, transdermal patches provide controlled delivery of the PAK inhibitor. The rate of absorption is optionally slowed by using rate-controlling membranes or by trapping the PAK inhibitor within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing the PAK inhibitor optionally with carriers, optionally a rate controlling barrier to deliver the PAK inhibitor to the skin of the host at a controlled and predetermined rate over a prolonged period of time, and means to secure the device to the skin.

Formulations that include a PAK inhibitor suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, a PAK inhibitor is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of the PAK inhibitor in water soluble form. Additionally, suspensions of the PAK inhibitor are optionally prepared as appropriate oily injection suspensions.

In some embodiments, the PAK inhibitor is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

The PAK inhibitor is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Examples of Methods of Dosing and Treatment Regimens

The PAK inhibitor is optionally used in the preparation of medicaments for the prophylactic and/or therapeutic treatment of v that would benefit, at least in part, from amelioration of symptoms. In addition, a method for treating any of the diseases or conditions described herein in an individual in need of such treatment, involves administration of pharmaceutical compositions containing at least one PAK inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

In the case wherein the patient's condition does not improve, upon the doctor's discretion the administration of the PAK inhibitor is optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the PAK inhibitor is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In some embodiments, the pharmaceutical compositions described herein are in unit dosage forms suitable for single administration of precise dosages. In unit dosage form, the formulation is divided into unit doses containing appropriate quantities of one or more PAK inhibitor. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

The daily dosages appropriate for the PAK inhibitor are from about 0.01 to about 2.5 mg/kg per body weight. An indicated daily dosage in the larger mammal, including, but not limited to, humans, is in the range from about 0.5 mg to about 1000 mg, conveniently administered in divided doses, including, but not limited to, up to four times a day or in extended release form. Suitable unit dosage forms for oral administration include from about 1 to about 500 mg active ingredient, from about 1 to about 250 mg of active ingredient, or from about 1 to about 100 mg active ingredient. The foregoing ranges are merely suggestive, as the number of variables in regard to an individual treatment regime is large, and considerable excursions from these recommended values are not uncommon Such dosages are optionally altered depending on a number of variables, not limited to the activity of the PAK inhibitor used, the disease or condition to be treated, the mode of administration, the requirements of an individual, the severity of the disease or condition being treated, and the judgment of the practitioner.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. PAK inhibitors exhibiting high therapeutic indices are preferred. The data obtained from cell culture assays and animal studies is optionally used in formulating a range of dosage for use in human. The dosage of such PAK inhibitors lies preferably within a range of circulating concentrations that include the ED50 with minimal toxicity. The dosage optionally varies within this range depending upon the dosage form employed and the route of administration utilized.

### Assays for Identification and Characterization of PAK Inhibitors

Small molecule PAK inhibitors are optionally identified in high-throughput in vitro or cellular assays as described in, e.g., Yu et al (2001), J Biochem (Tokyo); 129(2):243-251; Rininsland et al (2005), BMC Biotechnol, 5:16; and Allen et al (2006), ACS Chem Biol; 1(6):371-376. PAK inhibitors suitable for the methods described herein are available from a variety of sources including both natural (e.g., plant extracts) and synthetic. For example, candidate PAK inhibitors are isolated from a combinatorial library, i.e., a collection of diverse chemical compounds generated by either chemical synthesis or biological synthesis by combining a number of chemical “building blocks.” For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks, as desired. Theoretically, the systematic, combinatorial mixing of 100 interchangeable chemical building blocks results in the synthesis of 100 million tetrameric compounds or 10 billion pentameric compounds. See Gallop et al. (1994), J. Med. Chem. 37(9), 1233. Each member of a library may be singular and/or may be part of a mixture (e.g. a “compressed library”). The library may comprise purified compounds and/or may be “dirty” (i.e., containing a quantity of impurities). Preparation and screening of combinatorial chemical libraries are documented methodologies. See Cabilly, ed., Methods in Molecular Biology, Humana Press, Totowa, N.J., (1998). Combinatorial chemical libraries include, but are not limited to: diversomers such as hydantoins, benzodiazepines, and dipeptides, as described in, e.g., Hobbs et al. (1993), Proc. Natl. Acad. Sci. U.S.A. 90, 6909; analogous organic syntheses of small compound libraries, as described in Chen et al. (1994), J. Amer. Chem. Soc., 116: 2661; Oligocarbamates, as described in Cho, et al. (1993), Science 261, 1303; peptidyl phosphonates, as described in Campbell et al. (1994), J. Org. Chem., 59: 658; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514). In addition, numerous combinatorial libraries are commercially available from, e.g., ComGenex (Princeton, N.J.); Asinex (Moscow, Russia); Tripos, Inc. (St. Louis, Mo.); ChemStar, Ltd. (Moscow, Russia); 3D Pharmaceuticals (Exton, Pa.); and Martek Biosciences (Columbia, Md.).

Devices for the preparation of combinatorial libraries are commercially available (see, e.g., 357 MPS, 390 MPS from Advanced Chem Tech, Louisville, Ky.; Symphony from Rainin, Woburn, Mass.; 433A from Applied Biosystems, Foster City, Calif.; and 9050 Plus from Millipore, Bedford, Mass.). A number of robotic systems have also been developed for solution phase chemistries. These systems include automated workstations like the automated synthesis apparatus developed by Takeda Chemical Industries, LTD (Osaka, Japan), and many robotic systems utilizing robotic arms (Zymate II). Any of the above devices are optionally used to generate combinatorial libraries for identification and characterization of PAK inhibitors which mimic the manual synthetic operations performed by small molecule PAK inhibitors suitable for the methods described herein. Any of the above devices are optionally used to identify and characterize small molecule PAK inhibitors suitable for the methods disclosed herein. In many of the embodiments disclosed herein, PAK inhibitors, PAK binding molecules, and PAK clearance agents are disclosed as polypeptides or proteins (where polypeptides comprise two or more amino acids). In these embodiments, the inventors also contemplate that PAK inhibitors, binding molecules, and clearance agents also include peptide mimetics based on the polypeptides, in which the peptide mimetics interact with PAK or its upstream or downstream regulators by replicating the binding or substrate interaction properties of PAK or its regulators. Nucleic acid aptamers are also contemplated as PAK inhibitors, binding molecules, and clearance agents, as are small molecules other than peptides or nucleic acids. For example, in some embodiments small molecule PAK binding partners, inhibitors, or clearance agents, or small molecule agonists or antagonists of PAK modulators or targets, are designed or selected based on analysis of the structure of PAK or its modulators or targets and binding interactions with interacting molecules, using “rational drug design” (see, for example Jacobsen et al. (2004) Molecular Interventions 4:337-347; Shi et al. (2007) Bioorg. Med. Chem. Lett. 17:6744-6749).

The identification of potential PAK inhibitors is determined by, for example, assaying the in vitro kinase activity of PAK in the presence of candidate inhibitors. In such assays, PAK and/or a characteristic PAK fragment produced by recombinant means is contacted with a substrate in the presence of a phosphate donor (e.g., ATP) containing radiolabeled phosphate, and PAK-dependent incorporation is measured. “Substrate” includes any substance containing a suitable hydroxyl moiety that can accept the γ-phosphate group from a donor molecule such as ATP in a reaction catalyzed by PAK. The substrate may be an endogenous substrate of PAK, i.e. a naturally occurring substance that is phosphorylated in unmodified cells by naturally-occurring PAK or any other substance that is not normally phosphorylated by PAK in physiological conditions, but may be phosphorylated in the employed conditions. The substrate may be a protein or a peptide, and the phosphrylation reaction may occur on a serine and/or threonine residue of the substrate. For example, specific substrates, which are commonly employed in such assays include, but are not limited to, histone proteins and myelin basic protein. In some embodiments, PAK inhibitors are identified using IMAP® technology.

Detection of PAK dependent phosphorylation of a substrate can be quantified by a number of means other than measurement of radiolabeled phosphate incorporation. For example, incorporation of phosphate groups may affect physiochemical properties of the substrate such as electrophoretic mobility, chromatographic properties, light absorbance, fluorescence, and phosphorescence. Alternatively, monoclonal or polyclonal antibodies can be generated which selectively recognize phosphorylated forms of the substrate from non-phosphorylated forms whereby allowing antibodies to function as an indicator of PAK kinase activity.

High-throughput PAK kinase assays can be performed in, for example, microtiter plates with each well containing PAK kinase or an active fragment thereof, substrate covalently linked to each well, P³² radiolabled ATP and a potential PAK inhibitor candidate. Microtiter plates can contain 96 wells or 1536 wells for large scale screening of combinatorial library compounds. After the phosphorylation reaction has completed, the plates are washed leaving the bound substrate. The plates are then detected for phosphate group incorporation via autoradiography or antibody detection. Candidate PAK inhibitors are identified by their ability to decease the amount of PAK phosphotransferase ability upon a substrate in comparison with PAK phosphotransferase ability alone.

The identification of potential PAK inhibitors may also be determined, for example, via in vitro competitive binding assays on the catalytic sites of PAK such as the ATP binding site and/or the substrate binding site. For binding assays on the ATP binding site, a known protein kinase inhibitor with high affinity to the ATP binding site is used such as staurosporine. Staurosporine is immobilized and may be fluorescently labeled, radiolabeled or in any manner that allows detection. The labeled staurosporine is introduced to recombinantly expressed PAK protein or a fragment thereof along with potential PAK inhibitor candidates. The candidate is tested for its ability to compete, in a concentration-dependant manner, with the immobilized staurosporine for binding to the PAK protein. The amount of staurosporine bound PAK is inversely proportional to the affinity of the candidate inhibitor for PAK. Potential inhibitors would decrease the quantifiable binding of staurosporine to PAK. See e.g., Fabian et al (2005) Nat. Biotech., 23:329. Candidates identified from this competitive binding assay for the ATP binding site for PAK would then be further screened for selectivity against other kinases for PAK specificity.

The identification of potential PAK inhibitors may also be determined, for example, by in cyto assays of PAK activity in the presence of the inhibitor candidate. Various cell lines and tissues may be used, including cells specifically engineered for this purpose. In cyto screening of inhibitor candidates may assay PAK activity by monitoring the downstream effects of PAK activity. Such effects include, but are not limited to, the formation of peripheral actin microspikes and or associated loss of stress fibers as well as other cellular responses such as growth, growth arrest, differentiation, or apoptosis. See e.g., Zhao et al., (1998) Mol. Cell. Biol. 18:2153. For example in a PAK yeast assay, yeast cells grow normally in glucose medium. Upon exposure to galactose however, intracellular PAK expression is induced, and in turn, the yeast cells die. Candidate compounds that inhibit PAK activity are identified by their ability to prevent the yeast cells from dying from PAK activation.

Alternatively, PAK-mediated phosphorylation of a downstream target of PAK can be observed in cell based assays by first treating various cell lines or tissues with PAK inhibitor candidates followed by lysis of the cells and detection of PAK mediated events. Cell lines used in this experiment may include cells specifically engineered for this purpose. PAK mediated events include, but are not limited to, PAK mediated phosphorylation of downstream PAK mediators. For example, phosphorylation of downstream PAK mediators can be detected using antibodies that specifically recognize the phosphorylated PAK mediator but not the unphosphorylated form. These antibodies have been described in the literature and have been extensively used in kinase screening campaigns. In some instances a phospho LIMK antibody is used after treatment of HeLa cells stimulated with EGF or sphingosine to detect downstream PAK signaling events.

The identification of potential PAK inhibitors may also be determined, for example, by in vivo assays involving the use of animal models, including transgenic animals that have been engineered to have specific defects or carry markers that can be used to measure the ability of a candidate substance to reach and/or affect different cells within the organism. For example, DISC1 knockout mice have defects in synaptic plasticity and behavior from increased numbers of dendritic spines and an abundance of long and immature spines. Thus, identification of PAK inhibitors can comprise administering a candidate to DISC1 knockout mice and observing for reversals in synaptic plasticity and behavior defects as a readout for PAK inhibition.

For example, fragile X mental retardation 1 (FMR1) knockout mice have defects in synaptic plasticity and behavior from increased numbers of dendritic spines and an abundance of long and immature spines. See e.g., Comery et al., (1997) Proc. Natl. Acad. Sci. USA, 94:5401-04. As PAK is a downstream effector of the FMR1 gene, the defects are reversed upon the use of dominant negative transgenes of PAK that inhibit endogenous PAK activity. See Hayashi et al. (2007) Proc. Natl. Acad. Sci. USA, 104:11489-94. Thus, identification of PAK inhibitors can comprise administering a candidate to FMR1 knockout mice and observing for reversals in synaptic plasticity and behavior defects as a readout for PAK inhibition.

For example, suitable animal models for Alzheimer's disease are knock-ins or transgenes of the human mutated genes including transgenes of the “swedish” mutation of APP (APPswe), transgenes expressing the mutant form of presenilin 1 and presenilin 2 found in familial/early onset AD. Thus, identification of PAK inhibitors can comprise administering a candidate to a knock-in animal and observing for reversals in synaptic plasticity and behavior defects as a readout for PAK inhibition.

Administration of the candidate to the animal is via any clinical or non-clinical route, including but not limited to oral, nasal, buccal and/or topical administrations. Additionally or alternatively, administration may be intratracheal instillation, bronchial instillation, intradermal, subcutaneous, intramuscular, intraperitoneal, inhalation, and/or intravenous injection.

Changes in spine morphology are detected using any suitable method, e.g., by use of 3D and/or 4D real time interactive imaging and visualization. In some instances, the Imaris suite of products (available from Bitplane Scientific Solutions) provides functionality for visualization, segmentation and interpretation of 3D and 4D microscopy datasets obtained from confocal and wide field microscopy data.

### EXAMPLES

The following specific examples are to be construed as merely illustrative, and not limitative of the remainder of the disclosure in any way whatsoever.

All synthetic chemistry was performed in standard laboratory glassware unless indicated otherwise in the examples. Commercial reagents were used as received.

Analytical LC/MS A was performed on an Agilent 1200 system with a variable wavelength detector and Agilent 6110 Single quadrupole mass spectrometer, alternating positive and negative ion scans. (AN/B)

Analytical LC/MS B was performed on an Agilent 1200 system with a variable wavelength detector and Agilent G1956A Single quadrupole mass spectrometer, positive or negative ion scans. (N)

Analytical LC/MS C was performed on an Agilent 1100 system with a variable wavelength detector and Agilent G1946D Single quadrupole mass spectrometer, positive or negative ion scans (AY)

Analytical LC/MS D was performed on an Agilent 1200 system with a variable wavelength detector and Agilent 6110 Single quadrupole mass spectrometer, positive or negative ion scans (AS/F)

Analytical LC/MS E was performed on an Agilent 1100 system with a variable wavelength detector and Agilent G1946A Single quadrupole mass spectrometer, positive or negative ion scans. (AX)

Analytical LC/MS F was performed on an Agilent 1100 system with a variable wavelength detector and Agilent G1946A Single quadrupole mass spectrometer, positive or negative ion scans. (I/E/W)

Analytical LC/MS G was performed on a SHIMADZU LC-20AB system with a variable wavelength detecto and SHIMADZU 2010EV Single quadrupole mass spectrometer, positive ion scans. (R)

Retention times were determined from the extracted 220 nm chromatogram. ¹H NMR was performed on a Bruker DRX-400 at 400 MHz. Microwave reactions were performed in a Biotage Initiator using the instrument software to control heating time and pressure. Silica gel chromatography was performed manually.

Preparative HPLC method A: Preparative HPLC was performed on a Waters 1525/2487 with UV detection at 220 nm and manual collection.

HPLC column: ASB-C18 21.2×150 mm.

HPLC Gradient: 25 mL/min, (0.01% HCL)water:acetonitrile; the gradient shape was optimized for individual separations.

Preparative HPLC method B:

HPLC column: Phenomenex 21.2×150 mm.

HPLC Gradient: 25 mL/min, (0.1% FA)water:acetonitrile; the gradient shape was optimized for individual separations.

### Example 1

### Synthesis of 6-(2-chloro-4-(6-methylpyrazin-2-yl)phenyl)-8-ethyl-2-((3-hydroxypropyl)amino)pyrido[2,3-d]pyrimidin-7(8H)-one (5)

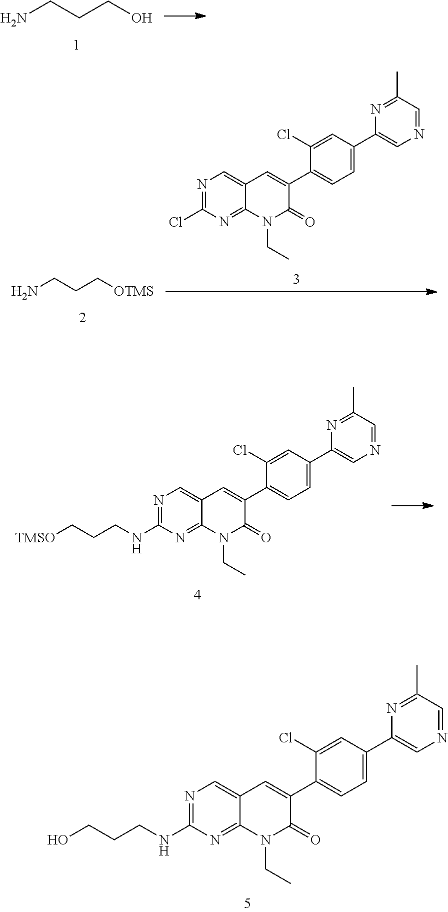

### Step 1: Synthesis of 3-(trimethylsilyloxy)propan-1-amine (2)

TMSCl (3.2 mL, 25.4 mmol) was added to a solution of 3-amino-1-propanol (1.5 mL, 20.0 mmol), Et₃N (2.42 g, 24.0 mmol, 1.2 eq.) and DMAP (24 mg, 1 mol %) in CH₂Cl₂ (60 mL) at 15° C. The reaction mixture was warmed to rt and stirred for 16 h. The reaction mixture was washed with water (30 mL), dried (Na₂SO₄), filtered through celite and concentrated to afford compound 2 (2.63 g, 89%) as a yellow liquid.

### Step 2: Synthesis of 6-(2-chloro-4-(6-methylpyrazin-2-yl)phenyl)-8-ethyl-2-((3-((trimethylsilyl)oxy)propyl)amino)pyrido[2,3-d]pyrimidin-7(8H)-one (4)

A mixture of 2-chloro-6-(2-chloro-4-(6-methylpyrazin-2-yl)phenyl)-8-ethylpyrido[2,3-d]pyrimidin-7(8H)-one 3 (0.5 g, 1.21 mmol), 3-((trimethylsilyl)oxy)propan-1-amine (0.36 g, 2.42 mmol) 2, and Et₃N (122 mg, 1.21 mol) in isopropanol (5 mL) was stirred at reflux for 18 h. The reaction was monitored by LCMS until the reaction was complete. This mixture was evaporated to afford 4 (0.5 g) as a yellow solid. The compound was used directly in the next step without further purification. LCMS m/z 523.10 (M+H)⁺.

### Step 3: Synthesis of 6-(2-chloro-4-(6-methylpyrazin-2-yl)phenyl)-8-ethyl-2-((3-hydroxypropyl)amino)pyrido[2,3-d]pyrimidin-7(8H)-one (5)

Preparative HCl-MeOH (10 mL, 4N) was added dropwise to a mixture of compound 4 (0.5 g, crude) in MeOH (5 mL). The mixture was stirred overnight under N₂, concentrated and purified by prep. HPLC to afford 5 (107 mg). LCMS m/z 451.2 (M+H)⁺. ¹H NMR (400 MHz, DMSO-d₆) □ 9.17 (s, 1H), 8.76 (s, 1H), 8.57 (s, 1H), 8.37-8.35 (br, 1H), 8.28 (s, 1H), 7.89 (s, 1H), 7.57-7.55 (dd, 1H), 4.33 (br, 2H), 3.53-3.48 (m, 4H), 2.69 (s, 3H), 1.76-1.70 (br, 2H), 1.24-1.20 (t, 3H).

The compounds in Table 1 were made using the method described in Example 1 using the appropriate phenylacetate, aldehyde and amine. Compounds were usually obtained after purification by prep. HPLC.

TABLE 1

| Ex. | Structure | MW | Method | m/z | Rt |
|---|---|---|---|---|---|
| 2 | 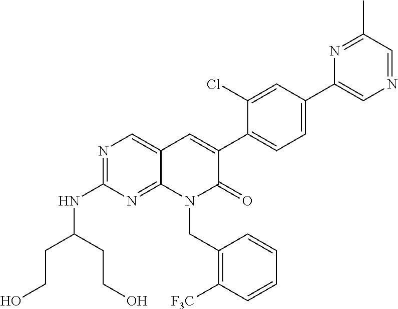 [SMILES: Cc1cncc(-c2ccc(-c3cc4cnc(NC(CCO)CCO)nc4n(Cc4ccccc4C(F)(F)F)c3=O)c(Cl)c2)n1] | 625.1 | A | 625.4 | 3.50 |
| 3 | 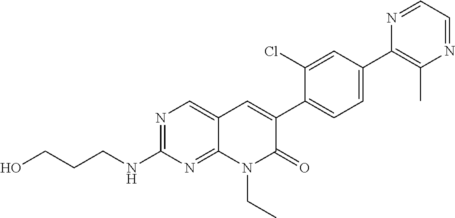 [SMILES: CCn1c(=O)c(-c2ccc(-c3nccnc3C)cc2Cl)cc2cnc(NCCCO)nc21] | 450.9 | A | 451.2 | 2.57 |
| 4 | 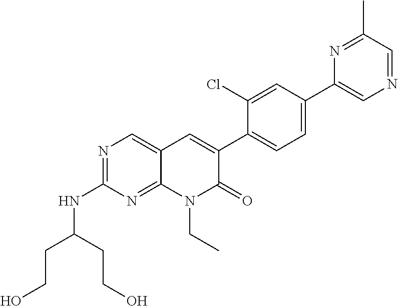 [SMILES: CCn1c(=O)c(-c2ccc(-c3cncc(C)n3)cc2Cl)cc2cnc(NC(CCO)CCO)nc21] | 495.0 | A | 495.3 | 2.53 |
| 5 | 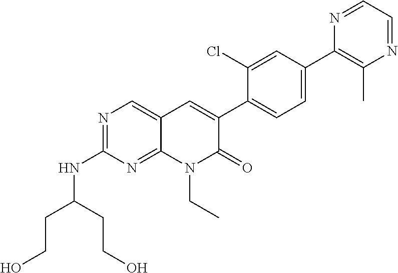 [SMILES: CCn1c(=O)c(-c2ccc(-c3nccnc3C)cc2Cl)cc2cnc(NC(CCO)CCO)nc21] | 495.0 | A | 495.0 | 2.32 |
| 6 | 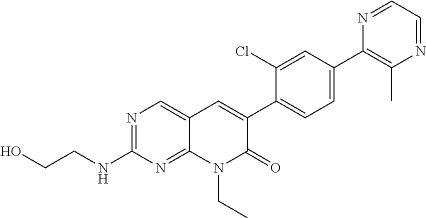 [SMILES: CCn1c(=O)c(-c2ccc(-c3nccnc3C)cc2Cl)cc2cnc(NCCO)nc21] | 436.9 | A | 437.2 | 2.45 |
| 7 | 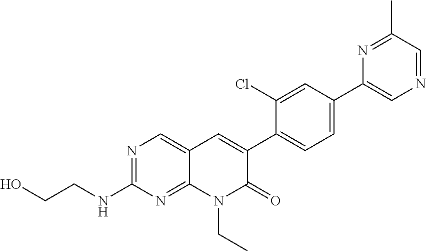 [SMILES: CCn1c(=O)c(-c2ccc(-c3cncc(C)n3)cc2Cl)cc2cnc(NCCO)nc21] | 436.9 | A | 437.1 | 2.76 |
| 8 | 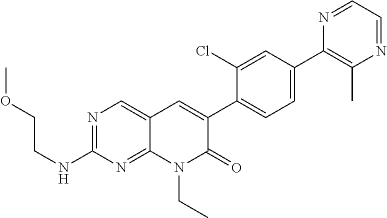 [SMILES: CCn1c(=O)c(-c2ccc(-c3nccnc3C)cc2Cl)cc2cnc(NCCOC)nc21] | 450.9 | A | 451.0 | 3.00 |
| 9 | 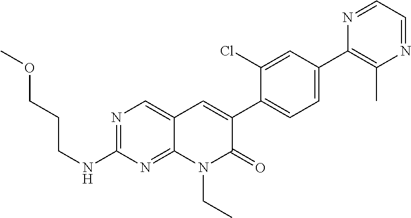 [SMILES: CCn1c(=O)c(-c2ccc(-c3nccnc3C)cc2Cl)cc2cnc(NCCCOC)nc21] | 465.0 | A | 465.2 | 3.13 |
| 10 | 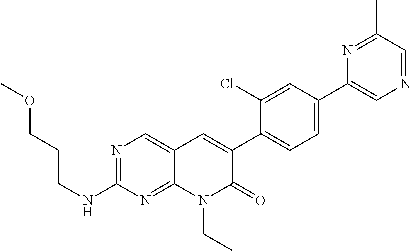 [SMILES: CCn1c(=O)c(-c2ccc(-c3cncc(C)n3)cc2Cl)cc2cnc(NCCCOC)nc21] | 465.0 | E | 465.1 | 1.11 |
| 11 | 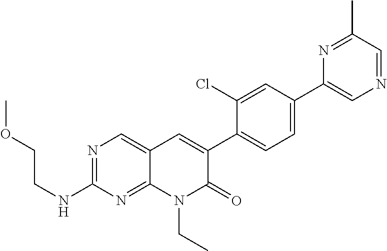 [SMILES: CCn1c(=O)c(-c2ccc(-c3cncc(C)n3)cc2Cl)cc2cnc(NCCOC)nc21] | 450.9 | A | 451.2 | 3.30 |

### BIOLOGICAL EXAMPLES

### Example 2

### In Vitro PAK Inhibition Assay

Assay Conditions

Compounds are screened in 1% DMSO (final) in the well. For 10 point titrations, 3-fold serial dilutions are conducted. All Peptide/Kinase Mixtures are diluted to a 2× working concentration in the appropriate Kinase Buffer

Kinase Specific Assay Conditions

PAK1

The 2× PAK1/Ser/Thr 19 mixture is prepared in 50 mM HEPES pH 7.5, 0.01% BRIJ-35, 10 mM MgCl2, 1 mM EGTA. The final 10 μL Kinase Reaction consists of 2.71-30.8 ng PAK1 and 2 μM Ser/Thr 19 in 50 mM HEPES pH 7.5, 0.01% BRIJ-35, 10 mM MgCl2, 1 mM EGTA. After the 1 hour Kinase Reaction incubation, 5 μL of a 1:128 dilution of Development Reagent A is added.

PAK2 (PAK65)

The 2× PAK2 (PAK65)/Ser/Thr 20 mixture is prepared in 50 mM HEPES pH 7.5, 0.01% BRIJ-35, 10 mM MgCl2, 1 mM EGTA. The final 10 μL Kinase Reaction consists of 0.29-6 ng PAK2 (PAK65) and 2 μM Ser/Thr 20 in 50 mM HEPES pH 7.5, 0.01% BRIJ-35, 10 mM MgCl2, 1 mM EGTA. After the 1 hour Kinase Reaction incubation, 5 μL of a 1:256 dilution of Development Reagent A is added.

PAK3

The 2× PAK3/Ser/Thr 20 mixture is prepared in 50 mM HEPES pH 7.5, 0.01% BRIJ-35, 10 mM MgCl2, 1 mM EGTA. The final 10 μL Kinase Reaction consists of 2.25-22 ng PAK3 and 2 μM Ser/Thr 20 in 50 mM HEPES pH 7.5, 0.01% BRIJ-35, 10 mM MgCl2, 1 mM EGTA. After the 1 hour Kinase Reaction incubation, 5 μL of a 1:256 dilution of Development Reagent A is added.

PAK4

The 2× PAK4/Ser/Thr 20 mixture is prepared in 50 mM HEPES pH 7.5, 0.01% BRIJ-35, 10 mM MgCl2, 1 mM EGTA. The final 10 μL Kinase Reaction consists of 0.1-0.75 ng PAK4 and 2 μM Ser/Thr 20 in 50 mM HEPES pH 7.5, 0.01% BRIJ-35, 10 mM MgCl2, 1 mM EGTA. After the 1 hour Kinase Reaction incubation, 5 μL of a 1:256 dilution of Development Reagent A is added.

### Assay Controls

The following controls are made for each individual kinase and are located on the same plate as the kinase:

0% Phosphorylation Control (100% Inhibition Control)

The maximum Emission Ratio is established by the 0% Phosphorylation Control (100% Inhibition Control), which contains no ATP and therefore exhibits no kinase activity. This control yields 100% cleaved peptide in the Development Reaction.

100% Phosphorylation Control

The 100% Phosphorylation Control, which consists of a synthetically phosphorylated peptide of the same sequence as the peptide substrate, is designed to allow for the calculation of percent phosphorylation.

This control yields a very low percentage of cleaved peptide in the Development Reaction.

The 0% Phosphorylation and 100% Phosphorylation Controls allow one to calculate the percent Phosphorylation achieved in a specific reaction well. Control wells do not include any kinase inhibitors.

0% Inhibition Control

The minimum Emission Ratio in a screen is established by the 0% Inhibition Control, which contains active kinase. This control is designed to produce a 10-50%* phosphorylated peptide in the Kinase Reaction.

Known Inhibitor

A known inhibitor control standard curve, 10 point titration, is run for each individual kinase on the same plate as the kinase to ensure the kinase is inhibited within an expected IC50 range previously determined

The following controls are prepared for each concentration of Test Compound assayed:

Development Reaction Interference

The Development Reaction Interference is established by comparing the Test Compound Control wells that do not contain ATP versus the 0% Phosphorylation Control (which does not contain the Test Compound). The expected value for a non-interfering compound should be 100%. Any value outside of 90% to 110% is flagged.

Test Compound Fluorescence Interference

The Test Compound Fluorescence Interference is determined by comparing the Test Compound Control wells that do not contain the Kinase/Peptide Mixture (zero peptide control) versus the 0% Inhibition Control. The expected value for a non-fluorescence compound should be 0%. Any value>20% is flagged.

Assay Protocol

Bar-coded Corning, low volume NBS, black 384-well plate (Corning Cat. #3676)
1. Add the following solutions to a well in a 384-well plate: 2.5 μL of 4× Test Compound OR (100 nL 100× Test Compound plus 2.4 μL kinase buffer)
   5 μL of 2× Peptide/Kinase (PAK) Mixture
   2.5 μL of 4× ATP Solution
2. Shake the plate for 30-seconds
3. Incubate the PAK Kinase Reaction at room temperature for 60-minutes
4. Add 5 μL of Development Reagent Solution to each well
5. Shake the plate for 30-seconds
6. Incubate the Development Reaction for 60-minutes
7. Determine the fluorescence using a fluorescence plate reader
8. Analyze the fluorescence data

Data Analysis

The following equations are used for each set of data points:

| | Equation |
|---|---|
| Correction for Background Fluorescence | FI_{Sample} − FI_{TCFI Ctl} |
| Emission Ratio (using values corrected for background fluorescence) | $\frac{Coumarin Emission ( 445 nm )}{Fluorescein Emission ( 520 nm )}$ |
| % Phosphorylation (% Phos) | $\{ 1 - \frac{( Emission Ratio \times {F}_{100 \%} ) - {C}_{100 \%}}{\begin{matrix}( {C}_{0 \%} - {C}_{100 \%} ) + \\ [ Emission Ratio \times ( {F}_{100 \%} - {F}_{0 \%} ) ]\end{matrix}} \} * 100$ |
| % Inhibition | $\{ 1 - \frac{\% {Phos}_{Sample}}{\% {Phos}_{0 \% Inhibition Ctl}} \} * 100$ |
| Z′ (using Emission Ratio values) | $1 - \frac{3 * {Stdev}_{0 \% Phos Ctl} + 3 * {Stdev}_{0 \% Inhibition}}{{Mean}_{0 \% Phos Ctl} - {Mean}_{0 \% Inhibition}}$ |
| Difference Between Data Points (single point only) | \| % Inhibition_{Point 1} − % Inhibition_{Point 2} \| |
| Development Reaction Interference (DRI) (no ATP control) | $\frac{Emission {Ratio}_{DRI Ctl}}{Emission {Ratio}_{0 \% Phos Ctl}}$ |
| Test Compound Fluorescence Interference (TCFI) (check both Coumarin and Fluorescein emissions) | $\frac{{FI}_{TCFI Ctl}}{{FI}_{0 \% Inhibitor Ctl}}$ |

FI = Fluorescence Intensity
C100% = Average Coumarin emission signal of the 100% Phos. Control
C0% = Average Coumarin emission signal of the 0% Phos. Control
F100% = Average Fluorescein emission signal of the 100% Phos. Control
F0% = Average Fluorescein emission signal of the 0% Phos. Control
DRI = Development Reaction Interference
TCFI = Test Compound Fluorescence Interference

Graphing Software

SelectScreen® Kinase Profiling Service uses XLfit from IDBS. The dose response curve is curve fit to model number 205 (sigmoidal dose-response model). If the bottom of the curve does not fit between −20% & 20% inhibition, it is set to 0% inhibition. If the top of the curve does not fit between 70% and 130% inhibition, it is set to 100% inhibition.

Table of Kinase ATP Km Bins and Inhibitor Validation

The table below provides specifications and data around each kinase. The representative IC50 value with a known inhibitor for each kinase was determined at the ATP bin nearest to the ATP Km app.

| | Z'-LYTE | ATP Km app | ATP Bin | | IC50 |
|---|---|---|---|---|---|
| Kinase | Substrate | (μM) | (μM) | Inhibitor | (nM) |
| PAK1 | Ser/Thr 19 | 48.5 | 50 | Staurosporine | 3.00 |
| PAK2 | Ser/Thr 20 | 89 | 75 | Staurosporine | 30.0 |
| (PAK65) | | | | | |
| PAK3 | Ser/Thr 20 | 101 | 100 | Staurosporine | 15.3 |
| PAK4 | Ser/Thr 20 | 3 | 5 | Staurosporine | 9.71 |

TABLE
PAK Inhibition IC50

| | PAK1 | PAK2 | PAK3 | PAK4 |
|---|---|---|---|---|
| Example | IC50 (nM) | IC50 (nM) | IC50 (nM) | IC50 (nM) |
| 1 | B | B | B | D |
| 2 | B | B | B | D |
| 3 | B | B | B | D |
| 4 | C | B | C | D |
| 5 | C | C | C | D |
| 6 | B | B | B | D |
| 7 | B | B | B | D |
| 8 | B | B | C | D |
| 9 | B | B | C | D |
| 10 | B | B | B | D |
| 11 | B | B | C | D |

A, IC50 < 50 nM; B, 50 nM ≦ IC50 ≦ 500 nM; C, 0.5 μM < IC50 < 5 μM; D, IC50 ≧ 5 μM

### Example 3

### Additional In Vitro PAK Inhibition Assay

Similar in vitro PAK1 and PAK4 inhibition assay was conducted in compounds for the treatment of Fragile X syndrome, but under ATP concentrations of 10 uM and 1 mM. The results are listed in the table below.

| | PAK1 IC50 | PAK1 IC50 | PAK4 IC50 |
|---|---|---|---|
| Compound | (10 μM ATP) | (1 mM ATP) | (10 μM ATP) |
| 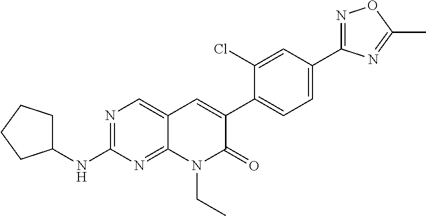 [SMILES: CCn1c(=O)c(-c2ccc(-c3noc(C)n3)cc2Cl)cc2cnc(NC3CCCC3)nc21] | C | D | C |
| 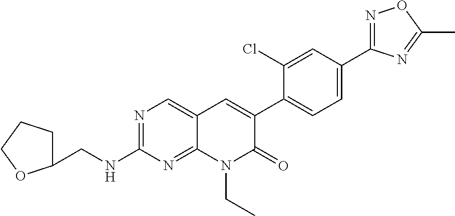 [SMILES: CCn1c(=O)c(-c2ccc(-c3noc(C)n3)cc2Cl)cc2cnc(NCC3CCCO3)nc21] | C | D | D |
| 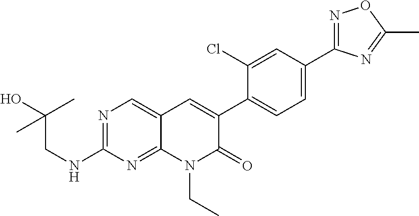 [SMILES: CCn1c(=O)c(-c2ccc(-c3noc(C)n3)cc2Cl)cc2cnc(NCC(C)(C)O)nc21] | C | D | D |
| 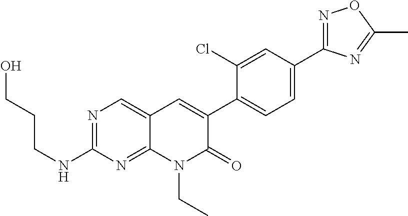 [SMILES: CCn1c(=O)c(-c2ccc(-c3noc(C)n3)cc2Cl)cc2cnc(NCCCO)nc21] | B | D | D |
| 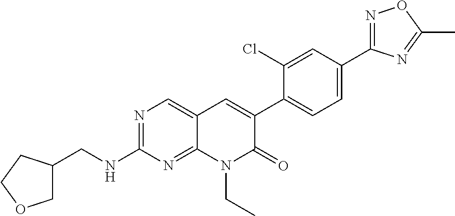 [SMILES: CCn1c(=O)c(-c2ccc(-c3noc(C)n3)cc2Cl)cc2cnc(NCC3CCOC3)nc21] | C | D | D |
| 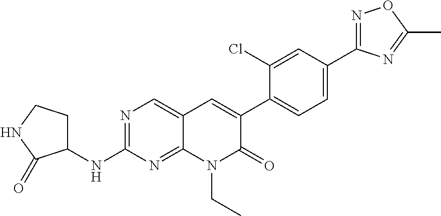 [SMILES: CCn1c(=O)c(-c2ccc(-c3noc(C)n3)cc2Cl)cc2cnc(NC3CCNC3=O)nc21] | D | D | D |
| 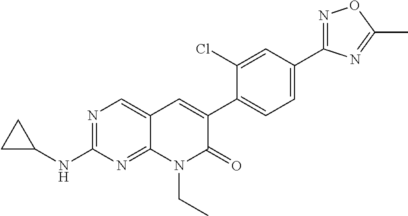 [SMILES: CCn1c(=O)c(-c2ccc(-c3noc(C)n3)cc2Cl)cc2cnc(NC3CC3)nc21] | B | D | D |
| 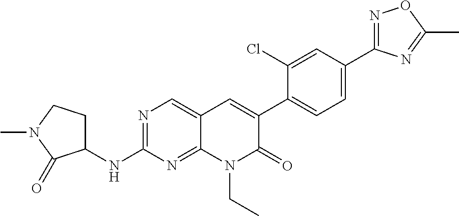 | D | D | D |
| 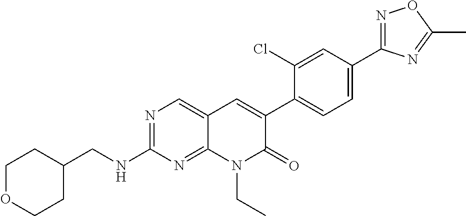 [SMILES: CCn1c(=O)c(-c2ccc(-c3noc(C)n3)cc2Cl)cc2cnc(NCC3CCOCC3)nc21] | C | D | D |
| 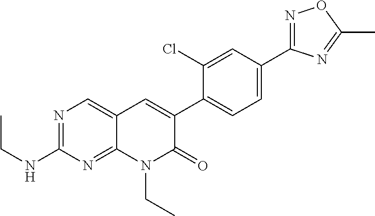 [SMILES: CCNc1ncc2cc(-c3ccc(-c4noc(C)n4)cc3Cl)c(=O)n(CC)c2n1] | C | D | D |
| 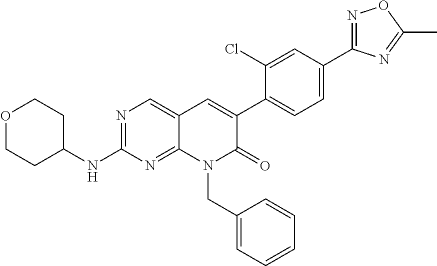 [SMILES: Cc1nc(-c2ccc(-c3cc4cnc(NC5CCOCC5)nc4n(Cc4ccccc4)c3=O)c(Cl)c2)no1] | C | D | D |
| 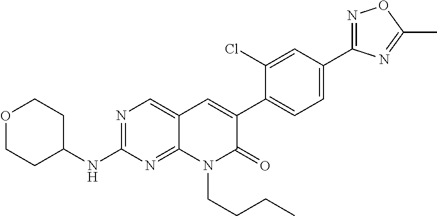 [SMILES: CCCCn1c(=O)c(-c2ccc(-c3noc(C)n3)cc2Cl)cc2cnc(NC3CCOCC3)nc21] | B | D | D |
| 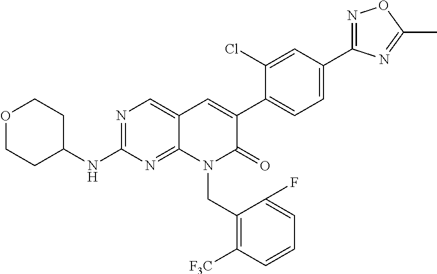 [SMILES: Cc1nc(-c2ccc(-c3cc4cnc(NC5CCOCC5)nc4n(Cc4c(F)cccc4C(F)(F)F)c3=O)c(Cl)c2)no1] | B | C | D |
| 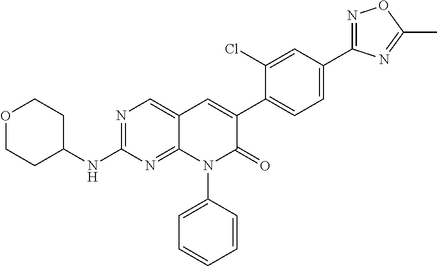 [SMILES: Cc1nc(-c2ccc(-c3cc4cnc(NC5CCOCC5)nc4n(-c4ccccc4)c3=O)c(Cl)c2)no1] | B | D | D |
| 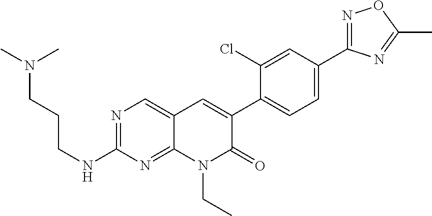 | C | D | D |
| 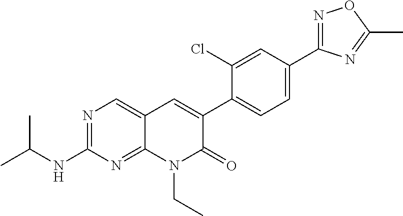 [SMILES: CCn1c(=O)c(-c2ccc(-c3noc(C)n3)cc2Cl)cc2cnc(NC(C)C)nc21] | C | D | D |
| 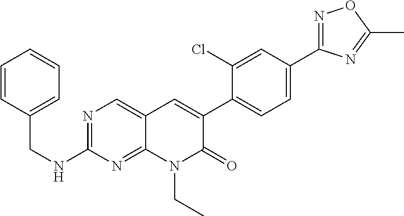 [SMILES: CCn1c(=O)c(-c2ccc(-c3noc(C)n3)cc2Cl)cc2cnc(NCc3ccccc3)nc21] | C | D | D |
| 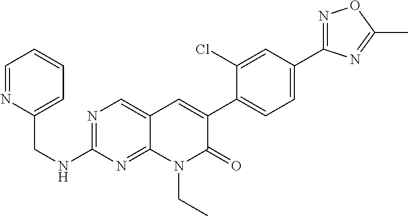 [SMILES: CCn1c(=O)c(-c2ccc(-c3noc(C)n3)cc2Cl)cc2cnc(NCc3ccccn3)nc21] | C | D | D |
| 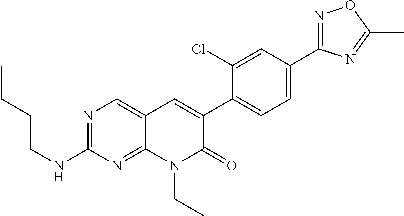 [SMILES: CCCCNc1ncc2cc(-c3ccc(-c4noc(C)n4)cc3Cl)c(=O)n(CC)c2n1] | C | D | D |
| 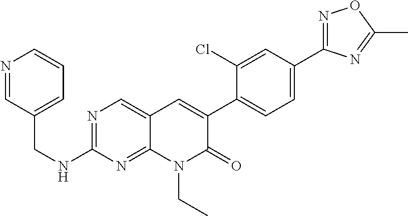 [SMILES: CCn1c(=O)c(-c2ccc(-c3noc(C)n3)cc2Cl)cc2cnc(NCc3cccnc3)nc21] | C | D | D |
| 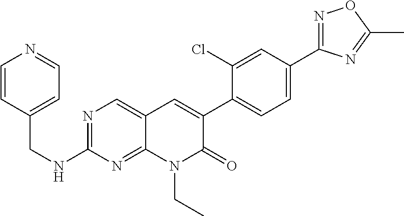 [SMILES: CCn1c(=O)c(-c2ccc(-c3noc(C)n3)cc2Cl)cc2cnc(NCc3ccncc3)nc21] | B | D | D |
| 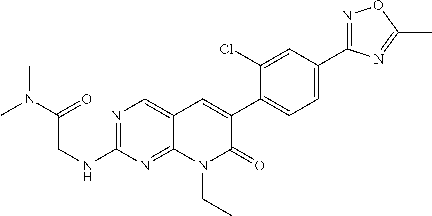 | D | D | B |
| 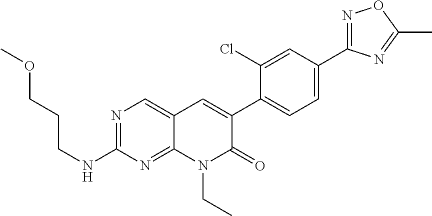 | C | D | D |
| 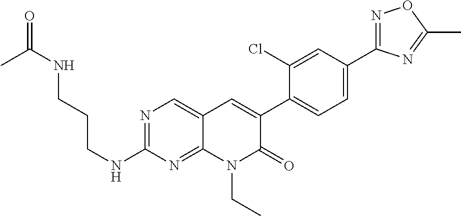 [SMILES: CCn1c(=O)c(-c2ccc(-c3noc(C)n3)cc2Cl)cc2cnc(NCCCNC(C)=O)nc21] | C | D | D |
| 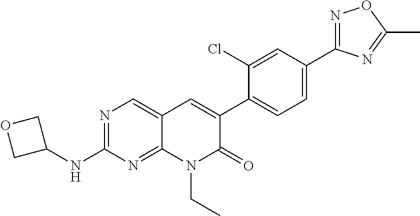 [SMILES: CCn1c(=O)c(-c2ccc(-c3noc(C)n3)cc2Cl)cc2cnc(NC3COC3)nc21] | B | D | D |
| 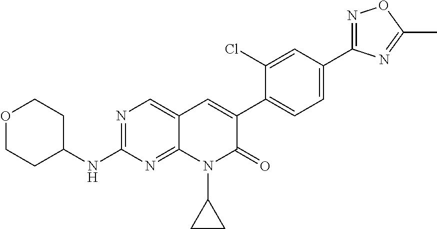 [SMILES: Cc1nc(-c2ccc(-c3cc4cnc(NC5CCOCC5)nc4n(C4CC4)c3=O)c(Cl)c2)no1] | B | D | D |
| 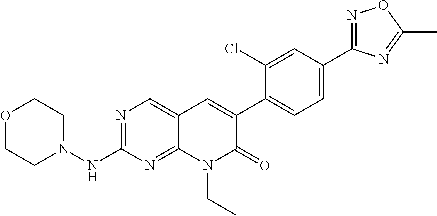 [SMILES: CCn1c(=O)c(-c2ccc(-c3noc(C)n3)cc2Cl)cc2cnc(NN3CCOCC3)nc21] | C | D | D |
| 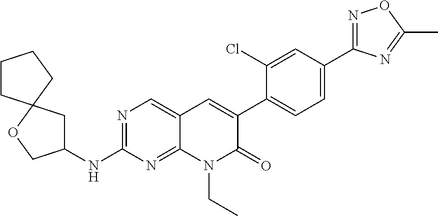 [SMILES: CCn1c(=O)c(-c2ccc(-c3noc(C)n3)cc2Cl)cc2cnc(NC3COC4(CCCC4)C3)nc21] | C | D | C |
| 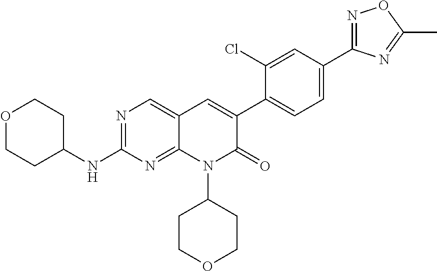 [SMILES: Cc1nc(-c2ccc(-c3cc4cnc(NC5CCOCC5)nc4n(C4CCOCC4)c3=O)c(Cl)c2)no1] | B | D | D |
| 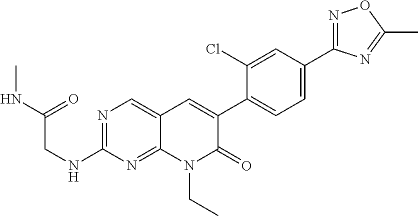 [SMILES: CCn1c(=O)c(-c2ccc(-c3noc(C)n3)cc2Cl)cc2cnc(NCC(=O)NC)nc21] | C | D | D |
| 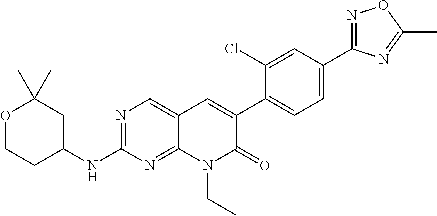 [SMILES: CCn1c(=O)c(-c2ccc(-c3noc(C)n3)cc2Cl)cc2cnc(NC3CCOC(C)(C)C3)nc21] | B | D | D |
| 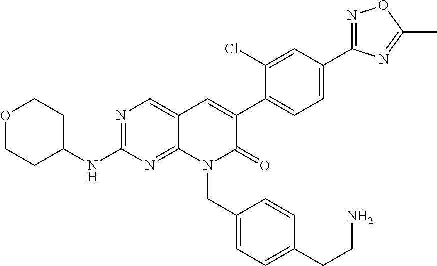 [SMILES: Cc1nc(-c2ccc(-c3cc4cnc(NC5CCOCC5)nc4n(Cc4ccc(CCN)cc4)c3=O)c(Cl)c2)no1] | A | C | D |
| 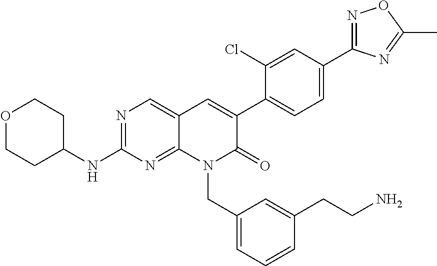 [SMILES: Cc1nc(-c2ccc(-c3cc4cnc(NC5CCOCC5)nc4n(Cc4cccc(CCN)c4)c3=O)c(Cl)c2)no1] | A | B | D |
| 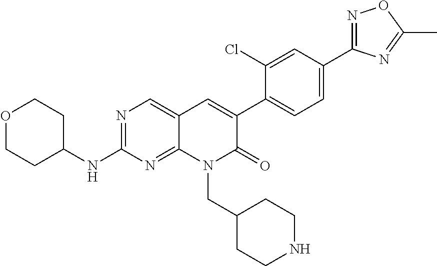 [SMILES: Cc1nc(-c2ccc(-c3cc4cnc(NC5CCOCC5)nc4n(CC4CCNCC4)c3=O)c(Cl)c2)no1] | A | C | D |
| 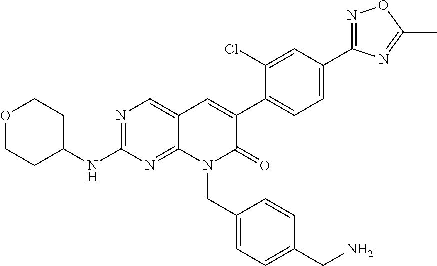 [SMILES: Cc1nc(-c2ccc(-c3cc4cnc(NC5CCOCC5)nc4n(Cc4ccc(CN)cc4)c3=O)c(Cl)c2)no1] | A | B | D |
| 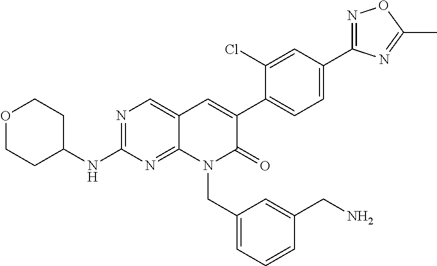 [SMILES: Cc1nc(-c2ccc(-c3cc4cnc(NC5CCOCC5)nc4n(Cc4cccc(CN)c4)c3=O)c(Cl)c2)no1] | A | B | D |
| 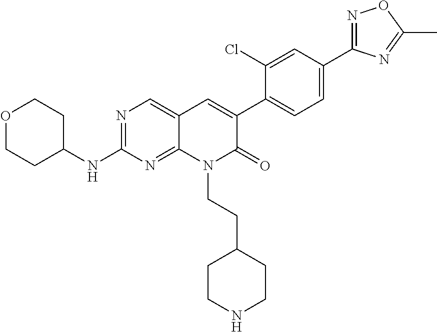 [SMILES: Cc1nc(-c2ccc(-c3cc4cnc(NC5CCOCC5)nc4n(CCC4CCNCC4)c3=O)c(Cl)c2)no1] | A | C | D |
| 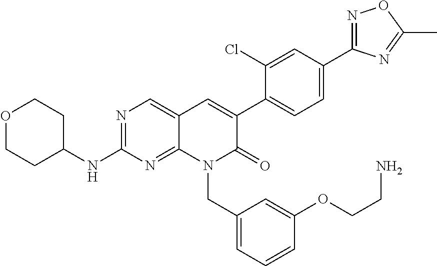 [SMILES: Cc1nc(-c2ccc(-c3cc4cnc(NC5CCOCC5)nc4n(Cc4cccc(OCCN)c4)c3=O)c(Cl)c2)no1] | A | C | D |
| 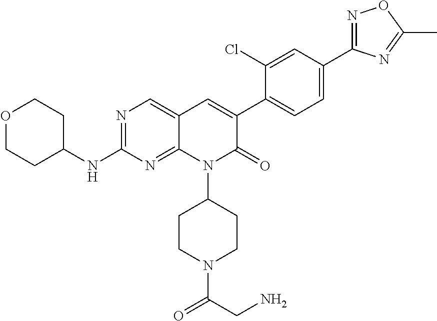 [SMILES: Cc1nc(-c2ccc(-c3cc4cnc(NC5CCOCC5)nc4n(C4CCN(C(=O)CN)CC4)c3=O)c(Cl)c2)no1] | B | D | D |
| 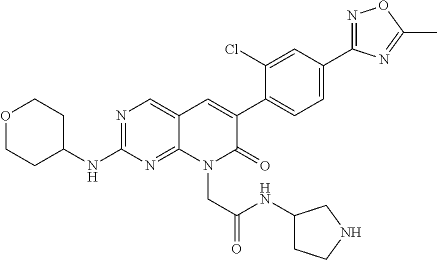 [SMILES: Cc1nc(-c2ccc(-c3cc4cnc(NC5CCOCC5)nc4n(CC(=O)NC4CCNC4)c3=O)c(Cl)c2)no1] | B | D | D |
| 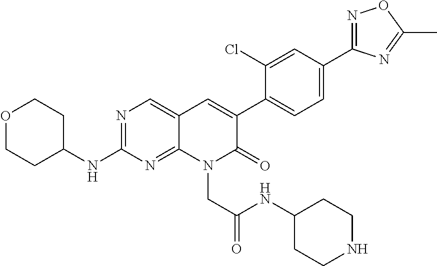 [SMILES: Cc1nc(-c2ccc(-c3cc4cnc(NC5CCOCC5)nc4n(CC(=O)NC4CCNCC4)c3=O)c(Cl)c2)no1] | C | D | D |
| 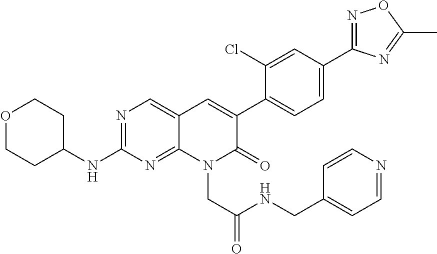 [SMILES: Cc1nc(-c2ccc(-c3cc4cnc(NC5CCOCC5)nc4n(CC(=O)NCc4ccncc4)c3=O)c(Cl)c2)no1] | B | D | D |
| 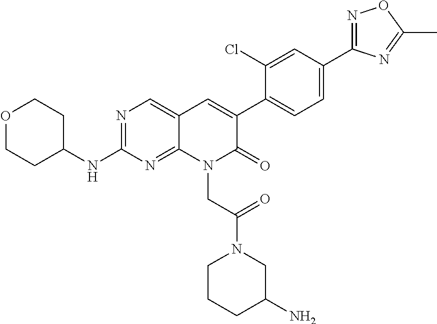 [SMILES: Cc1nc(-c2ccc(-c3cc4cnc(NC5CCOCC5)nc4n(CC(=O)N4CCCC(N)C4)c3=O)c(Cl)c2)no1] | B | D | D |
| 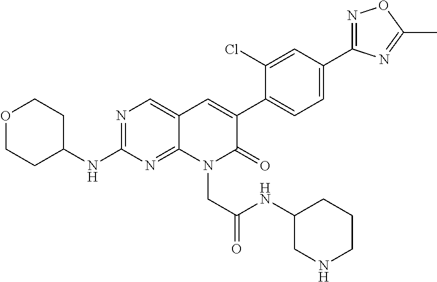 [SMILES: Cc1nc(-c2ccc(-c3cc4cnc(NC5CCOCC5)nc4n(CC(=O)NC4CCCNC4)c3=O)c(Cl)c2)no1] | B | D | D |
| 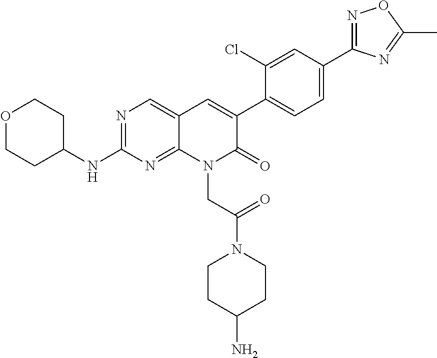 [SMILES: Cc1nc(-c2ccc(-c3cc4cnc(NC5CCOCC5)nc4n(CC(=O)N4CCC(N)CC4)c3=O)c(Cl)c2)no1] | B | D | D |
| 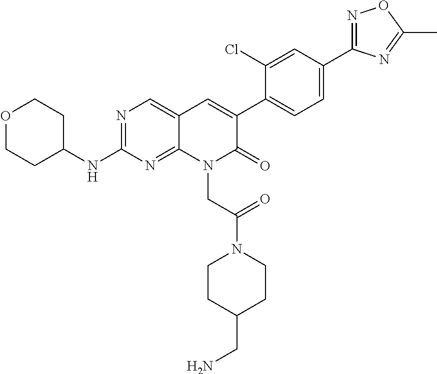 [SMILES: Cc1nc(-c2ccc(-c3cc4cnc(NC5CCOCC5)nc4n(CC(=O)N4CCC(CN)CC4)c3=O)c(Cl)c2)no1] | A | D | D |
| 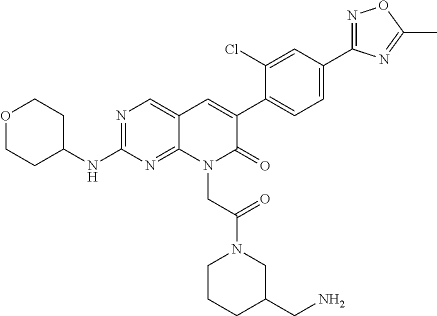 [SMILES: Cc1nc(-c2ccc(-c3cc4cnc(NC5CCOCC5)nc4n(CC(=O)N4CCCC(CN)C4)c3=O)c(Cl)c2)no1] | B | D | D |
| 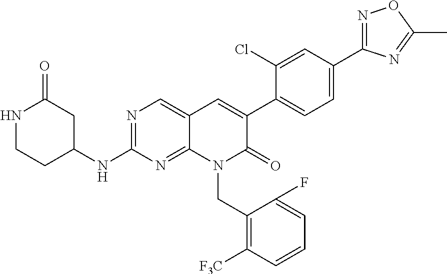 [SMILES: Cc1nc(-c2ccc(-c3cc4cnc(NC5CCNC(=O)C5)nc4n(Cc4c(F)cccc4C(F)(F)F)c3=O)c(Cl)c2)no1] | A | C | D |
| 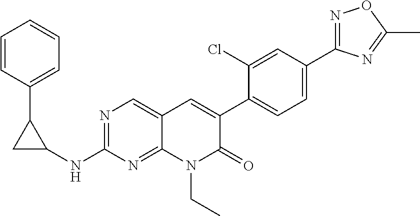 [SMILES: CCn1c(=O)c(-c2ccc(-c3noc(C)n3)cc2Cl)cc2cnc(NC3CC3c3ccccc3)nc21] | C | C | C |
| 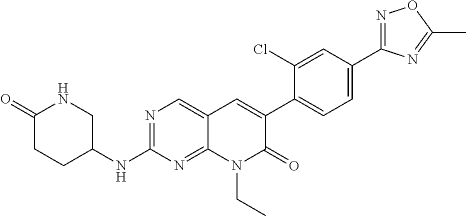 [SMILES: CCn1c(=O)c(-c2ccc(-c3noc(C)n3)cc2Cl)cc2cnc(NC3CCC(=O)NC3)nc21] | C | D | D |
| 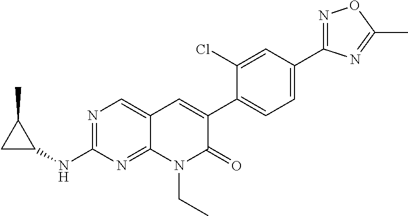 | B | A | A |
| 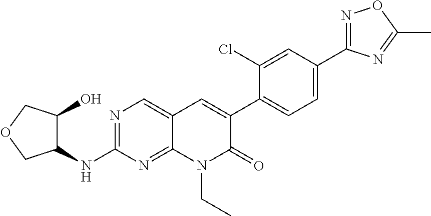 | C | D | D |
| 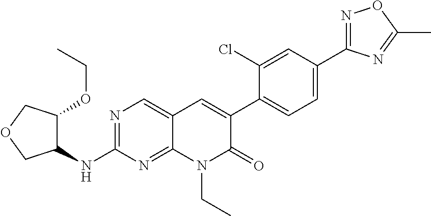 | C | D | D |
| 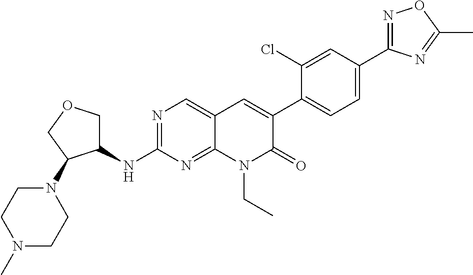 | B | D | D |
| 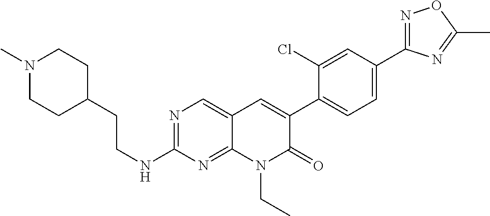 | B | D | D |
| 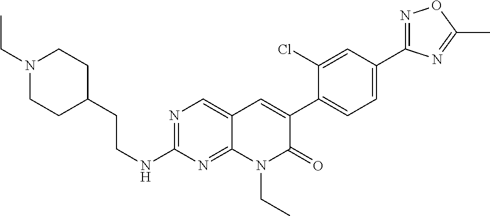 [SMILES: CCN1CCC(CCNc2ncc3cc(-c4ccc(-c5noc(C)n5)cc4Cl)c(=O)n(CC)c3n2)CC1] | B | C | D |
| 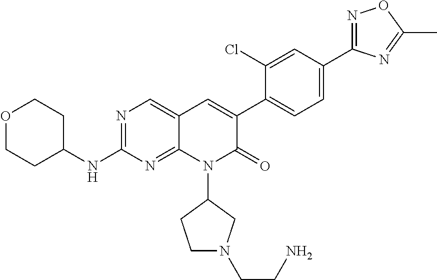 [SMILES: Cc1nc(-c2ccc(-c3cc4cnc(NC5CCOCC5)nc4n(C4CCN(CCN)C4)c3=O)c(Cl)c2)no1] | B | D | D |
| 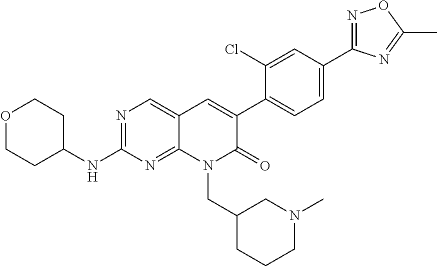 [SMILES: Cc1nc(-c2ccc(-c3cc4cnc(NC5CCOCC5)nc4n(CC4CCCN(C)C4)c3=O)c(Cl)c2)no1] | B | D | D |
| 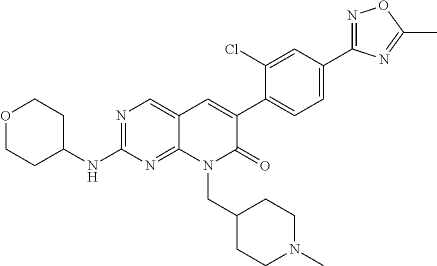 [SMILES: Cc1nc(-c2ccc(-c3cc4cnc(NC5CCOCC5)nc4n(CC4CCN(C)CC4)c3=O)c(Cl)c2)no1] | B | D | D |
| 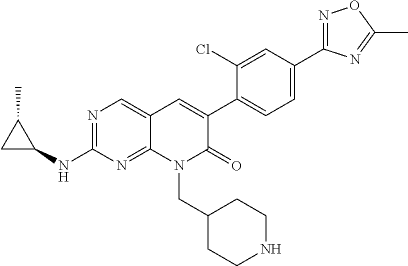 [SMILES: Cc1nc(-c2ccc(-c3cc4cnc(N[C@H]5C[C@@H]5C)nc4n(CC4CCNCC4)c3=O)c(Cl)c2)no1] | A | C | D |
| 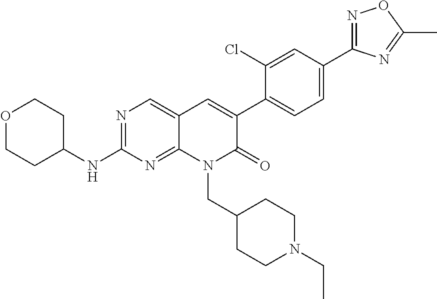 [SMILES: CCN1CCC(Cn2c(=O)c(-c3ccc(-c4noc(C)n4)cc3Cl)cc3cnc(NC4CCOCC4)nc32)CC1] | B | D | D |
| 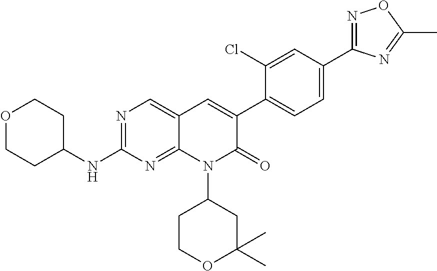 [SMILES: Cc1nc(-c2ccc(-c3cc4cnc(NC5CCOCC5)nc4n(C4CCOC(C)(C)C4)c3=O)c(Cl)c2)no1] | A | C | D |
| 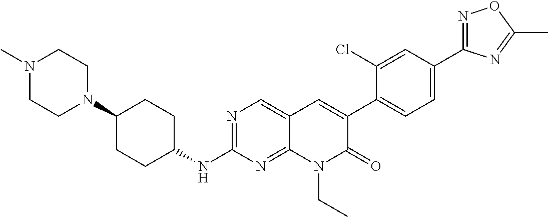 | C | D | D |
| 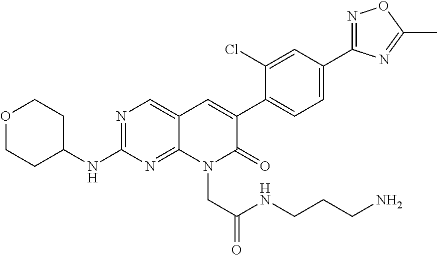 [SMILES: Cc1nc(-c2ccc(-c3cc4cnc(NC5CCOCC5)nc4n(CC(=O)NCCCN)c3=O)c(Cl)c2)no1] | A | C | D |
| 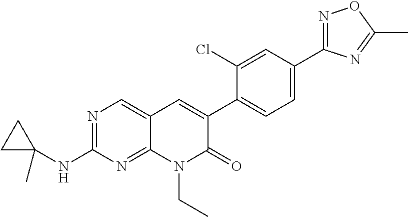 [SMILES: CCn1c(=O)c(-c2ccc(-c3noc(C)n3)cc2Cl)cc2cnc(NC3(C)CC3)nc21] | C | D | D |
| 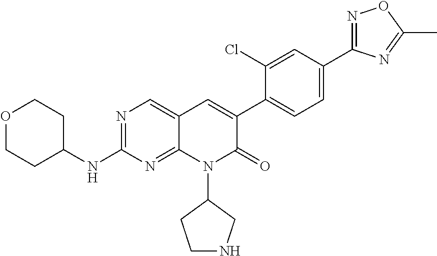 [SMILES: Cc1nc(-c2ccc(-c3cc4cnc(NC5CCOCC5)nc4n(C4CCNC4)c3=O)c(Cl)c2)no1] | C | D | D |
| 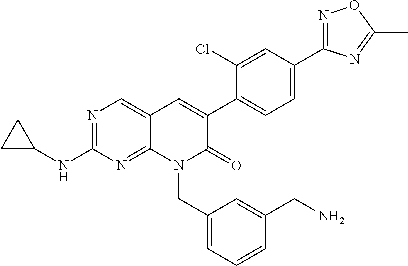 [SMILES: Cc1nc(-c2ccc(-c3cc4cnc(NC5CC5)nc4n(Cc4cccc(CN)c4)c3=O)c(Cl)c2)no1] | A | C | D |
| 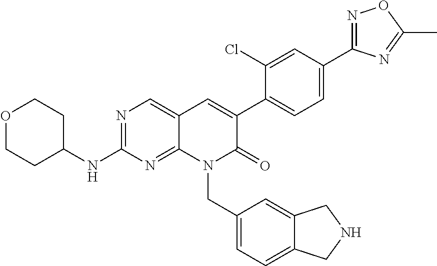 [SMILES: Cc1nc(-c2ccc(-c3cc4cnc(NC5CCOCC5)nc4n(Cc4ccc5c(c4)CNC5)c3=O)c(Cl)c2)no1] | B | D | D |
| 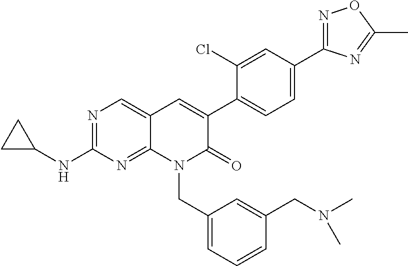 [SMILES: Cc1nc(-c2ccc(-c3cc4cnc(NC5CC5)nc4n(Cc4cccc(CN(C)C)c4)c3=O)c(Cl)c2)no1] | B | C | D |
| 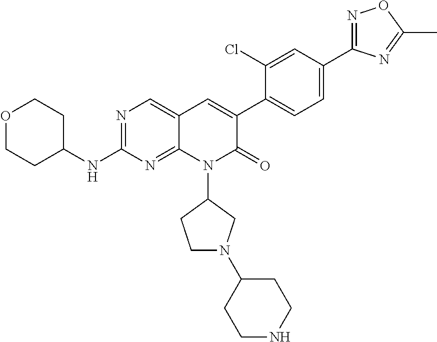 [SMILES: Cc1nc(-c2ccc(-c3cc4cnc(NC5CCOCC5)nc4n(C4CCN(C5CCNCC5)C4)c3=O)c(Cl)c2)no1] | B | D | D |
| 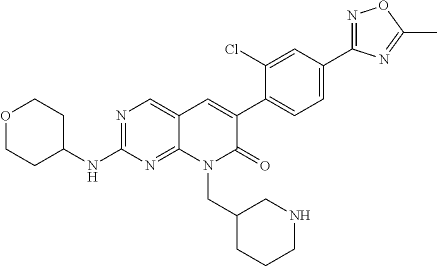 [SMILES: Cc1nc(-c2ccc(-c3cc4cnc(NC5CCOCC5)nc4n(CC4CCCNC4)c3=O)c(Cl)c2)no1] | B | D | D |

A, IC50 < 50 nM;
B, 50 nM ≦ IC50 ≦ 500 nM;
C, 0.5 μM < IC50 < 5 μM;
D, IC50 ≧ 5 μM

### Example 4

### In Vitro p-PAK1(S144) and p-MEK1(S298) Cellular Assay

Some of the compounds for treatment of Fragile X syndrome are subject to in vitro cellular HTRF assay for p-PAK1(S144) and p-MEK1(5298). The cell line is RT4-D6P2T. The HTRF assay kits are obtained from Cisbio, 135 South Road, Bedford, Mass. 01730, USA. The results are listed in the table below.

| | pMEK1(S298) | pPAK1(S144) |
|---|---|---|
| Compound | (μM) | (μM) |
| 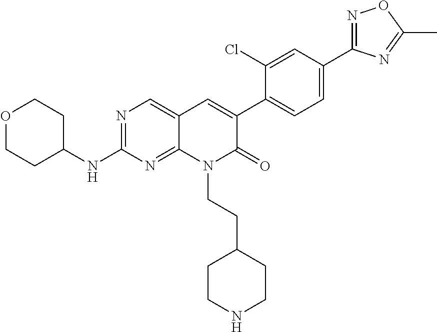 [SMILES: Cc1nc(-c2ccc(-c3cc4cnc(NC5CCOCC5)nc4n(CCC4CCNCC4)c3=O)c(Cl)c2)no1] | C | |
| 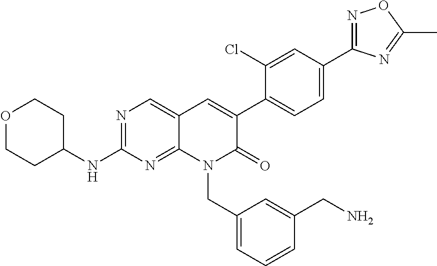 [SMILES: Cc1nc(-c2ccc(-c3cc4cnc(NC5CCOCC5)nc4n(Cc4cccc(CN)c4)c3=O)c(Cl)c2)no1] | C | B |
| 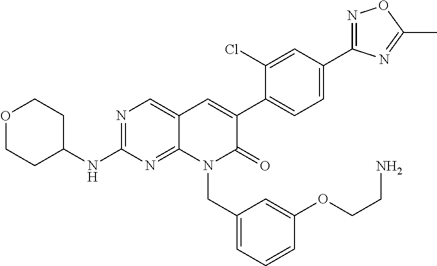 [SMILES: Cc1nc(-c2ccc(-c3cc4cnc(NC5CCOCC5)nc4n(Cc4cccc(OCCN)c4)c3=O)c(Cl)c2)no1] | C | C |
| 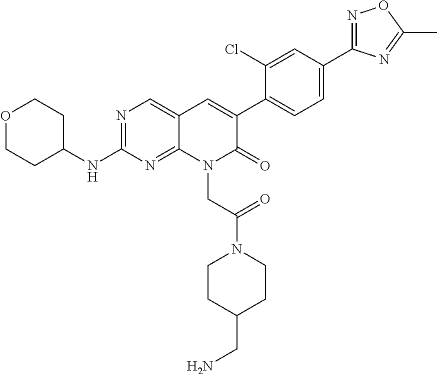 [SMILES: Cc1nc(-c2ccc(-c3cc4cnc(NC5CCOCC5)nc4n(CC(=O)N4CCC(CN)CC4)c3=O)c(Cl)c2)no1] | D | |
| 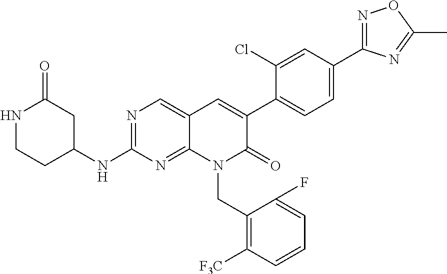 [SMILES: Cc1nc(-c2ccc(-c3cc4cnc(NC5CCNC(=O)C5)nc4n(Cc4c(F)cccc4C(F)(F)F)c3=O)c(Cl)c2)no1] | D | C |
| 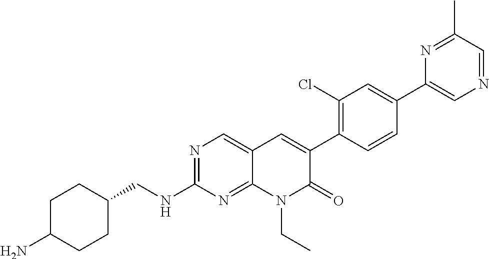 | C | B |
| 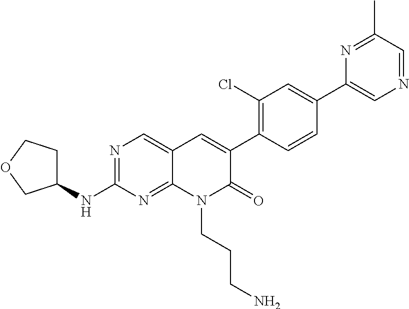 [SMILES: Cc1cncc(-c2ccc(-c3cc4cnc(N[C@@H]5CCOC5)nc4n(CCCN)c3=O)c(Cl)c2)n1] | C | C |
| 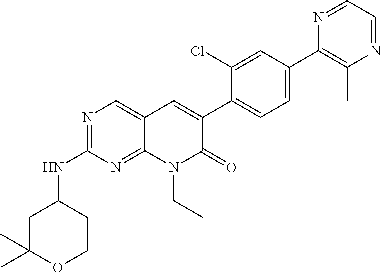 [SMILES: CCn1c(=O)c(-c2ccc(-c3nccnc3C)cc2Cl)cc2cnc(NC3CCOC(C)(C)C3)nc21] | C | C |
| 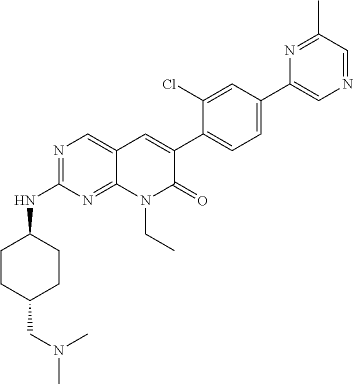 [SMILES: CCn1c(=O)c(-c2ccc(-c3cncc(C)n3)cc2Cl)cc2cnc(N[C@H]3CC[C@H](CN(C)C)CC3)nc21] | C | C |
| 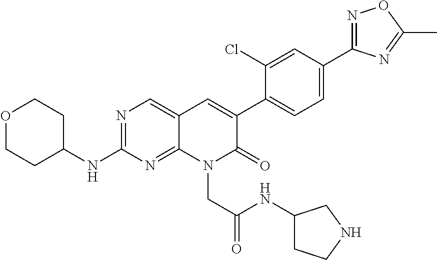 [SMILES: Cc1nc(-c2ccc(-c3cc4cnc(NC5CCOCC5)nc4n(CC(=O)NC4CCNC4)c3=O)c(Cl)c2)no1] | D | |
| 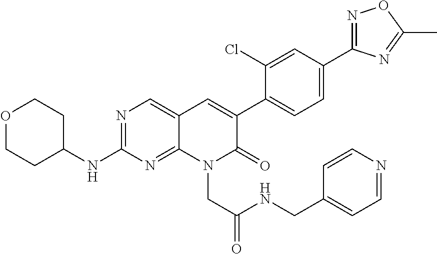 [SMILES: Cc1nc(-c2ccc(-c3cc4cnc(NC5CCOCC5)nc4n(CC(=O)NCc4ccncc4)c3=O)c(Cl)c2)no1] | D | C |
| 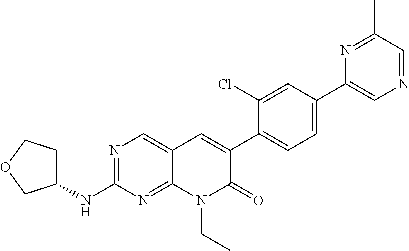 [SMILES: CCn1c(=O)c(-c2ccc(-c3cncc(C)n3)cc2Cl)cc2cnc(N[C@H]3CCOC3)nc21] | D | C |
| 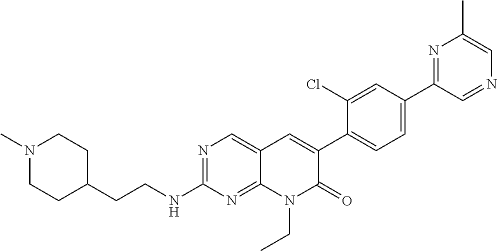 [SMILES: CCn1c(=O)c(-c2ccc(-c3cncc(C)n3)cc2Cl)cc2cnc(NCCC3CCN(C)CC3)nc21] | B | B |
| 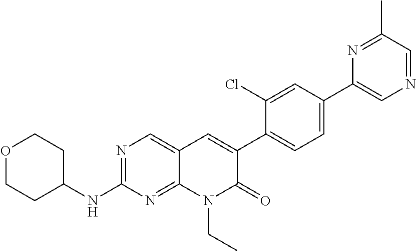 [SMILES: CCn1c(=O)c(-c2ccc(-c3cncc(C)n3)cc2Cl)cc2cnc(NC3CCOCC3)nc21] | C | B |
| 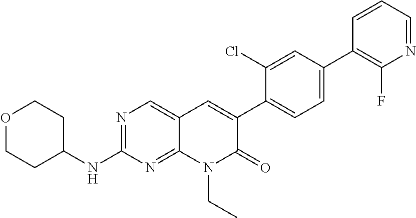 [SMILES: CCn1c(=O)c(-c2ccc(-c3cccnc3F)cc2Cl)cc2cnc(NC3CCOCC3)nc21] | C | C |
| 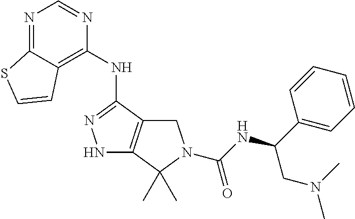 [SMILES: CN(C)C[C@@H](NC(=O)N1Cc2c(Nc3ncnc4sccc34)n[nH]c2C1(C)C)c1ccccc1] | C | B |
| 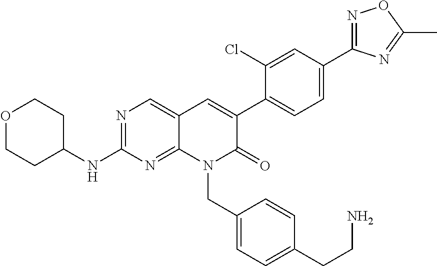 [SMILES: Cc1nc(-c2ccc(-c3cc4cnc(NC5CCOCC5)nc4n(Cc4ccc(CCN)cc4)c3=O)c(Cl)c2)no1] | C | B |
| 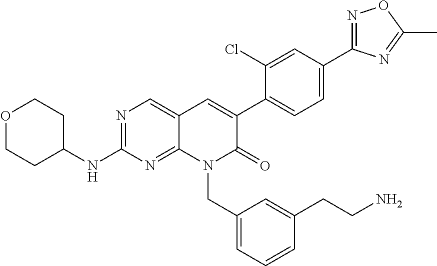 [SMILES: Cc1nc(-c2ccc(-c3cc4cnc(NC5CCOCC5)nc4n(Cc4cccc(CCN)c4)c3=O)c(Cl)c2)no1] | B | B |
| 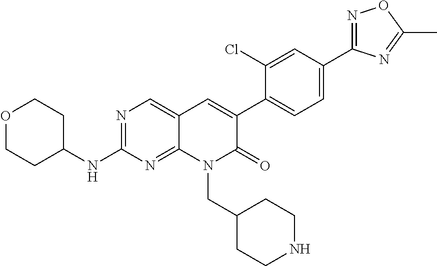 [SMILES: Cc1nc(-c2ccc(-c3cc4cnc(NC5CCOCC5)nc4n(CC4CCNCC4)c3=O)c(Cl)c2)no1] | C | B |
| 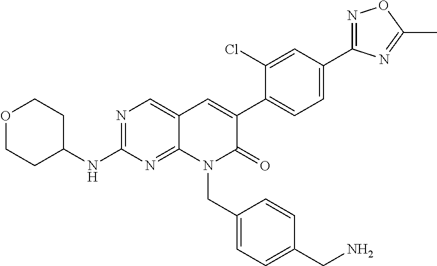 [SMILES: Cc1nc(-c2ccc(-c3cc4cnc(NC5CCOCC5)nc4n(Cc4ccc(CN)cc4)c3=O)c(Cl)c2)no1] | C | B |

A, IC50 < 50 nM;
B, 50 nM ≦ IC50 ≦ 500 nM;
C, 0.5 μM < IC50 < 5 μM;
D, IC50 ≧ 5 μM

### Example 5

### In Vivo Monitoring of Dendritic Spine Plasticity in Double Transgenic GFP-M/DN-DISC1 Mice Treated with a PAK Inhibitor Compound Disclosed Herein

In the following experiment, dendritic spine plasticity is directly monitored in vivo by two photon laser scanning microscopy (TPLSM) in double transgenic GFP-M/DN-DISC1 mice treated with a compound disclosed herein or a placebo. Mice (C57BL/6) expressing GFP in a subset of cortical layer 5 neurons (transgenic line GFP-M described in Feng et al, 2000, Neuron 28:41-51) are crossed with DN-DISC1 C57BL/6 DN-DISC1 mice (Hikida et al (2007), Proc Natl Acad Sci USA, 104(36):14501-14506) to obtain heterozygous transgenic mice, which are then crossed to obtain homozygous double transgenic GFPM/DN-DISC1 mice used in this study.

GFP-M/DN-DISC1 animals aged 28-61 d are anesthetized using avertin (16 μl/g body weight; Sigma, St. Louis, Mo.). The skull is exposed, scrubbed, and cleaned with ethanol. Primary visual, somatosensory, auditory, and motor cortices are identified based on stereotaxic coordinates, and their location is confirmed with tracer injections (see below).

Long-term imaging experiments are started at P40. The skull is thinned over the imaging area as described in Grutzendler et al, (2002), Nature, 420:812-816. A small metal bar is affixed to the skull. The metal bar is then screwed into a plate that connected directly to the microscope stage for stability during imaging. The metal bar also allows for maintaining head angle and position during different imaging sessions. At the end of the imaging session, animals are sutured and returned to their cage. Thirty animals previously imaged at P40 are then divided into a control group receiving a 1% sugar solution (oral gavage once per day) and a treatment group administered a compound disclosed herein, in 0.1% DMSO (oral gavage. 1 mg/kg, once per day). During the subsequent imaging sessions (at P45, P50, P55, or P70), animals are reanesthetized and the skull is rethinned. The same imaging area is identified based on the blood vessel pattern and gross dendritic pattern, which generally remains stable over this time period.

At the end of the last imaging session, injections of cholera toxin subunit B coupled to Alexa Fluor 594 are made adjacent to imaged areas to facilitate identification of imaged cells and cortical areas after fixation. Mice are transcardially perfused and fixed with paraformaldehyde, and coronal sections are cut to verify the location of imaged cells. Sections are then mounted in buffer, coverslipped, and sealed. Images are collected using a Fluoview confocal microscope (Olympus Optical, Melville, N.Y.).

For in vivo two photon imaging, a two-photon laser scanning microscope is used as described in Majewska et al, (2000), Pflügers Arch, 441:398-408. The microscope consists of a modified Fluoview confocal scan head (Olympus Optical) and a titanium/sulphur laser providing 100 fs pulses at 80 MHz at a wavelength of 920 nm (Tsunami; Spectra-Physics, Menlo Park, Calif.) pumped by a 10 W solid-state source (Millenia; Spectra-Physics). Fluorescence is detected using photomultiplier tubes (HC125-02; Hamamatsu, Shizouka, Japan) in whole-field detection mode. The craniotomy over the visual cortex is initially identified under whole-field fluorescence illumination, and areas with superficial dendrites are identified using a 20×, 0.95 numerical aperture lens (IR2; Olympus Optical). Spiny dendrites are further identified under digital zoom (7-10×) using two-photon imaging, and spines 50-200 μm below the pial surface are studied. Image acquisition is accomplished using Fluoview software. For motility measurements, Z stacks taken 0.5-1 μm apart are acquired every 5 min for 2 h. For synapse turnover experiments, Z stacks of dendrites and axons are acquired at P40 and then again at P50 or P70. Dendrites and axons located in layers 1-3 are studied. Although both layer 5 and layer 6 neurons are labeled in the mice used in this study, only layer 5 neurons send a clear apical dendrite close to the pial surface thus, the data will come from spines on the apical tuft of layer 5 neurons and axons in superficial cortical layers.

Images are exported to Matlab (MathWorks, Natick, Mass.) in which they are processed using custom-written algorithms for image enhancement and alignment of the time series. For motility measurements (see Majewska et al, (2003), Proc Natl Acad Sci USA, 100:16024-16029) spines are analyzed on two-dimensional projections containing between 5 and 30 individual images; therefore, movements in the z dimension are not analyzed. Spine motility is defined as the average change in length per unit time (micrometers per minute). Lengths are measured from the base of the protrusion to its tip. The position of spines is compared on different imaging days. Spines that are farther than 0.5 μm laterally from their previous location are considered to be different spines. Values for stable spines are defined as the percentage of the original spine population present on the second day of imaging. Only areas that show high signal-to-noise ratio in all imaging sessions will be considered for analysis. Analysis is performed blind with respect to animal age and sensory cortical area. Spine motility (e.g., spine turnover), morphology, and density are then compared between control and treatment groups. It is expected that treatment with a compound disclosed herein will rescue defective spine morphology relative to that observed in untreated control animals.

### Example 6

### Pharmaceutical Compositions

### Example 6a

### Parenteral Composition

To prepare a parenteral pharmaceutical composition suitable for administration by injection, 100 mg of a water-soluble salt of a compound of Formula I-IV and A-D is dissolved in DMSO and then mixed with 10 mL of 0.9% sterile saline. The mixture is incorporated into a dosage unit form suitable for administration by injection.

### Example 6b

### Oral Composition

To prepare a pharmaceutical composition for oral delivery, 100 mg of a compound of Formula I-IV and A-D is mixed with 750 mg of starch. The mixture is incorporated into an oral dosage unit for, e.g., a hard gelatin capsule, which is suitable for oral administration.

### Example 6c

### Sublingual (Hard Lozenge) Composition

To prepare a pharmaceutical composition for buccal delivery, such as a hard lozenge, mix 100 mg of a compound of Formula I-IV and A-D with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 6d

### Fast-Disintegrating Sublingual Tablet

A fast-disintegrating sublingual tablet is prepared by mixing 48.5% by weigh of a compound of Formula I-IV and A-D, 44.5% by weight of microcrystalline cellulose (KG-802), 5% by weight of low-substituted hydroxypropyl cellulose (50 μm), and 2% by weight of magnesium stearate. Tablets are prepared by direct compression (AAPS PharmSciTech. 2006; 7(2):E41). The total weight of the compressed tablets is maintained at 150 mg. The formulation is prepared by mixing the amount of compound of Formula I-IV and A-D with the total quantity of microcrystalline cellulose (MCC) and two-thirds of the quantity of low-substituted hydroxypropyl cellulose (L-HPC) by using a three dimensional manual mixer (Inversina®, Bioengineering AG, Switzerland) for 4.5 minutes. All of the magnesium stearate (MS) and the remaining one-third of the quantity of L-HPC are added 30 seconds before the end of mixing.

### Example 6e

### Inhalation Composition

To prepare a pharmaceutical composition for inhalation delivery, 20 mg of a compound of Formula I-IV and A-D is mixed with 50 mg of anhydrous citric acid and 100 mL of 0.9% sodium chloride solution. The mixture is incorporated into an inhalation delivery unit, such as a nebulizer, which is suitable for inhalation administration.

### Example 6f

### Rectal Gel Composition

To prepare a pharmaceutical composition for rectal delivery, 100 mg of a compound of Formula I-IV and A-D is mixed with 2.5 g of methylcellulose (1500 mPa), 100 mg of methylparapen, 5 g of glycerin and 100 mL of purified water. The resulting gel mixture is then incorporated into rectal delivery units, such as syringes, which are suitable for rectal administration.

### Example 6g

### Topical Gel Composition

To prepare a pharmaceutical topical gel composition, 100 mg of a compound of Formula I-IV and A-D is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Example 6h

### Ophthalmic Solution Composition

To prepare a pharmaceutical ophthalmic solution composition, 100 mg of a compound of Formula I-IV and A-D is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Example 6i

### Nasal Spray Solution

To prepare a pharmaceutical nasal spray solution, 10 g of a compound of Formula I-IV and A-D is mixed with 30 mL of a 0.05M phosphate buffer solution (pH 4.4). The solution is placed in a nasal administrator designed to deliver 100 μl of spray for each application.

While some embodiments of the present disclosure have been shown and described herein, such embodiments are provided by way of example only. It is intended that the following claims define the scope of the present disclosure and that methods and structures within the scope of these claims and their equivalents be covered thereby.

What is claimed is:

1. A compound having the structure of Formula I, Formula II, or Formula III, or a pharmaceutically acceptable salt or N-oxide thereof: 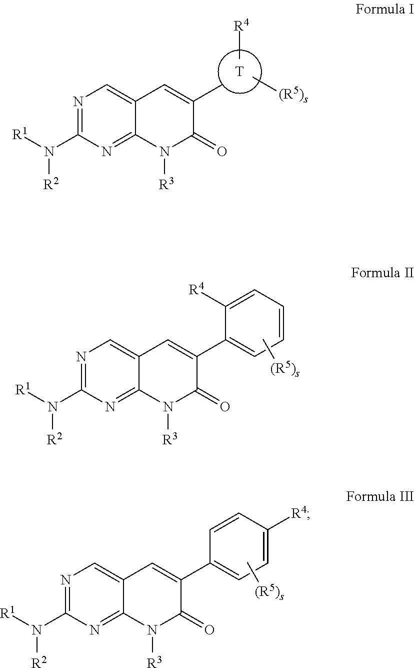
wherein:
ring T is an aryl or heteroaryl ring;
R¹ is H, or substituted or unsubstituted alkyl;
R² is alkyl substituted with —OH, —OMe, —SH, —SMe, or halogen;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted heteroaryl attached to ring T or the phenyl ring via a carbon atom of R⁴, or substituted or unsubstituted heterocycloalkyl attached to ring T or the phenyl ring via a carbon atom of R⁴;
each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

2. The compound of claim 1 having the structure of Formula I.

3. The compound of claim 2 having the structure of Formula Ia:

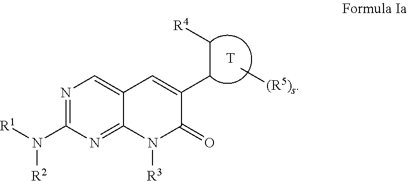

4. The compound of claim 2 having the structure of Formula Ib: 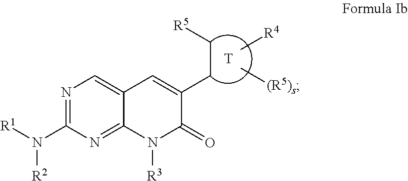
wherein s is 0-3.

5. The compound of claim 1, wherein ring T is selected from pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl.

6. The compound of claim 1 having the structure of Formula II.

7. The compound of claim 1 having the structure of Formula III.

8. The compound of claim 7 having the structure of Formula IIIa: 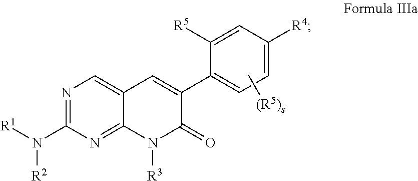
wherein s is 0-3.

9. The compound of claim 7 having the structure of Formula Mb: 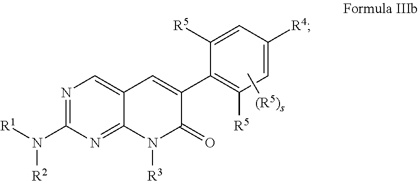
wherein s is 0-2.

10. A compound having the structure of Formula IV, or a pharmaceutically acceptable salt or N-oxide thereof: 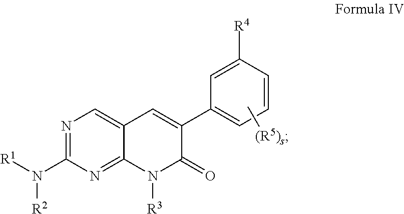
wherein:
R¹ is H, or substituted or unsubstituted alkyl;
R² is alkyl substituted with —OH, —OMe, —SH, —SMe, or halogen;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted 6-membered monocyclic heteroaryl ring attached to the phenyl ring via a carbon atom of R⁴, substituted or unsubstituted bicyclic heteroaryl ring attached to the phenyl ring via a carbon atom of R⁴, or substituted or unsubstituted heterocycloalkyl attached to the phenyl ring via a carbon atom of R⁴;
each R⁵ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

11. The compound of claim 10, wherein R⁴ is a substituted or unsubstituted C-linked 6-membered monocyclic heteroaryl ring or a substituted or unsubstituted C-linked bicyclic heteroaryl ring.

12. The compound of claim 11, wherein R⁴ is pyridine, pyridazinyl, pyrimidinyl, pyrazinyl, indolyl, benzofuranyl, benzimidazolyl, indazolyl, pyrrolopyridinyl, or imidazopyridinyl.

13. The compound of claim 1, wherein R⁴ is a substituted or unsubstituted C-linked heteroaryl.

14. The compound of claim 13 wherein R⁴ is selected from pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, and imidazopyridinyl.

15. The compound of claim 1, wherein R⁴ is a C-linked heterocycloalkyl.

16. The compound of claim 15, wherein heterocycloalkyl is pyrrolidinyl, tetrahydrofuranyl, piperidinyl, tetrahydropyranyl, tetrahydrothiopyranyl, morpholinyl, or piperazinyl.

17. The compound of any one of claim 1, wherein each R⁵ is independently halogen, —CN, —OH, —OCF₃, —OCF₃, —OCF₂H, —CF₃, —SR⁸, —N(R¹⁰)₂, a substituted or unsubstituted alkyl, or a substituted or unsubstituted alkoxy.

18. The compound of claim 17, wherein each R⁵ is independently halogen, —N(R¹⁰)₂, or a substituted or unsubstituted alkyl.

19. The compound of claim 18 wherein s is 0.

20. The compound of claim 18 wherein s is 1.

21. The compound of claim 18 wherein s is 2.

22. The compound of claim 1, wherein R³ is H.

23. The compound of claim 1, wherein R³ is a substituted or unsubstituted alkoxy, or a substituted or unsubstituted amino.

24. The compound of claim 1, wherein R³ is a substituted or unsubstituted alkyl, or a substituted or unsubstituted heteroalkyl.

25. The compound of claim 1, wherein R³ is a substituted or unsubstituted cycloalkyl, or a substituted or unsubstituted heterocycloalkyl.

26. The compound of claim 25, wherein cycloalkyl is cyclopropyl, cyclobutyl, cyclopentyl, cyclohexyl or cycloheptyl.

27. The compound of claim 25, wherein heterocycloalkyl is pyrrolidinyl, tetrahydrofuranyl, piperidinyl, tetrahydropyranyl, tetrahydrothiopyranyl, morpholinyl, or piperazinyl.

28. The compound of claim 1, wherein R³ is a substituted or unsubstituted cycloalkylalkyl, or a substituted or unsubstituted heterocycloalkylalkyl.

29. The compound of claim 1, wherein R³ is a substituted or unsubstituted aryl, or a substituted or unsubstituted heteroaryl.

30. The compound of claim 29, wherein aryl is phenyl.

31. The compound of claim 29, wherein heteroaryl is pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, triazinyl, indolyl, benzofuranyl, benzimidazolyl, indazolyl, pyrrolopyridinyl, or imidazopyridinyl.

32. The compound of claim 1, wherein R³ is a substituted or unsubstituted arylalkyl, or a substituted or unsubstituted heteroarylalkyl.

33. The compound of claim 1, wherein R² is C₁-C₄alkyl substituted with hydroxy or C1-C₄alkyl substituted with methoxy.

34. The compound of claim 1, wherein R² is —CH(CH₂CH₂OH)₂.

35. The compound of claim 1, wherein R¹ is H.

36. The compound of claim 1, wherein R¹ is substituted or unsubstituted alkyl.

37. A compound selected from the group consisting of: 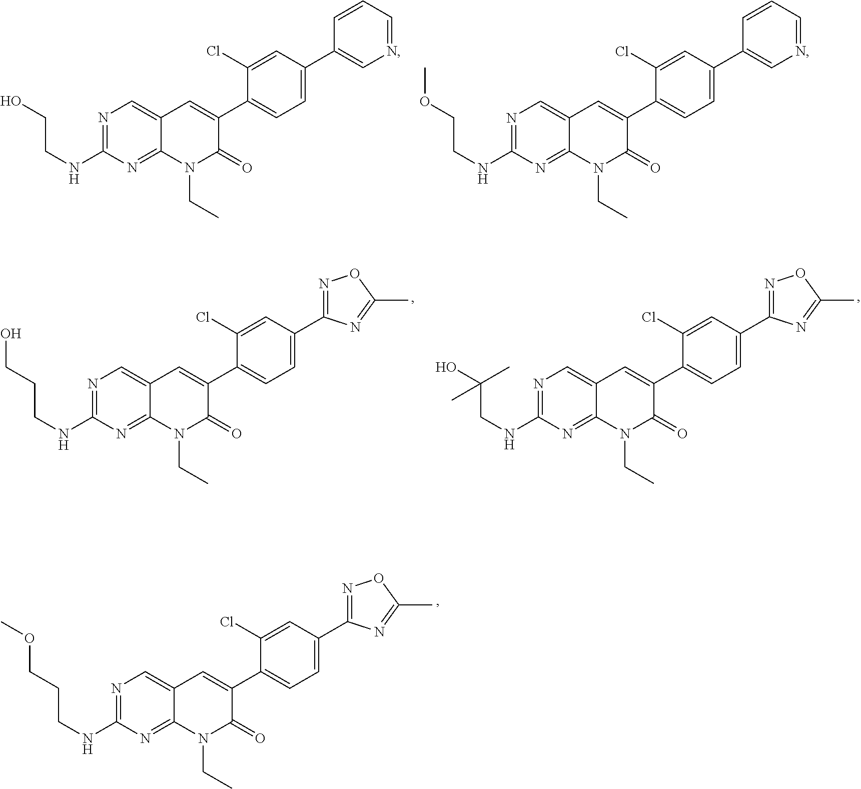 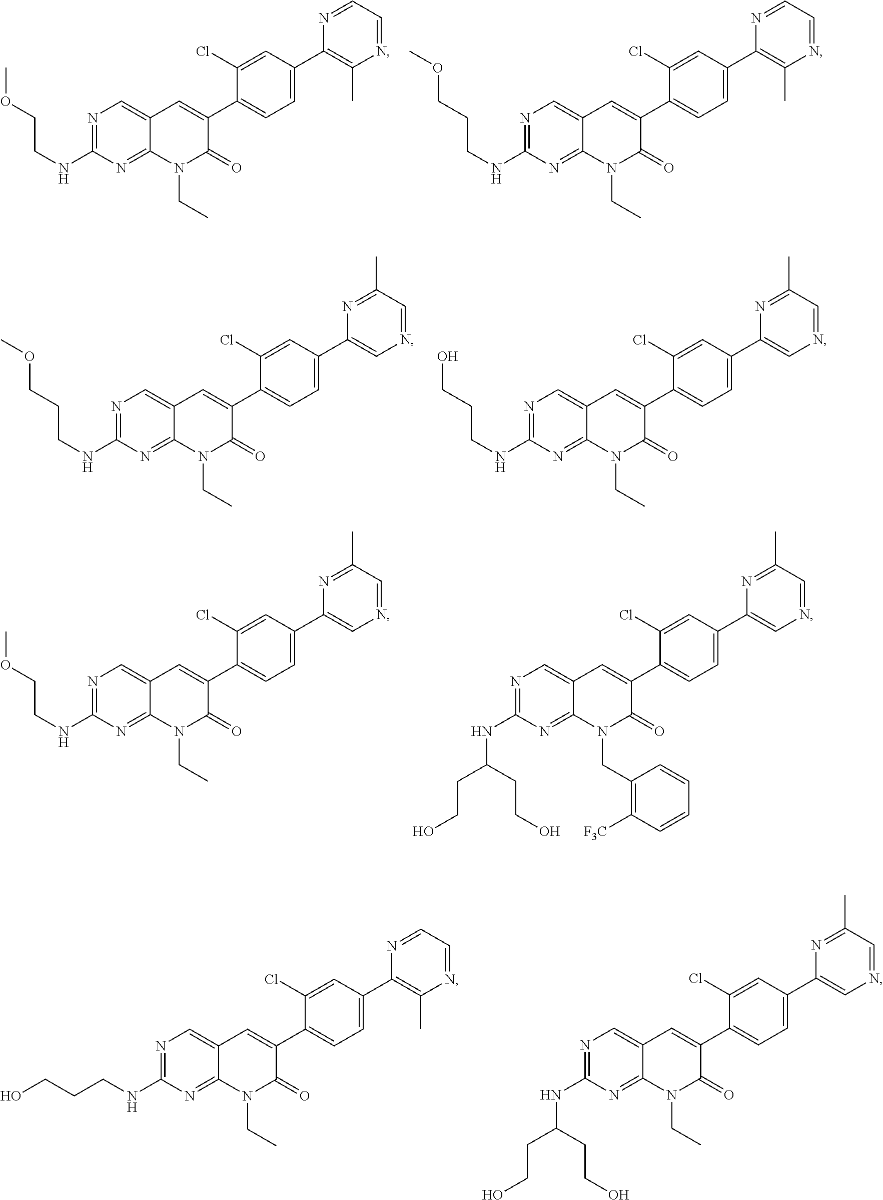 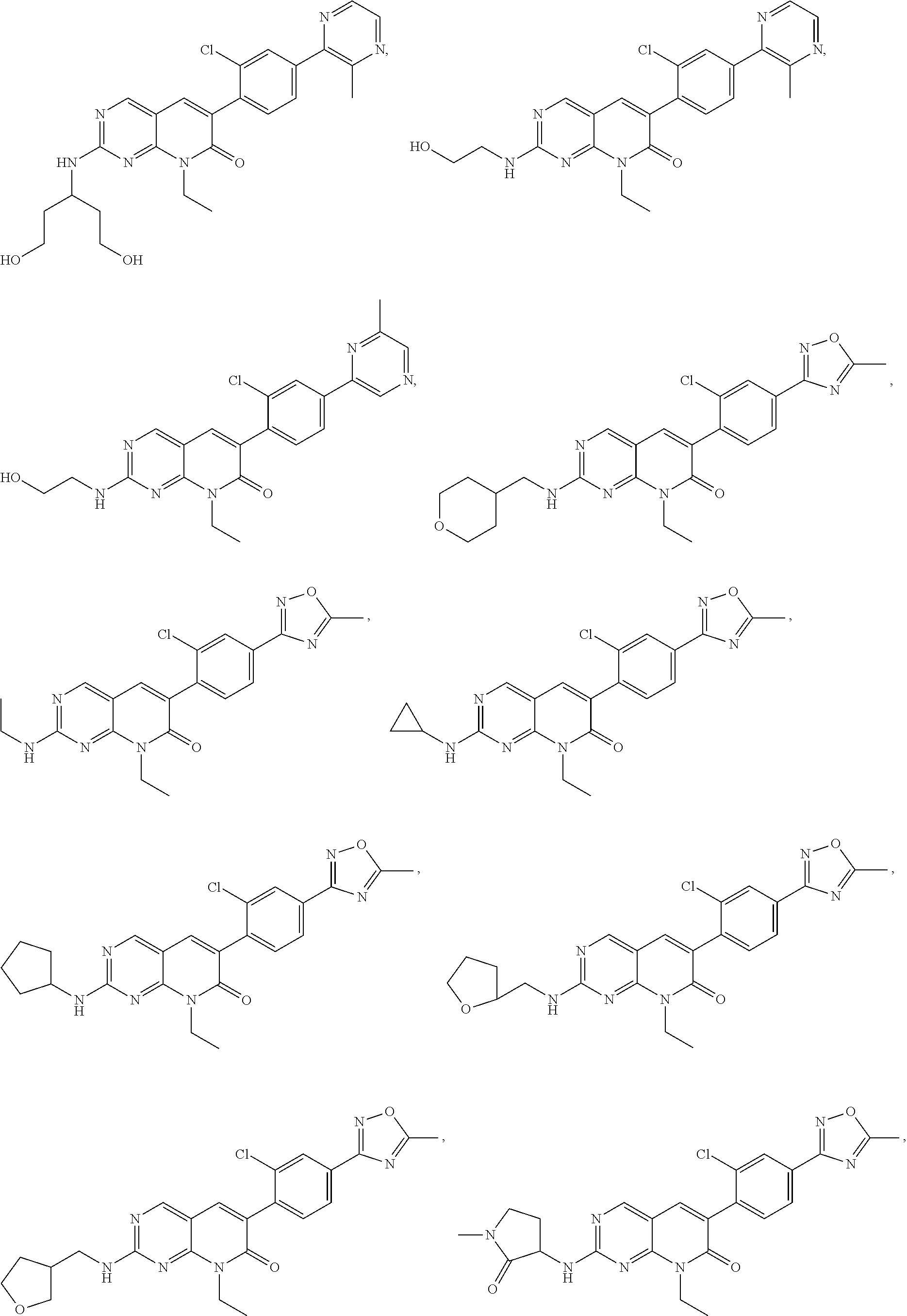 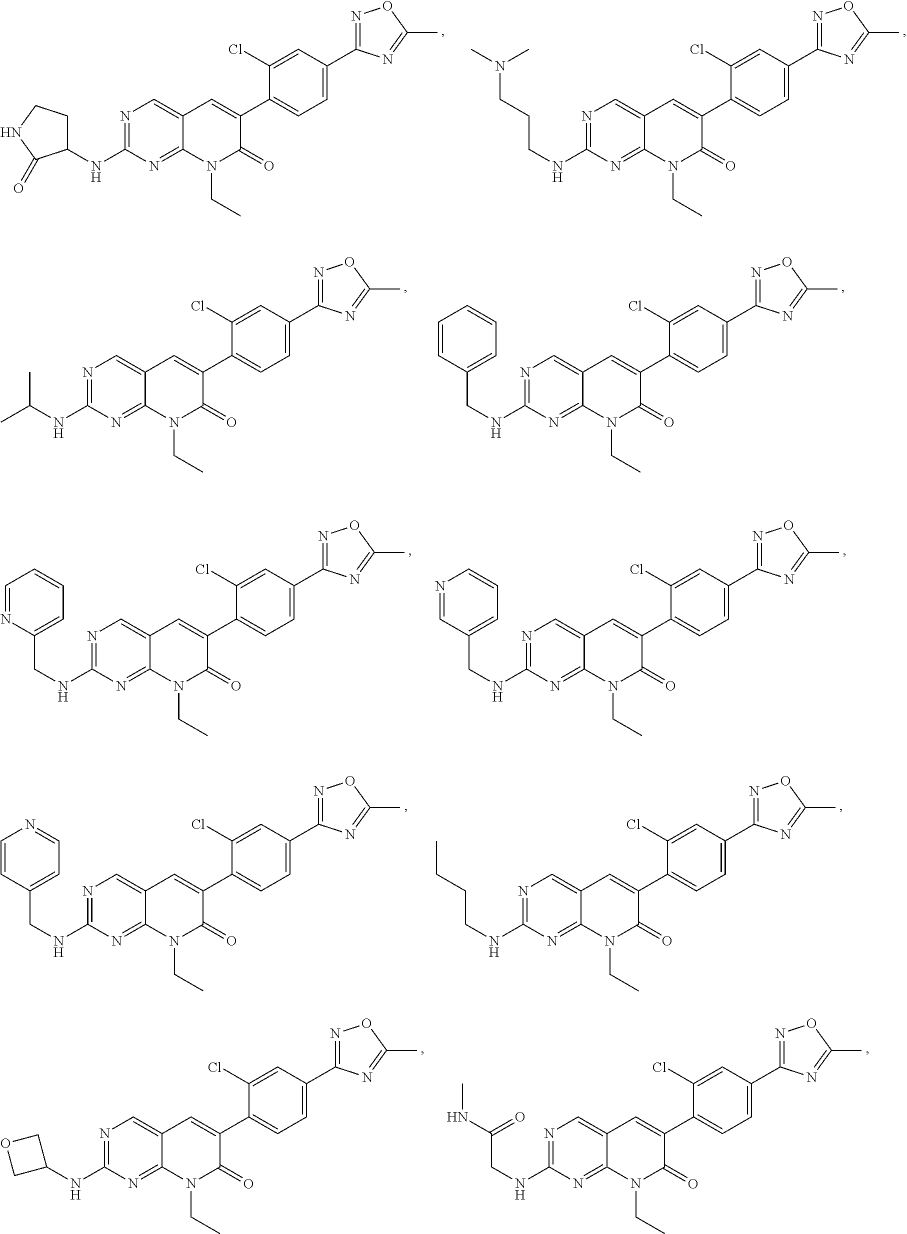 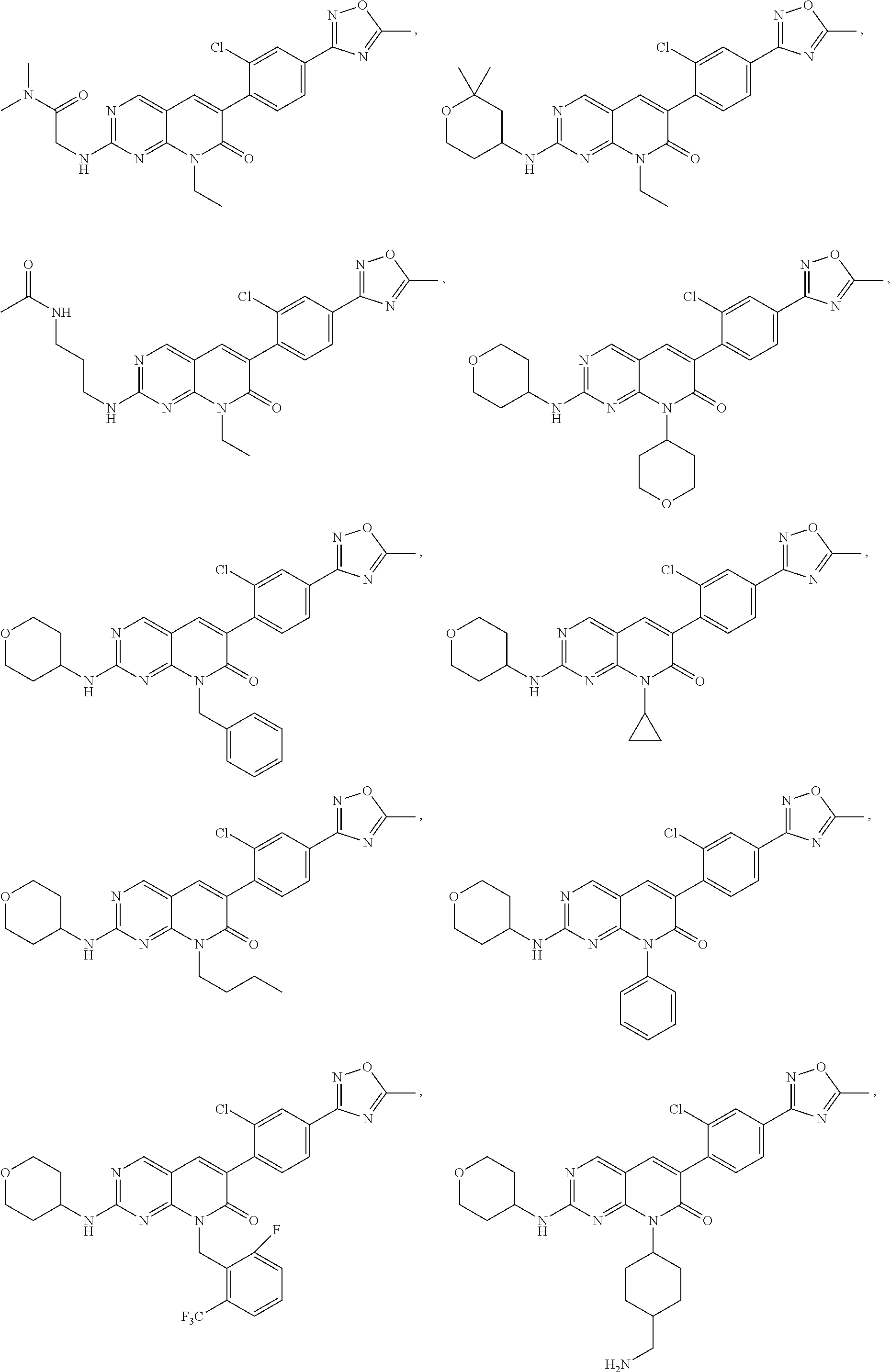 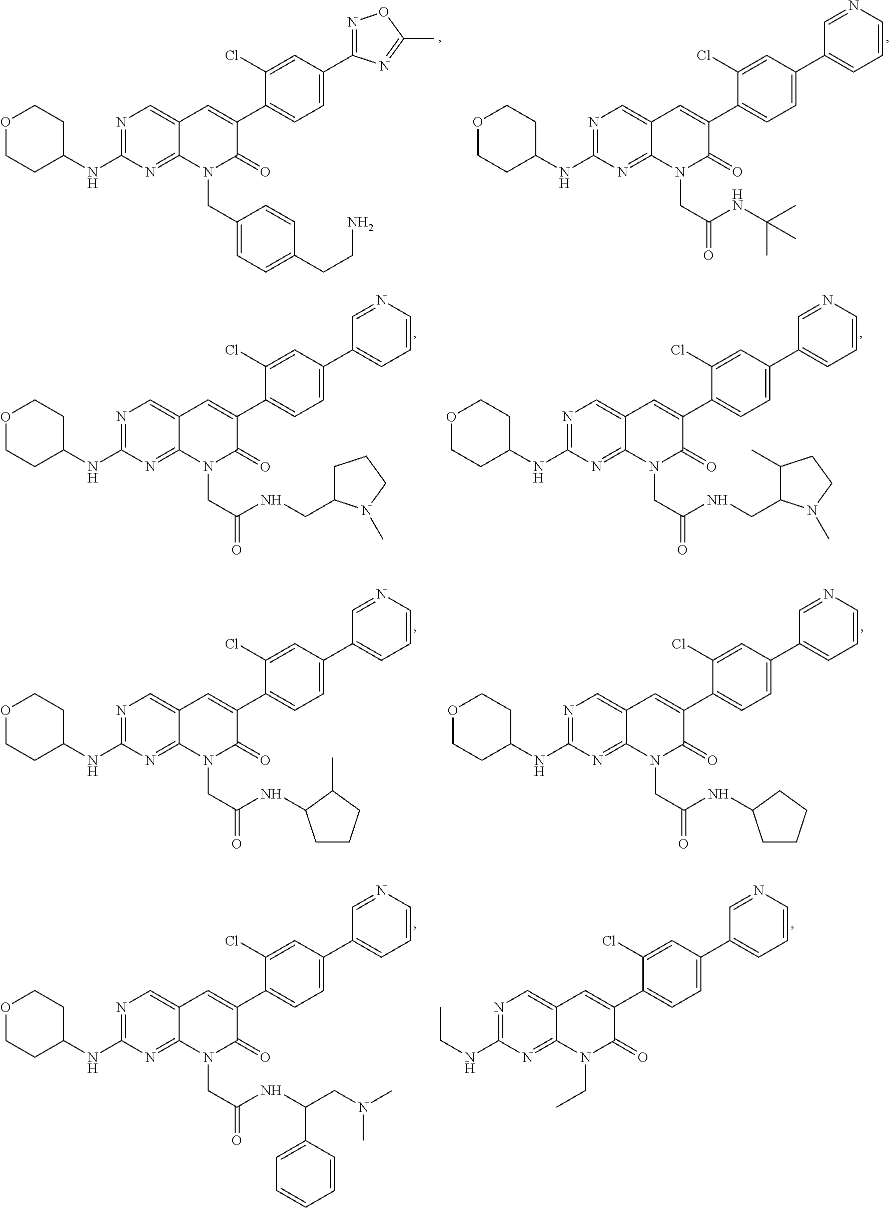 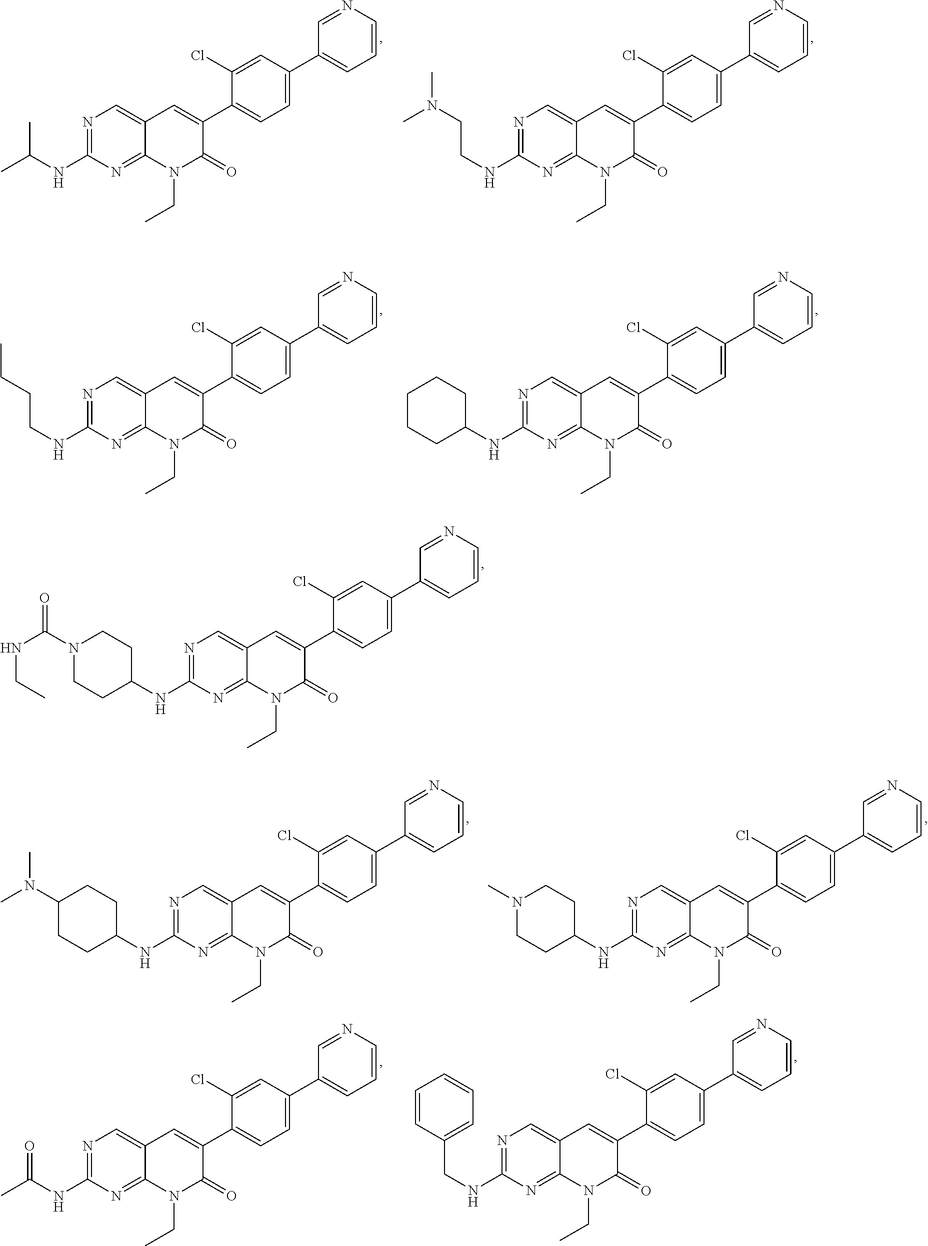 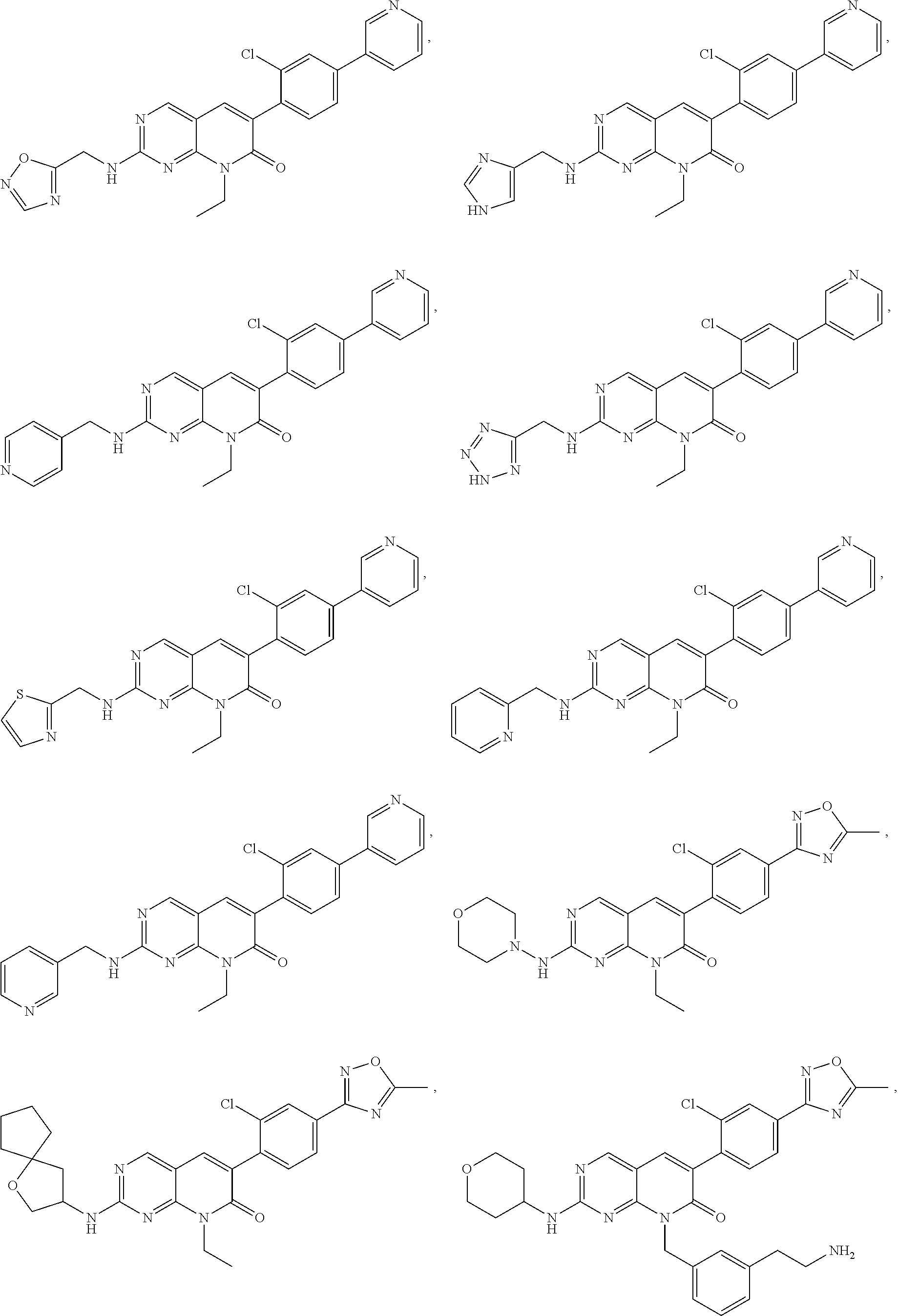 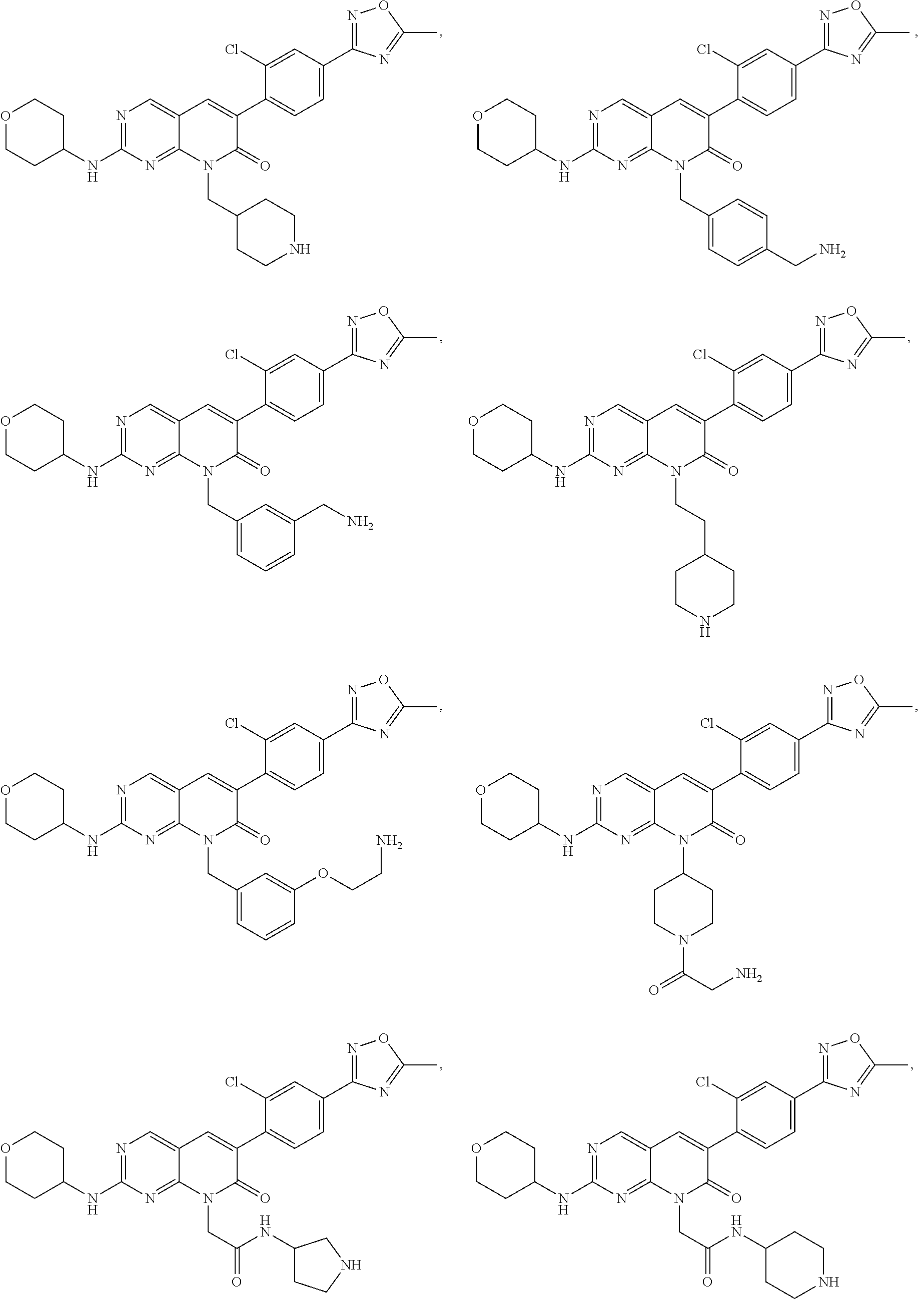 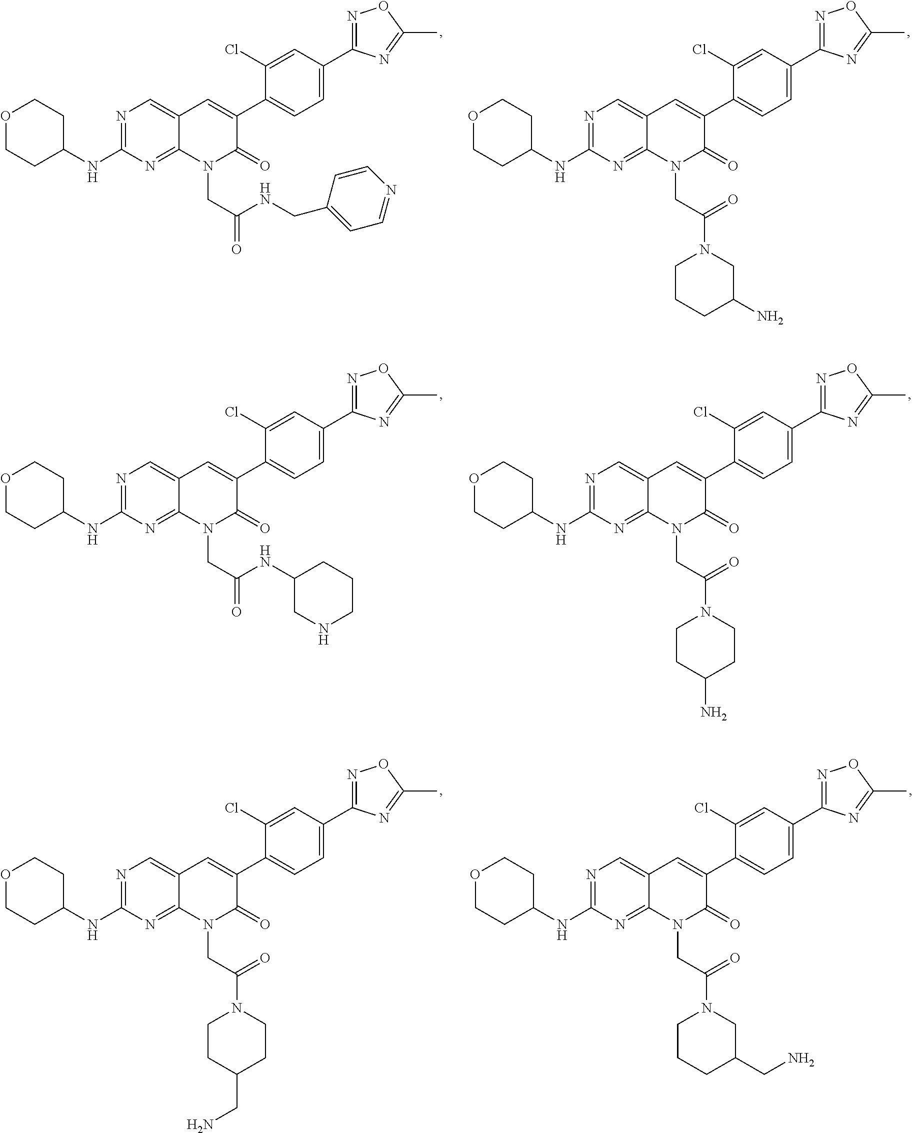 [SMILES: Cc1nc(-c2ccc(-c3cc4cnc(NC5CCOCC5)nc4n(CC(=O)NCc4ccncc4)c3=O)c(Cl)c2)no1 ; Cc1nc(-c2ccc(-c3cc4cnc(NC5CCOCC5)nc4n(CC(=O)N4CCCC(N)C4)c3=O)c(Cl)c2)no1 ; Cc1nc(-c2ccc(-c3cc4cnc(NC5CCOCC5)nc4n(CC(=O)NC4CCCNC4)c3=O)c(Cl)c2)no1 ; Cc1nc(-c2ccc(-c3cc4cnc(NC5CCOCC5)nc4n(CC(=O)N4CCC(N)CC4)c3=O)c(Cl)c2)no1 ; Cc1nc(-c2ccc(-c3cc4cnc(NC5CCOCC5)nc4n(CC(=O)N4CCC(CN)CC4)c3=O)c(Cl)c2)no1 ; Cc1nc(-c2ccc(-c3cc4cnc(NC5CCOCC5)nc4n(CC(=O)N4CCCC(CN)C4)c3=O)c(Cl)c2)no1] 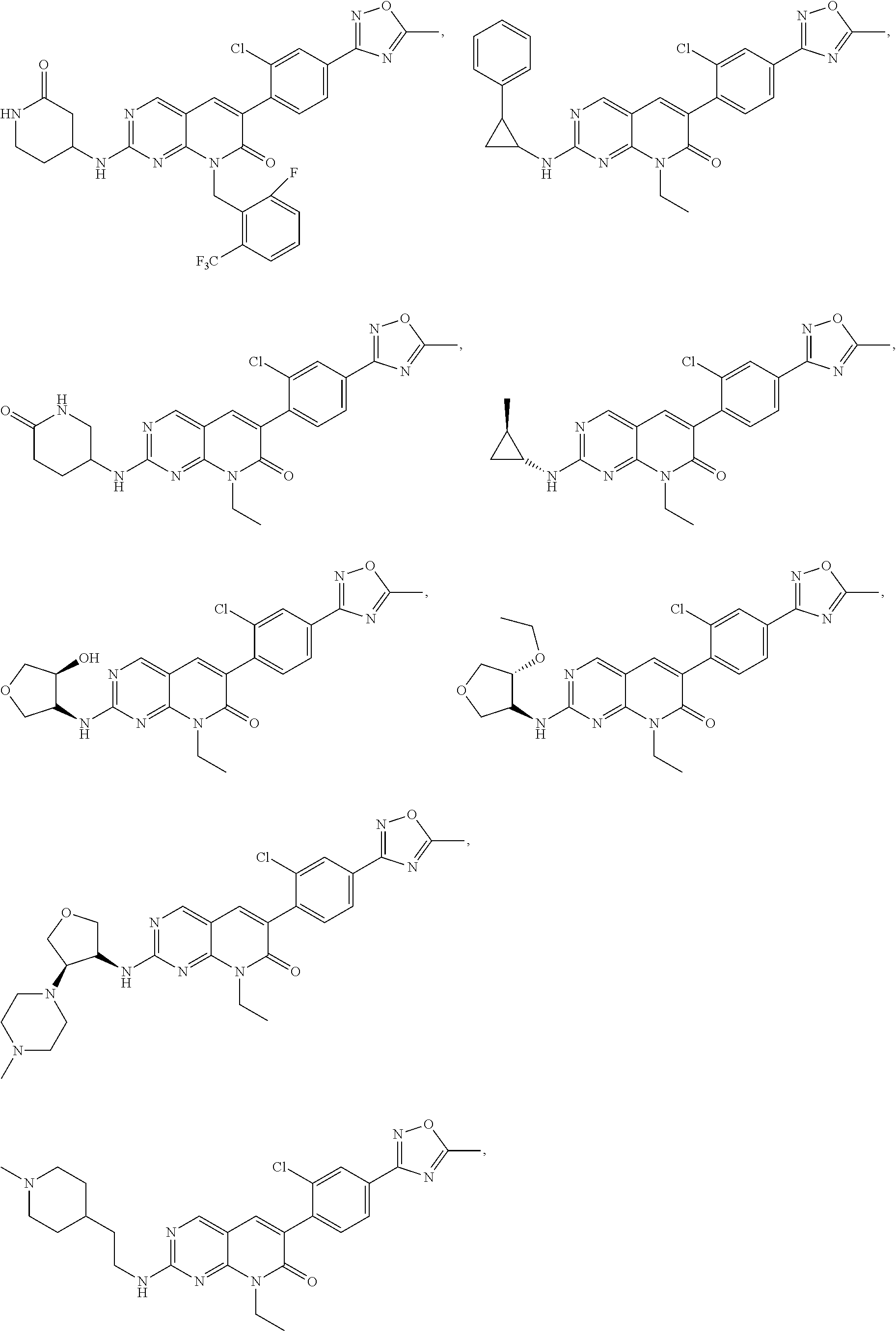 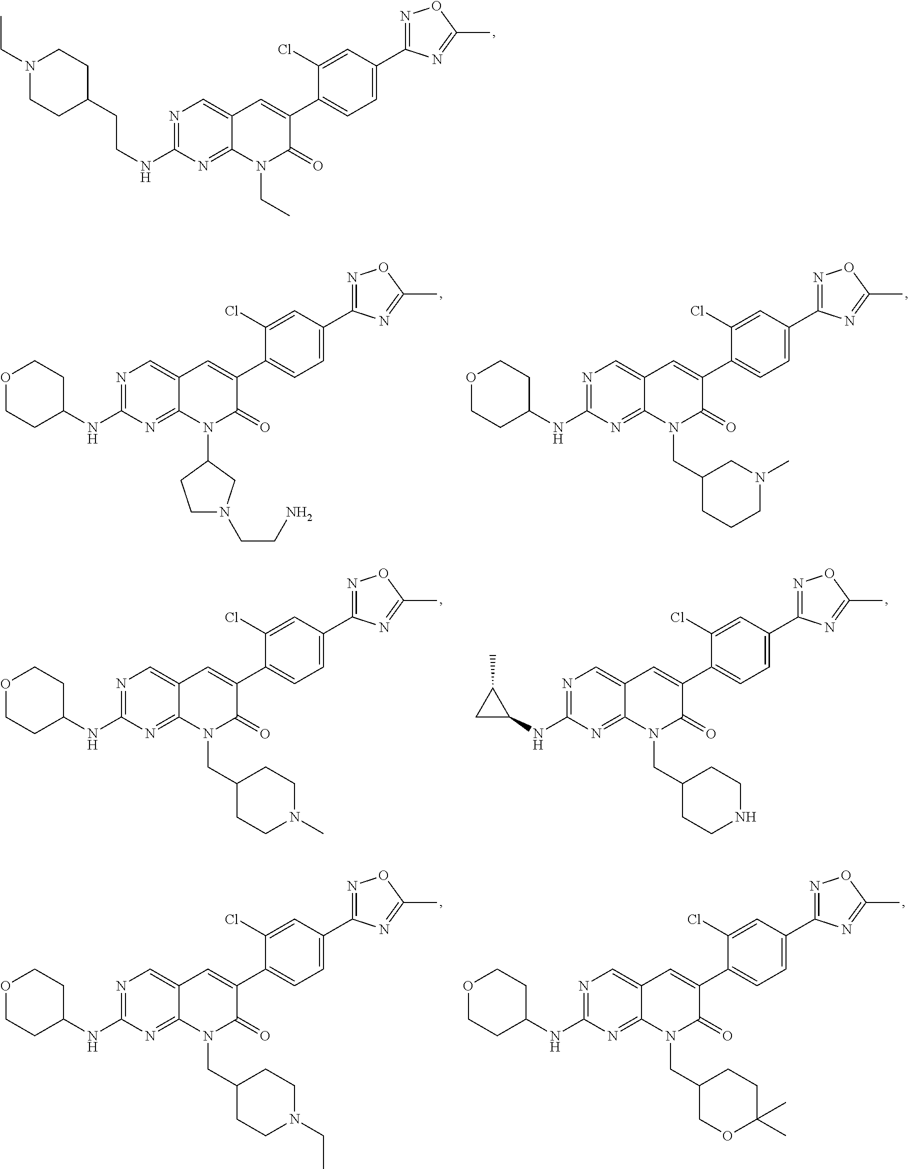 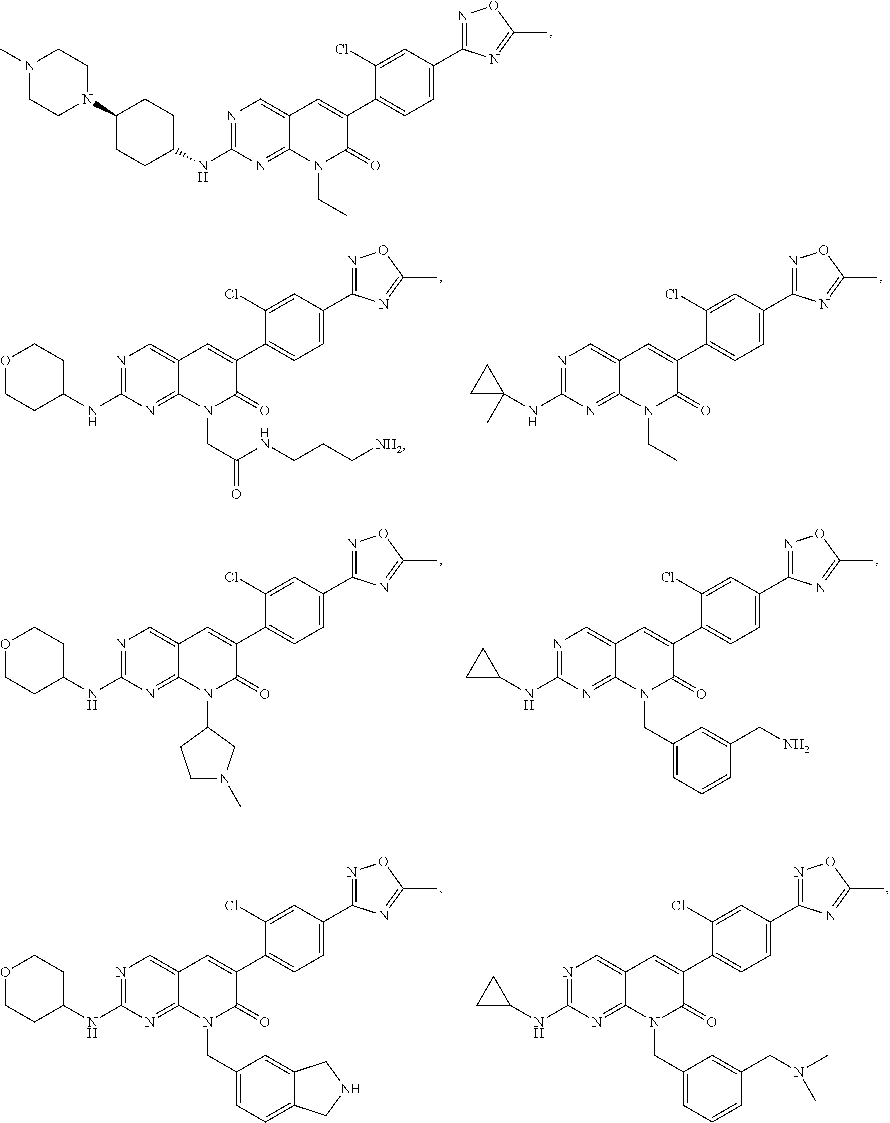 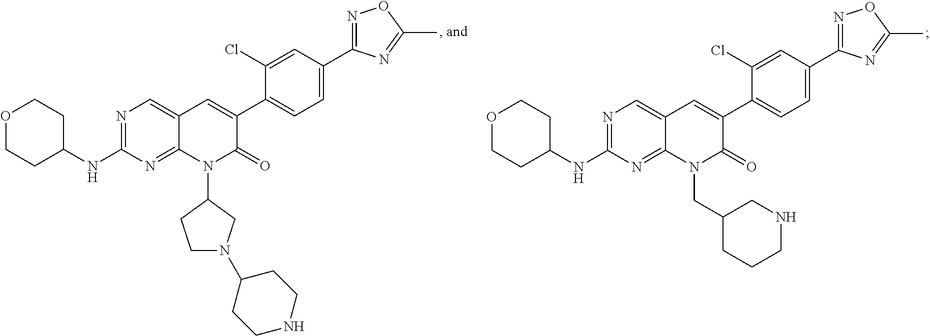 [SMILES: Cc1nc(-c2ccc(-c3cc4cnc(NC5CCOCC5)nc4n(C4CCN(C5CCNCC5)C4)c3=O)c(Cl)c2)no1 ; Cc1nc(-c2ccc(-c3cc4cnc(NC5CCOCC5)nc4n(CC4CCCNC4)c3=O)c(Cl)c2)no1]
or a pharmaceutically acceptable salt or N-oxide thereof.

38. A pharmaceutical composition comprising a compound of claim 1 and a pharmaceutically acceptable excipient, carrier, or binder thereof.

39. A method for treating Fragile X syndrome in an individual in need thereof, comprising administering to the subject a therapeutically effective amount of a compound having the structure of Formula I, Formula II, or Formula III, or a pharmaceutically acceptable salt or N-oxide thereof: 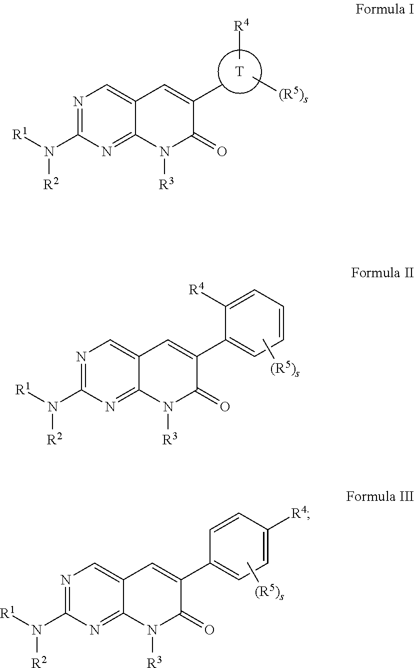
wherein:
ring T is an aryl or heteroaryl ring;
R¹ is H, or substituted or unsubstituted alkyl;
R² is substituted or unsubstituted alkyl, substituted or unsubstituted alkoxy, substituted or unsubstituted aralkoxy, substituted or unsubstituted heteroalkyl, substituted or unsubstituted cycloalkyl, substituted or unsubstituted cycloalkylalkyl, substituted or unsubstituted heterocycloalkyl, substituted or unsubstituted heterocycloalkylalkyl, spiro-cycloakyl-heterocycloalkyl, -alkylene-S(═O)R⁹, -alkylene-S(═O)₂R⁹, —S(═O)₂R⁹;
R³ is H, substituted or unsubstituted alkyl, substituted or unsubstituted alkoxy, substituted or unsubstituted amino, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted aralkyl, substituted or unsubstituted heteroaryl, or substituted or unsubstituted heteroarylalkyl;
R⁴ is substituted or unsubstituted heteroaryl attached to ring T or the phenyl ring via a carbon atom of R4, or substituted or unsubstituted heterocycloalkyl attached to ring T or the phenyl ring via a carbon atom of R⁴;
each R⁵ is independently halogen, —CN, —NO₂, —OH, —OCF₃, —OCH₂F, —OCF₂H, —CF₃, —NR¹⁰S(═O)₂R⁹, —S(═O)₂N(R¹⁰)₂, —S(═O)R⁹, —S(═O)₂R⁹, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted heterocycloalkyl; or substituted or unsubstituted cycloalkyl; or substituted or unsubstituted aryl; or substituted or unsubstituted heteroaryl;
each R⁸ is independently H or R⁹;
each R⁹ is independently substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, or substituted or unsubstituted heteroaryl; or two R¹⁰, together with the atoms to which they are attached form a heterocycle; and
s is 0-4.

40. The method of claim 39, wherein administration of a therapeutically effective amount of the compound normalizes or partially normalizes aberrant synaptic plasticity associated with Fragile X syndrome.

41. The method of claim 39, wherein administration of a therapeutically effective amount of the compound normalizes or partially normalizes aberrant long term depression (LTD) associated with Fragile X syndrome.

42. The method of claim 39 wherein administration of a therapeutically effective amount of the compound normalizes or partially normalizes aberrant long term potentiation (LTP) associated with Fragile X syndrome.
